SEQUENCE LISTING (13 sequences)

SEQ ID NO: 1 - protein, 451 aa, Escherichia coli
MIFSDWPWRHWRQVRGETIALRLNDEQLNWRELCARVDELASGFAVQGVVEGSGVMLRAWNTPQTLLAWLALLQCGARVLPVNPQLPQPLLEELLPNLTLQFALVPDGENTFPALTSLHIQLVEGAHAATWQPTRLCSMTLTSGSTGLPKAAVHTYQAHLASAQGVLSLIPFGDHDDWLLSLPLFHVSGQGIMWRWLYAGARMTVRDKQPLEQMLAGCTHASLVPTQLWRLLVNRSSVSLKAVLLGGAAIPVELTEQAREQGIRCFCGYGLTEFASTVCAKEADGLADVGSPLPGREVKIVNNEVWLRAASMAEGYWRNGQLVSLVNDEGWYATRDRGEMHNGKLTIVGRLDNLFFSGGEGIQPEEVERVIAAHPAVLQVFNVPVADKEFGHRPVAVMEYDHESVDLSEWVKDKLARFQQPVRWLTLPPELKNGGIKISRQALKEWVQRQQ

SEQ ID NO: 2 - protein, 486 aa, Bacillus subtilis
MLTEQPNWLMQRAQLTPERIALIYEDQTVTFAELFAASKRMAEQLAAHSVRKGDTAAILLQNRAEMVYAVHACFLLGVKAVLLNTKLSTHERLFQLEDSGSGFLLTDSSFEKKEYEHIVQTIDVDELMKEAAEEIEIEAYMQMDATATLMYTSGTTGKPKGVQQTFGNHYFSAVSSALNLGITEQDRWLIALPLFHISGLSALFKSVIYGMTVVLHQRFSVSDVLHSINRHEVTMISAVQTMLASLLEETNRCPESIRCILLGGGPAPLPLLEECREKGFPVFQSYGMTETCSQIVTLSPEFSMEKLGSAGKPLFSCEIKIERDGQVCEPYEHGEIMVKGPNVMKSYFNRESANEASFQNGWLKTGDLGYLDNEGFLYVLDRRSDLIISGGENIYPAEVESVLLSHPAVAEAGVSGAEDKKWGKVPHAYLVLHKPVSAGELTDYCKERLAKYKIPAKFFVLDRLPRNASNKLLRNQLKDARKGELL

SEQ ID NO: 3 - protein, 362 aa, Mycobacterium tuberculosis
MLGGSDPALVAVPTQHESLLGALRVGEQIDDDVALVVTTSGTTGPPKGAMLTAAALTASASAAHDRLGGPGSWLLAVPPYHIAGLAVLVRSVIAGSVPVELNVSAGFDVTELPNAIKRLGSGRRYTSLVAAQLAKALTDPAATAALAELDAVLIGGGPAPRPILDAAAAAGITVVRTYGMSETSGGCVYDGVPLDGVRLRVLAGGRIAIGGATLAKGYRNPVSPDPFAEPGWFHTDDLGALESGDSGVLTVLGRADEAISTGGFTVLPQPVEAALGTHPAVRDCAVFGLADDRLGQRVVAAIVVGDGCPPPTLEALRAHVARTLDVTAAPRELHVVNVLPRRGIGKVDRAALVRRFAGEADQ

SEQ ID NO: 4 - protein, 492 aa, Staphylococcus aureus
MDFWLYKQAQQNGHHIAITDGQESYTYQNLYCEASLLAKRLKAYQQSRVGLYIDNSIQSIILIHACWLANIEIAMINTRLTPNEMTNQMKSIDVQLIFCTLPLELRGFQIVSLDDIEFAGRDITTNSLLDNTMGIQYETSNETVVPKESPSNILNTSFNLDDIASIMFTSGTTGPQKAVPQTFRNHYASAIGCKESLGFDRDTNWLSVLPIYHISGLSVLLRAVIEGFTVRIVDKFNAEQILTMIKNERITHISLVPQTLNWLMQQGLHEPYNLQKILLGGAKLSATMIETALQYNLPIYNSFGMTETCSQFLTATPEMLHARPDTVGMPSANVDVKIKNPNKEGHGELMIKGANVMNVYLYPTDLTGTFENGYFNTGDIAEIDHEGYVMIYDRRKDLIISGGENIYPYQIETVAKQFPGISDAVCVGHPDDTWGQVPKLYFVSESDISKAQLIAYLSQHLAKYKVPKHFEKVDTLPYTSTGKLQRNKLYRG

SEQ ID NO: 5 - DNA, 26 bp, Artificial Sequence
  Synthetic oligonucleotide
ggaattatgtggaagtggttatacgc

SEQ ID NO: 6 - DNA, 26 bp, Artificial Sequence
  Synthetic Oligonucleotide
gcgttaaaccacttccacataattcc

SEQ ID NO: 7 - DNA, 26 bp, Artificial Sequence
  Synthetic oligonucleotide
ggaattatgtggcagtggttatacgc

SEQ ID NO: 8 - DNA, 25 bp, Artificial Sequence
  Synthetic oligonucleotide
gcgtataaccactgccacataattc

SEQ ID NO: 9 - protein, 68 aa, Escherichia coli
SGSTGLPKAAVHTYQAHLASAQGVLSLIPFGDHDDWLLSLPLFHVSGQGIMWRWLYAGARMTVRDKQP

SEQ ID NO: 10 - protein, 68 aa, Staphylococcus aureus
SGTTGPQKAVPQTFRNHYASAIGCKESLGFDRDTNWLSVLPIYHISGLSVLLRAVIEGFTVRIVDKFN

SEQ ID NO: 11 - protein, 69 aa, Mycobacterium tuberculosis
SGTTGPPKGAMLTAAALTASASAAHDRLGGPGSWLLAVPPYHIAGLAVLVRSVIAGSVPVELNVSAGFD

SEQ ID NO: 12 - protein, 10 aa, Artificial Sequence
  Synthetic polypeptide
GRVDDMIISG

SEQ ID NO: 13 - protein, 7 aa, Artificial Sequence
  Synthetic polypeptide
PKNALNK

(19) United States
(12) Patent Application Publication
(10) Pub. No.: US 2018/0273573 A1
(43) Pub. Date: Sep. 27, 2018

(54) INHIBITORS OF MENAQUINONE BIOSYNTHESIS

(71) Applicants: Memorial Sloan-Kettering Cancer Center, New York, NY (US); The Research Foundation for The State University of New York, Albany, NY (US)

(72) Inventors: Derek Shieh Tan, New York, NY (US); Christopher E. Evans, New York, NY (US); Indrajeet Sharma, Norman, OK (US); Peter J. Tonge, Newbury, Berkshire (GB); Joe S. Matarlo, Stony Brook, NY (US)

(73) Assignees: Memorial Sloan-Kettering Cancer Center, New York, NY (US); The Research Foundation for The State University of New York, Albany, NY (US)

(21) Appl. No.: 15/764,613

(22) PCT Filed: Oct. 3, 2016
(86) PCT No.: PCT/US16/55136

§ 371 Date: Mar. 29, 2018

Related U.S. Application Data
(60) Provisional application No. 62/236,077, filed on Oct. 1, 2015.

(51) Int. Cl. C07H 19/16; A61P 31/04

(52) U.S. Cl. CPC C07H 19/16; A61P 31/04

(57) ABSTRACT

Provided herein are compounds of Formula (I) and pharmaceuticals acceptable salts, solvates, hydrates, polymorphs, co-crystals, tautomers, stereoisomers, and prodrugs thereof. Also provided are pharmaceutical compositions, kits, and methods involving the inventive compounds for the treatment of an infectious disease (e.g., bacterial infection (e.g., tuberculosis, methicillin-resistant Staphylococcus aureus).

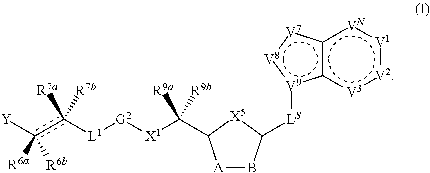

### RELATED APPLICATIONS

This application claims priority under 35 U.S.C. § 119(e) to U.S. provisional patent application, U.S. Ser. No. 62/236,077, filed Oct. 1, 2015, which is incorporated herein by reference.

### GOVERNMENT SUPPORT

This invention was made with Government support under AI068038, GM100477, GM102864, GM073546 and CA008748 awarded by the National Institutes of Health. The Government has certain rights in the invention.

### BACKGROUND

The spread of infections due to drug-resistant pathogenic bacteria, such as multi-drug-resistant and extensively-resistant Mycobacterium tuberculosis and methicillin-resistant Staphylococcus aureus (MRSA), is a serious threat to the populations of both developing and developed countries. Approximately one-third of the world's population is infected with active or latent M. tuberculosis (see, e.g., Harper, Nat. Med. (2007) 13, 309-312; Nathan, Nat. Med. (2014), 20, 121-123; Keener, Nat. Med. (2014) 20, 976-978), and community-acquired MRSA is the cause of more than 7 million hospitalizations due to skin and soft tissue infections annually in the United States alone (see, e.g., McKenna, Nature (2012) 482, 23-25; Hersh et al., Arch. Intern. Med. (2008), 168, 1585-1591). There is a need for novel therapeutic agents to treat infections of pathogenic bacteria, particularly as new drug-resistant strains continue to emerge.

### SUMMARY

Menaquinone, also known as Vitamin K₂, is a lipid-soluble electron carrier used in the electron transport chain of cellular respiration. Menaquinone consists of a 2-methyl-1,4-naphthoquinone group attached to an isoprenoid side chain. The side chain typically consists of between 4 and 13 isoprene units (i.e., n=4-13), and the length varies based on the biosynthetic pathway utilized to produce menaquinone in a particular species. For example, in M. tuberculosis the major vitamin K₂ species is MK-9, menaquinone with nine isoprene units (n=9), whereas the major species synthesized by S. aureus is menaquinone with eight isoprenes (MK-8, n=8).

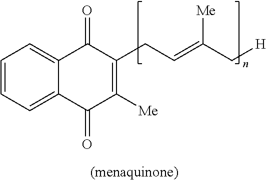

Bacteria of the genus Mycobacterium, most Gram-positive bacteria, and some Gram-negative bacteria rely solely on menaquinone for electron transport, and this reliance extends to all species of bacteria growing under anaerobic conditions (see, e.g., Collins et al., J. Gen. Microbiol. (1979) 110, 127-136; Nahaie et al. J. Gen. Microbiol. (1984) 130, 2427-2437; Hiratsuka et al. Science (2008) 321, 1670-1673). The reliance of certain pathogens on menaquinone for cellular respiration thus makes menaquinone biosynthesis a target for treatments of infectious disease. Such treatments would extend to latent infections (e.g., nonreplicating M. tuberculosis), since the latent pathogen must still respire. Since humans and other hosts lack the menaquinone biosynethetic pathway, treatments that target this pathway should by highly selective for the pathogen over the host. Menaquinone is synthesized by bacteria from chorismate via a biosynthetic pathway involving at least nine distinct enzymes, including MenA, MenB, MenC, MenD, MenE, MenF, MenH, MenI, and UbiE.

MenE, also known as o-succinylbenzoate-CoA synthetase, is an acyl-CoA synthetase that shares similarity with several families of adenylate-forming enzymes. These families include acyl-CoA synthetases, aryl-CoA synthetases, firefly luciferases, and the adenylation domains of non-ribosomal peptide synthetases (NRPSs), and have been grouped into a proposed superfamily of ANL enzymes (ANL stands for Acyl-CoA synthetases, NRPS adenylation domains, and Luciferase enzymes) (see, e.g., Gulick, ACS Chem. Biol. (2009) 62, 347-352). Members of these families catalyze two partial reactions, the initial adenylation of a carboxylate to form an acyl-AMP intermediate, and the subsequent coupling of the acyl group to a nucleophile (e.g., CoA) with release of an adenylate (e.g., AMP) (see, e.g., Gulick,). MenE catalyzes adenylation of o-succinylbenzoate with ATP, and the subsequent ligation of CoA to o-succinylbenzoate with release of AMP. FIG. 1 shows the menaquinone biosynthetic pathway including the steps catalyzed by MenE.

MenE inhibitors have been described by Tan, Tonge, and co-workers in Lu et al. Bioorg. Med. Chem. Lett. (2008) 18, 5963-5966, Lu et al. ChemBioChem (2012) 13, 129-136, and Matarlo et al. Biochemistry (2015) 54, 6514-6524, each of which is incorporated herein by reference. Inhibitors of MenE have also been previously described by Mesecar and co-workers (see Tian et al. Biochemistry (2008) 47, 12434-12447).

Compounds of the present invention may be capable of inhibiting ligases and adenylate-forming enzymes. In certain embodiments, the compounds of the invention are capable of inhibiting o-succinylbenzoate synthetase (MenE). In certain embodiments, the compounds of the invention are capable of inhibiting MenA, MenB, MenC, MenD, MenF, MenH, MenI, and/or UbiE. The compounds provided are analogs of the MenE intermediate o-succinylbenzoate-adensosinemonophosphate (OSB-AMP). In certain embodiments, the analogs comprise a linker (e.g., a sulfonyl moiety) that mimics the phosphate between the o-succinylbenzoate and adenosine moieties in OSB-AMP.

Compounds of the present invention are of Formula (I):

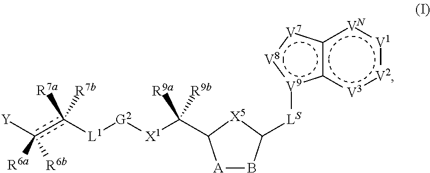

wherein, in certain embodiments, the o-benzoate moiety of OSB-AMP is replaced with group Y. Group Y comprises either an aryl or bicyclic moiety as shown below:

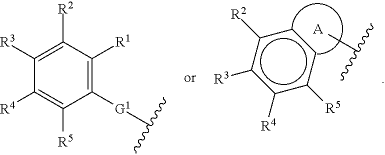

In certain embodiments, a compound provided comprises a sulfamide linker, sulfamate linker, or vinylsulfonamide linker, as shown below:

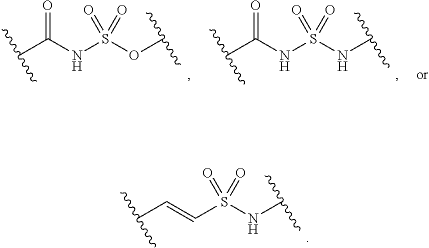 [SMILES: *OS(=O)(=O)NC(*)=O ; *NS(=O)(=O)NC(*)=O ; */C=C/S(=O)(=O)N*]

In certain embodiments, a provided compound is of Formula (III), (IV), or (V):

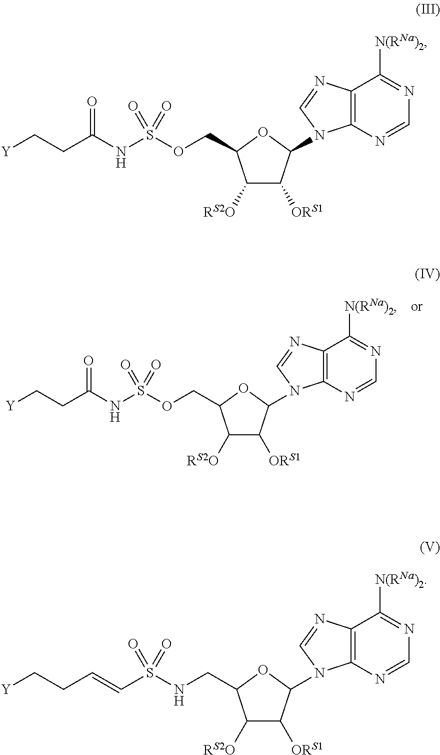

Pharmaceutical compositions of the compounds are also provided, in addition to methods of preventing and/or treating an infectious disease using the compound or compositions thereof. The infectious disease may be a bacterial infection. The methods provided may be for treatment of an infection with a Gram-positive and/or Gram-negative bacteria, such as a Staphylococcus, Bacillus, or Escherichia bacteria. The methods may be for treatment of a mycobacterial infection, such as tuberculosis. The pharmaceutical compositions and methods may be useful in the treatment of drug-resistant tuberculosis infections or drug-resistant Staphylococcus aureus infections (e.g., MRSA, VRSA).

The invention also provides methods useful for inhibiting ligases and adenylate-forming enzymes (e.g., o-succinylbenzoate-CoA synthetase (MenE)) or inhibiting menaquinone biosynthesis in an infectious microorganism by contacting the microorganism with a compound provided herein. Additionally provided are methods for inhibiting o-succinylbenzoate-CoA synthetase (MenE) or inhibiting menaquinone biosynthesis in an infectious microorganism in a subject by administering to the subject a compound provided herein.

The details of certain embodiments of the invention are set forth in the Detailed Description of Certain Embodiments, as described below. Other features, objects, and advantages of the invention will be apparent from the Definitions, Examples, Figures, and Claims.

### BRIEF DESCRIPTION OF THE DRAWINGS

The accompanying drawings, which constitute a part of this specification, illustrate several embodiments of the invention and together with the description, serve to explain the principles of the invention.

FIG. 1 shows the classical de novo menaquinone biosynthesis pathway. This pathway consists of at least nine enzymes that catalyze the formation of menaquinone from chorismate. The fifth enzyme, MenE, is an acyl-CoA synthetase, which ligates CoA to o-succinylbenzoate (OSB) via an OSB-AMP intermediate.

FIG. 2 shows the effect of OSB-AMS (15.6 μM) on menaquinone levels in MRSA. The 1959 Blight/Dyer lipid extraction protocol was followed. Menaquinone levels were quantified by LC-MS/MS using standard curves generated with MK4 and MK9. A distribution of MKs are present in untreated MRSA with MK8 most abundant. Treatment with OSB-AMS at half-MIC results in a decrease in MK levels consistent with MenE inhibition.

FIG. 3A shows a sequence alignment of MenE homologs from pathogenic bacteria (E. coli, S. aureus, and M. tuberculosis). The rectangular box indicates a conserved arginine in the active site, identified by docking of OSB-AMS to the crystal structure of saMenE. FIG. 3B shows a CD spectra of wild-type ecMenE (top left panel), and ecMenE mutants R195K (top right panel) and R195Q (bottom panel).

FIG. 4. (a) Menaquinone biosynthetic pathway. ^{a}n=4-13; n=9 in M. tuberculosis; n=8 in S. aureus and E. coli. (b) MenE inhibitors that mimic the tightly-bound OSB-AMP reaction intermediate. OSB-AMS and difluoroindanediol mixture inhibits MenE (IC₅₀) and bacterial growth (MIC). Additional data for inhibitors can be found, e.g., in Table E1.

FIG. 5 shows a stereoselective retrosynthesis of difluoroindanediol-based inhibitor 2. PG=protecting group.

FIG. 6 shows computational docking of diastereomeric difluoroindanediols 2 (black) to E. coli MenE R195K (PDB: 5C5H), overlaid with cocrystallized OSB-AMS (grey), with key binding residues and conserved waters. Schrödinger Glide docking scores shown for each diastereomer (arbitrary units). OSB-AMS docked with a score of −13.9 (see FIG. 9).

FIG. 7A shows a synthesis of 1R,3S-syn-difluoroindanediol (1R,3S)-2. FIG. 7B shows a synthesis of 1S,3R-syn-difluoroindanediol (1S,3R)-2. FIG. 7C shows a synthesis of 1R,3R-anti-difluoroindanediol (1R,3R)-2. FIG. 7D shows a synthesis of 1S,3S-anti-difluoroindanediol (1S,3S)-2. In FIGS. 7A-7D, DMAP=4-(dimethylamino)pyridine; DMF=N,N-dimethylformamide; DMSO=dimethyl sulfoxide; EDCI=1-ethyl-3-(3-dimethyl-aminopropyl)carbodiimide hydrochloride; LiHMDS=lithium hexamethyldisilazide; MeOH=methanol; TASF=tris(dimethylamino)sulfonium difluorotrimethylsilicate; TFA=2,2,2-trifluoroacetic acid; THF=tetrahydrofuran.

FIG. 8 shows computational docking of OSB-AMS (1) (grey) and diastereomeric difluoroindanediols 2 (black) to E. coli MenE R195K (PDB: 5C5H), overlaid with cocrystallized OSB-AMS, with key binding residues and conserved waters. Schrödinger Glide docking scores shown for each ligand (arbitrary units, expressed in kcal/mol). RMSD value shown for docked and cocrystallized OSB-AMS (1). Difluoroindanediol panels are expanded versions of those shown in FIG. 6.

FIG. 9 shows menaquinone-8 levels in methicillin-resistant Staphylococcus aureus treated with MenE inhibitors. Standard error shown for two independent experiments. *p≤0.05, **p≤0.01.

FIG. 10 shows X-ray crystal structure of syn-diol (1S,3R)-14 (left) with (R)-α-methyl-4-nitrobenzylamine (right, two NO₂ rotamers) and MeOH (lower left).

FIG. 11 shows antibicrobial and cytotoxic activity of compounds provided herein: ^{a}MIC values were obtained against E. coli (K-12), B. subtilis (ATCC 6057), methicillin-resistant S. aureus (ATCC BAA-1762), and M. tuberculosis (H37Rv). Inoculum levels for each MIC measurement ranged from 1×106 to 2×106 cells/mL. All MICs were determined in technical and experimental triplicate. ND=not determined. ^{b}MICs determined with exogenous 10 μg/mL MK4 added to the synthetic growth medium. ^{c}Cytotoxicity values were obtained against Vero (monkey kidney epithelial) cells. Measurements were performed in technical and experimental triplicate.

FIG. 12 shows overlaid structures of OSB-AMS:R195K ecMenE and apo saMenE. Structural overlay of the OSB-AMS:ecMenE complex with apo saMenE (PDB entry 3IPL). These structures differ in the relative orientation of large domain 1 and small domain 2 (showing E. coli and S. aureus) but represent the adenylate-bound conformation in which G358 or G402 in the A8 core motif is removed from the active site whereas K437 or K483 is located in the active site. G358 and K437 are residues from E. coli MenE. G402 and K483 are residues from S. aureus. K483 is disordered in the S. aureus structure.

FIG. 13 shows X-ray crystal structure of OSB-AMS:R195K ecMenE showing interactions with OSB-AMS. Panel A shows Overall structure of ecMenE:OSB-AMS shown with the larger N-terminal (domain I) and the smaller C-terminal (domain II) domains highlighted by transparent surface representations in dark grey and light grey, respectively. The ligand is shown in ball-and-stick representation. Panel B shows structure of the bound ligand, OSB-AMS, shown in the active site. The ligand is shown in ball-and-stick representation. Panel C shows schematic of OSB-AMS in the ecMenE active site. The putative hydrogen bonding interactions between the ligand and the residues are illustrated with dashed lines.

FIG. 14 shows binding isotherm for E. coli MenE binding with Compound 109 using direct fluorescent binding assay. Difluoroindandiol 109 was titrated into a solution of wild-type E. coli MenE and the change in fluorescence was measured using a Quanta Master fluorimeter at excitation and emission wavelengths of 280 and 332 nm, respectively. Data was analyzed using the Morrison equation. The K_{d} was determined to be 120±23 nM.

### DETAILED DESCRIPTION OF CERTAIN EMBODIMENTS

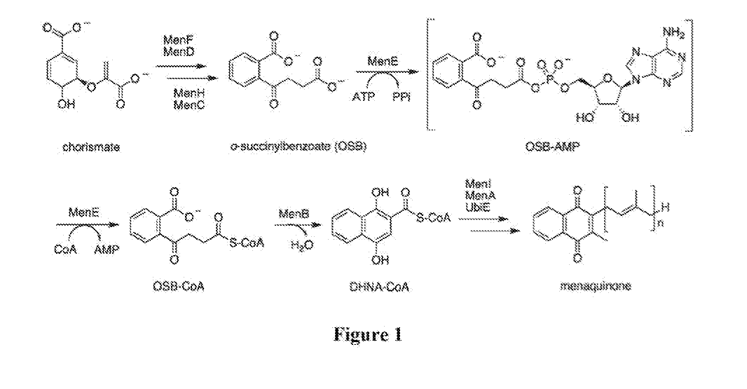

Provided herein are compounds which may inhibit ligases and adenylate-forming enzymes. In certain embodiments, the compounds of the invention inhibit o-succinylbenzoate-CoA synthetase (MenE). The compounds may interact with MenE so as to disrupt the activity of MenE in converting o-succinylbenzoate (OSB) to o-succinylbenzoate-CoA (OSB-CoA). MenE catalyzes two transformations in tandem (see FIG. 1). The first reaction combines OSB and ATP to form the intermediate OSB-AMP and pyrophosphate as a by-product. In the second reaction CoA is conjugated to OSB to form OSB-CoA, and AMP is released. In some embodiments, a provided compound affects the ability of MenE to form OSB-AMP, i.e., inhibits the first transformation. In some embodiments, a provided compound affects the ability of MenE to form OSB-CoA, i.e., inhibits the second transformation. In some embodiments, the compound may inhibit both the first and second transformation.

In the biosynthesis of menaquinone, OSB-CoA is subsequently converted to 1,4-dihydroxy-2-napthoyl-CoA (DHNA-CoA), and ultimately to menaquinone. Thus, a compound of the invention may inhibit menaquinone biosynthesis. In some embodiments, a compound provided inhibits menaquinone biosynthesis by inhibiting MenE. In some embodiments, a compound provided inhibits menaquinone biosynthesis by inhibiting the formation of OSB-CoA.

Without wishing to be bound by a particular theory, the compounds provided may inhibit MenE based on its structural similarity to OSB-AMP. The phosphate/carbonyl bond of OSB-AMP is cleaved during the conversion of OSB-AMP to OSB-CoA. The compounds provided replace the phosphate linker with a sulfonyl group, which is not readily cleaved or displaced by CoA. For example, OSB-AMS (o-succinylbenzoate-adenenosinemonosulfamate) is a structural analog of OSB-AMP (o-succinylbenzoate-adenosinemonophosphate), in which the phosphate group is replaced with a sulfamate moiety.

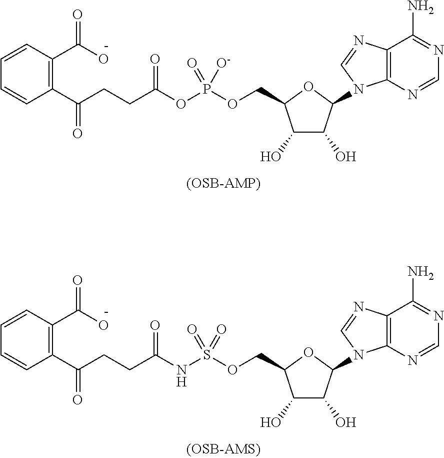 [SMILES: Nc1ncnc2c1ncn2[C@@H]1O[C@H](COP(=O)([O-])OC(=O)CCC(=O)c2ccccc2C(=O)O)[C@@H](O)[C@H]1O ; Nc1ncnc2c1ncn2[C@@H]1O[C@H](COS(=O)(=O)NC(=O)CCC(=O)c2ccccc2C(=O)O)[C@@H](O)[C@H]1O]

In certain embodiments, the linker is a sulfamate or sulfamide linker. In certain embodiments, the linker is a vinylsulfonamide. In some embodiments, an inhibitor comprising a vinyl sulfonamide linker forms a covalent attachment with CoA in the presence of MenE and CoA.

In certain embodiments, the compound is of Formula (Z):

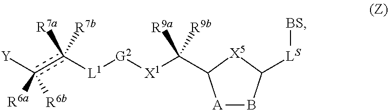

or a pharmaceutically acceptable salt, solvate, hydrate, polymorph, co-crystal, tautomer, stereoisomer, prodrug, or isotopically labeled derivative thereof, wherein:
BS is optionally substituted heterocyclyl, or optionally substituted heteroaryl, or an optionally substituted nucleobase or nucleobase analog;
G2 is —S(═O)₂—, —P(═O)(R^{e}), —P(═O)(OR^{e})—, —P(═O)(N(R^{e})₂)—, —P(═S)(R^{e})—, —P(═S)(OR^{e})—, —P(═S)(N(R^{e})₂)—, —Si(OR^{e})₂—, —C(═O)—, —C(═S)—, —C(═NR^{f})—, —(CH₂)ₕ—,

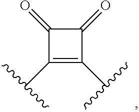 [SMILES: *c1c(*)c(=O)c1=O]

or optionally substituted monocyclic 5- or 6-membered heteroarylene, wherein 1, 2, 3, or 4 atoms in the heteroarylene ring system are independently oxygen, nitrogen, or sulfur;
A-B is —(R^{A})₂C—C(R^{B})₂— or —R^{A}C═CR^{B}—, wherein each occurrence of R^{A} is independently hydrogen, halogen, optionally substituted alkyl, optionally substituted acyl, —OR^{S1}, or —N(R^{e})₂, and each occurrence of R^{B} is independently hydrogen, halogen, optionally substituted alkyl, optionally substituted acyl, —OR^{S2}, or —N(R^{e})₂;
X⁵ is —O—, —S—, —C(R^{d})₂—, or —NR^{f}—;
Y is of formula:

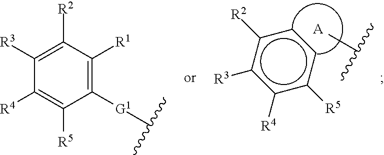

G is —C(R^{G1})(R^{G2})—, —C(═O)—, —C(═S)—, —C(═NR^{f})—, —C(═C(R^{G1})(R^{G2}))—, or —C(OR^{G1})(OR^{G2})—;
each of R^{G1} and R^{G2} is independently hydrogen, halogen, optionally substituted alkyl, optionally substituted alkenyl, optionally substituted alkynyl, —OR^{e}, or —N(R^{e})₂, or R^{G1} and R^{G2} are joined to form an optionally substituted carbocyclic ring or optionally substituted heterocyclic ring;
Ring A is an optionally substituted carbocyclic, optionally substituted heterocyclic, optionally substituted aryl, or optionally substituted heteroaryl ring;
L¹ is a bond or of formula:

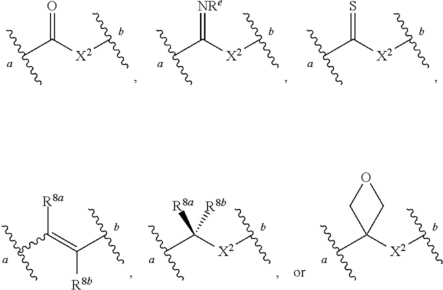

wherein L¹ is oriented such that the position labeled a is attached a carbon atom and the position labeled b is attached to G²;
X¹ is a bond, —O—, —C(R^{d})₂—, —(CH₂)_{q}—, or —NR^{f}—;
X² is a bond, —O—, —C(R^{d})₂—, —(CH₂)ₜ—, or —NR^{f}—;
R¹ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted boronyl, —NO₂, —CN, —OR^{e}, —N(R^{e})₂, —C(═NR^{e})R^{e}, —C(═NR^{e})OR^{e}, —C(═NR^{e})N(R^{e})₂, —C(═O)R^{e}, —C(═O)OR^{e}, —C(═O)N(R^{e})₂, —NR^{e}C(═O)R^{e}, —NR^{e}C(═O)OR^{e}, —NR^{e}C(═O)N(R^{e})₂, —OC(═O)R^{e}, —OC(═O)OR^{e}, or —OC(═O)N(R^{e})₂;
each of R², R³, and R⁴ are independently hydrogen, halogen, optionally substituted C₁₋₆ alkyl, optionally substituted acyl, —NO₂, —CN, —OR^{e}, or —N(R^{e})₂;
R⁵ is hydrogen, halogen, optionally substituted C₁₋₆ alkyl, —NO₂, —CN, —OR^{e}, or —N(R^{e})₂;
each of R^{6a} and R^{6b} is independently hydrogen, halogen, or optionally substituted C₁₋₆ alkyl;
each of R^{7a} and R^{7b} is independently hydrogen, halogen, or optionally substituted C₁₋₆ alkyl;
each of R^{8a} and R^{8b} is independently hydrogen, halogen, or optionally substituted C₁₋₆ alkyl;
each of R^{9a} and R^{9b} is independently hydrogen, halogen, optionally substituted C₁₋₆ alkyl, —OR^{e}, or —N(R^{e})₂;
each of R^{S1} and R^{S2} is independently hydrogen, optionally substituted C₁₋₆ alkyl, optionally substituted acyl, or an oxygen protecting group, or R^{S1} and R^{S2} are joined to form an optionally substituted heterocyclic ring;
L^{S} is a bond, —O—, —NR^{f}—, optionally substituted alkylene, optionally substituted alkenylene, optionally substituted alkynylene, optionally substituted acylene, or optionally substituted arylene;
each of V¹, V², V³, V⁷, V⁸, and V⁹ is independently N, NR^{V}, or CR^{V};
each occurrence of R^{V} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —NO₂, —CN, —OR^{e}, or —N(R^{e})₂;
V^{N} is N, NR^{N}, or CR^{N};
R^{N} is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —NO₂, —CN, —OR^{e}, or —N(R^{Na})₂;
each occurrence of R^{Na} independently hydrogen, optionally substituted C₁₋₆ alkyl, optionally substituted acyl, or a nitrogen protecting group, or both R^{Na} are joined to form and optionally substituted heterocyclic or optionally substituted heteroaryl ring;
each occurrence of R^{d} is independently hydrogen, halogen, optionally substituted C₁₋₆ alkyl, —OR^{e}, or —N(R^{e})₂
each occurrence of R^{e} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, an oxygen protecting group when attached to an oxygen atom, a nitrogen protecting group when attached to a nitrogen atom, or two R^{e} are joined to form and optionally substituted heterocyclic or optionally substituted heteroaryl ring;
each R^{f} is independently hydrogen, optionally substituted C₁₋₆ alkyl, optionally substituted acyl, or a nitrogen protecting group;
each of h, q, and t is independently 1, 2, or 3; and
[symbol] is a single, double, or triple bond, wherein R^{6b} and R^{7b} are absent when [symbol] is a double bond, and R^{6a}, R^{6b}, R^{7a}, and R^{7b} are absent when [symbol] is a triple bond.

In certain embodiments, the compound is of Formula (I):

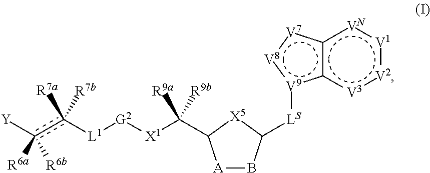

or a pharmaceutically acceptable salt, solvate, hydrate, polymorph, co-crystal, tautomer, stereoisomer, prodrug, or isotopically labeled derivative thereof, wherein:
G² is —S(═O)₂—, —P(═O)(R^{e})—, —P(═O)(OR^{e})—, —P(═O)(N(R^{e})₂)—, —P(═S)(R^{e})—, —P(═S)(OR^{e})—, —P(═S)(N(R^{e})₂)—, —Si(OR^{e})₂—, —C(═O)—, —C(═S)—, —C(═NR^{f})—, —(CH₂)ₕ—,

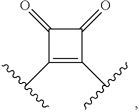 [SMILES: *c1c(*)c(=O)c1=O]

or optionally substituted monocyclic 5- or 6-membered heteroarylene, wherein 1, 2, 3, or 4 atoms in the heteroarylene ring system are independently oxygen, nitrogen, or sulfur;
A-B is —(R^{A})₂C═C(R^{B})₂— or —R^{A}C═CR^{B}—, wherein each occurrence of R^{A} is independently hydrogen, halogen, optionally substituted alkyl, optionally substituted acyl, —OR^{S1}, or —N(R^{e})₂, and each occurrence of R^{B} is independently hydrogen, halogen, optionally substituted alkyl, optionally substituted acyl, —OR^{S2}, or —N(R^{e})₂;
X⁵ is —O—, —S—, —C(R^{d})₂—, or —NR^{f}—;
Y is of formula:

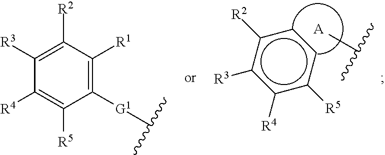

G1 is —C(R^{G1})(R^{G2})—, —C(═O)—, —C(═S)—, —C(═NR^{f})—, or —C(═C(R^{G1})(R^{G2}))—, or —C(OR^{G1})(OR^{G2})—;
each of R^{G1} and R^{G2} is independently hydrogen, halogen, optionally substituted alkyl, optionally substituted alkenyl, optionally substituted alkynyl, —OR^{e}, or —N(R^{e})₂, or R^{G1} and R^{G2} are joined to form an optionally substituted carbocyclic ring or optionally substituted heterocyclic ring;
Ring A is an optionally substituted carbocyclic, optionally substituted heterocyclic, optionally substituted aryl, or optionally substituted heteroaryl ring;
L¹ is a bond or of formula:

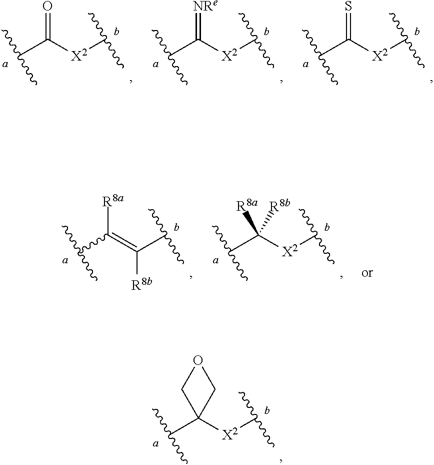

wherein L¹ is oriented such that the position labeled a is attached a carbon atom and the position labeled b is attached to G²;
X¹ is a bond, —O—, —C(R^{d})₂—, —(CH₂)_{q}—, or —NR^{f}—;
X² is a bond, —O—, —C(R^{d})₂—, —(CH₂)ₜ—, or —NR^{f}—;
R¹ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted boronyl, —NO₂, —CN, —OR^{e}, —N(R^{e})₂, —C(═NR^{e})R^{e}, —C(═NR^{e})OR^{e}, —C(═NR^{e})N(R^{e})₂, —C(═O)R^{e}, —C(═O)OR^{e}, —C(═O)N(R^{e})₂, —NR^{e}C(═O)R^{e}, —NR^{e}C(═O)OR^{e}, —NR^{e}C(═O)N(R^{e})₂, —OC(═O)R^{e}, —OC(═O)OR^{e}, or —OC(═O)N(R^{e})₂;
each of R², R³, and R⁴ are independently hydrogen, halogen, optionally substituted C₁₋₆ alkyl, optionally substituted acyl, —NO₂, —CN, —OR^{e}, or —N(R^{e})₂;
R⁵ is hydrogen, halogen, optionally substituted C₁₋₆ alkyl, —NO₂, —CN, —OR^{e}, or —N(R^{e})₂;
each of R^{6a} and R^{6b} is independently hydrogen, halogen, or optionally substituted C₁₋₆ alkyl;
each of R^{7a} and R^{7b} is independently hydrogen, halogen, or optionally substituted C₁₋₆ alkyl;
each of R^{8a} and R^{8b} is independently hydrogen, halogen, or optionally substituted C₁₋₆ alkyl;
each of R^{9a} and R^{9b} is independently hydrogen, halogen, optionally substituted C₁₋₆ alkyl, —OR^{e}, or —N(R^{e})₂;
each of R^{S1} and R^{S2} is independently hydrogen, optionally substituted C₁₋₆ alkyl, optionally substituted acyl, or an oxygen protecting group, or R^{S1} and R^{S2} are joined to form an optionally substituted heterocyclic ring;
L^{S} is a bond, —O—, —NR^{f}—, optionally substituted alkylene, optionally substituted alkenylene, optionally substituted alkynylene, optionally substituted acylene, or optionally substituted arylene;
each of V¹, V², V³, V⁷, V⁸, and V⁹ is independently N, NR^{V}, or CR^{V};
each occurrence of R^{V} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —NO₂, —CN, —OR^{e}, or —N(R^{e})₂;
V^{N} is N, NR^{N}, or CR^{N};
R^{N} is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —NO₂, —CN, —OR^{e}, or —N(R^{Na})₂;
each occurrence of R^{Na} independently hydrogen, optionally substituted C₁₋₆ alkyl, optionally substituted acyl, or a nitrogen protecting group, or both R^{Na} are joined to form and optionally substituted heterocyclic or optionally substituted heteroaryl ring; each occurrence of R^{d} is independently hydrogen, halogen, optionally substituted C₁₋₆ alkyl, —OR^{e}, or —N(R^{e})₂;
each occurrence of R^{e} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, an oxygen protecting group when attached to an oxygen atom, a nitrogen protecting group when attached to a nitrogen atom, or two R^{e} are joined to form and optionally substituted heterocyclic or optionally substituted heteroaryl ring;
each R^{f} is independently hydrogen, optionally substituted C₁₋₆ alkyl, optionally substituted acyl, or a nitrogen protecting group;
each of h, q, and t is independently 1, 2, or 3;
[symbol] is a single, double, or triple bond, wherein R^{6b} and R^{7b} are absent when [symbol] is a double bond, and R^{6a}, R^{6b}, R^{7a}, and R^{7b} are absent when [symbol] is a triple bond; and [symbol] indicates that each bond of the ring is a single or double bond.

In certain embodiments, the compound is not a compound of formula:

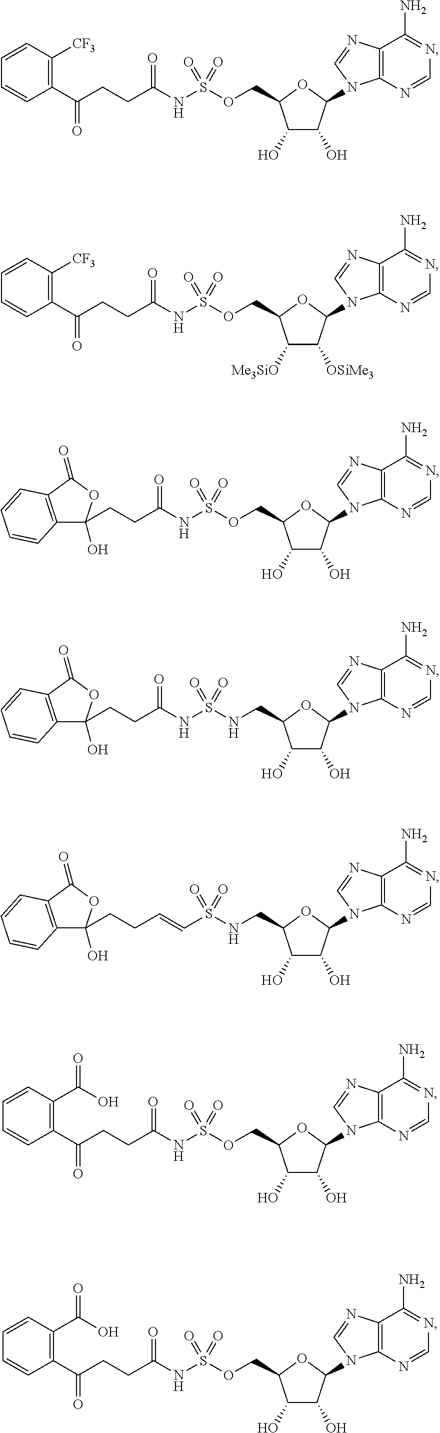

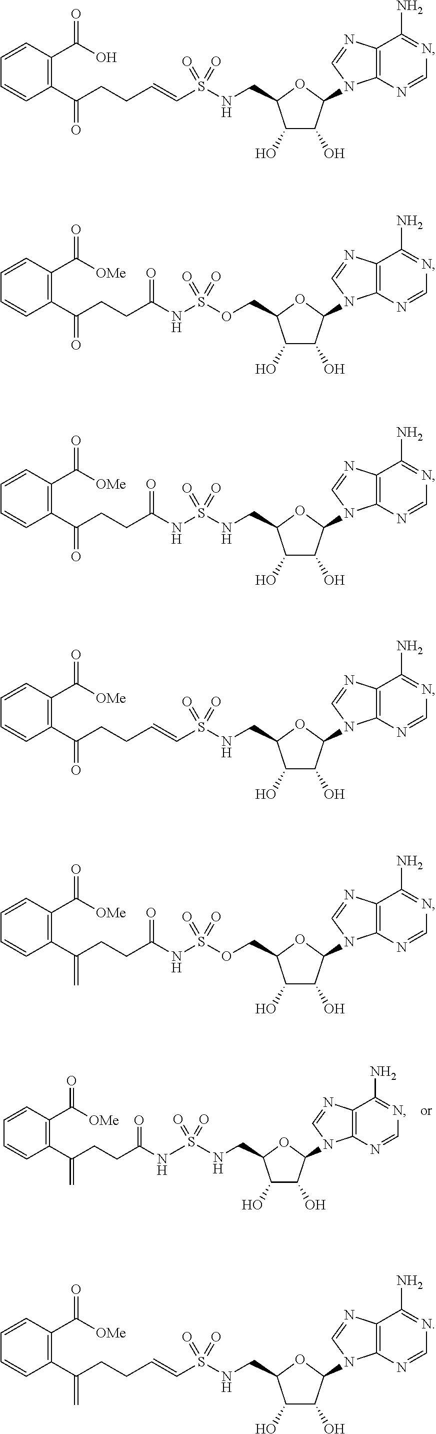

In certain embodiments, the compound is not a compound disclosed in: Tian et al., Biochemistry (2008) 47, 12434-12447; Lu et al., Bioorg. Med. Chem. Lett. (2008) 18, 5963-5966; Lu et al., ChemBioChem (2012) 13, 129-136; Davis et al., ACS Chem. Bio. (2014), 9, 2535-2544; U.S. Pat. No. 8,461,128; U.S. Pat. No. 8,946,188; U.S. patent application Ser. No. 11/911,525, filed Jul. 2, 2009; U.S. patent application Ser. No. 13/897,807, filed Jan. 23, 2014; or WIPO Application No. PCT/US2006/014394, filed Apr. 14, 2006. In certain embodiments, the compounds is not a compound disclosed in: U.S. Pat. No. 6,989,430; U.S. application Ser. No. 12/096,463, filed Nov. 27, 2008; or WIPO Application No. PCT/US2006/046433, filed Jun. 14, 2007.

In certain embodiments, a compound of Formula (Z) is a compound of Formula (I). In certain embodiments, a compounds of Formula (Z) is not a compound of Formula (I).

Unless otherwise stated, any formulae described herein are also meant to include salts, solvates, hydrates, polymorphs, co-crystals, tautomers, stereoisomers, prodrugs, and isotopically labeled derivatives thereof. In certain embodiments, the provided compound is a salt of any of the formulae described herein. In certain embodiments, the provided compound is a pharmaceutically acceptable salt of any of the formulae described herein. In certain embodiments, the provided compound is a solvate of any of the formulae described herein. In certain embodiments, the provided compound is a hydrate of any of the formulae described herein. In certain embodiments, the provided compound is a polymorph of any of the formulae described herein. In certain embodiments, the provided compound is a co-crystal of any of the formulae described herein. In certain embodiments, the provided compound is a tautomer of any of the formulae described herein. In certain embodiments, the provided compound is a stereoisomer of any of the formulae described herein. In certain embodiments, the provided compound is of an isotopically labeled form of any of the formulae described herein. For example, compounds having the present structures except for the replacement of hydrogen by deuterium or tritium, replacement of ¹⁹F with ¹⁸F, or the replacement of a ¹²C by a ¹³C or ¹⁴C are within the scope of the disclosure. In certain embodiments, the provided compound is a deuterated form of any of the formulae described herein.

A provided compound may be any possible stereoisomer of Formula (I). The ribose or ribose analog ring of Formula (I) may comprise four chiral centers, which each may independently be in either the (R)- or (S)-configuration. In certain embodiments, a compound of Formula (I) is a stereoisomer of formula:

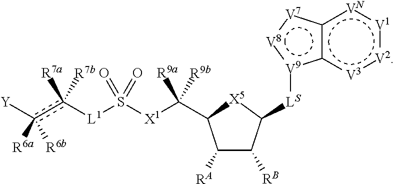

In some embodiments, a compound of Formula (I) is a stereoisomer of formula:

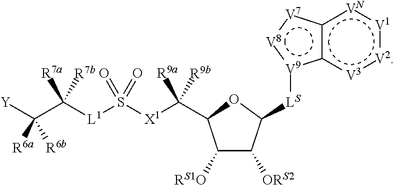

In certain embodiments, the compound of Formula (I) is a compound of Formula (II):

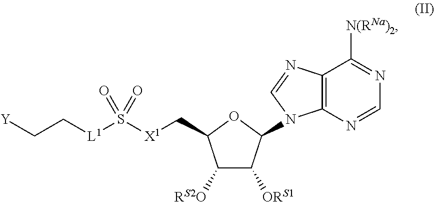

or a pharmaceutically acceptable salt, stereoisomer, or tautomer thereof, wherein Y, L¹, X¹, R^{S1}, R^{S2}, and R^{Na} are as described herein.

In certain embodiments, the compound of Formula (I) is a compound of Formula (III):

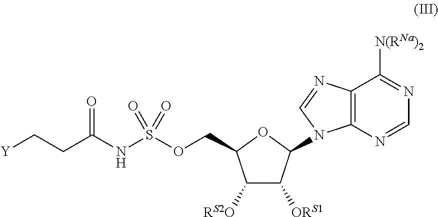

or a pharmaceutically acceptable salt, stereoisomer, or tautomer thereof, wherein Y, R^{S1}, R^{S2} and R^{Na} are as described herein.

In certain embodiments, the compound of Formula (I) is a compound of Formula (IV):

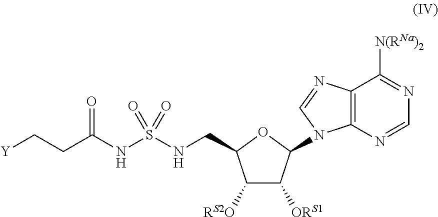

or a pharmaceutically acceptable salt, stereoisomer, or tautomer thereof, wherein Y, R^{S1}, R^{S2} and R^{Na} are as described herein.

In certain embodiments, the compound of Formula (I) is a compound of Formula (V):

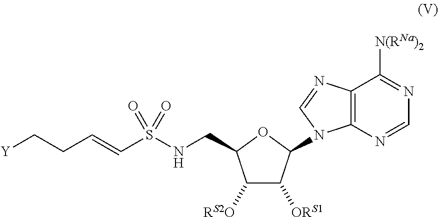

or a pharmaceutically acceptable salt, stereoisomer, or tautomer thereof, wherein Y, R^{S1}, R^{S2}, and R^{Na} are as described herein.

In certain embodiments, the compound of Formula (I) is a compound of Formula (VI):

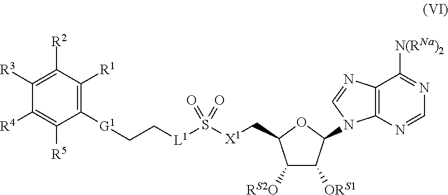

or a pharmaceutically acceptable salt, stereoisomer, or tautomer thereof, wherein G¹, L¹, X¹, R¹, R², R³, R⁴, R⁵, R^{S1}, R^{S2}, and R^{Na} are as described herein.

In certain embodiments, the compound of Formula (I) is a compound of Formula (VI′):

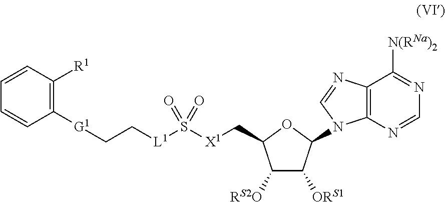

or a pharmaceutically acceptable salt, stereoisomer, or tautomer thereof, wherein G¹, L¹, X¹, R, R^{S1}, R^{S2}, and R^{Na} are as described herein.

In certain embodiments, the compound of Formula (I) is a compound of Formula (VII):

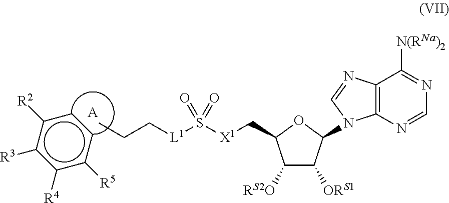

or a pharmaceutically acceptable salt, stereoisomer, or tautomer thereof, wherein Ring A, L¹, X¹, R², R³, R⁴, R⁵, R^{S1}, R^{S2}, and R^{Na} are as described herein.

In certain embodiments, Y is:

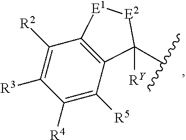

wherein:
E¹ is —C(═O)—, —C(═S)—, —C(═NR^{f})—, —C(R^{E1})₂—, —O—, or —NR^{f}—; and each R^{E1} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —OR^{e}, —SR^{e}, or —N(R^{e})₂;
E² is —C(═O)—, —C(═S)—, —C(═NR^{f})—, —C(R^{E2})₂—, —O—, or —NR^{f}—; and each R^{E2} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —OR^{e}, —SR^{e}, or —N(R^{e})₂; and
R^{Y} is hydrogen, halogen, optionally substituted alkyl, optionally substituted alkenyl, optionally substituted alkynyl, —OR^{e}, or —N(R^{e})₂.

In certain embodiments, the compound of Formula (I) is a compound of Formula (VII′):

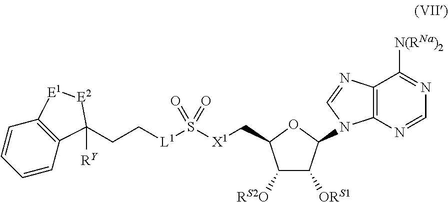

or a pharmaceutically acceptable salt, stereoisomer, or tautomer thereof, wherein E¹, E², L¹, X, R^{Y}, R^{S1}, R^{S2}, and R^{Na} are as described herein.

### Group Y

As generally defined herein, Y is of formula:

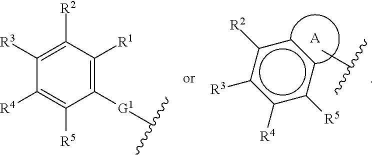

In certain embodiments, Y is of formula:

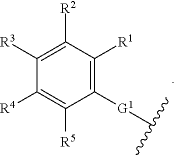

In certain embodiments, Y is of formula:

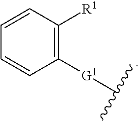

In certain embodiments, Y is of formula:

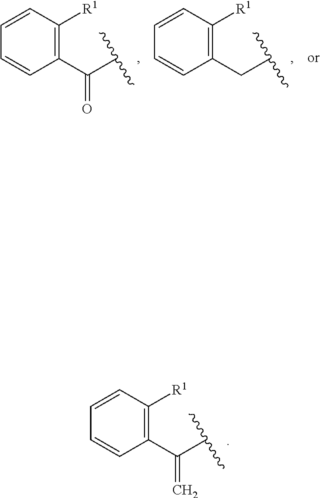

In certain embodiments, Y is of formula:

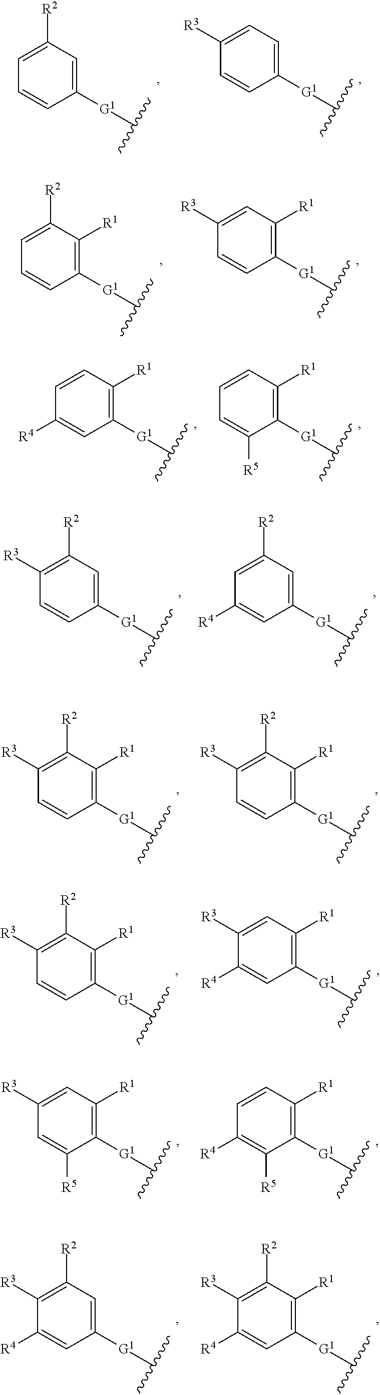

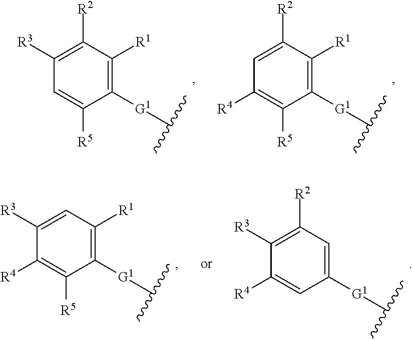

As generally defined herein, G¹ is —C(R^{G1})(R^{G2})—, C(O)—, —C(═S)—, —C(═NR^{f})—, or —C(═C(R^{G1})(R²))_, or —C(OR^{G1})(OR^{G2})—, wherein each of R^{G1} and R^{G2} is independently hydrogen, halogen, optionally substituted alkyl, optionally substituted alkenyl, optionally substituted alkynyl, —OR^{e}, or —N(R^{e})₂. In certain embodiments, G¹ is —C(═O)—. In certain embodiments, G¹ is —C(═S)—. In certain embodiments, G¹ is —C(═NR^{f})—. In some embodiments, G¹ is —C(═NH)—. In certain embodiments, G¹ is —C(═C(R^{G1})(R^{G2}))—. In some embodiments, G¹ is —C(═CH₂)—. In certain embodiments, G¹ is —C(R^{G1})(R^{G2})—. In some embodiments, G¹ is —C(R^{G1})(R^{G2})—, and both R^{G1} and R^{G2} are optionally substituted alkyl. In some embodiments, G¹ is —C(R^{G1})(R^{G2})—, and at least one of R^{G1} and R^{G2} is halogen (e.g., —F). In some embodiments, G¹ is —C(OR^{e})(OR^{e})—. In some embodiments, G¹ is —CH₂—. In some embodiments, G¹ is —CH(R^{G2})—. In some embodiments, G¹ is —CH(R^{G2})—, and R^{G2} is optionally substituted alkyl. In some embodiments, G¹ is —CH(OR^{e})—. In some embodiments, G¹ is —CH(N(R^{e})₂)—. In some embodiments, G¹ is —CH(OH)—. In some embodiments, G¹ is —CH(NH(R^{e}))₂—. In some embodiments, G¹ is —CH(NH₂)—. In some embodiments, G¹ is —C(OR^{G1})(OR^{G2})—. In some embodiments, G1 is —C(OR^{G1})(OR^{G2})—, wherein each of R^{G1} and R^{G2} is independently H or substituted or unsubstituted C₁₋₆ alkyl. In some embodiments, G¹ is —C(OR^{G1})(OR^{G2})—, wherein R^{G1} and R^{G2} are joined to form an optionally substituted heterocyclic ring.

In certain embodiments, R^{G1} is hydrogen. In certain embodiments, R^{G1} is halogen. In certain embodiments, R^{G1} is optionally substituted alkyl (e.g., optionally substituted C₁₋₆ alkyl), optionally substituted alkenyl (e.g., optionally substituted C₁₋₆ alkenyl), or optionally substituted alkynyl (e.g., optionally substituted C₁₋₆ alkynyl). In certain embodiments, R^{G1} is —OR^{e} (e.g., —OH or —O(substituted or unsubstituted C₁₋₆ alkyl)) or —N(R^{e})₂ (e.g., —NH₂, —NH (substituted or unsubstituted C₁₋₆ alkyl), or —N(substituted or unsubstituted C₁₋₆ alkyl)₂). In certain embodiments, R^{G2} is hydrogen. In certain embodiments, R^{G2} is halogen. In certain embodiments, R^{G2} is optionally substituted alkyl (e.g., optionally substituted C₁₋₆ alkyl), optionally substituted alkenyl (e.g., optionally substituted C₁₋₆ alkenyl), or optionally substituted alkynyl (e.g., optionally substituted C₁₋₆ alkynyl). In certain embodiments, R^{G2} is —OR^{e} (e.g., —OH or —O(substituted or unsubstituted C₁₋₆ alkyl)) or —N(R^{e})₂ (e.g., —NH₂, —NH (substituted or unsubstituted C₁₋₆ alkyl), or —N(substituted or unsubstituted C₁₋₆ alkyl)₂). In certain embodiments, R^{G1} and R^{G2} are joined to form an optionally substituted carbocyclic ring. In certain embodiments, R^{G1} and R^{G2} are joined to form an optionally substituted heterocyclic ring.

### Group R¹

As generally defined herein R¹ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted boronyl, —NO₂, —CN, —OR^{e}, —N(R^{e})₂, —C(═NR^{e})R^{e}, —C(═NR^{e})OR^{e}, —C(═NR^{e})N(R^{e})₂, —C(═O)R^{e}, —C(═O)OR^{e}, —C(═O)N(R^{e})₂, —NR^{e}C(═O)R^{e}, —NR^{e}C(═O)OR^{e}, —NR^{e}C(═O)N(R^{e})₂, —OC(═O)R^{e}, —OC(═O)OR^{e}, or —OC(═O)N(R^{e})₂. In certain embodiments, R¹ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted boronyl, —NO₂, —CN, —OR^{e}, or —N(R^{e})₂. In certain embodiments, R¹ is hydrogen, halogen, optionally substituted alkenyl, optionally substituted alkynyl, optionally substituted carbocyclyl, optionally substituted heterocyclyl, optionally substituted aryl, optionally substituted heteroaryl, —NO₂, —CN, —OR^{e}, or —N(R^{e})₂. In certain embodiments, R¹ is optionally substituted carbocyclyl, optionally substituted heterocyclyl, optionally substituted aryl, or optionally substituted heteroaryl.

In certain embodiments, R¹ is hydrogen. In certain embodiments, R¹ is halogen. In certain embodiments, R¹ is —F. In certain embodiments, R¹ is —Cl, —Br, or —F. In certain embodiments, R¹ is —NO₂. In certain embodiments, R¹ is —CN. In certain embodiments, R¹ is —OR^{e} (e.g. —OH, —OMe, —O(C₁₋₆ alkyl)). In certain embodiments, R¹ is —OR^{e}, and R^{e} is an oxygen protecting group. In certain embodiments, R¹ is —N(R^{e})₂ (e.g., —NH₂, —NMe₂, —NH(C₁₋₆ alkyl)). In certain embodiments, R¹ is —NH(R^{e})₂, and R^{e} is a nitrogen protecting group.

In certain embodiments, R¹ is optionally substituted alkyl, e.g., optionally substituted C₁₋₆ alkyl, optionally substituted C₁₋₂ alkyl, optionally substituted C₂₋₃ alkyl, optionally substituted C₃₋₄ alkyl, optionally substituted C₄₋₅ alkyl, or optionally substituted C₅₋₆ alkyl. In certain embodiments, R¹ is methyl. In certain embodiments, R¹ is ethyl, propyl, or butyl. In certain embodiments, R¹ is optionally substituted alkenyl, e.g., optionally substituted C₂₋₆ alkenyl. In certain embodiments, R¹ is vinyl, allyl, or prenyl. In certain embodiments, R¹ is optionally substituted alkynyl, e.g., C₂₋₆ alkynyl.

In certain embodiments, R¹ is optionally substituted carbocyclyl, e.g., optionally substituted C₃₋₆ carbocyclyl, optionally substituted C₃₋₄ carbocyclyl, optionally substituted C₄₋₅ carbocyclyl, or optionally substituted C₅₋₆ carbocyclyl. In certain embodiments R¹ is optionally substituted heterocyclyl, e.g., optionally substituted 3-6 membered heterocyclyl, optionally substituted 3-4 membered heterocyclyl, optionally substituted 4-5 membered heterocyclyl, or optionally substituted 5-6 membered heterocyclyl.

In certain embodiments, R¹ is optionally substituted aryl, e.g., optionally substituted phenyl. In certain embodiments, R¹ is optionally substituted heteroaryl, e.g., optionally substituted 5-6 membered heteroaryl, or optionally substituted 9-10 membered bicyclic heteroaryl. In certain embodiments, R¹ is pyrrolyl, furanyl, thiophenyl, imidazolyl, pyrazolyl, oxazolyl, isoxazolyl, thiazolyl, isothiazolyl, triazolyl, oxadiazolyl, thiadiazolyl, or tetrazolyl, each of which is independently optionally substituted. In certain embodiments, R¹ is optionally substituted aralkyl, e.g., optionally substituted benzyl. In certain embodiments, R¹ is optionally substituted heteroaralkyl, e.g., methyl substituted with a 5-6-membered heteroaryl ring.

In certain embodiments, R¹ is optionally substituted boronyl (e.g., —B(OH)₂). In certain embodiments, R¹ is —B(R^{aa})₂, wherein R^{aa} is as defined herein. In certain embodiments, R¹ is —B(OR^{cc})₂, wherein R^{cc} is as defined herein. In some embodiments, R^{cc} is hydrogen, methyl, ethyl, propyl, or butyl. In some embodiments, two R^{cc} are joined to form an optionally substituted heterocyclic ring (e.g., a pinacol borane or catechol borane).

In certain embodiments, R¹ is optionally substituted alkyl, wherein the carbon directly attached to the phenyl ring is substituted with at least one hydroxy or alkoxy group. In certain embodiments, R¹ is —CR^{EWG}(OH), wherein R^{EWG} is an electron withdrawing group. In some embodiments, the electron withdrawing group is halogen (e.g., F, Cl, Br), haloalkyl (e.g., trifluoromethyl, trichloromethyl), cyano, optionally substituted acyl, optionally substituted sulfonyl, or nitro. In some embodiments, the electron withdrawing group is trifluoromethyl.

In certain embodiments, R¹ is:

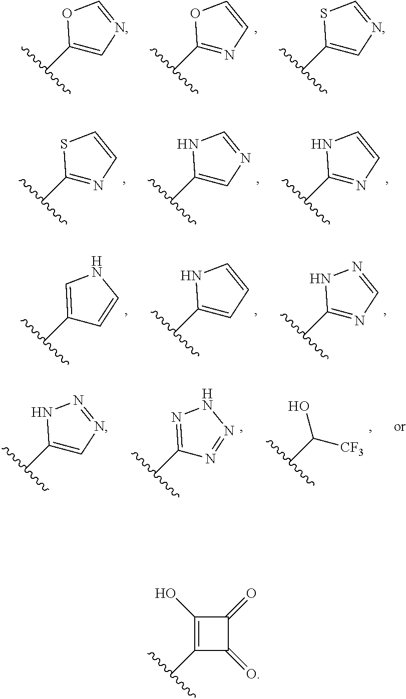

In certain embodiments, R¹ is

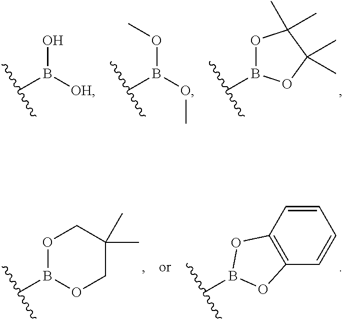

In certain embodiments, R¹ is —C(═NR^{e})R^{e}, —C(═NR^{e})OR^{e}, —C(═NR^{e})N(R^{e})₂, —C(═O)R^{e}, —C(═O)OR^{e}, or —C(═O)N(R^{e})₂, optionally wherein each instance of R^{e} is independently H, substituted or unsubstituted C₁₋₆ alkyl, an oxygen protecting group when attached to an oxygen atom, or a nitrogen protecting group when attached to a nitrogen atom. In certain embodiments, R¹ is —NR^{e}C(═O)R^{e}, —NR^{e}C(═O)OR^{e}, —NR^{e}C(═O)N(R^{e})₂, —OC(═O)R^{e}, —OC(═O)OR^{e}, or —OC(═O)N(R^{e})₂, optionally wherein each instance of R^{e} is independently H, substituted or unsubstituted C₁₋₆ alkyl, an oxygen protecting group when attached to an oxygen atom, or a nitrogen protecting group when attached to a nitrogen atom.

In certain embodiments, R¹ is not —C(═O)OMe or —C(═O)OH. In certain embodiments, R¹ is not —C(═O)OR^{e}, wherein R^{e} is hydrogen or unsubstituted C₁₋₆ alkyl.

In certain embodiments, Y is of formula:

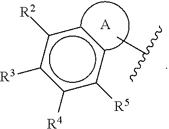

In certain embodiments, Y is of formula:

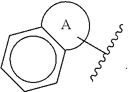

In certain embodiments, Y is of formula:

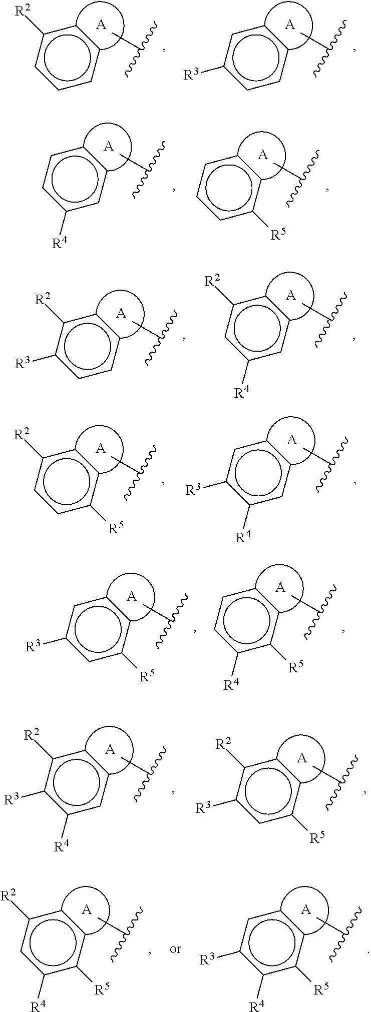

In certain embodiments, Ring A is an optionally substituted carbocyclic ring (e.g., an optionally substituted 5- to 6-membered carbocyclic ring). In certain embodiments, Ring A is a optionally substituted heterocyclic ring (e.g., an optionally substituted 5- to 6-membered heterocyclic ring, comprising 0 to 3 heteroatoms independently selected from O, N, and S). In certain embodiments, Ring A is an optionally substituted aryl ring (e.g., an optionally substituted phenyl ring). In certain embodiments, Ring A is an optionally substituted heteroaryl ring (e.g., an optionally substituted 5- to 6-membered heteroaryl ring, comprising 0 to 3 heteroatoms independently selected from O, N, and S).

In certain embodiments, Y is of formula:

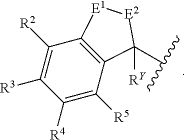

In certain embodiments, Y is of formula:

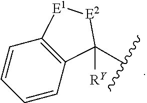

In certain embodiments, Y is of formula:

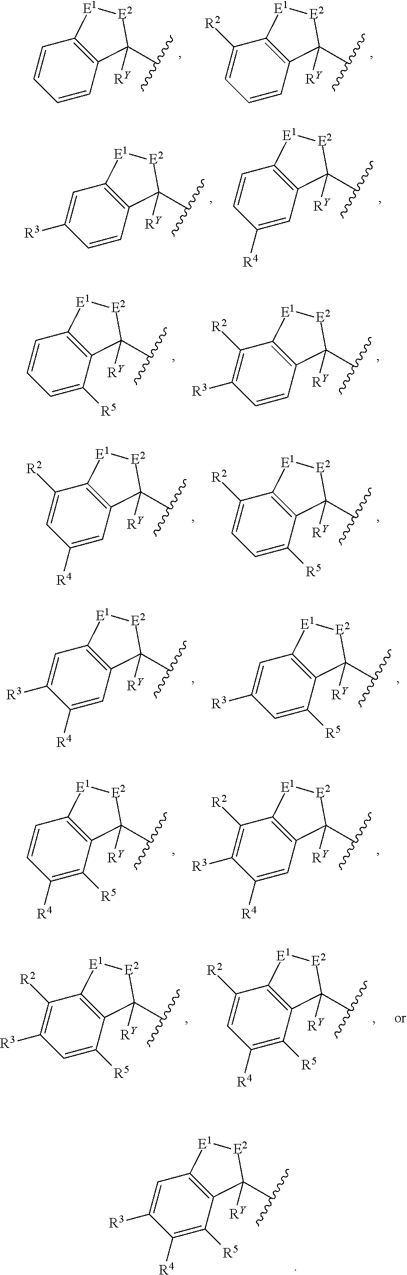

In certain embodiments, Y is of formula:

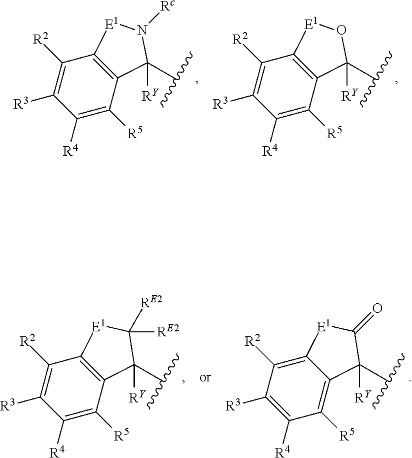

In certain embodiments, Y is of formula:

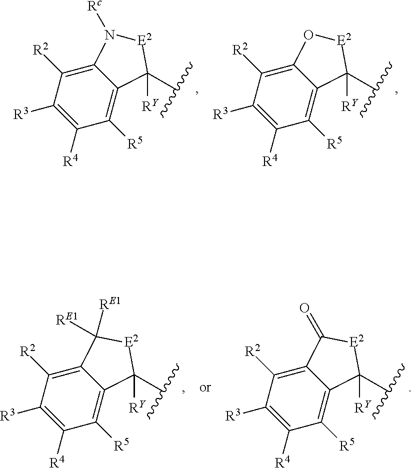

In certain embodiments, Y is of formula:

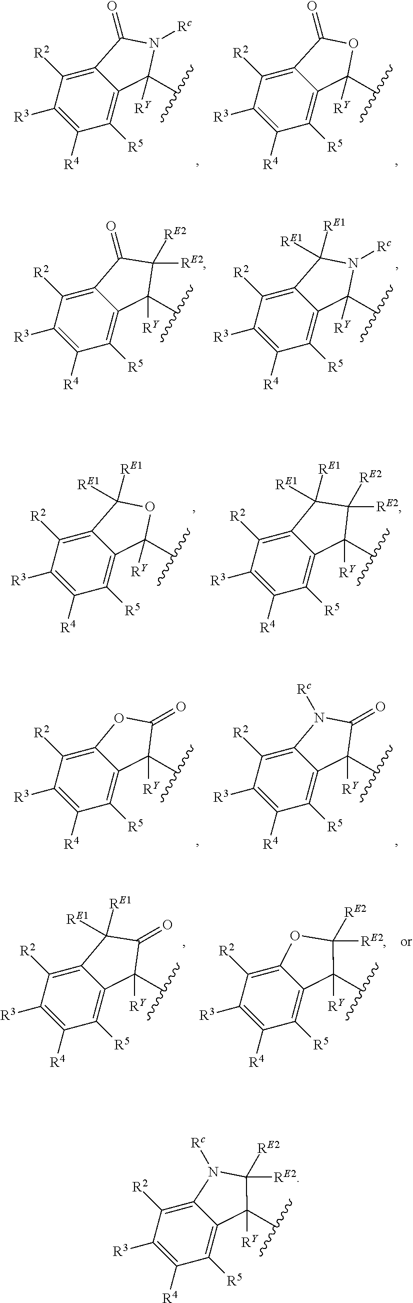

In certain embodiments, Y is of formula:

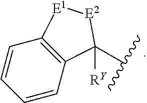

In certain embodiments, Y is of formula:

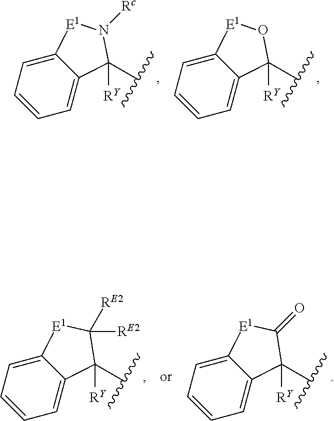

In certain embodiments, Y is of formula:

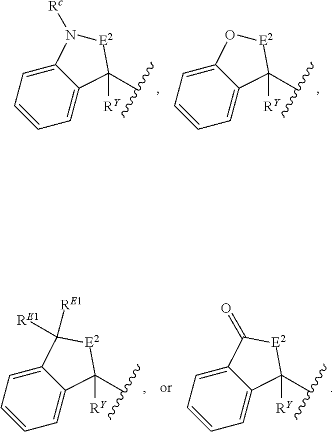

In certain embodiments, Y is:

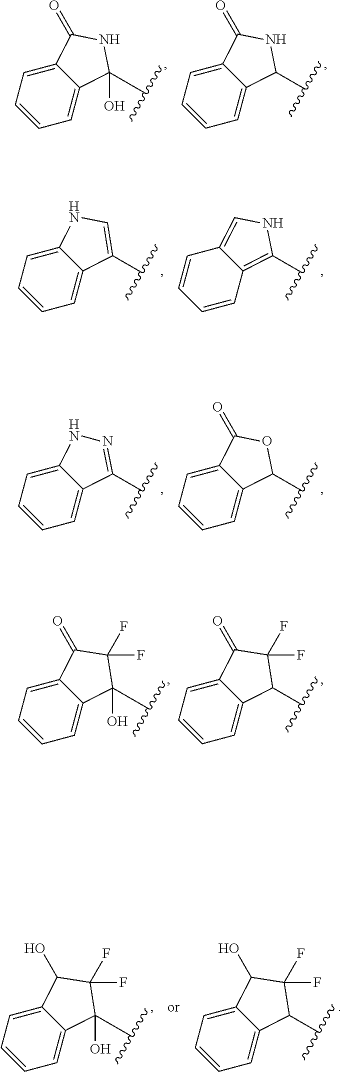

In certain embodiments, Y is:

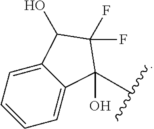 [SMILES: *C1(O)c2ccccc2C(O)C1(F)F]

In certain embodiments, Y is of one of the following formulae:

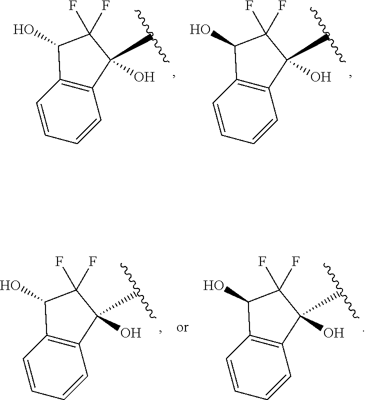 [SMILES: *[C@@]1(O)c2ccccc2[C@H](O)C1(F)F ; *[C@@]1(O)c2ccccc2[C@@H](O)C1(F)F ; *[C@]1(O)c2ccccc2[C@H](O)C1(F)F ; *[C@]1(O)c2ccccc2[C@@H](O)C1(F)F]

Groups E¹, E², and R^{Y}

As generally defined herein, E¹ is —C(═O)—, —C(═S)—, —C(═NR^{f})—, —C(R^{E1})₂—, —O—, or —NR^{f}—; and each R^{E1} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —OR^{e}, —SR^{e}, or —N(R^{e})₂. When E¹ is —C(R^{E1})₂—, the carbon to which both R^{E1} are attached may be of either the (R)- or (S)-configuration.

In certain embodiments, E¹ is —C(═O)—. In certain embodiments, E¹ is —C(═S)—. In certain embodiments, E¹ is —C(═NR^{f})— (e.g., —C(═NH)—). In certain embodiments, E¹ is —C(R^{E1}₂— (e.g., —CH₂—, —CH(R^{E2})—). In some embodiments, E¹ is —CH(OR^{e})⁻ (e.g., —CH(OH)—). In some embodiments, E¹ is —C(R^{E1})₂, wherein at least one occurrence of R^{E1} is halogen. In some embodiments, E¹ is —CF₂—, —CCl₂—, —CBr₂—, or —CI₂—. In certain embodiments, E¹ is —O—. In certain embodiments, E¹ is —NR^{f}— (e.g., —NH—, —NMe-).

As generally defined herein, E² is —C(═O)—, —C(═S)—, —C(═NR^{f})—, —C(R^{E2})₂—, —O—, or —NR^{f}—; and each R^{E2} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —OR^{e}, or —N(R^{e})₂. When E² is —C(R^{E2})₂—, the carbon to which both R^{E2} are attached may be of either the (R) or (S) configuration.

In certain embodiments, E² is —C(═O)—. In certain embodiments, E² is —C(═S)—. In certain embodiments, E² is —C(═NR^{f})— (e.g., —C(═NH)—). In certain embodiments, E² is —C(R^{E2})₂— (e.g., —CH₂—, —CH(R^{E2})—). In some embodiments, E² is —CH(OR^{e})⁻ (e.g., —CH(OH)—). In some embodiments, E² is —C(R^{E2})₂, wherein at least one occurrence of R^{E2} is halogen. In some embodiments, E² is —CF₂—, —CCl₂—, —CBr₂—, or —CI₂—. In certain embodiments, E is —O—. In certain embodiments, E is —NR^{f}— (e.g., —NH—, —NMe—).

R^{Y} is hydrogen, halogen, optionally substituted alkyl, optionally substituted alkenyl, optionally substituted alkynyl, —OR^{e}, or —N(R^{e})₂. The carbon to which R^{Y} is attached may be of either the (R)- or (S)-configuration.

In certain embodiments, R^{Y} is hydrogen. In certain embodiments, R^{Y} is halogen. In certain embodiments, R^{Y} is —F. In certain embodiments, R^{Y} is —Cl, —Br, or —F. In certain embodiments, R^{Y} is —NO₂. In certain embodiments, R^{Y} is —CN. In certain embodiments, R^{Y} is —OR^{e} (e.g. —OH, —OMe, —O(C₁₋₆ alkyl)) In certain embodiments, R^{Y} is —OR^{e}, and R^{e} is an oxygen protecting group. In certain embodiments, R^{Y} is —N(R^{e})₂ (e.g., —NH₂, —NMe₂, —NH(C₁₋₆ alkyl)). In certain embodiments, R^{Y} is —NHR^{e}, and R^{e} is a nitrogen protecting group.

In certain embodiments, R^{Y} is optionally substituted alkyl, e.g., optionally substituted C₁₋₆ alkyl, optionally substituted C₁₋₂ alkyl, optionally substituted C₂₋₃ alkyl, optionally substituted C₃₋₄ alkyl, optionally substituted C₄₋₅ alkyl, or optionally substituted C₅₋₆ alkyl. In certain embodiments, R^{Y} is methyl. In certain embodiments, R^{Y} is ethyl, propyl, or butyl. In certain embodiments, R^{Y} is optionally substituted alkenyl, e.g., optionally substituted C₂₋₆ alkenyl. In certain embodiments, R^{Y} is vinyl, allyl, or prenyl. In certain embodiments, R^{Y} is optionally substituted alkynyl, e.g., C₂₋₆ alkynyl.

Groups R², R³, R⁴, and R⁵

As generally defined herein R² is hydrogen, halogen, optionally substituted C₁₋₆ alkyl, optionally substituted acyl, —NO₂, —CN, —OR^{e}, or —N(R^{e})₂. In certain embodiments, R² is hydrogen. In certain embodiments, R² is halogen. In certain embodiments, R² is —F. In certain embodiments, R² is —Cl, —Br, or —F. In certain embodiments, R² is optionally substituted C₁₋₆ alkyl. In certain embodiments, R² is unsubstituted C₁₋₆ alkyl. In certain embodiments, R² is methyl. In certain embodiments, R² is ethyl, propyl, or butyl. In certain embodiments, R² is —NO₂. In certain embodiments, R² is —CN. In certain embodiments, R² is —OR^{e} (e.g. —OH, —OMe, —O(C₁₋₆ alkyl)). In certain embodiments, R² is —OR^{e}, and R^{e} is an oxygen protecting group. In certain embodiments, R² is —N(R^{e})₂ (e.g., —NH₂, —NMe₂, —NH(C₁₋₆ alkyl)). In certain embodiments, R² is —NHR^{e}, and R^{e} is a nitrogen protecting group. In certain embodiments, R² is optionally substituted acyl (e.g., —C(═O)(R^{e}), —C(═O)O(R^{e}), —C(═O)NH(R^{e}), —C(═O)N(R^{e})₂). In some embodiments, R² is —C(═O)OMe. In some embodiments, R² is —C(═O)OH.

As generally defined herein R³ is hydrogen, halogen, optionally substituted C₁₋₆ alkyl, optionally substituted acyl, —NO₂, —CN, —OR^{e}, or —N(R^{e})₂. In certain embodiments, R³ is hydrogen. In certain embodiments, R³ is halogen. In certain embodiments, R³ is —F. In certain embodiments, R³ is —Cl, —Br, or —F. In certain embodiments, R³ is optionally substituted C₁₋₆ alkyl. In certain embodiments, R³ is unsubstituted C₁₋₆ alkyl. In certain embodiments, R³ is methyl. In certain embodiments, R³ is ethyl, propyl, or butyl. In certain embodiments, R³ is —NO₂. In certain embodiments, R³ is —CN. In certain embodiments, R³ is —OR^{e} (e.g. —OH, —OMe, —O(C₁₋₆ alkyl)) In certain embodiments, R³ is —OR^{e}, and R^{e} is an oxygen protecting group. In certain embodiments, R³ is —N(R^{e})₂ (e.g., —NH₂, —NMe₂, —NH(C₁₋₆ alkyl)). In certain embodiments, R³ is —NHR^{e}, and R^{e} is a nitrogen protecting group. In certain embodiments, R³ is optionally substituted acyl (e.g., —C(═O)(R^{e}), —C(═O)O(R^{e}), —C(═O)NH(R^{e}), —C(═O)N(R^{e})₂). In some embodiments, R³ is —C(═O)OMe. In some embodiments, R³ is —C(═O)OH.

As generally defined herein R⁴ is hydrogen, halogen, optionally substituted C₁₋₆ alkyl, optionally substituted acyl, —NO₂, —CN, —OR^{e}, or —N(R^{e})₂. In certain embodiments, R⁴ is hydrogen. In certain embodiments, R⁴ is halogen. In certain embodiments, R⁴ is —F. In certain embodiments, R⁴ is —Cl, —Br, or —F. In certain embodiments, R⁴ is optionally substituted C₁₋₆ alkyl. In certain embodiments, R⁴ is unsubstituted C₁₋₆ alkyl. In certain embodiments, R⁴ is methyl. In certain embodiments, R⁴ is ethyl, propyl, or butyl. In certain embodiments, R⁴ is —NO₂. In certain embodiments, R⁴ is —CN. In certain embodiments, R⁴ is —OR^{e} (e.g. —OH, —OMe, —O(C₁₋₆ alkyl)) In certain embodiments, R⁴ is —OR^{e}, and R^{e} is an oxygen protecting group. In certain embodiments, R⁴ is —N(R^{e})₂ (e.g., —NH₂, —NMe₂, —NH(C₁₋₆ alkyl)). In certain embodiments, R⁴ is —NHR^{e} and R^{e} is a nitrogen protecting group. In certain embodiments, R⁴ is optionally substituted acyl (e.g., —C(═O)(R^{e}), —C(═O)O(R^{e}), —C(═O)NH(R^{e}), —C(═O)N(R^{e})₂). In some embodiments, R⁴ is —C(═O)OMe. In some embodiments, R⁴ is —C(═O)OH.

As generally defined herein R⁵ is hydrogen, halogen, optionally substituted C₁₋₆ alkyl, optionally substituted acyl, —NO₂, —CN, —OR^{e}, or —N(R^{e})₂. In certain embodiments, R⁵ is hydrogen. In certain embodiments, R⁵ is halogen. In certain embodiments, R⁵ is —F. In certain embodiments, R⁵ is —Cl, —Br, or —F. In certain embodiments, R⁵ is optionally substituted C₁₋₆ alkyl. In certain embodiments, R⁵ is unsubstituted C₁₋₆ alkyl. In certain embodiments, R⁵ is methyl. In certain embodiments, R⁵ is ethyl, propyl, or butyl. In certain embodiments, R⁵ is —NO₂. In certain embodiments, R⁵ is —CN. In certain embodiments, R⁵ is —OR^{e} (e.g. —OH, —OMe, —O(C₁₋₆ alkyl)) In certain embodiments, R⁵ is —OR^{e}, and R^{e} is an oxygen protecting group. In certain embodiments, R⁵ is —N(R^{e})₂ (e.g., —NH₂, —NMe₂, —NH(C₁₋₆ alkyl)). In certain embodiments, R is —NHR^{e} and R^{e} is a nitrogen protecting group.

Linker L¹, X¹ and X²

As generally defined herein, X¹ is a bond, —O—, —C(R^{d})₂—, —(CH₂)_{q}—, or —NR^{f}—. In certain embodiments, X¹ is a bond. In certain embodiments, X¹ is —O—. In certain embodiments, X¹ is —NH—. In certain embodiments, X¹ is —NR^{f}—, and R^{d} is optionally substituted C₁₋₆ alkyl. In certain embodiments, X¹ is —NR^{f}—, and R^{f} is unsubstituted C₁₋₆ alkyl. In certain embodiments, X¹ is —NR^{f}—, and R^{f} is methyl. In certain embodiments, X¹ is —NR^{f}—, and R^{d} is ethyl, propyl, or butyl. In certain embodiments, X¹ is —NR^{f-}, and R^{f} is optionally substituted acyl (e.g., —C(═O)(R^{e}), —C(═O)O(R^{e}), —C(═O)NH(R^{e}), —C(═O)N(R^{e})₂). In certain embodiments, X¹ is —NR^{f}—, and R^{f} is a nitrogen protecting group. In certain embodiments, X¹ is —C(R^{d})₂. In certain embodiments, X¹ is —CH₂—. In certain embodiments, X¹ is —C(R^{d})₂—, and both R^{d} are halogen. In certain embodiments, X¹ is —CF₂—.

In certain embodiments, X¹ is —(CH₂)_{q}—, wherein q is 1, 2, or 3. In some embodiments, X¹ is —(CH₂)_{q}—, wherein q is 1. In some embodiments, X¹ is —(CH₂)_{q}—, wherein q is 2 or 3.

As generally defined herein, L¹ is a bond or of formula:

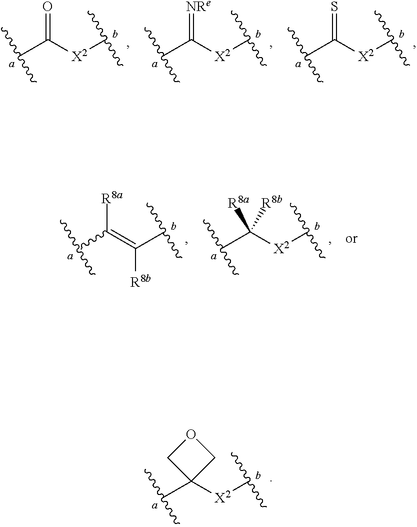

wherein L¹ is oriented such that the position labeled a is attached a carbon atom and the position labeled b is attached to a sulfur atom; and X² is —O—, —C(R^{d})₂—, or —NR^{f}—. In certain embodiments, L¹ is a bond.

In certain embodiments, L¹ is of formula:

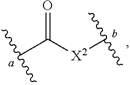

wherein the position labeled a is attached a carbon atom and the position labeled b is attached to a sulfur atom. In some embodiments, X² is a bond. In some embodiments, X² is —O—. In some embodiments, X² is —NH—. In some embodiments, X² is —NR^{f}—, and R^{f} is optionally substituted C₁₋₆ alkyl. In some embodiments, X² is —NR^{f}—, and R is unsubstituted C₁₋₆ alkyl. In some embodiments, X² is —NR^{f}—, and R^{f} is methyl. In some embodiments, X² is —NR^{f}—, and R^{f} is ethyl, propyl, or butyl. In some embodiments, X² is —NR^{f}—, and R^{f} is optionally substituted acyl (e.g., —C(═O)(R^{e}), —C(═O)O(R^{e}), —C(═O)NH(R^{e}), —C(═O)N(R^{e})₂). In some embodiments, X² is —NR^{f}—, and R^{f} is a nitrogen protecting group. In certain embodiments, X² is —C(R^{d})₂—. In certain embodiments, X² is —CH₂—. In certain embodiments, X² is —C(R^{d})₂—, and both R^{d} are halogen. In certain embodiments, X² is —CF₂—. In certain embodiments, X² is —(CH₂)_{q}—, wherein q is 1, 2, or 3. In some embodiments, X² is —(CH₂)_{q}—, wherein q is 1. In some embodiments, X² is —(CH₂)_{q}—, wherein q is 2 or 3.

In certain embodiments, L¹ is of formula:

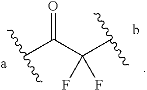 [SMILES: *C(=O)C(*)(F)F]

In certain embodiments, L¹ is of formula:

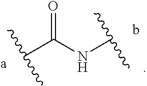 [SMILES: *NC(*)=O]

In certain embodiments, L¹ is of formula:

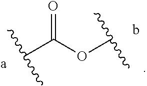 [SMILES: *OC(*)=O]

In certain embodiments, L¹ is of formula:

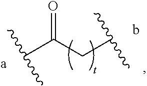

wherein t is 1, 2, or 3. In some embodiments, t is 1. In some embodiments, t is 2 or 3.

In certain embodiments, L¹ is of formula:

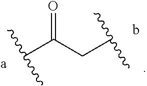 [SMILES: *CC(*)=O]

In certain embodiments, L¹ is of formula:

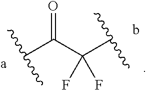 [SMILES: *C(=O)C(*)(F)F]

In certain embodiments, L¹ is of formula:

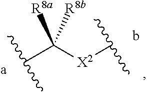

wherein the position labeled a is attached a carbon atom and the position labeled b is attached to a sulfur atom. In some embodiments, X² is a bond. In some embodiments, X² is —O—. In some embodiments, X² is —NH—. In some embodiments, X² is —NR—, and R^{f} is optionally substituted C₁₋₆ alkyl. In some embodiments, X² is —NR^{f}—, and R is unsubstituted C₁₋₆ alkyl. In some embodiments, X² is —NR^{f}—, and R^{f} is methyl. In some embodiments, X² is —NR^{f}—, and R^{f} is ethyl, propyl, or butyl. In some embodiments, X² is —NR^{f}—, and R^{f} is optionally substituted acyl (e.g., —C(═O)(R^{e}), —C(═O)O(R^{e}), —C(═O)NH(R^{e}), —C(═O)N(R^{e})₂). In some embodiments, X² is —NR^{f}—, and R^{f} is a nitrogen protecting group. In certain embodiments, X² is —C(R^{d})₂—. In certain embodiments, X² is —CH₂—. In certain embodiments, X² is —C(R^{d})₂—, and both R^{d} are halogen. In certain embodiments, X² is —CF₂—. In certain embodiments, X² is —(CH₂)_{q}—, wherein q is 1, 2, or 3. In some embodiments, X² is —(CH₂)_{q}—, wherein q is 1. In some embodiments, X² is —(CH₂)_{q}—, wherein q is 2 or 3.
In certain embodiments, L¹ is of formula:

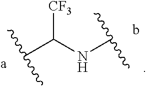 [SMILES: *NC(*)C(F)(F)F]

In certain embodiments, L¹ is of formula:

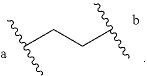 [SMILES: *CC*]

In certain embodiments, L¹ is of formula:

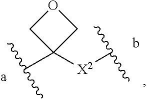

wherein the position labeled a is attached a carbon atom and the position labeled b is attached to a sulfur atom. In some embodiments, X² is a bond. In some embodiments, X² is —O—. In some embodiments, X² is —NH—. In some embodiments, X² is —NR—, and R^{f} is optionally substituted C₁₋₆ alkyl. In some embodiments, X² is —NR^{f}—, and R is unsubstituted C₁₋₆ alkyl. In some embodiments, X² is —NR^{f}—, and R^{f} is methyl. In some embodiments, X² is —NR^{f}—, and R^{f} is ethyl, propyl, or butyl. In some embodiments, X² is —NR^{f}—, and R^{f} is optionally substituted acyl (e.g., —C(═O)(R^{e}), —C(═O)O(R^{e}), —C(═O)NH(R^{e}), —C(═O)N(R^{e})₂). In some embodiments, X² is —NR^{f}—, and R^{f} is a nitrogen protecting group. In certain embodiments, X² is —C(R^{d})₂—. In certain embodiments, X² is —CH₂—. In certain embodiments, X² is —C(R^{d})₂—, and both R^{d} are halogen. In certain embodiments, X² is —CF₂—. In certain embodiments, X² is —(CH₂)_{q}—, wherein q is 1, 2, or 3. In some embodiments, X² is —(CH₂)_{q}—, wherein q is 1. In some embodiments, X² is —(CH₂)_{q}—, wherein q is 2 or 3.

In certain embodiments, L¹ is of formula:

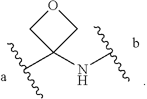 [SMILES: *NC1(*)COC1]

In certain embodiments, both X¹ and X² are bonds. In certain embodiments, both X¹ and X² are —O—. In certain embodiments, both X¹ and X² are —NR^{f-}. In certain embodiments, both X¹ and X² are —NH—. In certain embodiments, both X¹ and X² are —C(R^{d})₂—. In certain embodiments, X¹ is —(CH₂)_{q}—, and X² is —(CH₂)ₜ—, wherein each of q and t is independently 1, 2, or 3. In certain embodiments, both X¹ and X² are —CH₂—. In certain embodiments, X¹ is a bond, and X² is —O—. In certain embodiments, X¹ is a bond, and X² is —NR^{f}—. In certain embodiments, X¹ is a bond, and X² is —NH—. In certain embodiments, X¹ is a bond, and X² is —C(R^{d})₂—. In certain embodiments, X¹ is a bond, and X² is —(CH₂)ₜ—. In certain embodiments, X¹ is —O—, and X² is a bond. In certain embodiments, X¹ is —O—, and X² is —NR^{f}—. In certain embodiments, X¹ is —O—, and X² is —NH—. In certain embodiments, X¹ is —O—, and X² is —C(R^{d})₂—. In certain embodiments, X¹ is —O—, and X² is —CH₂—. In certain embodiments, X¹ is —O—, and X² is —(CH₂)ₜ—. In certain embodiments, X¹ is —NR^{f}—, and X² is a bond. In certain embodiments, X¹ is —NH—, and X² is a bond. In certain embodiments, X¹ is —NR^{f}—, and X² is —O—. In certain embodiments, X¹ is —NH—, and X² is —O—. In certain embodiments, X¹ is —NR^{f}—, and X² is —C(R^{d})₂⁻. In certain embodiments, X¹ is —NR^{f}—, and X² is —CH₂—. In certain embodiments, X¹ is —NR^{f}—, and X² is —(CH₂)ₜ—. In certain embodiments, X¹ is —NH—, and X² is —C(R^{d})₂—. In certain embodiments, X¹ is —NH—, and X² is —CH₂—. In certain embodiments, X¹ is —NH—, and X² is —(CH₂)ₜ—. In certain embodiments, X¹ is —C(R^{d})₂—, and X² is a bond. In certain embodiments, X¹ is —C(R^{d})₂—, and X² is —NR^{f}—. In certain embodiments, X¹—C(R^{d})₂—, and X² is —NH—. In certain embodiments, X¹ is —C(R^{d})₂—, and X² is —O—. In certain embodiments, X¹ is —C(R^{d})₂—, and X² is —(CH₂)ₜ—. In certain embodiments, X¹ is —CH₂—, and X² is a bond. In certain embodiments, X¹ is —CH₂—, and X² is —NR^{f}—. In certain embodiments, X¹—CH₂—, and X² is —NH—. In certain embodiments, X¹ is —CH₂—, and X² is —O—. In certain embodiments, X¹ is —(CH₂)_{q}—, and X² is a bond. In certain embodiments, X¹ is —(CH₂)_{q}—, and X² is —O—. In certain embodiments, X¹ is —(CH₂)_{q}—, and X² is a —NR^{f}— bond. In certain embodiments, X¹ is —(CH₂)_{q}—, and X² is —NH—. In certain embodiments, X¹ is —(CH₂)_{q}—, and X² is —C(R^{d})₂⁻.

In certain embodiments, L¹ is of formula:

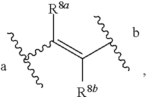

wherein the position labeled a is attached a carbon atom and the position labeled b is attached to a sulfur atom, and [symbol] indicates either a cis or trans configuration for the double bond with respect to positions a and b. In some embodiments, X¹ is —O—. In some embodiments, X¹ is —NR^{f}—. In some embodiments, X¹ is —NH—.

In some embodiments, L¹ is of formula:

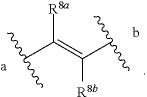

In some embodiments, L¹ is of formula:

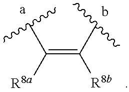

In certain embodiments, L¹ is of formula:

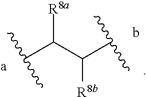

The carbon to which R^{8a} is attached may be in either the (R) or (S) configuration. The carbon to which R8b is attached may be in either the (R) or (S) configuration.

In certain embodiments, L¹ is of formula:

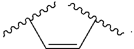 [SMILES: */C=C\*]

In certain embodiments, L¹ is of formula:

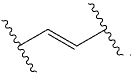 [SMILES: */C=C/*]

In certain embodiments, at least one of R^{8a} and R^{8b} is hydrogen. In certain embodiments, at least one of R^{8a} and R^{8b} is halogen. In some embodiments, at least one of R^{8a} and R^{8b} is —F. In some embodiments, at least one of R^{8a} and R^{8b} is —Cl, —Br, or —I. In certain embodiments, at least one of R8^{a} and R^{8b} is optionally substituted C₁₋₆ alkyl. In certain embodiments, at least one of R^{8a} and R^{8b} is unsubstituted C₁₋₆ alkyl. In certain embodiments, at least one of R^{8a} and R^{8b} is methyl. In certain embodiments, at least one of R^{8a} and R^{8b} is ethyl, propyl, or butyl.

In certain embodiments, both R^{8a} and R^{8b} are hydrogen. In certain embodiments, both R^{8a} and R^{8b} are halogen. In some embodiments, both R^{8a} and R^{8b} are —F. In some embodiments, both R^{8a} and R^{8b} are —Cl, —Br, or —I. In certain embodiments, both R^{8a} and R^{8b} are optionally substituted C₁₋₆ alkyl. In certain embodiments, both R^{8a} and R^{8b} are unsubstituted C₁₋₆ alkyl. In certain embodiments, both R^{8a} and R^{8b} are methyl. In certain embodiments, both R^{8a} and R^{8b} are ethyl, propyl, or butyl.

In certain embodiments, R^{8a} is hydrogen. In certain embodiments, R^{8a} is halogen. In some embodiments, R^{8a} is —F. In some embodiments, at least one of R^{8a} is —Cl, —Br, or —I. In certain embodiments, R^{8a} is optionally substituted C₁₋₆ alkyl. In certain embodiments, R^{8a} is unsubstituted C₁₋₆ alkyl. In certain embodiments, R^{8a} is methyl. In certain embodiments, R^{8a} is ethyl, propyl, or butyl. In certain embodiments, R^{8a} is —OR^{e}, e.g., —OH. In certain embodiments, R^{8a} is —N(R^{e})₂. In certain embodiments, R^{8a} is —NHR^{e}, e.g., —NH₂.

In certain embodiments, R^{8b} is hydrogen. In certain embodiments, R^{8b} is halogen. In some embodiments, R^{8b} is —F. In some embodiments, at least one of R^{8b} is —Cl, —Br, or —I. In certain embodiments, R^{8b} is optionally substituted C₁₋₆ alkyl. In certain embodiments, R^{8b} is unsubstituted C₁₋₆ alkyl. In certain embodiments, R^{8b} is methyl. In certain embodiments, R^{8b} is ethyl, propyl, or butyl. In certain embodiments, R^{8b} is —OR^{e}, e.g., —OH. In certain embodiments, R^{8b} is —N(R^{e})₂. In certain embodiments, R^{8b} is —NHR^{e}, e.g., —NH₂.

### Group G²

As generally defined herein, G² is —S(═O)₂—, —P(═O)(R^{e}), —P(═O)(OR^{e})—, —P(═O)(N(R^{e})₂)—, —P(═S)(R^{e}), —P(═S)(OR^{e})—, —P(═S)(N(R^{e})₂)—, —Si(OR^{e})₂—, —C(═O)—, —C(═S)—, —C(═NR^{f})—, —(CH₂)ₕ—,

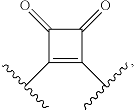 [SMILES: *c1c(*)c(=O)c1=O]

or optionally substituted monocyclic 5- or 6-membered heteroarylene, wherein 1, 2, 3, or 4 atoms in the heteroarylene ring system are independently oxygen, nitrogen, or sulfur.

In certain embodiments, G² is —S(═O)₂—, —P(═O)(OR^{e})—, —P(═O)(N(R^{e})₂)—, —Si(OR^{e})₂—, or is of formula:

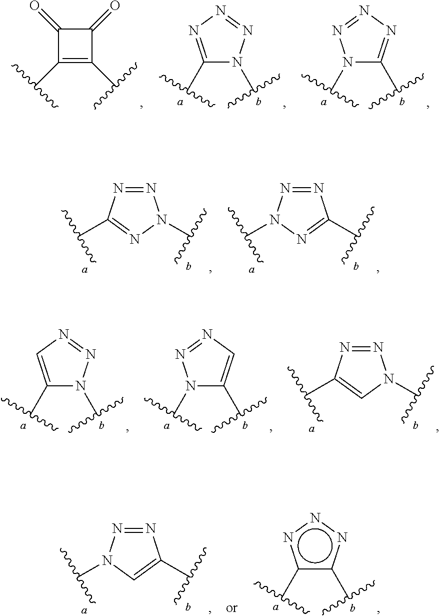

wherein G² is oriented such that the position labeled a is attached to L, and the position labeled b is attached to X¹.

In certain embodiments, G² is —S(═O)₂— or is of formula:

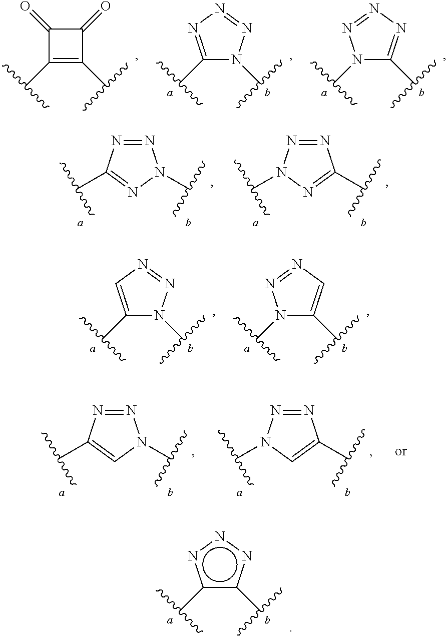

In certain embodiments, G² is —S(═O)₂—.

In certain embodiments, G² is —P(═O)(R^{e})—. In certain embodiments, G² is —P(═O)(OR^{e})—. In certain embodiments, G² is —P(═O)(OH)—. In certain embodiments, G² is —P(═O)(OR^{e})—, and R^{e} is optionally substituted alkyl. In certain embodiments, G² is —P(═O)(OR^{e})—, and R^{e} is unsubstituted C₁₋₆ alkyl. In certain embodiments, G² is —P(═O)(OMe)-. In certain embodiments, G² is —P(═O)(OR^{e})—, and R^{e} is optionally substituted acyl. In certain embodiments, G² is —P(═O)(OR^{e})—, and R^{e} is an oxygen protecting group (e.g., silyl, TBDPS, TBDMS, TIPS, TES, TMS, MOM, THP, t-Bu, Bn, allyl, acetyl, pivaloyl, or benzoyl).

In certain embodiments, G² is —P(═O)(N(R^{e})₂)—. In certain embodiments, G² is —P(═O)(NHR^{e})—. In certain embodiments, G² is —P(═O)(NH₂)—. In certain embodiments, G² is —P(═O)(N(R^{e})₂)—, and each R^{e} is independently optionally substituted alkyl. In certain embodiments, G² is —P(═O)(N(R^{e})₂)—, and each R^{e} is independently unsubstituted C₁₋₆ alkyl. In certain embodiments, G² is —P(═O)(NHR^{e})—, and R^{e} is optionally substituted alkyl. In certain embodiments, G² is —P(═O)(NHR^{e})—, and R^{e} is unsubstituted C₁₋₆ alkyl. In certain embodiments, G² is —P(═O)(NHR^{e})—, and R^{e} is optionally substituted acyl. In certain embodiments, G² is —P(═O)(NHR^{e})—, and R^{e} is a nitrogen protecting group (e.g., Bn, Boc, Cbz, Fmoc, trifluoroacetyl, triphenylmethyl, acetyl, tosyl, nosyl, brosyl, mesyl, or triflyl). In certain embodiments, G² is —P(═O)(N(R^{e})₂)—, and both R^{e} are joined to form an optionally substituted heterocyclic ring (e.g., piperidinyl, piperizinyl). In certain embodiments, G² is —P(═S)(R^{e}), —P(═S)(OR^{e})—, or —P(═S)(N(R^{e})₂)—.

In certain embodiments, G² is —Si(OR^{e})₂⁻. In certain embodiments, G² is —Si(OH)₂—. In certain embodiments, G² is —Si(OR^{e})(OH)—. In certain embodiments, G² is —Si(OMe)(OH)—. In certain embodiments, G² is —Si(OMe)₂-. In certain embodiments, G² is —Si(OR^{e})₂—, and each R^{e} is independently optionally substituted alkyl. In certain embodiments, G is —Si(OR^{e})₂—, and each R^{e} is independently unsubstituted C₁₋₆ alkyl. In certain embodiments, G² is —Si(OR^{e})(OH)—, and R^{e} is optionally substituted alkyl. In certain embodiments, G² is —Si(OR^{e})(OH)—, and R^{e} is unsubstituted C₁₋₆ alkyl. In certain embodiments, G² is —Si(OR^{e})₂—, each R^{e} is an oxygen protecting group (e.g., silyl, TBDPS, TBDMS, TIPS, TES, TMS, MOM, THP, t-Bu, Bn, allyl, acetyl, pivaloyl, or benzoyl). In certain embodiments, G² is —Si(OR^{e})₂—, and both R^{e} are joined to form an optionally substituted heterocyclic ring.

In certain embodiments, G² is —C(═O)—. In certain embodiments, G² is —C(═S)—. In certain embodiments, G² is —C(═NR^{f})—. In certain embodiments, G² is —C(═NH)—.

In certain embodiments, G² is —(CH₂)ₕ—, and h is 1. In certain embodiments, G² is —(CH₂)ₕ—, and h is 2. In certain embodiments, G² is —(CH₂)ₕ—, and h is 3.

In certain embodiments, G² is of formula:

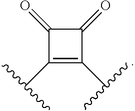 [SMILES: *c1c(*)c(=O)c1=O]

In certain embodiments, G² is optionally substituted monocyclic 5- or 6-membered heteroarylene, wherein 1, 2, 3, or 4 atoms in the heteroarylene ring system are independently oxygen, nitrogen, or sulfur. In certain embodiments, G² is furanylene, thienylene, pyrrolylene, oxazolylene, isoxazolylene, thiazolylene, isothiazolylene, imidazolylene, or pyrazolylene. In certain embodiments, G² is of formula:

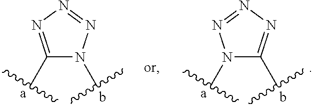 [SMILES: *c1nnnn1* ; *c1nnnn1*]

In certain embodiments, G² is of formula:

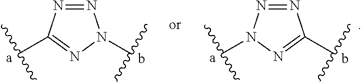 [SMILES: *c1nnn(*)n1 ; *c1nnn(*)n1]

In certain embodiments, G² is of formula:

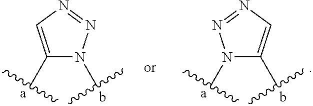 [SMILES: *c1cnnn1* ; *c1cnnn1*]

In certain embodiments, G² is of formula:

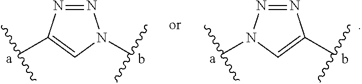 [SMILES: *c1cn(*)nn1 ; *c1cn(*)nn1]

In certain embodiments, G is of formula:

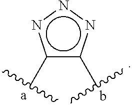

Groups R^{6a} and R^{6b}

As generally defined herein, each of R^{6a} and R^{6b} is independently hydrogen, halogen, or optionally substituted C₁₋₆ alkyl. The carbon to which R^{6a} and R^{6b} is attached may be in either the (R) or (S) configuration. In certain embodiments, at least one of R^{6a} and R^{6b} is hydrogen. In certain embodiments, at least one of R^{6a} and R^{6b} is halogen. In some embodiments, at least one of R^{6a} and R^{6b} is —F. In some embodiments, at least one of R^{6a} and R^{6b} is —Cl, —Br, or —I. In certain embodiments, at least one of R^{6a} and R^{6b} is optionally substituted C₁₋₆ alkyl. In certain embodiments, at least one of R^{6a} and R^{6b} is unsubstituted C₁₋₆ alkyl. In certain embodiments, at least one of R^{6a} and R^{6b} is methyl. In certain embodiments, at least one of R^{6a} and R^{6b} is ethyl, propyl, or butyl.

In certain embodiments, both R^{6a} and R^{6b} are hydrogen. In certain embodiments, both R^{6a} and R^{6b} are halogen. In some embodiments, both R^{6a} and R^{6b} are —F. In some embodiments, both R^{6a} and R^{6b} are —Cl, —Br, or —I. In certain embodiments, both R^{6a} and R^{6b} are optionally substituted C₁₋₆ alkyl. In certain embodiments, both R^{6a} and R^{6b} are unsubstituted C₁₋₆ alkyl. In certain embodiments, both R^{6a} and R^{6b} are methyl. In certain embodiments, both R^{6a} and R^{6b} are ethyl, propyl, or butyl.

In certain embodiments, R^{6a} is hydrogen. In certain embodiments, R^{6a} is halogen. In some embodiments, R^{6a} is —F. In some embodiments, at least one of R^{6a} is —Cl, —Br, or —I. In certain embodiments, R^{6a} is optionally substituted C₁₋₆ alkyl. In certain embodiments, R^{6a} is unsubstituted C₁₋₆ alkyl. In certain embodiments, R^{6a} is methyl. In certain embodiments, R^{6a} is ethyl, propyl, or butyl.

In certain embodiments, R^{6b} is hydrogen. In certain embodiments, R^{6b} is halogen. In some embodiments, R^{6b} is —F. In some embodiments, at least one of R^{6b} is —Cl, —Br, or —I. In certain embodiments, R^{6b} is optionally substituted C₁₋₆ alkyl. In certain embodiments, R^{6b} is unsubstituted C₁₋₆ alkyl. In certain embodiments, R^{6b} is methyl. In certain embodiments, R^{6b} is ethyl, propyl, or butyl.

Groups R^{7a} and R^{7b}

As generally defined herein, each of R^{7a} and R^{7b} is independently hydrogen, halogen, or optionally substituted C₁₋₆ alkyl. The carbon to which R^{7a} and R^{7b} is attached may be in either the (R) or (S) configuration. In certain embodiments, at least one of R^{7a} and R^{7b} is hydrogen. In certain embodiments, at least one of R^{7a} and R^{7b} is halogen. In some embodiments, at least one of R^{7a} and R^{7b} is —F. In some embodiments, at least one of R^{7a} and R^{7b} is —Cl, —Br, or —I. In certain embodiments, at least one of R^{7a} and R^{7b} is optionally substituted C₁₋₆ alkyl. In certain embodiments, at least one of R^{7a} and R^{7b} is unsubstituted C₁₋₆ alkyl. In certain embodiments, at least one of R^{7a} and R^{7b} is methyl. In certain embodiments, at least one of R^{7a} and R^{7b} is ethyl, propyl, or butyl.

In certain embodiments, both R^{7a} and R^{7b} are hydrogen. In certain embodiments, both R^{7a} and R^{7b} are halogen. In some embodiments, both R^{7a} and R^{7b} are —F. In some embodiments, both R^{7a} and R^{7b} are —Cl, —Br, or —I. In certain embodiments, both R^{7a} and R^{7b} are optionally substituted C₁₋₆ alkyl. In certain embodiments, both R^{7a} and R^{7b} are unsubstituted C₁₋₆ alkyl. In certain embodiments, both R^{7a} and R^{7b} are methyl. In certain embodiments, both R^{7a} and R^{7b} are ethyl, propyl, or butyl.

In certain embodiments, R^{7a} is hydrogen. In certain embodiments, R^{7a} is halogen. In some embodiments, R^{7a} is —F. In some embodiments, at least one of R^{7a} is —Cl, —Br, or —I. In certain embodiments, R^{7a} is optionally substituted C₁₋₆ alkyl. In certain embodiments, R^{7a} is unsubstituted C₁₋₆ alkyl. In certain embodiments, R^{7a} is methyl. In certain embodiments, R^{7a} is ethyl, propyl, or butyl.

In certain embodiments, R^{7b} is hydrogen. In certain embodiments, R^{7b} is halogen. In some embodiments, R^{7b} is —F. In some embodiments, at least one of R^{7b} is —Cl, —Br, or —I. In certain embodiments, R^{7b} is optionally substituted C₁₋₆ alkyl. In certain embodiments, R^{7b} is unsubstituted C₁₋₆ alkyl. In certain embodiments, R^{7b} is methyl. In certain embodiments, R^{7b} is ethyl, propyl, or butyl.

Groups R^{9a} and R^{9b}

As generally defined herein, each of R^{9a} and R^{9b} is independently hydrogen, halogen, optionally substituted C₁₋₆ alkyl, —OR^{e}, or —N(R^{e})₂. The carbon to which R^{9a} and R^{9b} is attached may be in either the (R) or (S) configuration. In certain embodiments, at least one of R^{9a} and R^{9b} is hydrogen. In certain embodiments, at least one of R^{9a} and R^{9b} is halogen. In some embodiments, at least one of R^{9a} and R^{9b} is —F. In some embodiments, at least one of R^{9a} and R^{9b} is —Cl, —Br, or —I. In certain embodiments, at least one of R^{9a} and R^{9b} is optionally substituted C₁₋₆ alkyl. In certain embodiments, at least one of R^{9a} and R^{9b} is unsubstituted C₁₋₆ alkyl. In certain embodiments, at least one of R^{9a} and R^{9b} is methyl. In certain embodiments, at least one of R^{9a} and R^{9b} is ethyl, propyl, or butyl.

In certain embodiments, both R^{9a} and R^{9b} are hydrogen. In certain embodiments, both R^{9a} and R^{9b} are halogen. In some embodiments, both R^{9a} and R^{9b} are —F. In some embodiments, both R^{9a} and R^{9b} are —Cl, —Br, or —I. In certain embodiments, both R^{9a} and R^{9b}are optionally substituted C₁₋₆ alkyl. In certain embodiments, both R^{9a} and R^{9b} are unsubstituted C₁₋₆ alkyl. In certain embodiments, both R^{9a} and R^{9b} are methyl. In certain embodiments, both R^{9a} and R^{9b} are ethyl, propyl, or butyl.

In certain embodiments, R^{9a} is hydrogen. In certain embodiments, R^{9a} is halogen. In some embodiments, R^{9a} is —F. In some embodiments, at least one of R^{9a} is —Cl, —Br, or —I. In certain embodiments, R^{9a} is optionally substituted C₁₋₆ alkyl. In certain embodiments, R^{9a} is unsubstituted C₁₋₆ alkyl. In certain embodiments, R^{9a} is methyl. In certain embodiments, R^{9a} is ethyl, propyl, or butyl. In certain embodiments, R^{9a} is —OR^{e}, e.g., —OH. In certain embodiments, R^{9a} is —N(R^{e})₂. In certain embodiments, R^{9a} is —NHR^{e}, e.g., —NH₂.

In certain embodiments, R^{9b} is hydrogen. In certain embodiments, R^{9b} is halogen. In some embodiments, R^{9b} is —F. In some embodiments, at least one of R^{9b} is —Cl, —Br, or —I. In certain embodiments, R^{9b} is optionally substituted C₁₋₆ alkyl. In certain embodiments, R^{9b} is unsubstituted C₁₋₆ alkyl. In certain embodiments, R^{9b} is methyl. In certain embodiments, R^{9b} is ethyl, propyl, or butyl. In certain embodiments, R^{9b} is —OR^{e}, e.g., —OH. In certain embodiments, R^{9b} is —N(R^{e})₂. In certain embodiments, R^{9b} is —NHR^{e}, e.g., —NH₂.

### Groups A-B and X⁵

As generally defined herein, A-B is —(R^{A})₂C—C(R^{B})₂— or —R^{A}C═CR^{B-}. In some embodiments, A-B is —(R^{A})₂C—C(R^{B})₂—. In some embodiments, A-B is —(R^{A})(H)C—C(H)(R^{B})—. In some embodiments, A-B is —R^{A}C═CR^{B}—. In some embodiments, A-B is —HC═CH—. In some embodiments, A-B is —(N(R^{e})₂)(H)C—C(H)(N(R^{e})₂)—. In some embodiments, A-B is —(NHR^{e})(H)C—C(H)(NHR^{e})—. In some embodiments, A-B is —(NH₂)(H)C—C(H)(NH₂)—. In some embodiments, A-B is —(OR^{S1})(H)C—C(H)(OR^{S2})—. In some embodiments, A-B is —(OH)(H)C—C(H)(OH)—. In some embodiments, A is —CF₂— or —CCl₂—. In some embodiments, B is —CF₂— or —CCl₂—. In some embodiments, A is —CHF— or —CHCl—. In some embodiments, B is —CHF— or —CHCl—.

As generally defined herein, each occurrence of R^{A} is independently hydrogen, halogen, optionally substituted alkyl, optionally substituted acyl, —OR^{S1}, or —N(R^{e})₂. In some embodiments, at least one R^{A} is hydrogen. In some embodiments, at least one R^{A} is halogen. In some embodiments, at least one R^{A} is unsubstituted C₁₋₆ alkyl, e.g., methyl. In some embodiments, at least one R^{A} is optionally substituted acyl. In some embodiments, at least one R^{A} is —OR^{S1}, e.g., —OH. In some embodiments, at least one R^{A} is —N(R^{e})₂, e.g., —NH₂.

As generally defined herein, each occurrence of R^{B} is independently hydrogen, halogen, optionally substituted alkyl, optionally substituted acyl, —OR^{S2}, or —N(R^{e})₂. In some embodiments, at least one R^{B} is hydrogen. In some embodiments, at least one R^{B} is halogen. In some embodiments, at least one R^{B} is unsubstituted C₁₋₆ alkyl, e.g., methyl. In some embodiments, at least one R^{B} is optionally substituted acyl. In some embodiments, at least one R^{B} is —OR^{S1}, e.g., —OH. In some embodiments, at least one R^{B} is —N(R^{e})₂, e.g., —NH₂.

As generally defined herein, each of R^{S1} and R^{S2} is independently hydrogen, optionally substituted C₁₋₆ alkyl, optionally substituted acyl, or an oxygen protecting group, or R^{S1} and R^{S2} are joined to form an optionally substituted heterocyclic ring. The carbon to which R^{S5} is attached may be in either the (R) or (S) configuration. The carbon to which R^{S2} is attached may be in either the (R) or (S) configuration.

In certain embodiments, at least one of R^{S1} and R^{S2} is hydrogen. In certain embodiments, at least one of R^{S1} and R^{S2} is optionally substituted C₁₋₆ alkyl. In certain embodiments, at least one of R^{S1} and R^{S2} is unsubstituted C₁₋₆ alkyl. In certain embodiments, at least one of R^{S1} and R^{S2} is methyl. In certain embodiments, at least one of R^{S1} and R^{S2} is ethyl, propyl, or butyl. In certain embodiments, at least one of R^{S1} and R^{S2} is acyl (e.g., —C(═O)(R^{e}), —C(═O)O(R^{e}), —C(═O)NH(R^{e}), —C(═O)N(R^{e})₂). In certain embodiments, at least one of R^{S1} and R^{S2} is an oxygen protecting group. In some embodiments, at least one of R^{S1} and R^{S2} is silyl (e.g., TMS, TBDMS, TIPS). In some embodiments, at least one of R^{S5} and R^{S2} is acetyl (Ac), benzyl (Bn), benzoyl (Bz), or methoxymethyl ether (MOM).

In certain embodiments, both R^{S1} and R^{S2} are hydrogen. In certain embodiments, both R^{S1} and R^{S2} are optionally substituted C₁₋₆ alkyl. In certain embodiments, both R^{S1} and R^{S2} are unsubstituted C₁₋₆ alkyl. In certain embodiments, both R^{S1} and R^{S2} are methyl. In certain embodiments, both R^{S1} and R^{S2} are ethyl, propyl, or butyl. In certain embodiments, both R^{S1} and R^{S2} are acyl (e.g., —C(═O)(R^{e}), —C(═O)O(R^{e}), —C(═O)NH(R^{e}), —C(═O)N(R^{e})₂). In certain embodiments, both R^{S1} and R^{S2} are oxygen protecting groups. In some embodiments, both R^{S1} and R^{S2} are silyl (e.g., TMS, TBDMS, TIPS). In some embodiments, both R^{S1} and R^{S2} are acetyl (Ac), benzyl (Bn), benzoyl (Bz), or methoxymethyl ether (MOM).

In certain embodiments, R^{S1} is hydrogen. In certain embodiments, R^{S1} is optionally substituted C₁₋₆ alkyl. In certain embodiments, R^{S1} is unsubstituted C₁₋₆ alkyl. In certain embodiments, R^{S1} is methyl. In certain embodiments, R^{S1} is ethyl, propyl, or butyl. In certain embodiments, R^{S1} is acyl (e.g., —C(═O)(R^{e}), —C(═O)O(R^{e}), —C(═O)NH(R^{e}), —C(═O)N(R^{e})₂). In certain embodiments, R^{S1} is an oxygen protecting group. In some embodiments, R^{S1} is silyl (e.g., TMS, TBDMS, TIPS). In some embodiments, R^{S1} is acetyl (Ac), benzyl (Bn), benzoyl (Bz), or methoxymethyl ether (MOM).

In certain embodiments, R^{S2} is hydrogen. In certain embodiments, R^{S2} is optionally substituted C₁₋₆ alkyl. In certain embodiments, R^{S2} is unsubstituted C₁₋₆ alkyl. In certain embodiments, R^{S2} is methyl. In certain embodiments, R^{S2} is ethyl, propyl, or butyl. In certain embodiments, R^{S2} is acyl (e.g., —C(═O)(R^{e}), —C(═O)O(R^{e}), —C(═O)NH(R^{e}), —C(═O)N(R^{e})₂). In certain embodiments, R^{S2} is an oxygen protecting group. In some embodiments, R^{S2} is silyl (e.g., TMS, TBDMS, TIPS). In some embodiments, R^{S2} is acetyl (Ac), benzyl (Bn), benzoyl (Bz), or methoxymethyl ether (MOM).

In certain embodiments, R^{S1} and R^{S2} are joined to form an optionally substituted heterocyclic ring. In certain embodiments, R^{S1} and R^{S2} are taken together to form a cyclic acetal (e.g., —C(CH₃)₂—).

As generally defined herein, X⁵ is —O—, —S—, —C(R^{d})₂—, or —NR^{f}—. In certain embodiments, X⁵ is —O—. In certain embodiments, X⁵ is —S—. In certain embodiments, X⁵ is —C(R^{d})₂—. In certain embodiments, X⁵ is —CH₂—, —CHMe-, or —CMe₂-. In certain embodiments, X⁵ is —NR^{f}—, e.g., —NH—. In some embodiments, X⁵ is —NR^{f}—, wherein R^{f} is a nitrogen protecting group, e.g., —NAc—. In certain embodiments, X⁵ is —C(R^{d})₂—, and both R^{d} are halogen. In certain embodiments, X⁵ is —CF₂—.

Groups L, V¹, V², V³, V⁷, V⁸, and V⁹.

As generally defined herein, L^{S} is a bond, —O—, —NR^{f}—, optionally substituted alkylene, optionally substituted alkenylene, optionally substituted alkynylene, optionally substituted acylene, or optionally substituted arylene. In certain embodiments, L^{S} is a bond. In certain embodiments, L^{S} is —O—. In certain embodiments, L^{S} is —NR^{f}—, e.g. —NH—. In certain embodiments, L^{S} is optionally substituted alkylene. In certain embodiments, L^{S} is optionally substituted arylene. In certain embodiments, L^{S} is unsubstituted C₁₋₄ alkylene, e.g., methylene, ethyelene. In certain embodiments, L^{S} is optionally substituted alkenylene, e.g., —HC═CH—. In certain embodiments, L^{S} is optionally substituted alkynylene, e.g., —C—C—. In certain embodiments, L^{S} is optionally substituted acylene. In some embodiments, L^{S} is —C(═O)—, —C(═O)O—, —OC(═O)—, —C(═O)NR^{f}—, —NR^{f}C(═O)—, —C(═O)NH—, or —NHC(═O)—.

As generally defined herein, each of V¹, V², V³, V⁷, V⁸, and V⁹ is independently N, NR^{V}, or CR^{V}, valence permitting depending on the other ring positions. In certain embodiments, V¹ is N. In certain embodiments, V¹ is CR^{V}. In certain embodiments, V¹ is NR^{V}. In some embodiments, V¹ is CH. In certain embodiments, V² is N. In certain embodiments, V is CR^{V}. In certain embodiments, V² is NR^{V}. In some embodiments, V² is CH. In certain embodiments, V³ is N. In certain embodiments, V³ is CR^{V}. In certain embodiments, V³ is NR^{V}. In some embodiments, V³ is CH. In certain embodiments, V⁷ is N. In certain embodiments, V⁷ is CR^{V}. In certain embodiments, V⁷ is NR^{V}. In some embodiments, V⁷ is CH. In certain embodiments, V⁸ is N. In certain embodiments, V⁸ is CR^{V}. In certain embodiments, V⁸ is NR^{V}. In some embodiments, V⁸ is CH. In certain embodiments, V⁹ is N. In certain embodiments, V⁹ is CR^{V}. In certain embodiments, V⁹ is NR^{V}. In some embodiments, V⁹ is CH.

For each occurrence of V¹, V², V³, V⁷, V⁸, and V⁹ which is NR^{V} or CR^{V}, R^{V} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —NO₂, —CN. In —OR^{e}, or —N(R^{e})₂. In certain embodiments, R^{V} is halogen. In certain embodiments, R^{V} is —F. In certain embodiments, R^{V} is —Cl, —Br, or —F. In certain embodiments, R^{V} is optionally substituted C₁₋₆ alkyl. In certain embodiments, R^{V} is unsubstituted C₁₋₆ alkyl. In certain embodiments, R^{V} is methyl. In certain embodiments, R^{V} is ethyl, propyl, or butyl. In certain embodiments, R^{V} is —NO₂. In certain embodiments, R^{V} is —CN. In certain embodiments, R^{V} is —OR^{e} (e.g. —OH, —OMe, —O(C₁₋₆ alkyl)) In certain embodiments, R^{V} is —OR^{e}, and R^{e} is an oxygen protecting group. In certain embodiments, R^{V} is —N(R^{e})₂ (e.g., —NH₂, —NMe₂, —NH(C₁₋₆ alkyl)). In certain embodiments, R^{V} is —NHR^{e}, and R^{e} is a nitrogen protecting group. In certain embodiments, R^{V} is optionally substituted acyl (e.g., —C(═O)(R^{e}), —C(═O)O(R^{e}), —C(═O)NH(R^{e}), —C(═O)N(R^{e})₂). In some embodiments, R^{V} is —C(═O)OMe. In some embodiments, R^{V} is —C(═O)OH.

In certain embodiments, R^{V} is optionally substituted alkyl, e.g., optionally substituted C₁₋₆ alkyl, optionally substituted C₁₋₂ alkyl, optionally substituted C₂₋₃ alkyl, optionally substituted C₃₋₄ alkyl, optionally substituted C₄₋₅ alkyl, or optionally substituted C₅₋₆ alkyl. In certain embodiments, R^{V} is methyl. In certain embodiments, R^{V} is ethyl, propyl, or butyl. In certain embodiments, R^{V} is optionally substituted alkenyl, e.g., optionally substituted C₂₋₆ alkenyl. In certain embodiments, R^{V} is vinyl, allyl, or prenyl. In certain embodiments, R^{V} is optionally substituted alkynyl, e.g., C₂₋₆ alkynyl.

In certain embodiments, R^{V} is optionally substituted carbocyclyl, e.g., optionally substituted C₃₋₆ carbocyclyl, optionally substituted C₃₋₄ carbocyclyl, optionally substituted C₄₋₅ carbocyclyl, or optionally substituted C₅₋₆ carbocyclyl. In certain embodiments R^{V} is optionally substituted heterocyclyl, e.g., optionally substituted 3-6 membered heterocyclyl, optionally substituted 3-4 membered heterocyclyl, optionally substituted 4-5 membered heterocyclyl, or optionally substituted 5-6 membered heterocyclyl.

In certain embodiments, R^{V} is optionally substituted aryl, e.g., optionally substituted phenyl. In certain embodiments, R^{V} is optionally substituted heteroaryl, e.g., optionally substituted 5-6 membered heteroaryl, or optionally substituted 9-10 membered bicyclic heteroaryl. In certain embodiments, R^{V} is optionally substituted aralkyl, e.g., optionally substituted benzyl. In certain embodiments, R^{V} is optionally substituted heteroaralkyl, e.g., methyl substituted with a 5-6-membered heteroaryl ring.

In certain embodiments, the group attached to L^{S} is of formula:

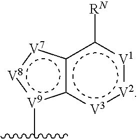

In certain embodiments, the group attached to L^{S} is of formula:

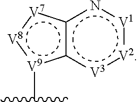

In certain embodiments, the group attached to L^{S} is of formula:

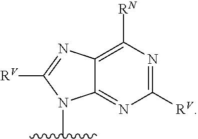

In certain embodiments, the group attached to L^{S} is of formula:

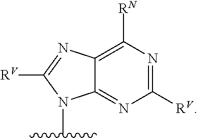

In certain embodiments, the group attached to L^{S} is of formula:

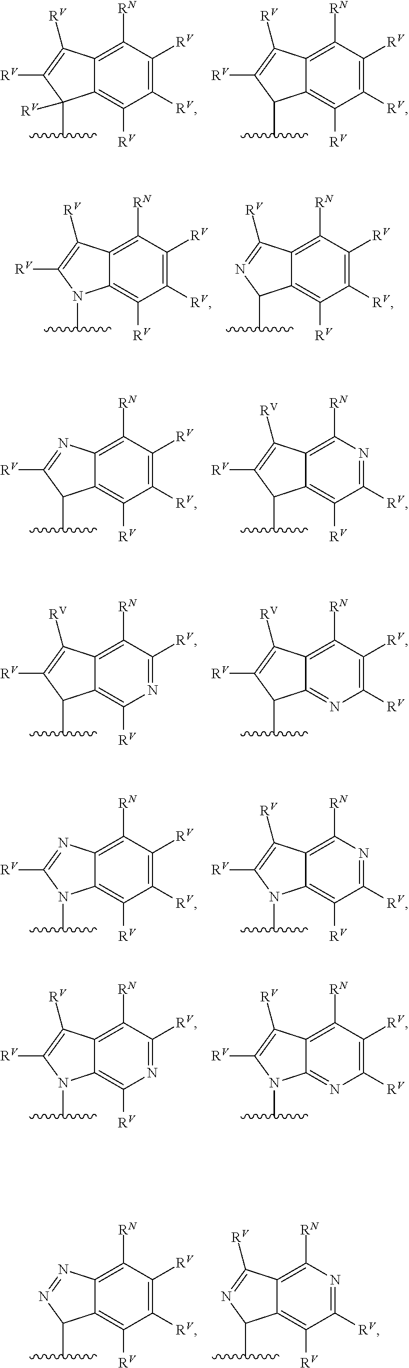

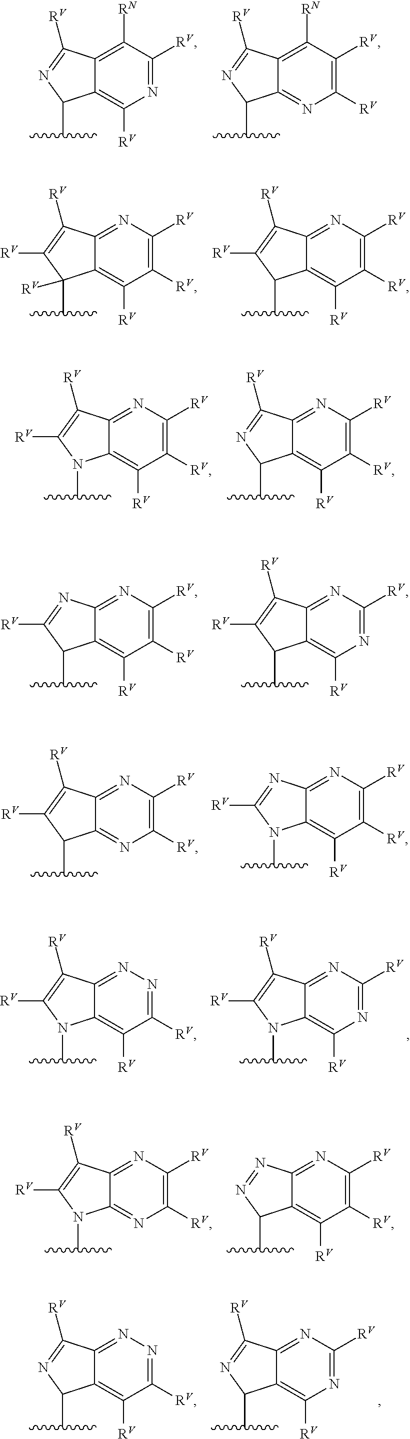

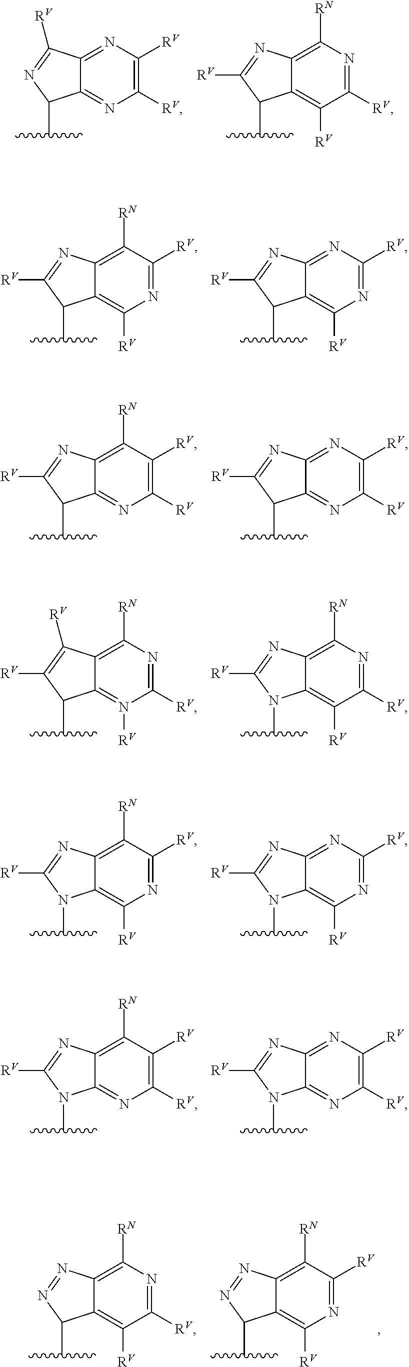

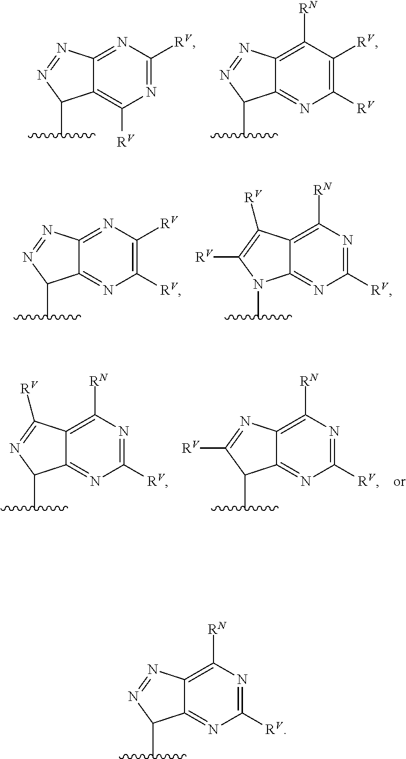

In certain embodiments, the group attached to LS is of formula:

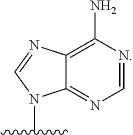 [SMILES: *n1cnc2c(N)ncnc21]

In certain embodiments, the group attached to L^{S} is of formula:

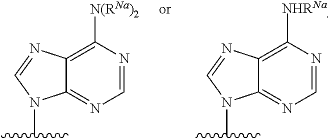

In certain embodiments, the group attached to L^{S} is of formula:

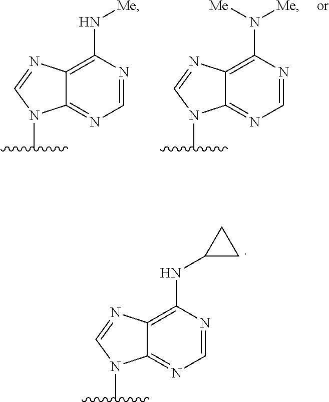 [SMILES: *n1cnc2c(NC)ncnc21 ; *n1cnc2c(N(C)C)ncnc21 ; *n1cnc2c(NC3CC3)ncnc21]

In certain embodiments, the group attached to L^{S} is of formula:

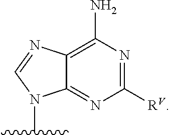

In certain embodiments, the group attached to L^{S} is of formula:

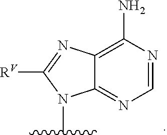

In certain embodiments, the group attached to L is of formula:

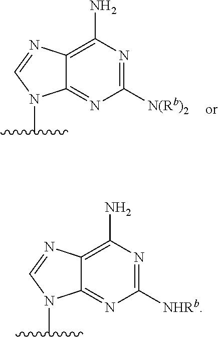

In certain embodiments, the group attached to L^{S} is of formula:

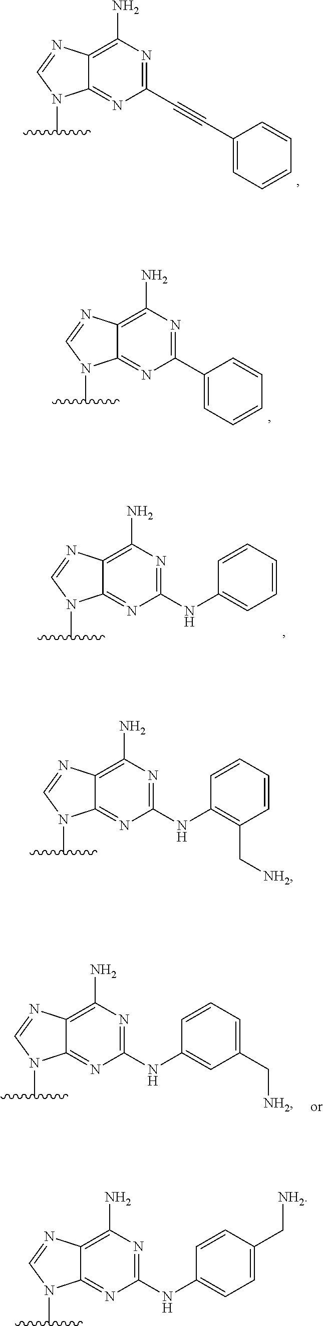 [SMILES: *n1cnc2c(N)nc(C#Cc3ccccc3)nc21 ; *n1cnc2c(N)nc(-c3ccccc3)nc21 ; *n1cnc2c(N)nc(Nc3ccccc3)nc21 ; *n1cnc2c(N)nc(Nc3ccccc3CN)nc21 ; *n1cnc2c(N)nc(Nc3cccc(CN)c3)nc21 ; *n1cnc2c(N)nc(Nc3ccc(CN)cc3)nc21]

Group V^{N} and R^{N}

As generally defined herein, V^{N} is N, NR^{V}, or CR^{V}, valence permitting depending on the other ring positions. In certain embodiments, V^{N} is N. In certain embodiment V^{N} is NR^{V}. In certain embodiments, V^{N} is CR^{V}. In certain embodiments, V^{N} is CH.

As generally defined herein, R^{N} is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —OR^{e}, or —N(R^{Na})₂.

In certain embodiments, R^{N} is hydrogen. In certain embodiments, R^{N} is halogen. In certain embodiments, R^{N} is —F. In certain embodiments, R^{N} is —Cl, —Br, or —F. In certain embodiments, R^{N} is —NO₂. In certain embodiments, R^{N} is —CN. In certain embodiments, R^{N} is —OR^{e} (e.g. —OH, —OMe, —O(C₁₋₆ alkyl)). In certain embodiments, R^{N} is —OR^{e}, and R^{e} is an oxygen protecting group. In certain embodiments, R^{N} is —N(R^{Na})₂ (e.g., —NH₂, —NMe₂, —NH(C₁₋₆ alkyl)). In certain embodiments, R^{N} is —NHR^{Na}, and R^{Na} is a nitrogen protecting group. In certain embodiments, R^{N} is optionally substituted acyl (e.g., —C(═O)(R^{e}), —C(═O)O(R^{e}), —C(═O)NH(R^{e}), —C(═O)N(R^{e})₂). In some embodiments, R^{N} is —C(═O)OMe. In some embodiments, R^{N} is —C(═O)OH.

In certain embodiments, R^{N} is optionally substituted alkyl, e.g., optionally substituted C₁₋₆ alkyl, optionally substituted C₁₋₂ alkyl, optionally substituted C₂₋₃ alkyl, optionally substituted C₃₋₄ alkyl, optionally substituted C₄₋₅ alkyl, or optionally substituted C₅₋₆ alkyl. In certain embodiments, R^{N} is methyl. In certain embodiments, R^{N} is ethyl, propyl, or butyl. In certain embodiments, R^{N} is optionally substituted alkenyl, e.g., optionally substituted C₂₋₆ alkenyl. In certain embodiments, R^{N} is vinyl, allyl, or prenyl. In certain embodiments, R^{N} is optionally substituted alkynyl, e.g., C₂₋₆ alkynyl.

In certain embodiments, R^{N} is optionally substituted carbocyclyl, e.g., optionally substituted C₃₋₆ carbocyclyl, optionally substituted C₃₋₄ carbocyclyl, optionally substituted C₄₋₅ carbocyclyl, or optionally substituted C₅₋₆ carbocyclyl. In certain embodiments R^{N} is optionally substituted heterocyclyl, e.g., optionally substituted 3-6 membered heterocyclyl, optionally substituted 3-4 membered heterocyclyl, optionally substituted 4-5 membered heterocyclyl, or optionally substituted 5-6 membered heterocyclyl.

In certain embodiments, R^{N} is optionally substituted aryl, e.g., optionally substituted phenyl. In certain embodiments, R^{N} is optionally substituted heteroaryl, e.g., optionally substituted 5-6 membered heteroaryl, or optionally substituted 9-10 membered bicyclic heteroaryl. In certain embodiments, R^{N} is optionally substituted aralkyl, e.g., optionally substituted benzyl. In certain embodiments, R^{N} is optionally substituted heteroaralkyl, e.g., methyl substituted with a 5-6-membered heteroaryl ring.

As generally defined herein, R^{Na} is independently hydrogen, optionally substituted C₁₋₆ alkyl, optionally substituted acyl, or a nitrogen protecting group, or both R^{Na} are joined to form and optionally substituted heterocyclic or optionally substituted heteroaryl ring. In certain embodiments, at least one occurrence of R^{Na} is hydrogen. In certain embodiments, at least one occurrence of R^{Na} is optionally substituted C₁₋₆ alkyl. In certain embodiments, at least one occurrence of R^{Na} is unsubstituted C₁₋₆ alkyl. In certain embodiments, at least one occurrence of R^{Na} is methyl. In certain embodiments, at least one occurrence of R^{Na} is ethyl, propyl, or butyl. In certain embodiments, at least one occurrence of R^{Na} is acyl (e.g., —C(═O)(R^{e}), —C(═O)O(R^{e}), —C(═O)NH(R^{e}), —C(═O)N(R^{e})₂). In certain embodiments, at least one occurrence of R^{Na} is a nitrogen protecting group. In some embodiments, at least one occurrence of R^{Na} is alkoxycarbonyl (e.g., Cbz, BOC, FMOC). In some embodiments, at least one occurrence of R^{Na} is acetyl (Ac), benzyl (Bn), or benzoyl (Bz). In some embodiments, at least one occurrence of R^{Na} is sulfonyl (e.g., tosyl, nosyl, mesyl).

In certain embodiments, both occurrences of R^{Na} are hydrogen. In certain embodiments, both occurrences of R^{Na} are optionally substituted C₁₋₆ alkyl. In certain embodiments, both occurrences of R^{Na} are unsubstituted C₁₋₆ alkyl. In certain embodiments, both occurrences of R^{Na} are methyl. In certain embodiments, both occurrences of R^{Na} are ethyl, propyl, or butyl. In certain embodiments, both occurrences of R^{Na} are acyl (e.g., —C(═O)(R^{e}), —C(═O)O(R^{e}), —C(═O)NH(R^{e}), —C(═O)N(R^{e})₂). In certain embodiments, both occurrences of R^{Na} are nitrogen protecting groups. In some embodiments, both occurrences of R^{Na} are alkoxycarbonyl (e.g., Cbz, BOC, FMOC). In some embodiments, both occurrences of R^{Na} are acetyl (Ac), benzyl (Bn), or benzoyl (Bz). In some embodiments, both occurrences of R^{Na} are sulfonyl (e.g., tosyl, nosyl, mesyl).

In certain embodiments, one occurrence of R^{Na} is hydrogen, and the other occurrence of R^{Na} is optionally substituted C₁₋₆ alkyl. In certain embodiments, one occurrence of R^{Na} is hydrogen, and the other occurrence of R^{Na} unsubstituted C₁₋₆ alkyl. In certain embodiments, one occurrence of R^{Na} is hydrogen, and the other occurrence of R^{Na} is methyl. In certain embodiments, one occurrence of R^{Na} is hydrogen, and the other occurrence of R^{Na} is ethyl, propyl, or butyl. In certain embodiments, one occurrence of R^{Na} is hydrogen, and the other occurrence of R^{Na} is acyl (e.g., —C(═O)(R^{e}), —C(═O)O(R^{e}), —C(═O)NH(R^{e}), —C(═O)N(R^{e})₂). In certain embodiments, one occurrence of R^{Na} is hydrogen, and the other occurrence of R^{Na} is a nitrogen protecting group. In some embodiments, one occurrence of R^{Na} is hydrogen, and the other occurrence of R^{Na} is alkoxycarbonyl (e.g., Cbz, BOC, FMOC). In some embodiments, one occurrence of R^{Na} is hydrogen, and the other occurrence of R^{Na} is acetyl (Ac), benzyl (Bn), or benzoyl (Bz). In some embodiments, one occurrence of R^{Na} is hydrogen, and the other occurrence of R^{Na} is sulfonyl (e.g., tosyl, nosyl, mesyl).

In certain embodiments, both occurrences of R^{Na} are joined to form an optionally substituted heterocyclic ring (e.g., a 5- to 6-membered optionally substituted heterocyclic ring). In certain embodiments, both occurrences of R^{Na} are joined to form an optionally substituted heteroaryl ring (e.g., a 5- to 6-membered optionally substituted heteroaryl ring).

In certain embodiments, the compound of Formula (I) is a compound listed in Table 1, or a pharmaceutically acceptable salt, solvate, hydrate, polymorph, co-crystal, tautomer, stereoisomer, or prodrug thereof.

TABLE 1
Exemplary compounds of Formula (I).

| No. | Structure |
|---|---|
| 102 | 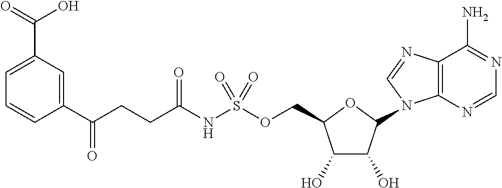 |
| 103 | 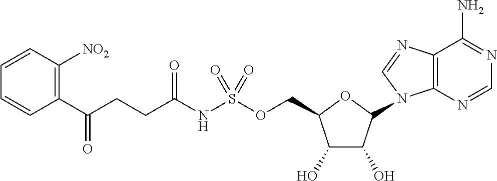 |
| 104 | 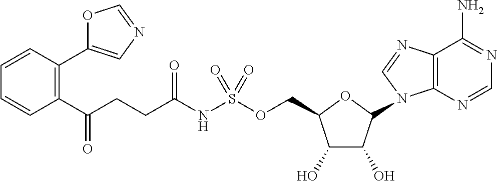 |
| 105 | 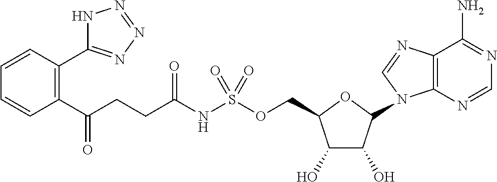 |
| 106 | 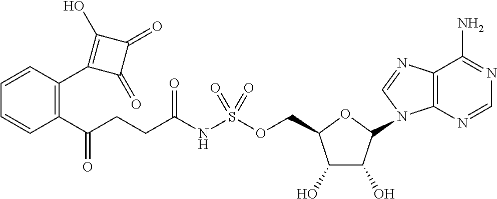 |
| 107 | 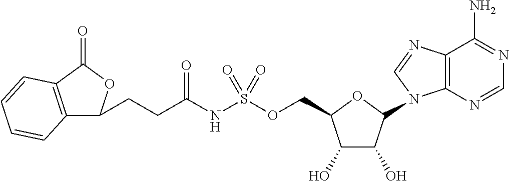 |
| 108 | 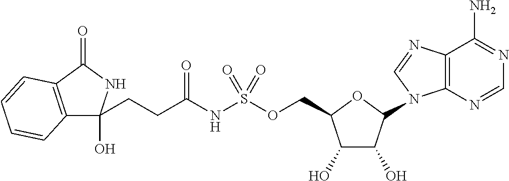 |
| 109 (also Cmpd. 2) | 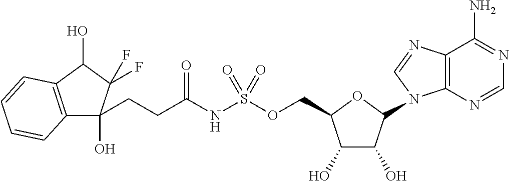 |
| 110 | 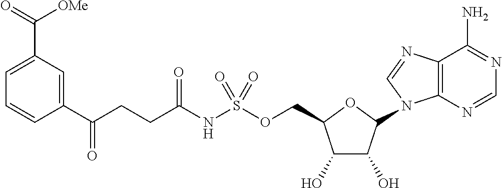 |
| 111 | 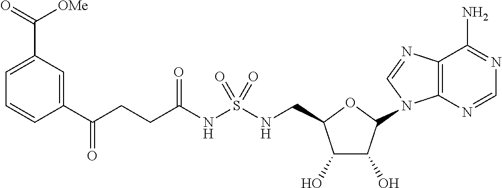 |
| 112 | 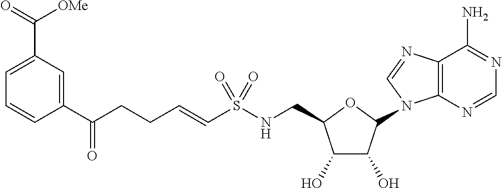 |
| 113 | 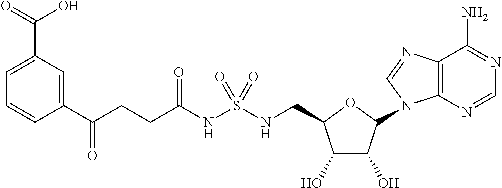 |
| 114 | 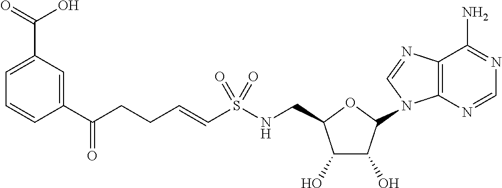 |
| 115 | 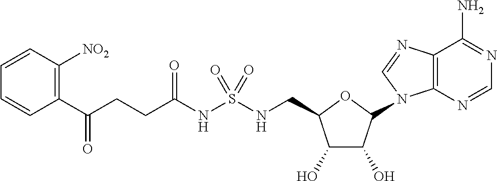 |
| 116 | 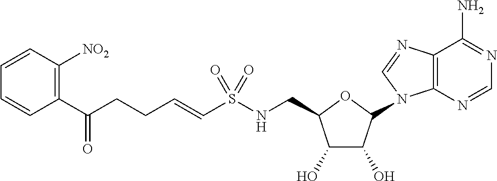 |
| 117 | 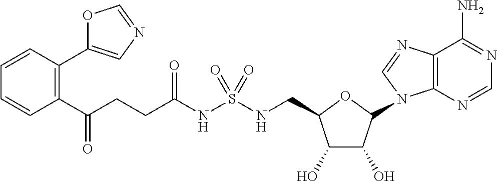 |
| 118 | 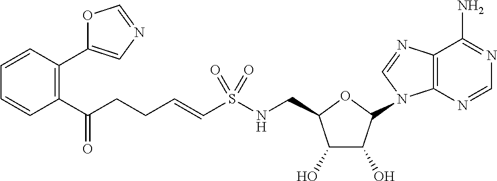 |
| 119 | 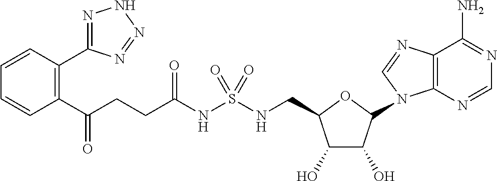 |
| 120 | 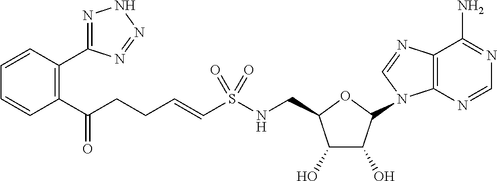 |
| 121 | 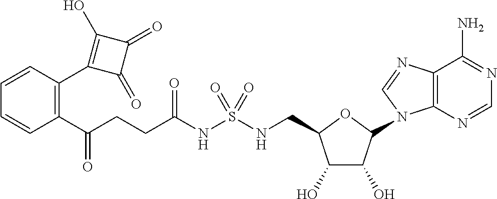 |
| 122 | 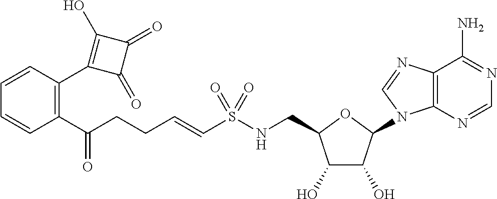 [SMILES: Nc1ncnc2c1ncn2[C@@H]1O[C@H](CNS(=O)(=O)/C=C/CCC(=O)c2ccccc2-c2c(O)c(=O)c2=O)[C@@H](O)[C@H]1O] |
| 123 | 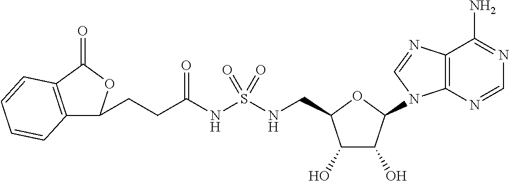 [SMILES: Nc1ncnc2c1ncn2[C@@H]1O[C@H](CNS(=O)(=O)NC(=O)CCC2OC(=O)c3ccccc32)[C@@H](O)[C@H]1O] |
| 124 | 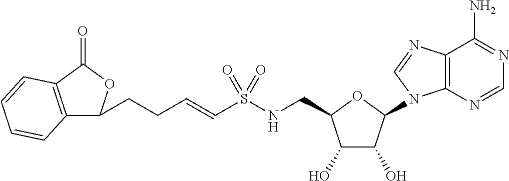 [SMILES: Nc1ncnc2c1ncn2[C@@H]1O[C@H](CNS(=O)(=O)/C=C/CCC2OC(=O)c3ccccc32)[C@@H](O)[C@H]1O] |
| 125 | 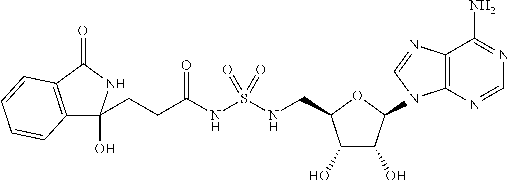 [SMILES: Nc1ncnc2c1ncn2[C@@H]1O[C@H](CNS(=O)(=O)NC(=O)CCC2(O)NC(=O)c3ccccc32)[C@@H](O)[C@H]1O] |
| 126 | 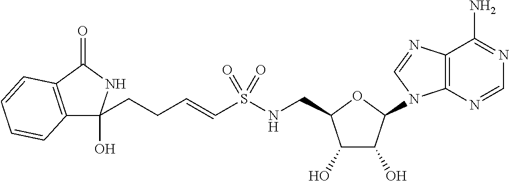 [SMILES: Nc1ncnc2c1ncn2[C@@H]1O[C@H](CNS(=O)(=O)/C=C/CCC2(O)NC(=O)c3ccccc32)[C@@H](O)[C@H]1O] |
| 127 | 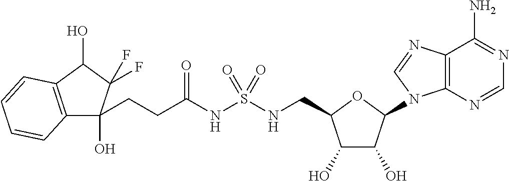 [SMILES: Nc1ncnc2c1ncn2[C@@H]1O[C@H](CNS(=O)(=O)NC(=O)CCC2(O)c3ccccc3C(O)C2(F)F)[C@@H](O)[C@H]1O] |
| 128 | 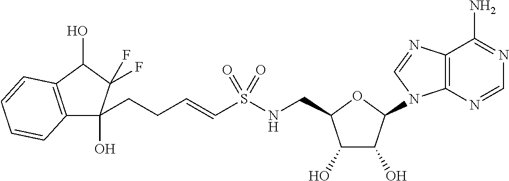 [SMILES: Nc1ncnc2c1ncn2[C@@H]1O[C@H](CNS(=O)(=O)/C=C/CCC2(O)c3ccccc3C(O)C2(F)F)[C@@H](O)[C@H]1O] |
| 129 | 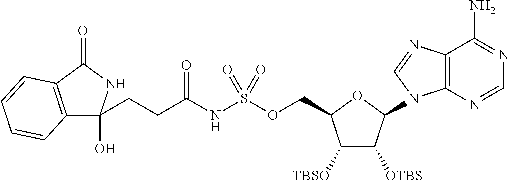 [SMILES: CC(C)(C)[Si](C)(C)O[C@@H]1[C@H](O[Si](C)(C)C(C)(C)C)[C@@H](COS(=O)(=O)NC(=O)CCC2(O)NC(=O)c3ccccc32)O[C@H]1n1cnc2c(N)ncnc21] |
| 130 | 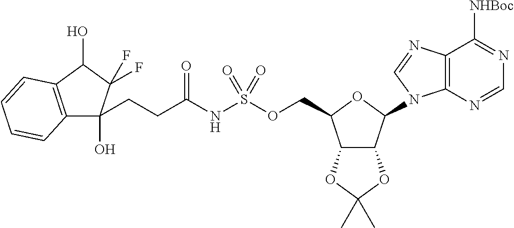 [SMILES: CC(C)(C)OC(=O)Nc1ncnc2c1ncn2[C@@H]1O[C@H](COS(=O)(=O)NC(=O)CCC2(O)c3ccccc3C(O)C2(F)F)[C@H]2OC(C)(C)O[C@H]21] |
| 131 | 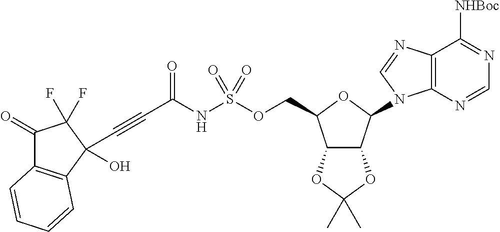 [SMILES: CC(C)(C)OC(=O)Nc1ncnc2c1ncn2[C@@H]1O[C@H](COS(=O)(=O)NC(=O)C#CC2(O)c3ccccc3C(=O)C2(F)F)[C@H]2OC(C)(C)O[C@H]21] |
| 132 | 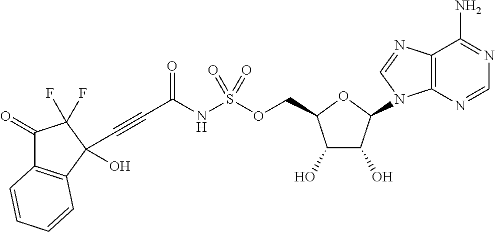 [SMILES: Nc1ncnc2c1ncn2[C@@H]1O[C@H](COS(=O)(=O)NC(=O)C#CC2(O)c3ccccc3C(=O)C2(F)F)[C@@H](O)[C@H]1O] |
| 133 | 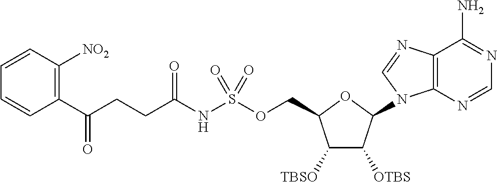 |
| 134 | 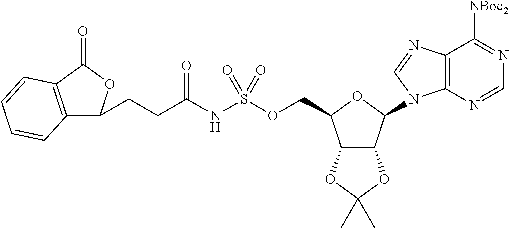 |
| 135 | 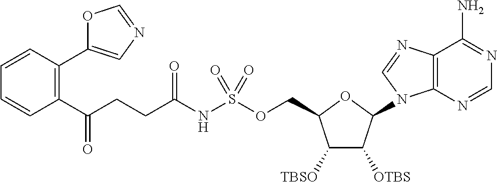 |
| 136 | 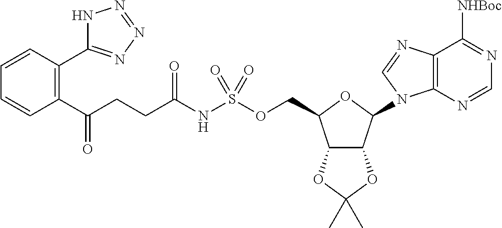 [SMILES: CC(C)(C)OC(=O)Nc1ncnc2c1ncn2[C@@H]1O[C@H](COS(=O)(=O)NC(=O)CCC(=O)c2ccccc2-c2nnn[nH]2)[C@H]2OC(C)(C)O[C@H]21] |
| 137 | 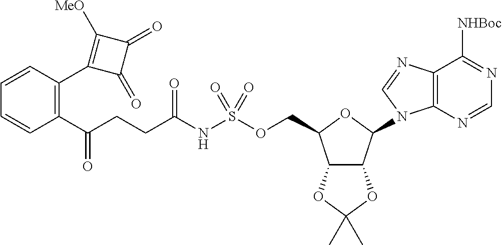 [SMILES: COc1c(-c2ccccc2C(=O)CCC(=O)NS(=O)(=O)OC[C@H]2O[C@@H](n3cnc4c(NC(=O)OC(C)(C)C)ncnc43)[C@@H]3OC(C)(C)O[C@@H]32)c(=O)c1=O] |
| 138 | 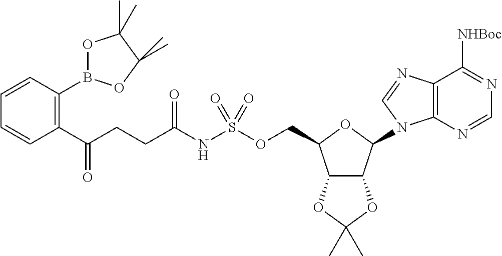 [SMILES: CC(C)(C)OC(=O)Nc1ncnc2c1ncn2[C@@H]1O[C@H](COS(=O)(=O)NC(=O)CCC(=O)c2ccccc2B2OC(C)(C)C(C)(C)O2)[C@H]2OC(C)(C)O[C@H]21] |
| 139 | 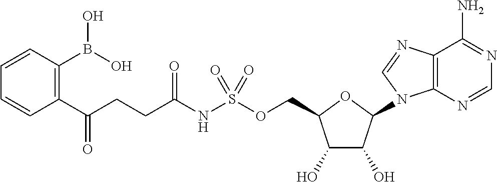 [SMILES: Nc1ncnc2c1ncn2[C@@H]1O[C@H](COS(=O)(=O)NC(=O)CCC(=O)c2ccccc2B(O)O)[C@@H](O)[C@H]1O] |
| 140 | 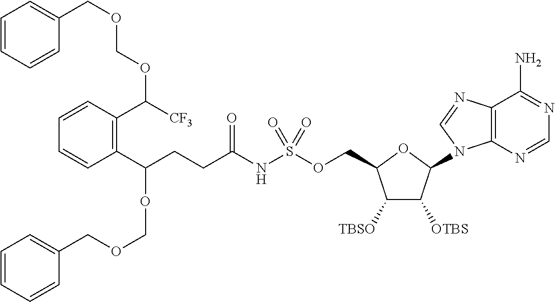 |
| 141 | 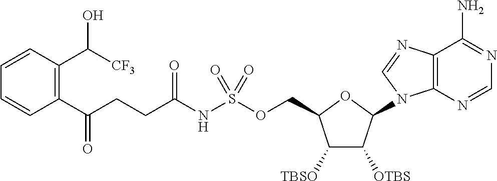 |
| 142 | 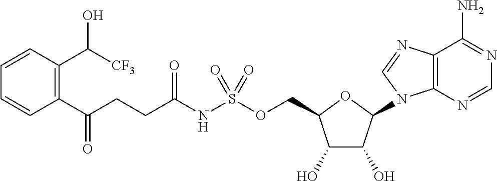 [SMILES: Nc1ncnc2c1ncn2[C@@H]1O[C@H](COS(=O)(=O)NC(=O)CCC(=O)c2ccccc2C(O)C(F)(F)F)[C@@H](O)[C@H]1O] |
| 143 | 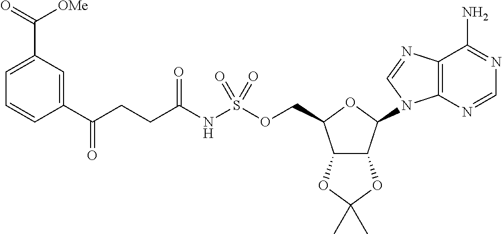 [SMILES: COC(=O)c1cccc(C(=O)CCC(=O)NS(=O)(=O)OC[C@H]2O[C@@H](n3cnc4c(N)ncnc43)[C@@H]3OC(C)(C)O[C@@H]32)c1] |
| 144 | 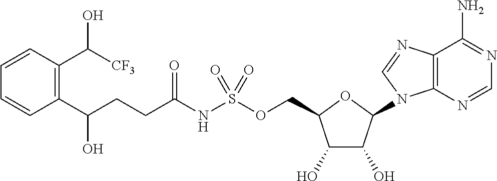 [SMILES: Nc1ncnc2c1ncn2[C@@H]1O[C@H](COS(=O)(=O)NC(=O)CCC(O)c2ccccc2C(O)C(F)(F)F)[C@@H](O)[C@H]1O] |

In certain embodiments, the compound of the invention is of formula:

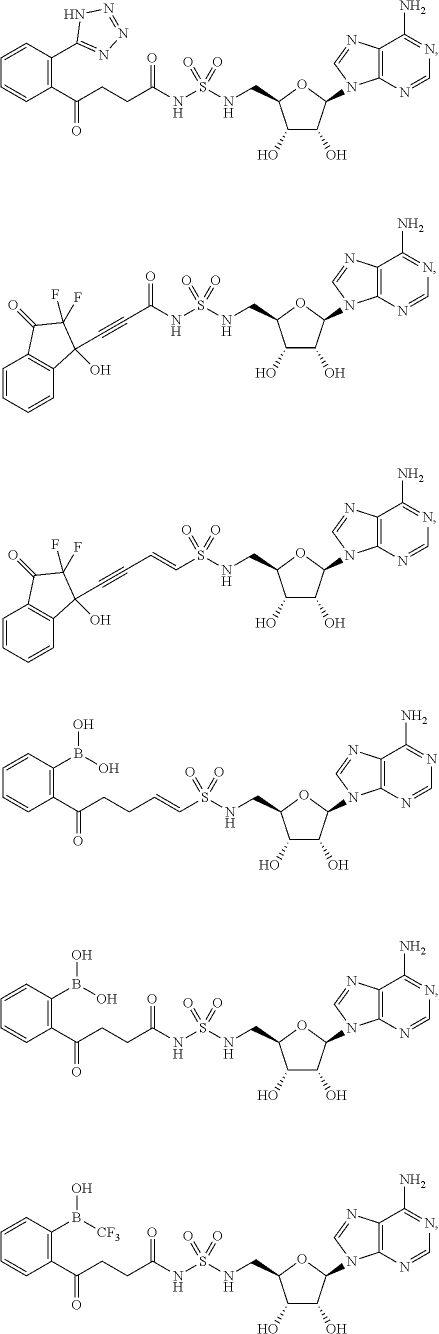

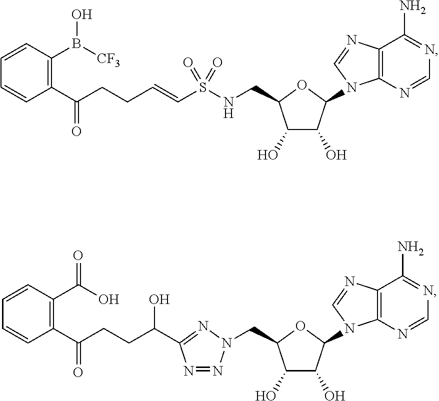 [SMILES: Nc1ncnc2c1ncn2[C@@H]1O[C@H](CNS(=O)(=O)/C=C/CCC(=O)c2ccccc2B(O)C(F)(F)F)[C@@H](O)[C@H]1O ; Nc1ncnc2c1ncn2[C@@H]1O[C@H](Cn2nnc(C(O)CCC(=O)c3ccccc3C(=O)O)n2)[C@@H](O)[C@H]1O]

or a pharmaceutically acceptable salt, solvate, hydrate, polymorph, co-crystal, tautomer, stereoisomer, or prodrug thereof. In certain embodiments, the compound of the invention is of formula:

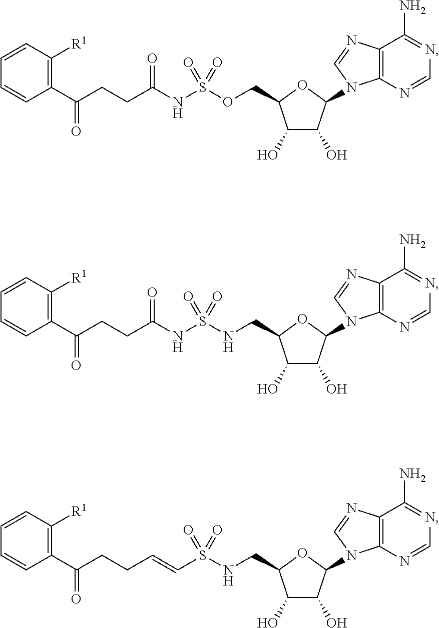

or a pharmaceutically acceptable salt, solvate, hydrate, polymorph, co-crystal, tautomer, stereoisomer, or prodrug thereof, wherein R¹ is unsubstituted isoxazolyl or unsubstituted tetrazolyl. In certain embodiments, the compound of the invention is of formula:

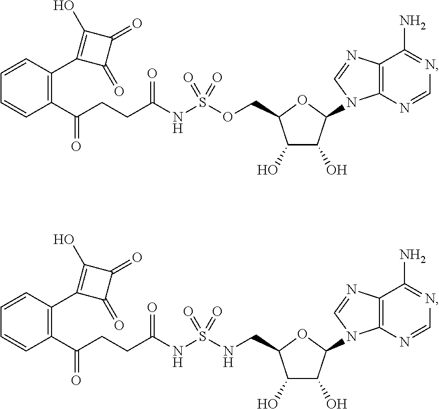 [SMILES: Nc1ncnc2c1ncn2[C@@H]1O[C@H](COS(=O)(=O)NC(=O)CCC(=O)c2ccccc2-c2c(O)c(=O)c2=O)[C@@H](O)[C@H]1O ; Nc1ncnc2c1ncn2[C@@H]1O[C@H](CNS(=O)(=O)NC(=O)CCC(=O)c2ccccc2-c2c(O)c(=O)c2=O)[C@@H](O)[C@H]1O]

or a pharmaceutically acceptable salt, solvate, hydrate, polymorph, co-crystal, tautomer, stereoisomer, or prodrug thereof. In certain embodiments, the compound of the invention is of formula:

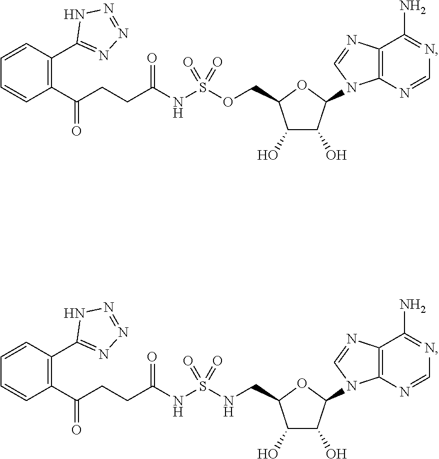 [SMILES: Nc1ncnc2c1ncn2[C@@H]1O[C@H](COS(=O)(=O)NC(=O)CCC(=O)c2ccccc2-c2nnn[nH]2)[C@@H](O)[C@H]1O ; Nc1ncnc2c1ncn2[C@@H]1O[C@H](CNS(=O)(=O)NC(=O)CCC(=O)c2ccccc2-c2nnn[nH]2)[C@@H](O)[C@H]1O]

or a pharmaceutically acceptable salt, solvate, hydrate, polymorph, co-crystal, tautomer, stereoisomer, or prodrug thereof. In certain embodiments, the compound of the invention is of formula:

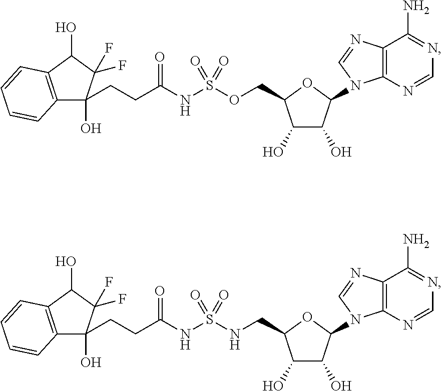 [SMILES: Nc1ncnc2c1ncn2[C@@H]1O[C@H](COS(=O)(=O)NC(=O)CCC2(O)c3ccccc3C(O)C2(F)F)[C@@H](O)[C@H]1O ; Nc1ncnc2c1ncn2[C@@H]1O[C@H](CNS(=O)(=O)NC(=O)CCC2(O)c3ccccc3C(O)C2(F)F)[C@@H](O)[C@H]1O]

or a pharmaceutically acceptable salt, solvate, hydrate, polymorph, co-crystal, tautomer, stereoisomer, or prodrug thereof. In certain embodiments, the pharmaceutically acceptable salt is an alkali metal salt (e.g., lithium salt, sodium salt, potassium salt). In certain embodiments, the pharmaceutically acceptable salt is a sodium salt.

In certain embodiments, the Compound 109 is selected from the group consisting of:

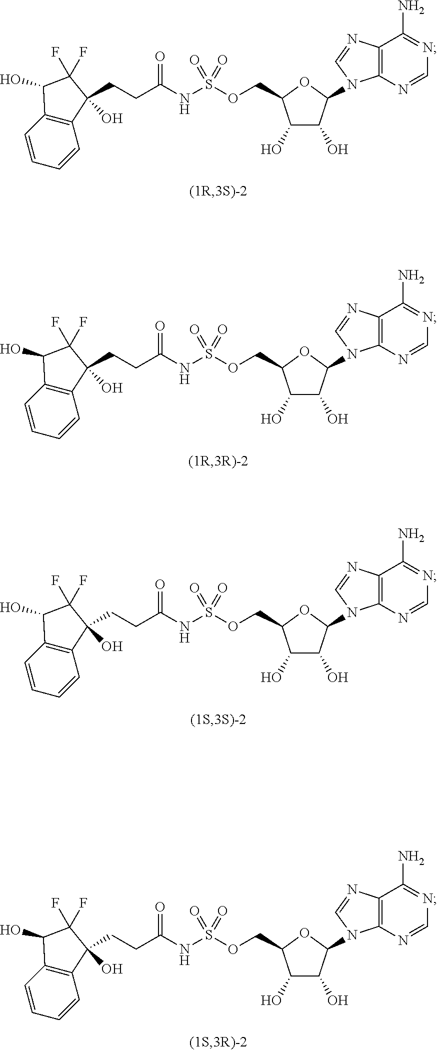 [SMILES: Nc1ncnc2c1ncn2[C@@H]1O[C@H](COS(=O)(=O)NC(=O)CC[C@@]2(O)c3ccccc3[C@H](O)C2(F)F)[C@@H](O)[C@H]1O ; Nc1ncnc2c1ncn2[C@@H]1O[C@H](COS(=O)(=O)NC(=O)CC[C@@]2(O)c3ccccc3[C@@H](O)C2(F)F)[C@@H](O)[C@H]1O ; Nc1ncnc2c1ncn2[C@@H]1O[C@H](COS(=O)(=O)NC(=O)CC[C@]2(O)c3ccccc3[C@H](O)C2(F)F)[C@@H](O)[C@H]1O ; Nc1ncnc2c1ncn2[C@@H]1O[C@H](COS(=O)(=O)NC(=O)CC[C@]2(O)c3ccccc3[C@@H](O)C2(F)F)[C@@H](O)[C@H]1O]

and pharmaceutically acceptable salts, solvates, hydrates, polymorphs, co-crystals, tautomers, stereoisomers, and prodrugs thereof; and mixtures thereof.

Compounds of Formula (I) and (Z) comprise a linker between the 5-membered ribose or ribose analog ring and the group of formula:

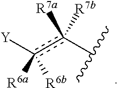

In certain embodiments, the linker is selected from Table 2.

TABLE 2
Exemplary linkers of compounds of Formula (I) or (Z).
Linker

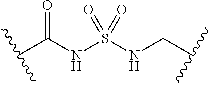 [SMILES: *CNS(=O)(=O)NC(*)=O]

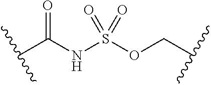 [SMILES: *COS(=O)(=O)NC(*)=O]

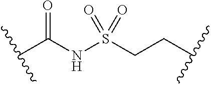 [SMILES: *CCS(=O)(=O)NC(*)=O]

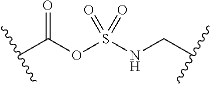 [SMILES: *CNS(=O)(=O)OC(*)=O]

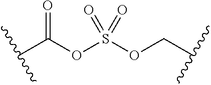 [SMILES: *COS(=O)(=O)OC(*)=O]

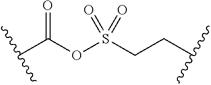 [SMILES: *CCS(=O)(=O)OC(*)=O]

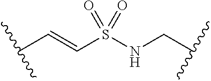 [SMILES: */C=C/S(=O)(=O)NC*]

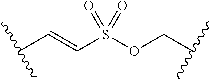 [SMILES: */C=C/S(=O)(=O)OC*]

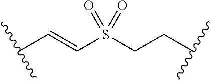 [SMILES: */C=C/S(=O)(=O)CC*]

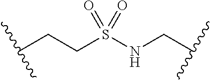 [SMILES: *CCS(=O)(=O)NC*]

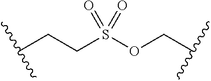 [SMILES: *CCS(=O)(=O)OC*]

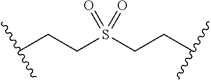 [SMILES: *CCS(=O)(=O)CC*]

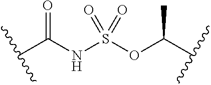 [SMILES: *C(=O)NS(=O)(=O)O[C@H](*)C]

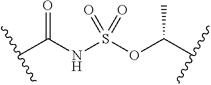 [SMILES: *C(=O)NS(=O)(=O)O[C@@H](*)C]

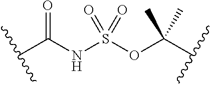

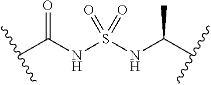 [SMILES: *C(=O)NS(=O)(=O)N[C@H](*)C]

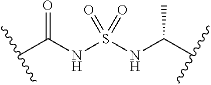 [SMILES: *C(=O)NS(=O)(=O)N[C@@H](*)C]

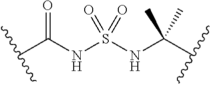

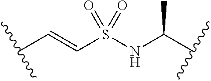 [SMILES: */C=C/S(=O)(=O)N[C@H](*)C]

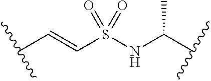 [SMILES: */C=C/S(=O)(=O)N[C@@H](*)C]

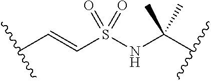

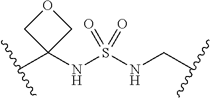 [SMILES: *CNS(=O)(=O)NC1(*)COC1]

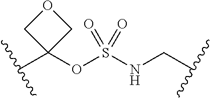 [SMILES: *CNS(=O)(=O)OC1(*)COC1]

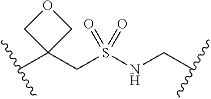 [SMILES: *CNS(=O)(=O)CC1(*)COC1]

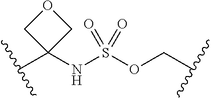 [SMILES: *COS(=O)(=O)NC1(*)COC1]

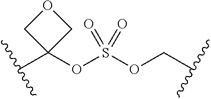 [SMILES: *COS(=O)(=O)OC1(*)COC1]

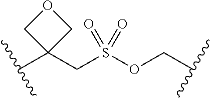 [SMILES: *COS(=O)(=O)CC1(*)COC1]

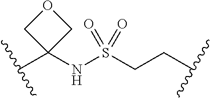 [SMILES: *CCS(=O)(=O)NC1(*)COC1]

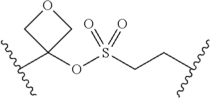 [SMILES: *CCS(=O)(=O)OC1(*)COC1]

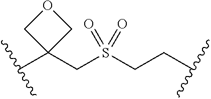 [SMILES: *CCS(=O)(=O)CC1(*)COC1]

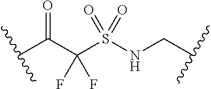 [SMILES: *CNS(=O)(=O)C(F)(F)C(*)=O]

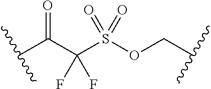 [SMILES: *COS(=O)(=O)C(F)(F)C(*)=O]

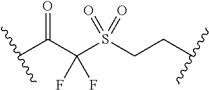 [SMILES: *CCS(=O)(=O)C(F)(F)C(*)=O]

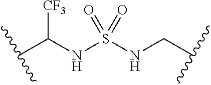 [SMILES: *CNS(=O)(=O)NC(*)C(F)(F)F]

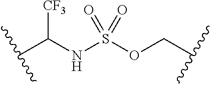 [SMILES: *COS(=O)(=O)NC(*)C(F)(F)F]

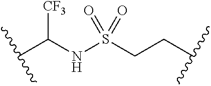 [SMILES: *CCS(=O)(=O)NC(*)C(F)(F)F]

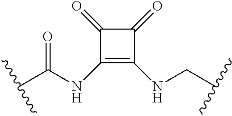 [SMILES: *CNc1c(NC(*)=O)c(=O)c1=O]

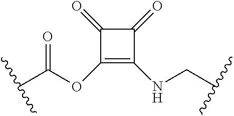 [SMILES: *CNc1c(OC(*)=O)c(=O)c1=O]

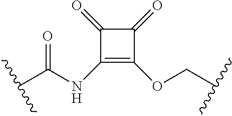 [SMILES: *COc1c(NC(*)=O)c(=O)c1=O]

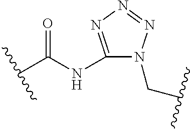 [SMILES: *Cn1nnnc1NC(*)=O]

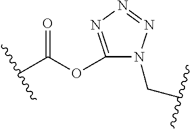 [SMILES: *Cn1nnnc1OC(*)=O]

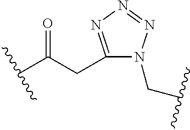 [SMILES: *Cn1nnnc1CC(*)=O]

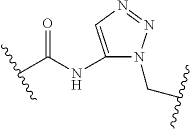 [SMILES: *Cn1nncc1NC(*)=O]

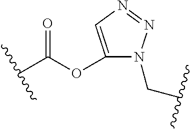 [SMILES: *Cn1nncc1OC(*)=O]

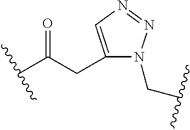 [SMILES: *Cn1nncc1CC(*)=O]

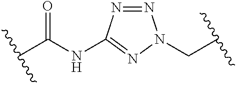 [SMILES: *Cn1nnc(NC(*)=O)n1]

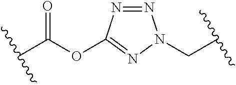 [SMILES: *Cn1nnc(OC(*)=O)n1]

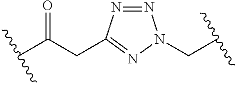 [SMILES: *Cn1nnc(CC(*)=O)n1]

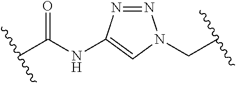 [SMILES: *Cn1cc(NC(*)=O)nn1]

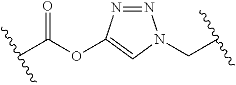 [SMILES: *Cn1cc(OC(*)=O)nn1]

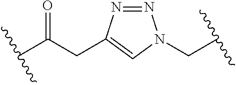 [SMILES: *Cn1cc(CC(*)=O)nn1]

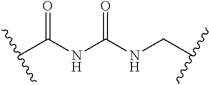 [SMILES: *CNC(=O)NC(*)=O]

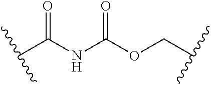 [SMILES: *COC(=O)NC(*)=O]

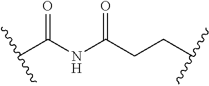 [SMILES: *CCC(=O)NC(*)=O]

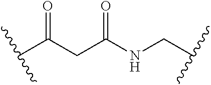 [SMILES: *CNC(=O)CC(*)=O]

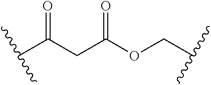 [SMILES: *COC(=O)CC(*)=O]

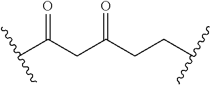 [SMILES: *CCC(=O)CC(*)=O]

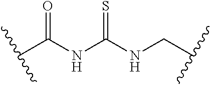 [SMILES: *CNC(=S)NC(*)=O]

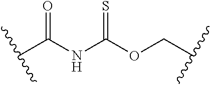 [SMILES: *COC(=S)NC(*)=O]

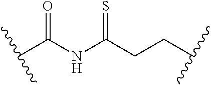 [SMILES: *CCC(=S)NC(*)=O]

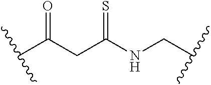 [SMILES: *CNC(=S)CC(*)=O]

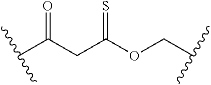 [SMILES: *COC(=S)CC(*)=O]

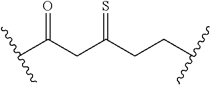 [SMILES: *CCC(=S)CC(*)=O]

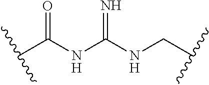 [SMILES: *CNC(=N)NC(*)=O]

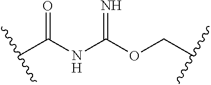 [SMILES: *COC(=N)NC(*)=O]

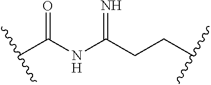 [SMILES: *CCC(=N)NC(*)=O]

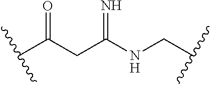 [SMILES: *CNC(=N)CC(*)=O]

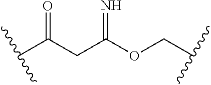 [SMILES: *COC(=N)CC(*)=O]

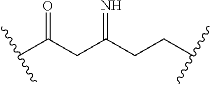 [SMILES: *CCC(=N)CC(*)=O]

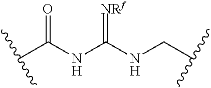

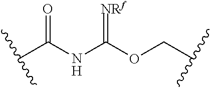

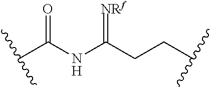

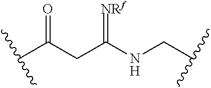

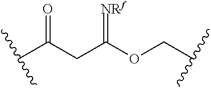

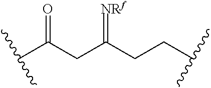

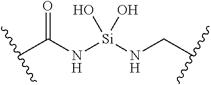 [SMILES: *CN[Si](O)(O)NC(*)=O]

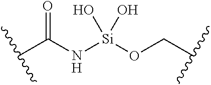 [SMILES: *CO[Si](O)(O)NC(*)=O]

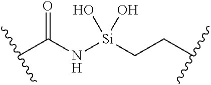 [SMILES: *CC[Si](O)(O)NC(*)=O]

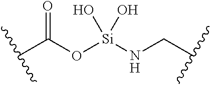 [SMILES: *CN[Si](O)(O)OC(*)=O]

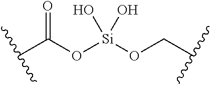 [SMILES: *CO[Si](O)(O)OC(*)=O]

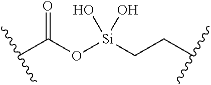 [SMILES: *CC[Si](O)(O)OC(*)=O]

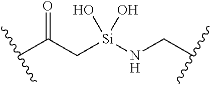 [SMILES: *CN[Si](O)(O)CC(*)=O]

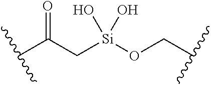 [SMILES: *CO[Si](O)(O)CC(*)=O]

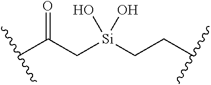 [SMILES: *CC[Si](O)(O)CC(*)=O]

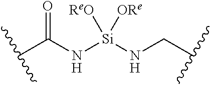

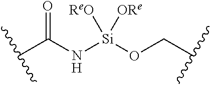

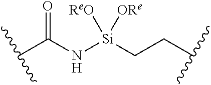

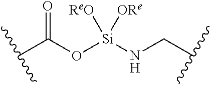

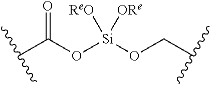

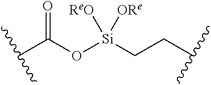

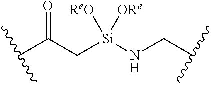

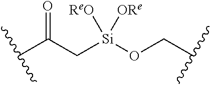

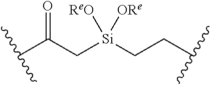

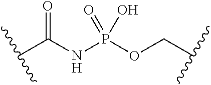 [SMILES: *COP(=O)(O)NC(*)=O]

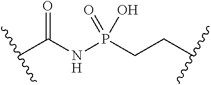 [SMILES: *CCP(=O)(O)NC(*)=O]

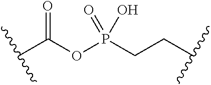 [SMILES: *CCP(=O)(O)OC(*)=O]

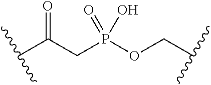 [SMILES: *COP(=O)(O)CC(*)=O]

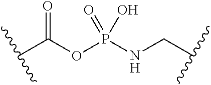 [SMILES: *CNP(=O)(O)OC(*)=O]

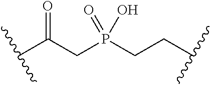 [SMILES: *CCP(=O)(O)CC(*)=O]

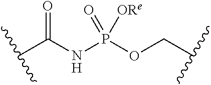

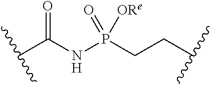

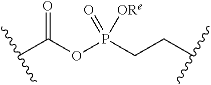

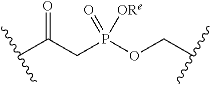

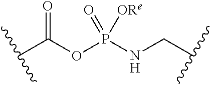

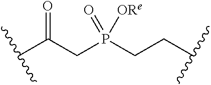

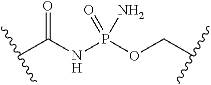 [SMILES: *COP(N)(=O)NC(*)=O]

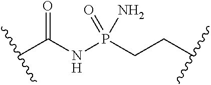 [SMILES: *CCP(N)(=O)NC(*)=O]

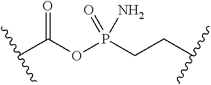 [SMILES: *CCP(N)(=O)OC(*)=O]

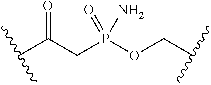 [SMILES: *COP(N)(=O)CC(*)=O]

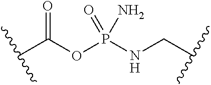 [SMILES: *CNP(N)(=O)OC(*)=O]

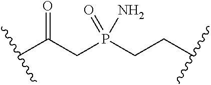 [SMILES: *CCP(N)(=O)CC(*)=O]

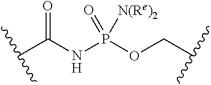

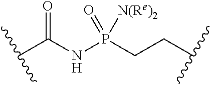

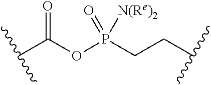

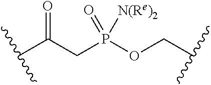

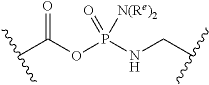

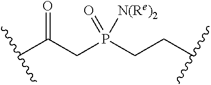

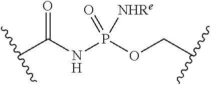

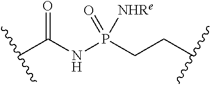

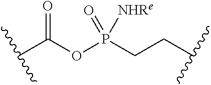

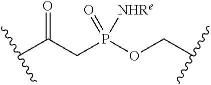

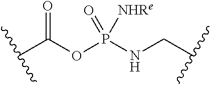

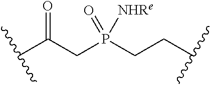

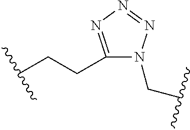 [SMILES: *CCc1nnnn1C*]

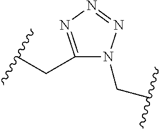 [SMILES: *Cc1nnnn1C*]

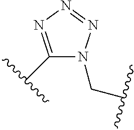 [SMILES: *Cn1nnnc1*]

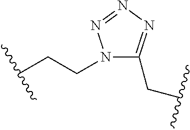 [SMILES: *CCn1nnnc1C*]

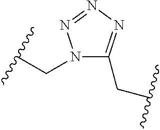 [SMILES: *Cc1nnnn1C*]

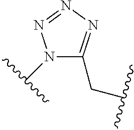 [SMILES: *Cc1nnnn1*]

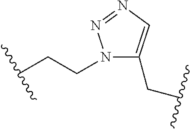 [SMILES: *CCn1nncc1C*]

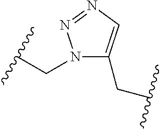 [SMILES: *Cc1cnnn1C*]

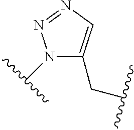 [SMILES: *Cc1cnnn1*]

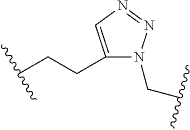 [SMILES: *CCc1cnnn1C*]

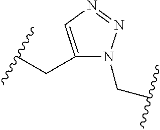 [SMILES: *Cc1cnnn1C*]

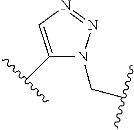 [SMILES: *Cn1nncc1*]

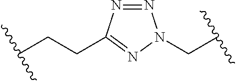 [SMILES: *CCc1nnn(C*)n1]

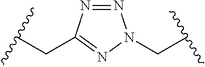 [SMILES: *Cc1nnn(C*)n1]

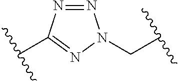 [SMILES: *Cn1nnc(*)n1]

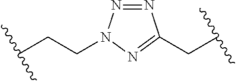 [SMILES: *CCn1nnc(C*)n1]

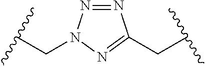 [SMILES: *Cc1nnn(C*)n1]

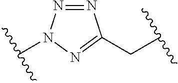 [SMILES: *Cc1nnn(*)n1]

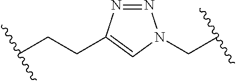 [SMILES: *CCc1cn(C*)nn1]

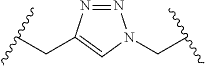 [SMILES: *Cc1cn(C*)nn1]

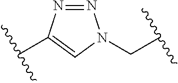 [SMILES: *Cn1cc(*)nn1]

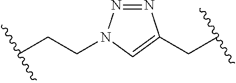 [SMILES: *CCn1cc(C*)nn1]

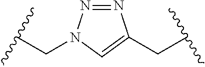 [SMILES: *Cc1cn(C*)nn1]

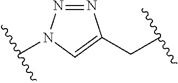 [SMILES: *Cc1cn(*)nn1]

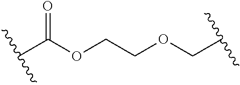 [SMILES: *COCCOC(*)=O]

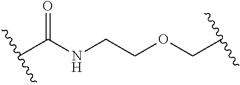 [SMILES: *COCCNC(*)=O]

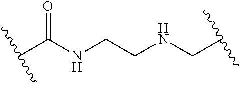 [SMILES: *CNCCNC(*)=O]

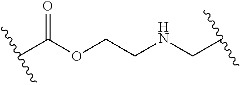 [SMILES: *CNCCOC(*)=O]

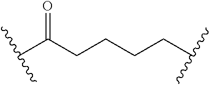 [SMILES: *CCCCC(*)=O]

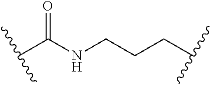 [SMILES: *CCCNC(*)=O]

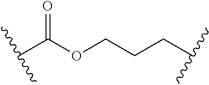 [SMILES: *CCCOC(*)=O]

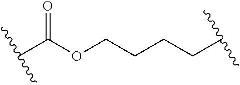 [SMILES: *CCCCOC(*)=O]

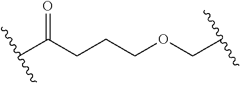 [SMILES: *COCCCC(*)=O]

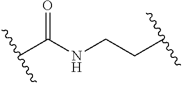 [SMILES: *CCNC(*)=O]

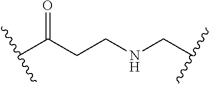 [SMILES: *CNCCC(*)=O]

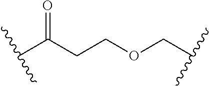 [SMILES: *COCCC(*)=O]

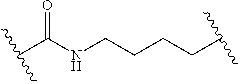 [SMILES: *CCCCNC(*)=O]

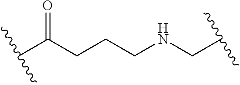 [SMILES: *CNCCCC(*)=O]

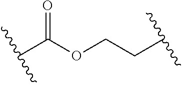 [SMILES: *CCOC(*)=O]

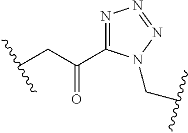 [SMILES: *CC(=O)c1nnnn1C*]

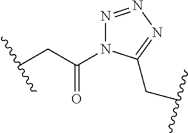 [SMILES: *CC(=O)n1nnnc1C*]

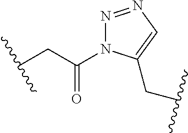 [SMILES: *CC(=O)n1nncc1C*]

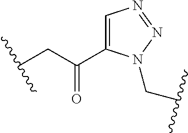 [SMILES: *CC(=O)c1cnnn1C*]

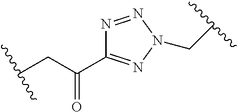 [SMILES: *CC(=O)c1nnn(C*)n1]

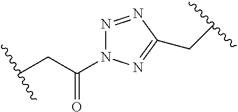 [SMILES: *CC(=O)n1nnc(C*)n1]

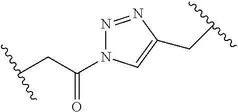 [SMILES: *CC(=O)n1cc(C*)nn1]

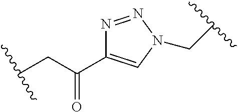 [SMILES: *CC(=O)c1cn(C*)nn1]

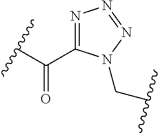 [SMILES: *Cn1nnnc1C(*)=O]

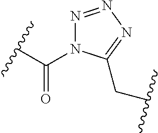 [SMILES: *Cc1nnnn1C(*)=O]

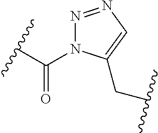 [SMILES: *Cc1cnnn1C(*)=O]

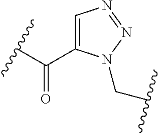 [SMILES: *Cn1nncc1C(*)=O]

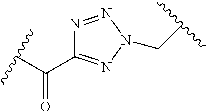 [SMILES: *Cn1nnc(C(*)=O)n1]

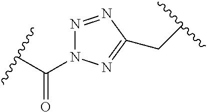 [SMILES: *Cc1nnn(C(*)=O)n1]

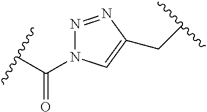 [SMILES: *Cc1cn(C(*)=O)nn1]

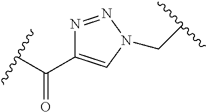 [SMILES: *Cn1cc(C(*)=O)nn1]

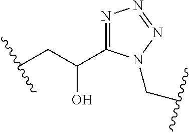 [SMILES: *CC(O)c1nnnn1C*]

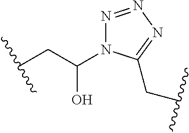 [SMILES: *Cc1nnnn1C(O)C*]

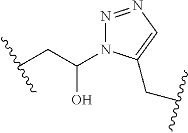 [SMILES: *Cc1cnnn1C(O)C*]

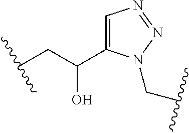 [SMILES: *CC(O)c1cnnn1C*]

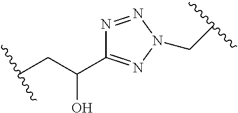 [SMILES: *CC(O)c1nnn(C*)n1]

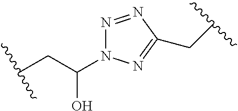 [SMILES: *Cc1nnn(C(O)C*)n1]

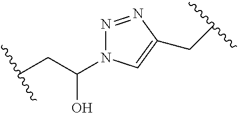 [SMILES: *Cc1cn(C(O)C*)nn1]

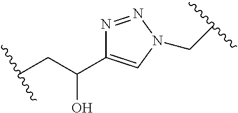 [SMILES: *CC(O)c1cn(C*)nn1]

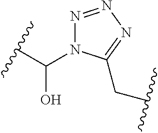 [SMILES: *Cc1nnnn1C(*)O]

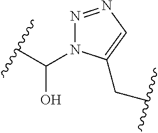 [SMILES: *Cc1cnnn1C(*)O]

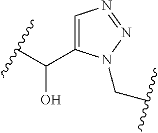 [SMILES: *Cn1nncc1C(*)O]

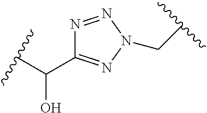 [SMILES: *Cn1nnc(C(*)O)n1]

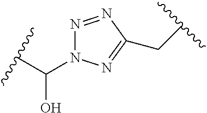 [SMILES: *Cc1nnn(C(*)O)n1]

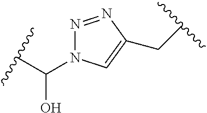 [SMILES: *Cc1cn(C(*)O)nn1]

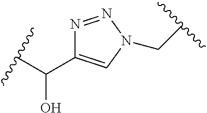 [SMILES: *Cn1cc(C(*)O)nn1]

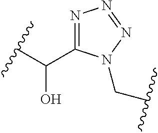 [SMILES: *Cn1nnnc1C(*)O]

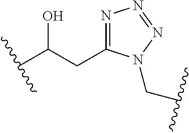 [SMILES: *Cn1nnnc1CC(*)O]

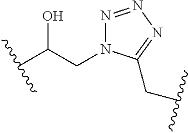 [SMILES: *Cc1nnnn1CC(*)O]

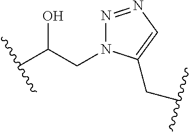 [SMILES: *Cc1cnnn1CC(*)O]

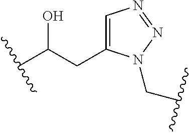 [SMILES: *Cn1nncc1CC(*)O]

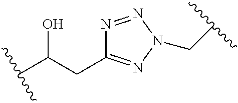 [SMILES: *Cn1nnc(CC(*)O)n1]

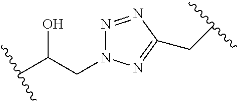 [SMILES: *Cc1nnn(CC(*)O)n1]

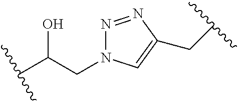 [SMILES: *Cc1cn(CC(*)O)nn1]

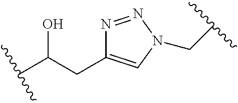 [SMILES: *Cn1cc(CC(*)O)nn1]

### Methods of Preparation

Compounds of the invention may be synthesized according to the schemes below and those presented in the Examples. The reagents and conditions described are intended to be exemplary and not limiting. As one of skill in the art would appreciate, various analogs may be prepared by modifying the synthetic reaction, for example, suing different starting materials, different reagents, different reaction conditions (e.g., temperature, solvent, concentration). The synthesis of sulfonyl AMP analogs is described in Lu et al., ChemBioChem (2012) 13, 129-136, Lu et al., Bioorg. Med. Chem. Lett. (2008) 18, 5963-5966, Matarlo et al. Biochemistry (2015) 54, 6514-6524, Cisar et al., J. Am. Chem. Soc. (2007) 129, 7752-7753, U.S. patent application Ser. No. 11/911,525, U.S. patent application Ser. No. 13/897,807, and PCT application PCT/US2006/014394, each of which is incorporated herein by reference.

In one aspect, the present invention provides methods for the preparation of compounds of Formula (I) and intermediates thereto. Exemplary synthetic methods are shown in Schemes 1 to 4. Unless otherwise stated, variables depicted in the schemes below are as defined for compounds of Formula (I).

P¹ is hydrogen, halogen, lithium, sodium, potassium, zinc halide, magnesium halide, silyl, stannyl, boronyl, acyl, or LG.

P² is hydrogen, halogen, lithium, sodium, potassium, zinc halide, magnesium halide, silyl, stannyl, boronyl, acyl, or LG.

P³ is hydrogen, optionally substituted C₁₋₆ alkyl, optionally substituted acyl, or an oxygen protecting group.

G² is —C(R^{G1})(R^{G2})—, —C(═O)—, —C(═NR^{f})—, or —C(═C(R^{G1})(R^{G2}))—.

LG is a leaving group. Exemplary leaving groups include, but are not limited to, halogen (e.g., F, Cl, Br, I), sulfonic acid ester (e.g., tosylate, mesylate, triflate), —OH, alkoxy, aryloxy, alkoxycarbonyloxy, aryloxycarbonyloxy, alkylcarbonyloxy, and arylcarbonyloxy.

Each of P, P^{E2}, and P^{E3} are hydrogen, substituted C₁₋₆ alkyl, optionally substituted acyl, or an oxygen protecting group.

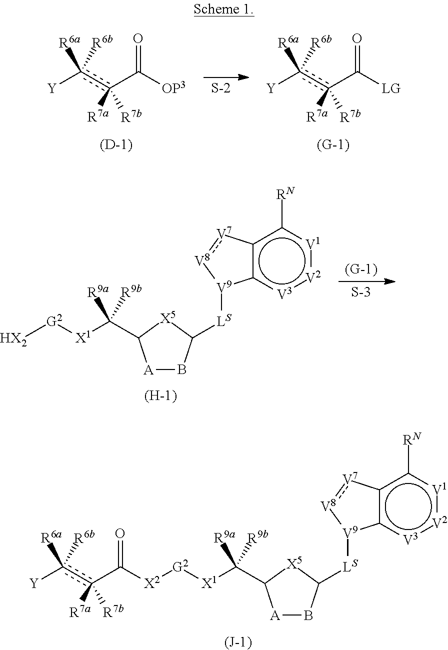

When L¹ is

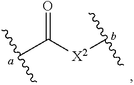

a compound of Formula (I) may be prepared according to Scheme 1. Step S-2 comprises converting a compound of Formula (D-1) to a compound of Formula (G-1). In some embodiments, LG is —OH. In some embodiments, the step of converting comprises deprotection of P³. In some embodiments, LG is halogen (e.g., —Cl, —Br, —I). In some embodiments, the step of converting is performed in the presence of an acid (e.g., TFA). In some embodiments, the step of converting is performed in the presence of a halogenating reagent (e.g., —Cl₂, —Br₂, —I₂, SOCl₂, POCl₃, N-halosuccinimide).

Step S-3 comprises coupling a compound of Formula (G-1) and a sulfonyl compound of Formula (H-1) to form a compound of Formula (J-1). A compound of Formula (J-1) is a compound of Formula (I). In some embodiments, X² is —O—. In some embodiments, X² is —NR^{f}—. In some embodiments, X² is —NH—. In some embodiments, LG is halogen (e.g., —Cl, —Br, —I). In some embodiments, LG is —OH. In some embodiments, LG is —OH, and X² is —O—. In some embodiments, LG is —OH, and X² is —NH—. In some embodiments, the step of coupling is performed in the presence of a carbodiimide (e.g., DCC, EDC). In some embodiments, the step of coupling is performed in the presence of a base (e.g., DMAP).

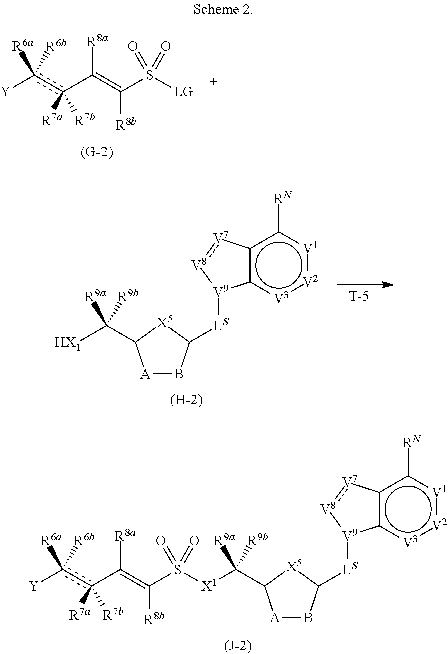

When L¹ is

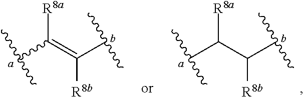

a compound of Formula (I) may be prepared according to Scheme 2. Step T-5 comprises coupling a sulfonyl compound of Formula (G-2) with a compound of Formula (H-2) to form a compound of Formula (J-2). A compound of Formula (J-2) is a compound of Formula (I). In some embodiments, X¹ is —O—. In some embodiments, X¹ is —NR^{f}—. In some embodiments, X¹ is —NH—. In some embodiments, LG is halogen (e.g., —Cl, —Br, —I). In some embodiments, LG is —OH. In some embodiments, LG is —Cl, and X¹ is —O—. In some embodiments, LG is —Cl, and X¹ is —NH—. In some embodiments, the step of coupling is performed in the presence a base (e.g., pyridine, lutidine, DMAP).

In certain embodiments, a method of preparing a compound of Formula (I) further comprises reducing the double bond of a compound of Formula (J-2) to a single bond.

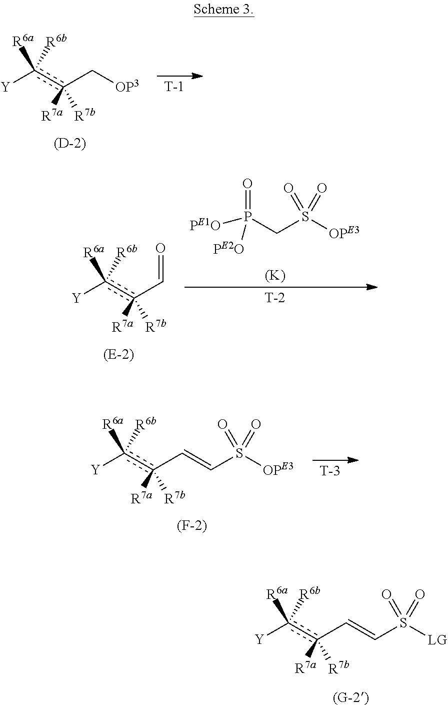

Intermediate (G-2′) may be prepared according to Scheme 3. Step T-1 comprises oxidizing a compound of Formula (D-2) to an aldehyde of Formula (E-2). In certain embodiments, P³ is H. In certain embodiments, P³ is a non-hydrogen group and Step T-1 further comprises deprotection of P³. In some embodiments, the step of oxidizing comprises a Swern oxidation, Pfitzner-Moffatt oxidation, Corey-Kim oxidation, or Dess-Martin oxidation. In some embodiments, the step of oxidizing is performed in the presence of pyridiniumchlorochromate (PCC), oxalyl chloride, a carbodiimide (e.g., DCC, EDC), an N-halosuccinimide (e.g., NCS, NBS, NIS), or Dess-Martin periodinane (DMP). In some embodiments, the step of oxidizing is performed in the presence of dimethylsulfoxide or dimethylsulfide.

Step T-2 comprises coupling an aldehyde of Formula (E-2) and a sulfonyl phosphonate of Formula (K) to form a sulfonate of Formula (F-2). In certain embodiments, P^{E1}, P^{E2}, and P^{E3} are unsubstituted C₁₋₆ alkyl (e.g., methyl, ethyl, propyl). In certain embodiments, P^{E1}, P^{E2}, and P^{E3} are ethyl. In some embodiments, the step of coupling comprises a Horner-Wadsworth-Emmons coupling. In some embodiments, the step of coupling is performed in the presence of a base (e.g., an organolithium species (e.g., n-BuLi, tert-BuLi).

Step T-3 comprises converting a sulfonate of Formula (F-2) to a sulfonyl compound of Formula (G-2′). A compound of Formula (G-2′) is a compound of Formula (G-2). In some embodiments, LG is —OH. In some embodiments, the step of converting comprises deprotection of P^{E3}. In some embodiments, LG is halogen (e.g., —Cl, —Br, —I). In some embodiments, the step of converting is performed in the presence of an acid (e.g., TFA). In some embodiments, the step of converting is performed in the presence of a halogenating reagent (e.g., —Cl₂, —Br₂, —I₂, SOCl₂, POCl₃, N-halosuccinimide).

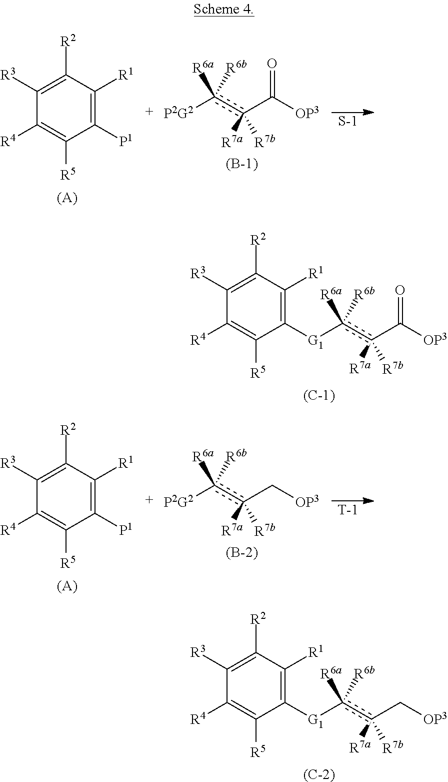

When Y is

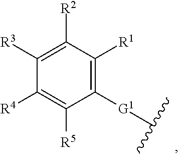

intermediate (D-1) is a compound of Formula (C-1), and intermediate (D-2) is a compound of Formula (C-2). Compounds of Formula (C-1) and (C-2) may be prepared according to Scheme 4.

Step S-1 comprises coupling a cyclic compound of Formula (A) with a compound of Formula (B-1) to form a compound of Formula (C-1). In some embodiments, P¹ is halogen (e.g., —Cl, —Br, —I). In some embodiments, P² is lithium, sodium, potassium, magnesium halide, zinc halide, stannyl, boronyl, or silyl. In some embodiments P¹ is halogen, and P² is lithium, sodium, potassium, magnesium halide, zinc halide, stannyl, boronyl, or silyl. In some embodiments, P² is halogen (e.g., —Cl, —Br, —I). In some embodiments, P¹ is zinc halide, stannyl, boronyl, or silyl. In some embodiments P² is halogen, and P¹ is zinc halide, stannyl, boronyl, or silyl. In some embodiments, P² is halogen (e.g., —Br), and P¹ is boronyl (e.g., —B(OH)₂). In some embodiments, the step of coupling is performed in the presence of palladium. In certain embodiments, G² is —C(═O)—. In certain embodiments, G² is —C(═CH₂)—. In certain embodiments, G² is —C(═CH₂)—, and the step of coupling further comprises oxidizing —C(═CH₂)— to —C(═O)—. In some embodiments, the step of oxidizing is done in the presence of ozone.

Step T-1 comprises coupling a cyclic compound of Formula (A) with a compound of Formula (B-2) to form a compound of Formula (C-2). In some embodiments, P¹ is halogen (e.g., —Cl, —Br, —I). In some embodiments, P² is lithium, sodium, potassium, magnesium halide, zinc halide, stannyl, boronyl, or silyl. In some embodiments P¹ is halogen, and P² is lithium, sodium, potassium, magnesium halide, zinc halide, stannyl, boronyl, or silyl. In some embodiments, P² is halogen (e.g., —Cl, —Br, —I). In some embodiments, P¹ is zinc halide, stannyl, boronyl, or silyl. In some embodiments P² is halogen, and P¹ is zinc halide, stannyl, boronyl, or silyl. In some embodiments, P² is halogen (e.g., —Br), and P¹ is boronyl (e.g., —B(OH)₂). In some embodiments, the step of coupling is performed in the presence of palladium. In certain embodiments, G² is —C(═O)—. In certain embodiments, G² is —C(═CH₂)—. In certain embodiments, G² is —C(═CH₂)—, and the step of coupling further comprises oxidizing —C(═CH₂)— to —C(═O)—. In some embodiments, the step of oxidizing is done in the presence of ozone.

The method of preparing a compound of Formula (I) or an intermediate thereto optionally further comprises one or more steps of protecting a nitrogen, oxygen, or sulfur atom, or deprotecting a nitrogen, oxygen, or sulfur atom. In certain embodiments, the step of deprotecting or protecting comprises replacing R^{S1}, R^{S2}, or both R^{S1} and R^{S2}. In certain embodiments, the step of deprotecting or protecting comprises replacing one R^{Na} or both R^{Na} of group R^{N}. In certain embodiments, the step of deprotecting or protecting comprises replacing both R^{S1} and R^{S2}, and replacing one R^{Na}, or both R^{Na}, or group R^{N}.

### Pharmaceutical Compositions and Administration

The present invention also provides pharmaceutical compositions comprising a compound described herein (e.g., a compound of Formula (I)), or a pharmaceutically acceptable salt, solvate, hydrate, polymorph, co-crystal, tautomer, stereoisomer, or prodrug thereof, and optionally a pharmaceutically acceptable excipient. In certain embodiments, the pharmaceutical composition described herein comprises a compound of Formula (I), or a pharmaceutically acceptable salt, stereoisomer, or tautomer thereof, and a pharmaceutically acceptable excipient.

In certain embodiments, the compound described herein is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating an infectious disease (e.g., bacterial infection, e.g., tuberculosis, MRSA)) in a subject in need thereof. In certain embodiments, the effective amount is an amount effective for preventing an infectious disease (e.g., bacterial infection, e.g., tuberculosis, MRSA)) in a subject in need thereof. In certain embodiments, the effective amount is an amount effective for reducing the risk of developing an infectious disease (e.g., bacterial infection, e.g., tuberculosis, MRSA)) in a subject in need thereof. In certain embodiments, the effective amount is an amount effective for inhibiting menaquinone biosynthesis (e.g., inhibiting o-succinylbenzoate-CoA synthetase (MenE)) in an infection in a subject. In certain embodiments, the effective amount is an amount effective for inhibiting cellular respiration in an infection in a subject. In certain embodiments, the effective amount is an amount effective for inhibiting cellular respiration in an infectious microorganism. In certain embodiments, the effective amount is an amount effective for inhibiting menaquinone biosynthesis (e.g., inhibiting o-succinylbenzoate-CoA synthetase (MenE)) in an infectious microorganism.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the effective amount is an amount effective for inhibiting menaquinone biosynthesis by at least about 10%, at least about 20%, at least about 30%, at least about 40%, at least about 50%, at least about 60%, at least about 70%, at least about 80%, at least about 90%, at least about 95%, or at least about 98%. In certain embodiments, the effective amount is an amount effective for inhibiting menaquinone biosynthesis by not more than 10%, not more than 20%, not more than 30%, not more than 40%, not more than 50%, not more than 60%, not more than 70%, not more than 80%, not more than 90%, not more than 95%, or not more than 98%. In certain embodiments, the effective amount is an amount effective for inhibiting an adenylate-forming enzyme (e.g., an acyl-CoA synthetase) by at least about 10%, at least about 20%, at least about 30%, at least about 40%, at least about 50%, at least about 60%, at least about 70%, at least about 80%, at least about 90%, at least about 95%, or at least about 98%. In certain embodiments, the effective amount is an amount effective for inhibiting adenylate-forming enzyme (e.g., an acyl-CoA synthetase) by not more than 10%, not more than 20%, not more than 30%, not more than 40%, not more than 50%, not more than 60%, not more than 70%, not more than 80%, not more than 90%, not more than 95%, or not more than 98%. In certain embodiments, the effective amount is an amount effective for inhibiting o-succinylbenzoate-CoA synthetase (MenE) by at least about 10%, at least about 20%, at least about 30%, at least about 40%, at least about 50%, at least about 60%, at least about 70%, at least about 80%, at least about 90%, at least about 95%, or at least about 98%. In certain embodiments, the effective amount is an amount effective for inhibiting o-succinylbenzoate-CoA synthetase (MenE) by not more than 10%, not more than 20%, not more than 30%, not more than 40%, not more than 50%, not more than 60%, not more than 70%, not more than 80%, not more than 90%, not more than 95%, or not more than 98%. In certain embodiments, the effective amount is an amount effective for a range of inhibition between a percentage described in this paragraph and another percentage described in this paragraph, inclusive.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting menaquinone biosynthesis in an infectious microorganism), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents.

Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., infectious disease, proliferative disease, hematological disease, or painful condition). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-diabetic agents, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-allergic agents, contraceptive agents, and pain-relieving agents. In certain embodiments, the additional pharmaceutical agent is an binder or inhibitor of an AMP-producing synthetase. In certain embodiments, the additional pharmaceutical agent is an binder or inhibitor of a ligase and/or adenylate-forming enzyme (e.g., o-succinybenzoate-CoA synthetase (MenE)). In certain embodiments, the additional pharmaceutical agent inhibits cellular respiration. In certain embodiments, the additional pharmaceutical agent inhibits menaquinone biosynthesis. In certain embodiments, the additional pharmaceutical agent is an antibiotic. In certain embodiments, the additional pharmaceutical agent is an anti-bacterial agent.

In certain embodiments, the additional pharmaceutical agent is a β-lactam antibiotic. Exemplary β-lactam antibiotics include, but are not limited to: β-lactamase inhibitors (e.g., avibactam, clavulanic acid, tazobactam, sulbactam); carbacephems (e.g., loracarbef); carbapenems (e.g., doripenem, imipenem, ertapenem, meropenem); cephalosporins (1^{st} generation) (e.g., cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, ceftezole, cephalosporin C); cephalosporins (2^{nd} generation) (e.g., cefaclor, cefamandole, cefbuperzone, cefmetazole, cefonicid, ceforanide, cefotetan, cefotiam, cefoxitin, cefminox, cefprozil, cefuroxime, cefuzonam); cephalosporins (3^{rd} generation) (e.g., cefcapene, cefdaloxime, cefdinir, cefditorin, cefetamet, cefixime, cefmenoxime, cefodizime, cefoperazone, cefotaxime, cefovecin, cefpimizole, cefpiramide, cefpodoxime, ceftamere, ceftazidime, cefteram, ceftibuten, ceftiofur, ceftiolene, ceftizoxime, ceftriaxone, latamoxef); cephalosporins (4^{th} generation) (e.g., cefepime, cefluprenam, cefoselis, cefozopran, cefpirome, cefquinome, flomoxef); cephalosporins (5^{th} generation) (e.g., ceftaroline fosamil, ceftobiprole, ceftolozane); cephems (e.g., cefaloram, cefaparole, cefcanel, cefedrolor, cefempidone, cefetrizole, cefivitril, cefmepidium cefoxazole, cefrotil, cefsulodin, cefsumide, ceftioline, ceftioxime, cefuracetime, nitrocefin); monobactams (e.g., aztreonam, carumonam, norcadicin A, tabtoxinine β-lactam, tigemonam); penicillins/penams (e.g., amoxicillin, amoxicillin/clavulanate, ampicillin, ampicillin/flucloxacillin, ampicillin/sulbactam, azidocillin, azlocillin, bacampacillin, benzathine benzylpenicillin, benzathine phenoxymethylpenicillin, carbenicillin, carindacillin, clometocillin, cloxacillin, dicloxacillin, epicillin, flucloxacillin, hetacllin, mecillinam, mezlocillin, meticillin, metampiciillin, nafcillin, oxacillin, penamacillin, penicillin G, penicillin V, phenaticillin, piperacillin, piperacillin/tazobactam, pivampicillin, pivmeclillinam, procaine benzylpenicillin, propicillin, sulbenicillin, talampicillin, temocllin, ticarcillin, ticarcillin/clavulanate); and penems/carbapenems (e.g., biapenem, doripenem, ertapenem, faropenem, imipenem, imipenem/cilastatin, lenapenem, meropenem, panipenem, razupenem, tebipenem, thienamycin, tomopenem).

In certain embodiments, the additional pharmaceutical agent is a non-β-lactam antibiotic. Exemplary non-β-lactam antibiotics include, but are not limited to: aminoglycosides (e.g., amikacin, dibekacin, gentamicin, kanamycin, neomycin, netilmicin, tobramycin, paromomycin, sisomicin, streptomycin, spectinomycin); ansamycins (e.g., geldanamycin, herbimycin); glycopeptides (e.g., belomycin, dalbavancin, oritavancin, ramoplanin, teicoplanin, telavancin, vancomycin); glycylcyclines (e.g., tigecycline); lincosamides (e.g., clindamycin, lincomycin); lipopeptides (e.g., anidulafungin, caspofungin, cilofungin, daptomycin, echinocandin B, micafungin, mycosubtilin); macrolides (e.g., azithromycin, carbomycin A, clarithromycin, dirithromycin, erythromycin, josmycin, kitasamycin, midecamycin, oleandomycin, roxithromycin, solithromycin, spiramycin, troleandomycin, telithromycin, tylosin); nitrofurans (e.g., furazolidone, furylfuramide, nitrofurantoin, nitrofurazone, nifuratel, nifurquinazol, nifurtoinol, nifuroxazide, nifurtimox, nifurzide, ranbezolid); nitroimidazoles (e.g., metronidazole, nimorazole, tinadazole); oxazolidinones (e.g., cycloserine, linezolid, posizolid radezolid, tedizolid); polypeptides (e.g., actinomycin, bacitracin, colistin, polymyxin B); quinolones (e.g., balofloxacin, besifloxacin, cinoxacin, ciprofloxacin, clinafloxacin, danofloxacin, delafloxacin, diflofloxacin, enoxacin, enrofloxacin, fleroxacin, flumequine, gatifloxacin, gemifloxacin, grepafloxacin, ibafloxacin, JNJ-Q2, levofloxacin, lomefloxacin, marbofloxacin, moxifloxacin, nadifloxacin, nalidixic acid, nemonoxacin, norfloxacin, ofloxacin, orbifloxacin, oxilinic acid, pazufloxacin, pefloxacin, piromidic acid, pipemidic acid, prulifloxacin, rosoxacin, rufloxacin, sarafloxacin, sparfloxacin, sitafloxacin, temafloxacin, tosufloxacin, trovafloxacin); rifamycins (e.g., rifamycin B, rifamycin S, rifamycin SV, rifampicin, rifabutin, rifapentine, rifalazil, rifaximin); sulfonamides (e.g., co-trimoxazole, mafenide, pediazole, sulfacetamide, sulfadiazine, silver sulfadiazine, sulfadimidine, sulfadimethoxine, sulfadoxine, sulfafurazole, sulfamethizole, sulfamethoxazole, sulfamethoxypyridazine, sulfametopyrazine, sulfametoxydiazine, sulfamoxole, sulfanilamide, sulfanitran, sulfasalazine, sulfisomidine, sulfonamidochrysoidine, trimethoprim); tetracyclines (e.g., 6-deoxytetracycline, aureomycin, chlortetracycline, demeclocycline, doxycycline, lymecycline, meclocycline, methacycline, minocycline, oxytetracycline, PTK-0796, sancycline, rolitetracycline, tetracycline, terramycin); tuberactinomycins (e.g., tuberactinomycin A, tuberactinomycin O, viomycin, enviomycin, capreomycin); arsphenamine; chloramphenicol; dalfoprisitin; fosfomycin; fusidic acid; fidaxomycin, gramicidin; lysozyme; mupirocin; platensimycin; pristinamycin; sparsomycin; quinupristin; quinupristin/dalfopristin; teixobactin; and thiamphenicol.

In certain embodiments, the additional pharmaceutical agent is an agent useful in the treatment of MRSA. Additional pharmaceutical agents useful in the treatment of MRSA include, but are not limited to, allicin, ceftaroline fosamil, ceftobiprole, co-trimioxazole, clindamycin, dalfopristin, daptomycin, delafloxacin, doxycycline, linezolid, JNJ-Q2, minocycline, quinipristin, teicoplanin, tigecycline, and vancomycin.

In certain embodiments, the additional pharmaceutical agent is an agent useful in the treatment of mycobacterial infections (e.g., tuberculosis). Additional pharmaceutical agents useful in the treatment of mycobacterial infections include, but are not limited to, amikacin, p-aminosalicyclic acid, arginine, bedaquiline, capreomycin, ciprofloxacin, clarithromycin, clavulanic acid, clofazimine, co-amoxiclav, cycloserine, dapsone, enviomycin, ethambutol, ethionamide, inipenem, isoniazid, interferon-γ, kanamycin, levofloxacin, linezolid, meropenem, metronidazole, moxifloxacin, PA-824, perchlorperazine, prothioamide, pyrazinamide, rifabutin, rifampicin, rifapentine, rifaximin, streptomycin, terizidone, thioazetazeone, thioridazine, vitamin D, and viomycin.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating an infectious disease (e.g., bacterial infection (e.g., tuberculosis, MRSA)) in a subject in need thereof. In certain embodiments, the kits are useful for preventing an infectious disease (e.g., bacterial infection (e.g., tuberculosis, MRSA)) in a subject in need thereof. In certain embodiments, the kits are useful for reducing the risk of developing an infectious disease (e.g., bacterial infection (e.g., tuberculosis, MRSA)) in a subject in need thereof. In certain embodiments, the kits are useful for inhibiting cellular respiration in an infection in a subject or in an infectious microorganism. In certain embodiments, the kits are useful for inhibiting menaquinone biosynthesis (e.g., inhibiting o-succinylbenzoate-CoA synthetase (MenE)) in an infection in a subject or in an infectious microorganism.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating an infectious disease (e.g., bacterial infection (e.g., tuberculosis, MRSA)) in a subject in need thereof. In certain embodiments, the kits and instructions provide for preventing an infectious disease (e.g., bacterial infection (e.g., tuberculosis, MRSA)) in a subject in need thereof. In certain embodiments, the kits and instructions provide for reducing the risk of developing an infectious disease (e.g., bacterial infection (e.g., tuberculosis, MRSA)) in a subject in need thereof. In certain embodiments, the kits and instructions provide for inhibiting cellular respiration in an infection in a subject or in an infectious microorganism. In certain embodiments, the kits and instructions provide for inhibiting menaquinone biosynthesis (e.g., inhibiting o-succinylbenzoate-CoA synthetase (MenE)) in an infection in a subject or in an infectious microorganism. A kit described herein may include one or more additional pharmaceutical agents described herein as a separate composition.

### Methods of Treatment and Uses

The present invention also provides methods that may be useful for the treatment or prevention of a disease. In certain embodiments, the disease is an infectious disease. In certain embodiments, the infectious disease is a bacterial infection. In certain embodiments, the infectious disease is a parasitic infection. In certain embodiments, the infectious disease may arise as complication of another disease or condition, for example, in subjects with a weakened immune system as a result of HIV infection, AIDS, lupus, cancer, cystic fibrosis or diabetes. In certain embodiments, the bacterial infection is an infection caused by Gram-positive bacteria. In certain, embodiments, the bacterial infection is an infection caused by Gram-negative bacteria. In certain embodiments, the bacterial infection in an infection caused by an anaerobically growing bacteria (e.g., a facultative anaerobe under anaerobic conditions). In certain embodiments, the bacterial infection is a Staphylococcus infection, a Bacillus infection, or an Escherichia infection. In certain embodiments, the bacterial infection is a mycobacterial infection. In some embodiments the bacterial infection is an atypical mycobacterial infection. In some embodiments, the infectious disease is tuberculosis. In some embodiments, the infectious disease is multi-drug resistant tuberculosis (MDR-TB). In some embodiments, the infectious disease is extensively drug-resistant tuberculosis (XDR-TB). In certain embodiments, the bacterial infection is a Staphylococcus infection. In some embodiments, the bacterial infection is a Staphylococcus aureus infection. In some embodiments, the bacterial infection is a methicillin-resistant Staphylococcus aureus (MRSA) infection. In some embodiments, the bacterial infection is healthcare-associated MRSA (HA-MRSA). In some embodiments, the bacterial infection is community-associated MRSA (CA-MRSA). In some embodiments, the bacterial infection is a vancomycin-intermediate Staphylococcus aureus (VISA) infection or a vancomycin-resistant Staphylococcus aureus (VRSA) infection.

The compounds described herein (e.g., compounds of Formula (I)), may exhibit inhibitory activity towards an adenylate-forming enzyme (e.g., an acyl-CoA synthetase), may exhibit the ability to inhibit o-succinyl-CoA synthetase (MenE), may exhibit the ability to inhibit cellular respiration in an infectious microorganism, may exhibit the ability to inhibit menaquinone biosynthesis, may exhibit a therapeutic effect and/or preventative effect in the treatment of infectious diseases (e.g., bacterial infections, e.g., tuberculosis, MRSA)), and/or may exhibit a therapeutic and/or preventative effect superior to existing agents for treatment of infectious disease.

The compounds described herein (e.g., compounds of Formula (I)), may exhibit selective inhibition of o-succinylbenzoate-CoA synthetase versus inhibition of other proteins.

In certain embodiments, the selectivity versus inhibition of another protein is between about 2 fold and about 10 fold. In certain embodiments, the selectivity is between about 10 fold and about 50 fold. In certain embodiments, the selectivity is between about 50 fold and about 100 fold. In certain embodiments, the selectivity is between about 100 fold and about 500 fold. In certain embodiments, the selectivity is between about 500 fold and about 1000 fold. In certain embodiments, the selectivity is between about 1000 fold and about 5000 fold. In certain embodiments. In certain embodiments, the selectivity is between about 5000 fold and about 10000 fold. In certain embodiments, or at least about 10000 fold.

The present invention provides methods that may be useful for the treatment of an infectious disease by administering a compound described herein, or pharmaceutically acceptable salt, solvate, hydrate, polymorph, co-crystal, tautomer, stereoisomer, or prodrug thereof, or pharmaceutical composition thereof, to a subject in need thereof. In certain embodiments, the compound is administered as a pharmaceutically acceptable salt, stereoisomer, or tautomer thereof. In certain embodiments, the compound is administered as a pharmaceutically acceptable salt of the compound. In certain embodiments, the compound is administered as a specific stereoisomer or mixture of stereoisomers of the compound. In certain embodiments, the compound is administered as a specific tautomer or mixture of tautomers of the compound. In certain embodiments, the compound is administered as a pharmaceutical composition as described herein comprising the compound.

The present invention also provides uses of the inventive compounds, and pharmaceutically acceptable salts, solvates, hydrates, polymorphs, co-crystals, tautomers, stereoisomers, prodrugs, and pharmaceutical compositions thereof, in the manufacture of medicaments for the treatment and prevention of diseases. In certain embodiments, the disease is an infectious disease. In certain embodiments, the infectious disease is a bacterial infection. In certain embodiments, the infectious disease is a parasitic infection. In certain embodiments, the infectious disease may arise as complication of another disease or condition, for example, in subjects with a weakened immune system as a result of HIV infection, AIDS, lupus, cancer, cystic fibrosis, or diabetes. In certain embodiments, the bacterial infection is an infection caused by Gram-positive bacteria. In certain, embodiments, the bacterial infection is an infection caused by Gram-negative bacteria. In certain embodiments, the bacterial infection in an infection caused by an anaerobically growing bacteria (e.g., a facultative anaerobe under anaerobic conditions). In certain embodiments, the bacterial infection is a Staphylococcus infection, a Bacillus infection, or an Escherichia infection. In certain embodiments, the bacterial infection is a mycobacterial infection. In some embodiments the bacterial infection is an atypical mycobacterial infection. In some embodiments, the infectious disease is tuberculosis. In some embodiments, the infectious disease is multi-drug resistant tuberculosis (MDR-TB). In some embodiments, the infectious disease is extensively drug-resistant tuberculosis (XDR-TB). In certain embodiments, the bacterial infection is a Staphylococcus infection. In some embodiments, the bacterial infection is a Staphylococcus aureus infection. In some embodiments, the bacterial infection is a methicillin-resistant Staphylococcus aureus (MRSA) infection. In some embodiments, the bacterial infection is healthcare-associated MRSA (HA-MRSA). In some embodiments, the bacterial infection is community-associated MRSA (CA-MRSA). In some embodiments, the bacterial infection is a vancomycin-intermediate Staphylococcus aureus (VISA) infection or a vancomycin-resistant Staphylococcus aureus (VRSA) infection.

Certain methods described herein include methods of treating a bacterial infection, methods of treating an infection in a subject, or methods of contacting an infectious microorganism with a compound described herein (e.g. a compound of Formula (I)). Any of these methods may involve a specific class of bacteria or type of bacteria. In certain embodiments, the bacteria is Gram-positive bacteria. In certain, embodiments, the bacterial infection is Gram-negative bacteria. In certain embodiments, the bacteria is an anaerobically growing bacteria (e.g., facultative anaerobe under anaerobic conditions). In certain embodiments the bacteria is from the genus Staphylococcus, Escherichia, or Bacillus. In certain embodiments the bacteria is from the genus Mycobacterium.

In certain embodiments, the microbial infection is an infection with a bacteria, i.e., a bacterial infection. In certain embodiments, the compounds of the invention exhibit anti-bacterial activity. For example, in certain embodiments, the compound has a mean inhibitory concentration, with respect to a particular bacterium, of less than 50 μg/mL, preferably less than 25 μg/mL, more preferably less than 5 μg/mL, and most preferably less than 1 μg/mL.

Exemplary bacteria include, but are not limited to, Gram positive bacteria (e.g., of the phylum Actinobacteria, phylum Firmicutes, or phylum Tenericutes); Gram negative bacteria (e.g., of the phylum Aquificae, phylum Deinococcus-Thermus, phylum Fibrobacteres/Chlorobi/Bacteroidetes (FCB), phylum Fusobacteria, phylum Gemmatimonadest, phylum Ntrospirae, phylum Planctomycetes/Verrucomicrobia/Chlamydiae (PVC), phylum Proteobacteria, phylum Spirochaetes, or phylum Synergistetes); or other bacteria (e.g., of the phylum Acidobacteria, phylum Chlroflexi, phylum Chrystiogenetes, phylum Cyanobacteria, phylum Deferrubacteres, phylum Dictyoglomi, phylum Thermodesulfobacteria, or phylum Thermotogae).

In certain embodiments, the bacteria is a member of the phylum Firmicutes and the genus Enterococcus, e.g., the bacterial infection is an Enterococcus infection. Exemplary Enterococci bacteria include, but are not limited to, E. avium, E. durans, E. faecalis, E. faecium, E. gallinarum, E. solitarius, E. casseliflavus, and E. raffinosus.

In certain embodiments, the bacteria is a member of the phylum Firmicutes and the genus Staphylococcus, e.g., the bacterial infection is a Staphylococcus infection. Exemplary Staphylococci bacteria include, but are not limited to, S. arlettae, S. aureus, S. auricularis, S. capitis, S. caprae, S. carnous, S. chromogenes, S. cohii, S. condimenti, S. croceolyticus, S. delphini, S. devriesei, S. epidermis, S. equorum, S. felis, S. fluroettii, S. gallinarum, S. haemolyticus, S. hominis, S. hyicus, S. intermedius, S. kloosii, S. leei, S. lenus, S. lugdunesis, S. lutrae, S. lyticans, S. massiliensis, S. microti, S. muscae, S. nepalensis, S. pasteuri, S. penttenkoferi, S. piscifermentans, S. psuedointermedius, S. psudolugdensis, S. pulvereri, S. rostri, S. saccharolyticus, S. saprophyticus, S. schleiferi, S. sciuri, S. simiae, S. simulans, S. stepanovicii, S. succinus, S. vitulinus, S. warneri, and S. xylosus. In some embodiments, the bacteria is S. aureus. In some embodiments, the bacteria is methicillin-resistant S. auereus (MRSA). In some embodiments, the bacteria is vancomycin-intermediate S. aureus (VISA) or vancomycin-resistant S. aureus (VRSA).

In certain embodiments, the bacteria is a member of the phylum Firmicutes and the genus Bacillus, e.g., the bacterial infection is a Bacillus infection. Exemplary Bacillus bacteria include, but are not limited to, B. alcalophilus, B. alvei, B. aminovorans, B. amyloliquefaciens, B. aneurinolyticus, B. anthracis, B. aquaemaris, B. atrophaeus, B. boroniphilus, B. brevis, B. caldolyticus, B. centrosporus, B. cereus, B. circulans, B. coagulans, B. firmus, B. flavothermus, B. fusiformis, B. globigii, B. infernus, B. larvae, B. laterosporus, B. lentus, B. licheniformis, B. megaterium, B. mesentericus, B. mucilaginosus, B. mycoides, B. natto, B. pantothenticus, B. polymyxa, B. pseudoanthracis, B. pumilus, B. schlegelii, B. sphaericus, B. sporothermodurans, B. stearothermophilus, B. subtilis, B. thermoglucosidasius, B. thuringiensis, B. vulgatis, and B. weihenstephanensis. In certain embodiments, the bacteria is B. subtilis.

In certain embodiments, the bacteria is a member of the phylum Firmicutes and the genus Strepococcus, e.g., the bacterial infection is a Strepococcus infection. Exemplary Strepococcus bacteria include, but are not limited to, S. agalactiae, S. anginosus, S. bovis, S. canis, S. constellatus, S. dysgalactiae, S. equinus, S. iniae, S. intermedius, S. mitis, S. mutans, S. oralis, S. parasanguinis, S. peroris, S. pneumoniae, S. pyogenes, S. ratti, S. salivarius, S. thermophilus, S. sanguinis, S. sobrinus, S. suis, S. uberis, S. vestibularis, S. viridans, and S. zooepidemicus. In certain embodiments, the baceteria is S. pyogenes. In certain embodiments, the bacteria is S. pneumoniae.

In certain embodiments, the bacteria is a member of the phylum Proteobacteria and the genus Escherichia, e.g., the bacterial infection is an Escherichia infection. Exemplary Escherichia bacteria include, but are not limited to, E. albertii, E. blattae, E. coli, E. fergusonii, E. hermannii, and E. vulneris. In certain embodiments, the bacteria is E. coli.

In certain embodiments, the bacteria is a member of the phylum Proteobacteria and the genus Haemophilus. i.e., the bacterial infection is an Haemophilus infection. Exemplary Haemophilus bacteria include, but are not limited to, H. aegyptius, H. aphrophilus, H. avium, H. ducreyi, H. felis, H. haemolyticus, H. influenzae, H. parainfluenzae, H. paracuniculus, H. parahaemolyticus, H. pittmaniae, Haemophilus segnis, and H. somnus. In certain embodiments, the bacteria is H. influenzae.

In certain embodiments, the bacteria is a member of the phylum Actinobacteria and the Mycobacterium. In some embodiments the bacteria is a baceteria associated with an atypical mycobacterial infection. Exemplary bacteria from genus Mycobacterium include, but are not limited to: M. abscessus, M. africanum, M. avium, M. bovis, M. caprae, M. canetti, M. chelonae, M. colombiense, M. flavescens, M. fortuitum, M. genavense, M. gordonae, M. haemophilum, M. intracellulare, M. kansasii, M. leprae, M. lepramatosis, M. malmoense, M. marinum, M. microti, M. parafortuitum, M. phlei, M. pinnipedii, M. scrofulaceum, M. simiae, M. smegmatis, M. szulgai, M. terrae, M. ulcerans, M. vaccae, and M. xenope. In some embodiments, the bacteria is a bacteria that can cause tuberculosis (e.g., a member of the Mycobacterium tuberculosis complex (e.g., M. tuberculosis, M. africanum, M. bovis, M bovis BCG, M. microti, M. canetti, M pinnipedii, M. suricattae, M. mungi). In some embodiments, the bacteria is M. tuberculosis. In some embodiments, the bacteria is a member of the Mycobacterium avium complex (e.g., M. avium, M. avium avium, M. avium paratuberculosis, M. avium silvaticum, M. avium hominissuis, M. colombiense, M. indicus pranii, M. intracellulare). In some embodiments, the bacteria is M. phlei. In some embodiments, the bacteria is M. smegmatis.

In certain embodiments, the methods of the invention include administering to the subject an effective amount of a compound described herein (e.g., a compound of Formula (I)), or a pharmaceutically acceptable salt, stereoisomer, or tautomer thereof, or a pharmaceutical composition thereof. In certain embodiments, the effective amount is a therapeutically effective amount. In certain embodiments, the effective amount is a prophylactically effective amount.

In another aspect, the present invention provides methods for inhibiting cellular respiration in an infection in a subject by administering to the subject a compound described herein (e.g., a compound of Formula (I)), or a pharmaceutically acceptable salt, stereoisomer, or tautomer thereof, or a pharmaceutical composition thereof.

In another aspect, the present invention provides methods for inhibiting cellular respiration in an infectious microorganism, by contacting the sample with a compound described herein (e.g., a compound of Formula (I)), or a pharmaceutically acceptable salt, stereoisomer, or tautomer thereof, or a pharmaceutical composition thereof.

In another aspect, the present invention provides methods for inhibiting menaquinone biosynthesis in an infection in a subject by administering to the subject a compound described herein (e.g., a compound of Formula (I)), or a pharmaceutically acceptable salt, stereoisomer, or tautomer thereof, or a pharmaceutical composition thereof.

In another aspect, the present invention provides methods for inhibiting menaquinone biosynthesis in an infectious microorganism, by contacting the sample with a compound described herein (e.g., a compound of Formula (I)), or a pharmaceutically acceptable salt, stereoisomer, or tautomer thereof, or a pharmaceutical composition thereof.

In another aspect, the present invention provides methods for inhibiting an adenylate-forming enzyme (e.g., an acyl-CoA synthetase) in an infection in a subject by administering to the subject a compound described herein (e.g., a compound of Formula (I)), or a pharmaceutically acceptable salt, stereoisomer, or tautomer thereof, or a pharmaceutical composition thereof.

In another aspect, the present invention provides methods for inhibiting an adenylate-forming enzyme (e.g., an acyl-CoA synthetase) in an infectious microorganism, by contacting the sample with a compound described herein (e.g., a compound of Formula (I)), or a pharmaceutically acceptable salt, stereoisomer, or tautomer thereof, or a pharmaceutical composition thereof.

In another aspect, the present invention provides methods for inhibiting a ligase and/or adenylate-forming enzyme (e.g., o-succinylbenzoate-CoA synthetase (MenE)) in an infection in a subject by administering to the subject a compound described herein (e.g., a compound of Formula (I)), or a pharmaceutically acceptable salt, stereoisomer, or tautomer thereof, or a pharmaceutical composition thereof.

In another aspect, the present invention provides methods for inhibiting a ligase and/or adenylate-forming enzyme (e.g., o-succinylbenzoate-CoA synthetase (MenE)) in an infectious microorganism, by contacting the sample with a compound described herein (e.g., a compound of Formula (I)), or a pharmaceutically acceptable salt, stereoisomer, or tautomer thereof, or a pharmaceutical composition thereof.

The present invention provides uses of compounds described herein (e.g., compounds of Formulae (I), (Z)), and pharmaceutically acceptable salts, solvates, hydrates, polymorphs, co-crystals, tautomers, stereoisomers, or prodrugs thereof, and pharmaceutical compositions thereof, in any of the methods described here (e.g., methods of treatment, inhibition).

The present invention also provides uses of compounds described herein (e.g., compounds of Formulae (I), (Z)), or pharmaceutically acceptable salts, solvates, hydrates, polymorphs, co-crystals, tautomers, stereoisomers, or prodrugs thereof, or pharmaceutical compositions thereof, in the manufacture of medicaments. The medicament may be used to treat any disease or condition described herein.

The present invention also provides methods of using a compound described herein (e.g., a compound of Formula (I)), or a pharmaceutically acceptable salt, solvate, hydrate, polymorph, co-crystal, tautomer, stereoisomer, or prodrug thereof, or pharmaceutical compositions thereof, in research studies in the field of disease pathology, biochemistry, cell biology, and other fields associated with infectious diseases. The compounds of the invention can be used to study the roles of biomolecules (e.g., o-succinylbenzoate-CoA synthetase, menaquinone, a Vitamin K, chorismate, o-succinyl benzoate, o-succinyl benzoate-AMP, o-succinylbenzoate-CoA, 1,4-dihydroxy-2-napthyol-CoA). The compounds of the invention can be used to study cellular respiration in a microorganism. In certain embodiments, the method comprises use of the compound or composition thereof to inhibit cellular respiration.

In certain embodiments, the method comprises use of the compound or composition thereof to inhibit menaquinone biosynthesis. In certain embodiments, the method comprises use of the compound or composition thereof to inhibit the ligase and/or adenylate-forming enzyme (e.g., o-succinylbenzoate-CoA synthetase (MenE)). In certain embodiments, the method comprises determining the concentration of a biomolecule in a subject or biological sample.

Certain methods described herein, may comprise administering one or more additional pharmaceutical agent in combination with the compounds described herein. The additional pharmaceutical agents include, but are not limited to, anti-diabetic agents, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-allergic agents, contraceptive agents, and pain-relieving agents. In certain embodiments, the additional pharmaceutical agent is an antibiotic. In certain embodiments, the additional pharmaceutical agent is an anti-bacterial agent. In certain embodiments, the additional pharmaceutical agent is an binder or inhibitor of an AMP-producing synthetase. In certain embodiments, the additional pharmaceutical agent is an binder or inhibitor of a ligase and/or adenylate-forming enzyme (e.g., o-succinybenzoate-CoA synthetase (MenE)). In certain embodiments, the additional pharmaceutical agent inhibits cellular respiration. In certain embodiments, the additional pharmaceutical agent inhibits menaquinone biosynthesis.

In certain embodiments, the additional pharmacetucial agent is a p-lactam antibiotic. Exemplary β-lactam antibiotics include, but are not limited to: P-lactamase inhibitors (e.g., avibactam, clavulanic acid, tazobactam, sulbactam); carbacephems (e.g., loracarbef); carbapenems (e.g., doripenem, imipenem, ertapenem, meropenem); cephalosporins (1^{st} generation) (e.g., cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, ceftezole, cephalosporin C); cephalosporins (2^{nd} generation) (e.g., cefaclor, cefamandole, cefbuperzone, cefmetazole, cefonicid, ceforanide, cefotetan, cefotiam, cefoxitin, cefminox, cefprozil, cefuroxime, cefuzonam); cephalosporins (3^{rd} generation) (e.g., cefcapene, cefdaloxime, cefdinir, cefditorin, cefetamet, cefixime, cefmenoxime, cefodizime, cefoperazone, cefotaxime, cefovecin, cefpimizole, cefpiramide, cefpodoxime, ceftamere, ceftazidime, cefteram, ceftibuten, ceftiofur, ceftiolene, ceftizoxime, ceftriaxone, latamoxef); cephalosporins (4^{th} generation) (e.g., cefepime, cefluprenam, cefoselis, cefozopran, cefpirome, cefquinome, flomoxef); cephalosporins (5^{th} generation) (e.g., ceftaroline fosamil, ceftobiprole, ceftolozane); cephems (e.g., cefaloram, cefaparole, cefcanel, cefedrolor, cefempidone, cefetrizole, cefivitril, cefmepidium cefoxazole, cefrotil, cefsulodin, cefsumide, ceftioline, ceftioxime, cefuracetime, nitrocefin); monobactams (e.g., aztreonam, carumonam, norcadicin A, tabtoxinine β-lactam, tigemonam); penicillins/penams (e.g., amoxicillin, amoxicillin/clavulanate, ampicillin, ampicillin/flucloxacillin, ampicillin/sulbactam, azidocillin, azlocillin, bacampacillin, benzathine benzylpenicillin, benzathine phenoxymethylpenicillin, carbenicillin, carindacillin, clometocillin, cloxacillin, dicloxacillin, epicillin, flucloxacillin, hetacllin, mecillinam, mezlocillin, meticillin, metampiciillin, nafcillin, oxacillin, penamacillin, penicillin G, penicillin V, phenaticillin, piperacillin, piperacillin/tazobactam, pivampicillin, pivmeclillinam, procaine benzylpenicillin, propicillin, sulbenicillin, talampicillin, temocllin, ticarcillin, ticarcillin/clavulanate); and penems/carbapenems (e.g., biapenem, doripenem, ertapenem, faropenem, imipenem, imipenem/cilastatin, lenapenem, meropenem, panipenem, razupenem, tebipenem, thienamycin, tomopenem).

In certain embodiments, the additional pharmacetucial agent is a non-β-lactam antibiotic. Exemplary non-β-lactam antibiotics include, but are not limited to: aminoglycosides (e.g., amikacin, dibekacin, gentamicin, kanamycin, neomycin, netilmicin, tobramycin, paromomycin, sisomicin, streptomycin, spectinomycin); ansamycins (e.g., geldanamycin, herbimycin); glycopeptides (e.g., belomycin, dalbavancin, oritavancin, ramoplanin, teicoplanin, telavancin, vancomycin); glycylcyclines (e.g., tigecycline); lincosamides (e.g., clindamycin, lincomycin); lipopeptides (e.g., anidulafungin, caspofungin, cilofungin, daptomycin, echinocandin B, micafungin, mycosubtilin); macrolides (e.g., azithromycin, carbomycin A, clarithromycin, dirithromycin, erythromycin, josmycin, kitasamycin, midecamycin, oleandomycin, roxithromycin, solithromycin, spiramycin, troleandomycin, telithromycin, tylosin); nitrofurans (e.g., furazolidone, furylfuramide, nitrofurantoin, nitrofurazone, nifuratel, nifurquinazol, nifurtoinol, nifuroxazide, nifurtimox, nifurzide, ranbezolid); nitroimidazoles (e.g., metronidazole, nimorazole, tinadazole); oxazolidinones (e.g., cycloserine, linezolid, posizolid radezolid, tedizolid); polypeptides (e.g., actinomycin, bacitracin, colistin, polymyxin B); quinolones (e.g., balofloxacin, besifloxacin, cinoxacin, ciprofloxacin, clinafloxacin, danofloxacin, delafloxacin, diflofloxacin, enoxacin, enrofloxacin, fleroxacin, flumequine, gatifloxacin, gemifloxacin, grepafloxacin, ibafloxacin, JNJ-Q2, levofloxacin, lomefloxacin, marbofloxacin, moxifloxacin, nadifloxacin, nalidixic acid, nemonoxacin, norfloxacin, ofloxacin, orbifloxacin, oxilinic acid, pazufloxacin, pefloxacin, piromidic acid, pipemidic acid, prulifloxacin, rosoxacin, rufloxacin, sarafloxacin, sparfloxacin, sitafloxacin, temafloxacin, tosufloxacin, trovafloxacin); rifamycins (e.g., rifamycin B, rifamycin S, rifamycin SV, rifampicin, rifabutin, rifapentine, rifalazil, rifaximin); sulfonamides (e.g., co-trimoxazole, mafenide, pediazole, sulfacetamide, sulfadiazine, silver sulfadiazine, sulfadimidine, sulfadimethoxine, sulfadoxine, sulfafurazole, sulfamethizole, sulfamethoxazole, sulfamethoxypyridazine, sulfametopyrazine, sulfametoxydiazine, sulfamoxole, sulfanilamide, sulfanitran, sulfasalazine, sulfisomidine, sulfonamidochrysoidine, trimethoprim); tetracyclines (e.g., 6-deoxytetracycline, aureomycin, chlortetracycline, demeclocycline, doxycycline, lymecycline, meclocycline, methacycline, minocycline, oxytetracycline, PTK-0796, sancycline, rolitetracycline, tetracycline, terramycin); tuberactinomycins (e.g., tuberactinomycin A, tuberactinomycin O, viomycin, enviomycin, capreomycin); arsphenamine; chloramphenicol; dalfoprisitin; fosfomycin; fusidic acid; fidaxomycin, gramicidin; lysozyme; mupirocin; platensimycin; pristinamycin; sparsomycin; quinupristin; quinupristin/dalfopristin; teixobactin; and thiamphenicol.

In certain embodiments, the additional pharmaceutical agent is an agent useful in the treatment of MRSA. Additional pharmaceutical agents useful in the treatment of MRSA include, but are not limited to, allicin, ceftaroline fosamil, ceftobiprole, co-trimioxazole, clindamycin, dalfopristin, daptomycin, delafloxacin, doxycycline, linezolid, JNJ-Q2, minocycline, quinipristin, teicoplanin, tigecycline, and vancomycin.

In certain embodiments, the additional pharmaceutical agent is an agent useful in the treatment of mycobacterial infections (e.g., tuberculosis). Additional pharmaceutical agents useful in the treatment of mycobacterial infections include, but are not limited to, amikacin, p-aminosalicyclic acid, arginine, bedaquiline, capreomycin, ciprofloxacin, clarithromycin, clavulanic acid, clofazimine, co-amoxiclav, cycloserine, dapsone, enviomycin, ethambutol, ethionamide, inipenem, isoniazid, interferon-γ, kanamycin, levofloxacin, linezolid, meropenem, metronidazole, moxifloxacin, PA-824, perchlorperazine, prothioamide, pyrazinamide, rifabutin, rifampicin, rifapentine, rifaximin, streptomycin, terizidone, thioazetazeone, thioridazine, vitamin D, and viomycin.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H. Tables of Resolving Agents and Optical Resolutions p. 268 (E. L. Eliel, Ed., Univ. of Notre Dame Press, Notre Dame, Ind. 1972). The invention additionally encompasses compounds as individual isomers substantially free of other isomers, and alternatively, as mixtures of various isomers.

In a formula, [symbol] is a single bond where the stereochemistry of the moities immediately attached thereto is not specified, [symbol] is absent or a single bond, [symbol] or [symbol] is a single or double bond, and [symbol] is a single, double, or triple bond. If drawn in a ring, [symbol] indicates that each bond of the ring is a single or double bond, valency permitting. The precise of arrangement of single and double bonds will be determined by the number, type, and substitution of atoms in the ring, and if the ring is multicyclic or polycyclic. In general, any ring atom (e.g., C or N), can have a double bond with a maximum of one adjacent atom.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen, e.g., fluoro, bromo, chloro, or iodo.

In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

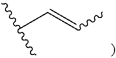

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 n electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Heteroaralkyl” is a subset of “alkyl” and refers to an alkyl group substituted by a heteroaryl group, wherein the point of attachment is on the alkyl moiety.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any one of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —C, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb})₂)₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}—NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)Rx, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any one of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{a}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a nitrogen protecting group described herein is Bn, Boc, Cbz, Fmoc, trifluoroacetyl, triphenylmethyl, acetyl, tosyl, nosyl, brosyl, mesyl, or triflyl.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In certain embodiments, a nitrogen protecting group is benzyl (Bn), tert-butyloxycarbonyl (BOC), carbobenzyloxy (Cbz), 9-flurenylmethyloxycarbonyl (Fmoc), trifluoroacetyl, triphenylmethyl, acetyl (Ac), benzoyl (Bz), p-methoxybenzyl (PMB), 3,4-dimethoxybenzyl (DMPM), p-methoxyphenyl (PMP), 2,2,2-trichloroethyloxycarbonyl (Troc), triphenylmethyl (Tr), tosyl (Ts), brosyl (Bs), nosyl (Ns), mesyl (Ms), triflyl (Tf), or dansyl (Ds).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, an oxygen protecting group described herein is silyl, TBDPS, TBDMS, TIPS, TES, TMS, MOM, THP, t-Bu, Bn, allyl, acetyl, pivaloyl, or benzoyl.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, an oxygen protecting group is silyl. In certain embodiments, an oxygen protecting group is t-butyldiphenylsilyl (TBDPS), t-butyldimethylsilyl (TBDMS), triisoproylsilyl (TIPS), triphenylsilyl (TPS), triethylsilyl (TES), trimethylsilyl (TMS), triisopropylsiloxymethyl (TOM), acetyl (Ac), benzoyl (Bz), allyl carbonate, 2,2,2-trichloroethyl carbonate (Troc), 2-trimethylsilylethyl carbonate, methoxymethyl (MOM), 1-ethoxyethyl (EE), 2-methyoxy-2-propyl (MOP), 2,2,2-trichloroethoxyethyl, 2-methoxyethoxymethyl (MEM), 2-trimethylsilylethoxymethyl (SEM), methylthiomethyl (MTM), tetrahydropyranyl (THP), tetrahydrofuranyl (THF), p-methoxyphenyl (PMP), triphenylmethyl (Tr), methoxytrityl (MMT), dimethoxytrityl (DMT), allyl, p-methoxybenzyl (PMB), t-butyl, benzyl (Bn), allyl, or pivaloyl (Piv).

The term “leaving group” is given its ordinary meaning in the art of synthetic organic chemistry and refers to an atom or a group capable of being displaced by a nucleophile. See, for example, Smith, March Advanced Organic Chemistry 6th ed. (501-502). Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some cases, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some cases, the leaving group is a brosylate, such as p-bromobenzenesulfonyloxy. In some cases, the leaving group is a nosylate, such as 2-nitrobenzenesulfonyloxy. The leaving group may also be a phosphineoxide (e.g., formed during a Mitsunobu reaction) or an internal leaving group such as an epoxide or cyclic sulfate.

Other non-limiting examples of leaving groups are water, ammonia, alcohols, ether moieties, thioether moieties, zinc halides, magnesium moieties, diazonium salts, and copper moieties.

Further exemplary leaving groups include, but are not limited to, halo (e.g., chloro, bromo, iodo) and activated substituted hydroxyl groups (e.g., —OC(═O)SR^{aa}, —OC(═O)R^{aa}, —OCO₂R, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F, Cl, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

The term “nucleobase” as used herein refers to naturally occurring nucleobases (e.g., adenine, guanine, cytosine, thymine, uracil) and non-naturally occurring analogs. A substituted nucleobase may be substituted with 1, 2, or 3, substitutents (e.g., optionally substituted C₁₋₆ alkyl, optionally substituted acyl, or a nitrogen protecting group). Naturally occurring nucleobases include adenine, guanine, thymine, cytosine, and uracil. A nucleobase analog may differ from the naturally occurring nucleobase by substitution at any position, substitution of an optionally substituted carbon atom for an optionally substituted nitrogen atom of equivalent valency, substitution of an optionally substituted nitrogen atom for an optionally substituted carbon atom of equivalent valency, a change in bond order between, or a combination thereof. Examples of analogs include, but are not limited to, N6-methyladenine, N⁶-tert-butyloxycarbonyladenine, N⁴,N⁴-ethanocytosine, 7-deazaxnathosine, 7-deazaguanosine, 8-oxo-N⁶-methyladenine, 4-acetylcytosine, 5-(carboxyhydroxymethyl)uracil, 5-fluorouracil, 5-bromouracil, 5-carboxymethylaminomethyl-2-thiouracil, 5-carboxymethylaminomethyluracil, inosine, N⁶-isopentyladenine, 1-methyladenine, 2-methylguanine, 5-methylcytosine, N⁶-methyladenine, 7-methylguanine, 5-methylaminomethyluracil, 5-methoxyaminomethyl-2-thiouracil, 5-methoxyuracil, psuedouracil, 5-methoxy-2-thiouracil, 5-(1-propynyl)-2-thiouracil, 5-(1-propynyl)-2-thiocytosine, 2-thiocytosine, and 2,6-diaminopurine.

These and other exemplary substituents are described in more detail in the Detailed Description, Examples, and Claims. The invention is not intended to be limited in any manner by the above exemplary listing of substituents.

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R.0.5 H₂O)), and polyhydrates (x is a number greater than 1, e.g., dihydrates (R.2H₂O) and hexahydrates (R.6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “co-crystal” refers to a crystalline structure composed of at least two components. In certain embodiments, a co-crystal contains a compound of the present invention and one or more other component, including but not limited to, atoms, ions, molecules, or solvent molecules. In certain embodiments, a co-crystal contains a compound of the present invention and one or more solvent molecules. In certain embodiments, a co-crystal contains a compound of the present invention and one or more acid or base. In certain embodiments, a co-crystal contains a compound of the present invention and one or more components related to said compound, including not limited to, an isomer, tautomer, salt, solvate, hydrate, synthetic precursor, synthetic derivative, fragment or impurity of said compound.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal “Disease,” “disorder,” and “condition” are used interchangeably herein.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a composition thereof, in or on a subject.

The terms “condition,” “disease,” and “disorder” are used interchangeably.

As used herein, and unless otherwise specified, the terms “treat,” “treating” and “treatment” contemplate an action that occurs while a subject is suffering from the specified disease or condition, which reduces the severity of the disease or condition, or retards or slows the progression of the disease or condition (i.e., “therapeutic treatment”), and also contemplates an action that occurs before a subject begins to suffer from the specified disease or condition (i.e., “prophylactic treatment”).

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for inhibiting menaquinone biosynthesis (e.g., inhibiting MenE). In certain embodiments, a therapeutically effective amount is an amount sufficient for treating a bacterial infection. In certain embodiments, a therapeutically effective amount is an amount sufficient for inhibiting menaquinone biosynthesis (e.g., inhibiting MenE) and for treating a bacterial infection.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for inhibiting menaquinone biosynthesis (e.g., inhibiting MenE). In certain embodiments, a prophylactically effective amount is an amount sufficient for preventing a bacterial infection. In certain embodiments, a prophylactically effective amount is an amount sufficient for inhibiting menaquinone biosynthesis (e.g., inhibiting MenE) and for preventing a bacterial infection.

As used herein the term “inhibit” or “inhibition” in the context of enzymes, for example, in the context of o-succinylbenzoate-CoA synthetase (MenE), refers to a reduction in the activity of the enzyme. In some embodiments, the term refers to a reduction of the level of enzyme activity, e.g., MenE activity, to a level that is statistically significantly lower than an initial level, which may, for example, be a baseline level of enzyme activity. In some embodiments, the term refers to a reduction of the level of enzyme activity, e.g., MenE activity, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of enzyme activity.

As used herein the term “infectious microorganism” refers to a species of infectious fungi, bacteria, or protista, or to a virus. In certain embodiments, the infectious microorganism is a fungi. In certain embodiments, the infectious microorganism is a bacteria. In certain embodiments, the infectious microorganism is a protista. In certain embodiments, the infectious microorganism is a virus.

An “infection” or “infectious disease” refers to an infection with a microorganism, such as a fungus, bacteria or virus. In certain embodiments, the infection is an infection with a fungus, i.e., a fungal infection. In certain embodiments, the infection is an infection with a virus, i.e., a viral infection. In certain embodiments, the infection is an infection with a bacteria, i.e., a bacterial infection. Various infections include, but are not limited to, skin infections, GI infections, urinary tract infections, genito-urinary infections, sepsis, blood infections, and systemic infections.

The term “tuberculosis” or “TB” refers to a infectious disease caused by a species of mycobacteria from the Mycobacterium tuberculosis complex. Most cases of tuberculosis are caused by M. tuberculosis, but may also be the result of infection with M. africanum, M. bovis, M. bovis BCG, M. canetti, M. caprae, M. microti, M. mung, M. pinnipedii, M. suricattae, or another member of Mycobacterium tuberculosis complex. Tuberculosis infections primarily develop in the lungs and are referred to as pulmonary tuberculosis.

Tuberculosis infections may also be extra-pulmonary. Examples of extra-pulmonary tuberculosis infections include, but are not limited to: tuberculosis pleurisy (infection of the pleura or pleural cavity); tuberculosis meningitis, tuberculosis cerebritis, and tuberculosis myeltitis (infections of the central nervous system); tuberculosis pericarditis (infection of the pericardium); scrofula (infection of the lymphatic system in the neck), urogenital tuberculosis, and Pott disease/tuberculosis spondylitis (infection of the intervertebral joints). Tuberculosis infections in a subject may be pulmonary, extra-pulmonary, or both pulmonary and extra-pulmonary. A subject may develop drug resistant forms of tuberculosis. Multi-drug-resistant tuberculosis (MDR-TB) is defined as tuberculosis that is resistant to the first-line TB drugs isoniazid and rifampicin. Extensively drug-resistant tuberculosis (XDR-TB) is a form of tuberculosis that is resistant to the first-line drugs, and additionally shows resistance to a second-line TB drug or drugs (e.g., amikacin, kanamycin, capreomycin, ciprofloxacin, levofloxacin, moxifloxacin).

Staphylococcus aureus is a pathogenic bacteria that can cause skin infections (e.g., pimples, impetigo, boils, cellulitis folliculitis, carbuncles, scaled skin syndrome, and abcesses), pneumonia, meningitis, osteomyelitis, endocarditis, toxic shock syndrome, bacteremia, sepsis, sinusitis, and other diseases. Methicillin-resistant S. aureus (MRSA) refers to strains of S. aureus that are resistant to methicillin. MRSA infections are typically resistant to most β-lactam antibiotics (e.g., penicllins, cephalosporins), not just methicillin. Strains of S. aureus that are susceptible to treatment with methicillin and other β-lactams are referred to as methicillin-sensitive S. aureus (MSSA). In some embodiments, healthcare acquired MRSA (HA-MRSA) refers to MRSA infection that are acquired by subject at hospitals and other healthcare facilities. In some embodiments, community associated MRSA (CA-MRSA) refers to MRSA infections that are acquired by subjects not exposed to healthcare facilities. Some strains of MRSA are also resistant to vancomycin (or other glycopeptide antibiotics), which is the antibiotic most commonly used to treat MRSA. Classes of vancomycin resistant strains include vancomycin-intermediate S. aureus (VISA) and vancomycin-resistant S. aureus (VRSA).

As used herein, the term “o-succinylbenzoate-CoA synthetase” or “MenE” refers to an enzyme of the menaquinone biosynthesis pathway which converts o-succinylbenzoate to o-succinylbenzoate-CoA. In some species, MenE or a MenE homolog may participate in pathways other than menaquinone biosynthesis (e.g., 1,4-dihydroxy-2-naphthoate biosynthesis in Arabidopsis thaliana). MenE and their respective encoding RNA and DNA sequences according to some aspects of this invention include MenE protein and encoding sequences from bacteria, as well as, in some embodiments, MenE proteins and encoding sequences from other species, for example, from plants (e.g., Arabidopsis). In some embodiments, a MenE inhibitor provided herein is specific for a MenE from a species, e.g., for E. coli MenE, S. aureus MenE, M. tuberculosis MenE, and so on. In some embodiments, a MenE inhibitor provided herein inhibits MenEs from more than one species, e.g., S. aureus MenE and M. tuberculosis MenE. In some embodiments, a MenE provided herein exhibits equipotent inhibition of MenEs from more than one species, e.g., equipotent inhibition of S. aureus and M. tuberculosis MenEs. The term MenE further includes, in some embodiments, sequence variants and mutations (e.g., naturally occurring or synthetic MenE sequence variants or mutations), and different MenE isoforms. In some embodiments, the term MenE includes protein or encoding sequences that are homologous to a MenE protein or encoding sequence, for example, a protein or encoding sequence having at least 80%, at least 85%, at least 90%, at least 91%, at least 92%, at least 93%, at least 94%, at least 95%, at least 96%, at least 97%, at least 98%, at least 99%, or at least 99.5% sequence identity with a MenE sequence, for example, with a MenE sequence provided herein. MenE protein and encoding gene sequences are well known to those of skill in the art, and exemplary protein sequences include, but are not limited to, the following sequences. Additional MenE sequences, e.g., MenE homologues from other bacteria species, will be apparent to those of skill in the art, and the invention is not limited to the exemplary sequences provided herein.

>gi|520813|ref|AAB04893.1| o-succinylbenzoate-CoA
[Escherichia coli]
(SEQ ID NO: 1)
MIFSDWPWRHWRQVRGETIALRLNDEQLNWRELCARVDELASGFAVQGVV
EGSGVMLRAWNTPQTLLAWLALLQCGARVLPVNPQLPQPLLEELLPNLTL
QFALVPDGENTFPALTSLHIQLVEGAHAATWQPTRLCSMTLTSGSTGLPK
AAVHTYQAHLASAQGVLSLIPFGDHDDWLLSLPLFHVSGQGIMWRWLYAG
ARMTVRDKQPLEQMLAGCTHASLVPTQLWRLLVNRSSVSLKAVLLGGAAI
PVELTEQAREQGIRCFCGYGLTEFASTVCAKEADGLADVGSPLPGREVKI
VNNEVWLRAASMAEGYWRNGQLVSLVNDEGWYATRDRGEMHNGKLTIVGR
LDNLFFSGGEGIQPEEVERVIAAHPAVLQVFNVPVADKEFGHRPVAVMEY
DHESVDLSEWVKDKLARFQQPVRWLTLPPELKNGGIKISRQALKEWVQRQ
Q
>gi|2293149|ref|AAC00227.1| o-succinylbenzoate-CoA
[Bacillus subtilis]
(SEQ ID NO: 2)
MLTEQPNWLMQRAQLTPERIALIYEDQTVTFAELFAASKRMAEQLAAHSV
RKGDTAAILLQNRAEMVYAVHACFLLGVKAVLLNTKLSTHERLFQLEDSG
SGFLLTDSSFEKKEYEHIVQTIDVDELMKEAAEEIEIEAYMQMDATATLM
YTSGTTGKPKGVQQTFGNHYFSAVSSALNLGITEQDRWLIALPLFHISGL
SALFKSVIYGMTVVLHQRFSVSDVLHSINRHEVTMISAVQTMLASLLEET
NRCPESIRCILLGGGPAPLPLLEECREKGFPVFQSYGMTETCSQIVTLSP
EFSMEKLGSAGKPLFSCEIKIERDGQVCEPYEHGEIMVKGPNVMKSYFNR
ESANEASFQNGWLKTGDLGYLDNEGFLYVLDRRSDLIISGGENIYPAEVE
SVLLSHPAVAEAGVSGAEDKKWGKVPHAYLVLHKPVSAGELTDYCKERLA
KYKIPAKFFVLDRLPRNASNKLLRNQLKDARKGELL
>gi|755917608|ref|AJK60576.1| o-succinylbenzoate-
CoA [Mycobacterium tuberculosis 18b]
(SEQ ID NO: 3)
MLGGSDPALVAVPTQHESLLGALRVGEQIDDDVALVVTTSGTTGPPKGAM
LTAAALTASASAAHDRLGGPGSWLLAVPPYHIAGLAVLVRSVIAGSVPVE
LNVSAGFDVTELPNAIKRLGSGRRYTSLVAAQLAKALTDPAATAALAELD
AVLIGGGPAPRPILDAAAAAGITVVRTYGMSETSGGCVYDGVPLDGVRLR
VLAGGRIAIGGATLAKGYRNPVSPDPFAEPGWFHTDDLGALESGDSGVLT
VLGRADEAISTGGFTVLPQPVEAALGTHPAVRDCAVFGLADDRLGQRVVA
AIVVGDGCPPPTLEALRAHVARTLDVTAAPRELHVVNVLPRRGIGKVDRA
ALVRRFAGEADQ
>gi|320143759|ref|EFW35535.1| o-succinylbenzoate-
CoA [Staphylococcus aureus subsp. aureus MRSA177]
(SEQ ID NO: 4)
MDFWLYKQAQQNGHHIAITDGQESYTYQNLYCEASLLAKRLKAYQQSRVG
LYIDNSIQSIILIHACWLANIEIAMINTRLTPNEMTNQMKSIDVQLIFCT
LPLELRGFQIVSLDDIEFAGRDITTNSLLDNTMGIQYETSNETVVPKESP
SNILNTSFNLDDIASIMFTSGTTGPQKAVPQTFRNHYASAIGCKESLGFD
RDTNWLSVLPIYHISGLSVLLRAVIEGFTVRIVDKFNAEQILTMIKNERI
THISLVPQTLNWLMQQGLHEPYNLQKILLGGAKLSATMIETALQYNLPIY
NSFGMTETCSQFLTATPEMLHARPDTVGMPSANVDVKIKNPNKEGHGELM
IKGANVMNVYLYPTDLTGTFENGYFNTGDIAEIDHEGYVMIYDRRKDLII
SGGENIYPYQIETVAKQFPGISDAVCVGHPDDTWGQVPKLYFVSESDISK
AQLIAYLSQHLAKYKVPKHFEKVDTLPYTSTGKLQRNKLYRG

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Synthesis of the Compounds

In some examples, the methods of synthesis are adapted from those described in References 1, 2, and 3, which are incorporated herein by reference. The synthesis of the OSB-AMP/OSB-AMS analogues generally proceeded by initial synthesis of the left hand acyl chain 10 (Scheme E1), followed by coupling of the acyl chain with the protected adenosinemonosulfamate (AMS) scaffold 11. The product was then globally deprotected to attain the desired compound 13. Other solvents, such as THF, may be used in place of dichloromethane for the amide formation and deprotection steps. Additionally the nucleoside (or nucleoside analog) fragment is not limited to the adenosinemonosulfamate as shown in Scheme 1. For example, other protecting groups may be used, and the nucleobase, ribose, and/or sulfamoyl moieties may be replaced with other moieties consistent with compounds of Formulae (I′) and (I). Using this general method, we were able to obtain compounds 103, 104, 105, 106, and 107. Alternative synthetic strategies were necessary to gain access to lactam (108) and difluoro (109) analogues due to reactivity associated with their individual structures. The syntheses and preparative details of specific OSB-AMS analogues are shown in Scheme E2-E11 and described below.

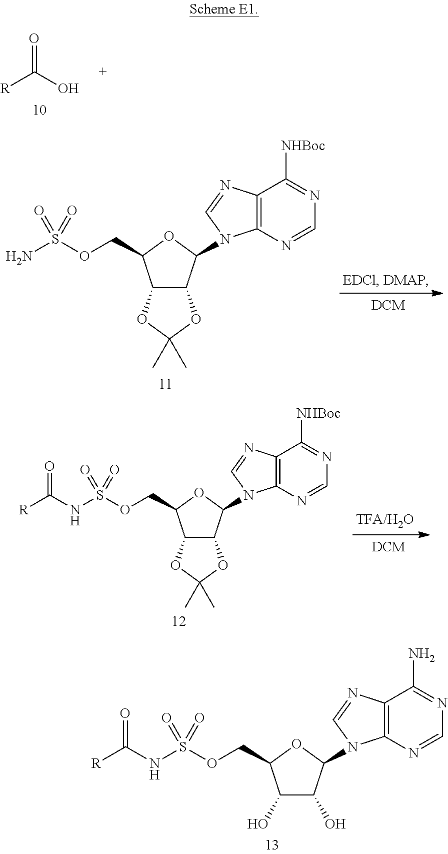

Synthesis of a m-Succinylbenzoate Analog (Compound 102)

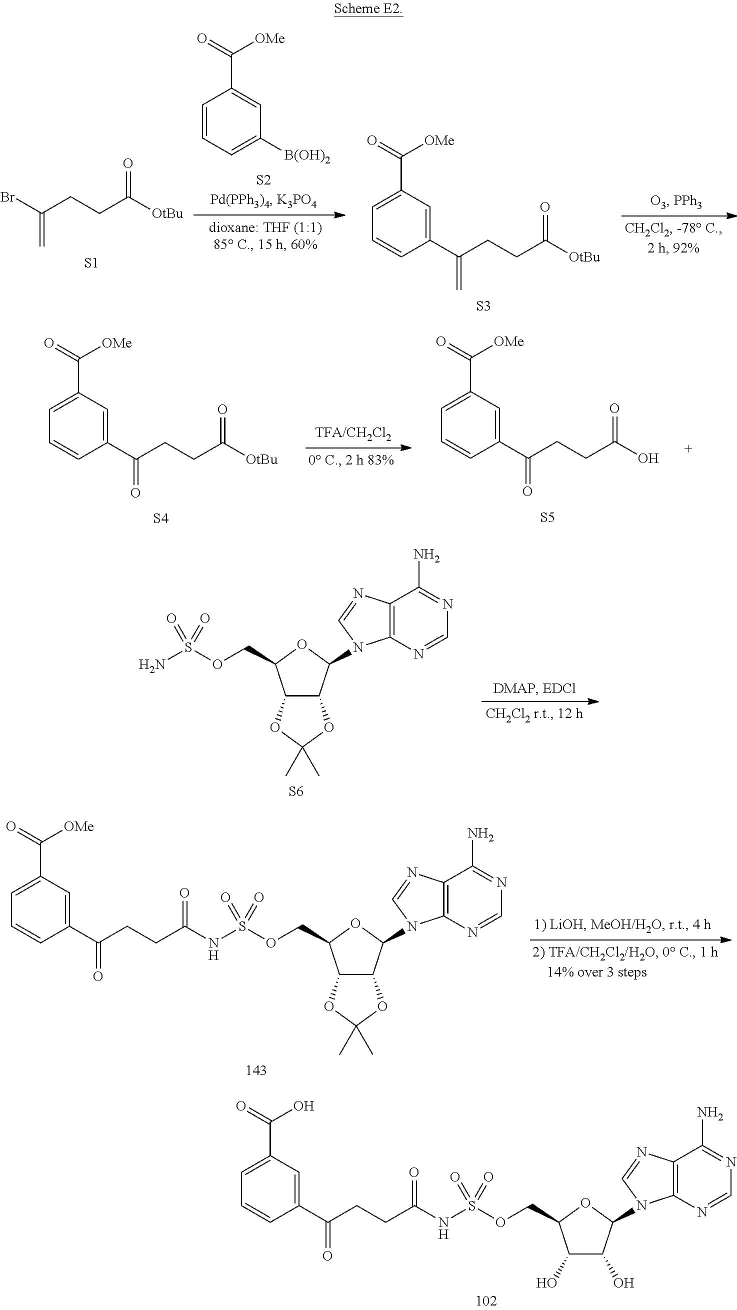

### Methyl 3-(5′-tert-butoxy-5′-oxopent-1′-en-2′-yl)benzoate (S3)

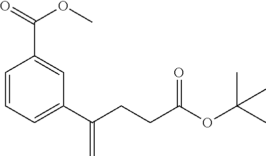 [SMILES: C=C(CCC(=O)OC(C)(C)C)c1cccc(C(=O)OC)c1]

Vinyl bromide S1 (1 g, 4.2532 mmol, 1 equiv.), boronic acid S2 (1.148 g, 6.380 mmol, 1.5 equiv.), Pd(PPh₃)₄ (491 mg, 0.42532 mmol, 0.1 equiv.), and K₃PO₄ (2.708 g, 12.760 mmol, 4.0 equiv.) were suspended in 40 mL of dioxane/THF (1:1) and stirred for 15 hours at 85° C. The reaction was then diluted with 100 mL water and extracted with Et₂O (4×100 mL). The combined organic extracts were dried (Na₂SO₄), filtered, and concentrated by rotary evaporation. Purification by silica flash chromatography (0%→20% EtOAc in hexanes) yielded styrene diester S3 as a clear and colorless oil (735 mg, 60%). IR (ATR): 2978.97, 1723.04, 1630.53, 1581.07, 1439.53, 1367.22, 1253.24, 1147.74, 985.13, 903.23, 846.92, 819.49, 763.84, 719.16. ¹H-NMR (600 MHz; CDCl3): δ 8.08 (t, J=1.7 Hz, 1H), 7.95 (d, J=7.7 Hz, 1H), 7.59 (d, J=7.8 Hz, 1H), 7.41 (t, J=7.7 Hz, 1H), 5.35 (s, 1H), 5.15 (s, 1H), 3.93 (s, 3H), 2.82 (t, J=7.7 Hz, 2H), 2.39 (dd, J=8.4, 7.0 Hz, 2H), 1.44 (s, 9H). ¹³C-NMR (151 MHz; CDCl3): δ 172.3, 167.1, 146.2, 141.1, 130.6, 130.2, 128.65, 128.48, 127.3, 113.7, 80.4, 52.2, 34.1, 30.4, 28.1. HRMS (ESI) m/z calcd for C₁₇H₂₂O₄Na ([M+Na]⁺) 313.1416; found 313.1419.

### Methyl 3-(4′-tert-butoxy-4′-oxobutanoyl)benzoate (S4)

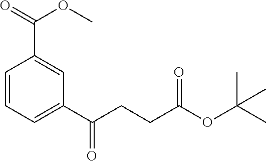 [SMILES: COC(=O)c1cccc(C(=O)CCC(=O)OC(C)(C)C)c1]

Styrene diester S3 (412 mg, 1.419 mmol, 1 equiv.) was dissolved in 15 mL CH₂Cl₂ and cooled to −78° C. Ozone was bubbled into the reaction at −78° C. until the solution remained a clear, light blue color. Nitrogen gas was then bubbled through the reaction until the blue color disappeared. PPh₃ (410 mg, 1.561 mmol, 1.1 equiv.) was added to the reaction slowly in one portion, then the mixture was allowed to warm to room temperature over 2 hours. Concentration by rotary evaporation and purification by silica flash chromatography (0→15% EtOAc in hexanes) yielded keto diester S4 as a clear and colorless oil (415 mg, 92%). IR (ATR): 2980.18, 1724.63, 1691.63, 1603.25, 1433.66, 1366.23, 1283.95, 1201.70, 1150.42, 963.58, 915.49, 847.03, 751.77, 685.46. ¹H-NMR (600 MHz; CDCl₃): δ 8.64 (t, J=1.5 Hz, 1H), 8.24 (dt, J=7.7, 1.4 Hz, 1H), 8.19 (ddd, J=7.8, 1.7, 1.3 Hz, 1H), 7.57 (t, J=7.7 Hz, 1H), 3.96 (s, 3H), 3.31 (t, J=6.6 Hz, 2H), 2.72 (t, J=6.5 Hz, 2H), 1.46 (s, 9H). ¹³C-NMR (151 MHz; CDCl₃): δ 197.6, 172.1, 166.3, 136.8, 134.0, 132.2, 130.6, 129.2, 128.9, 80.8, 52.5, 33.6, 29.3, 28.1. HRMS (ESI) m/z calcd for C₁₆H₂₀O₅Na ([M+Na]⁺) 315.1208; found 315.1203.

### 4-(3′-[Methoxycarbonyl]phenyl)-4-oxobutanoic acid (S5)

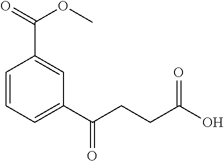 [SMILES: COC(=O)c1cccc(C(=O)CCC(=O)O)c1]

Keto diester S4 (300 mg, 1.0262 mmol, 1.0 equiv.) was dissolved in 5 mL CH₂Cl₂ and cooled to 0° C., then 5 mL TFA was added and the reaction stirred for 2 hours. Concentration by rotary evaporation and purification by silica flash chromatography (50% EtOAc in hexanes with 1% AcOH) yielded keto acid S5 as a white semisolid (200 mg, 83%). IR (ATR): 2954.60, 1718.83, 1689.82, 1603.42, 1432.81, 1362.35, 1299.69, 1205.13, 1107.05, 961.91, 810.60, 751.23, 684.51. ¹H-NMR (600 MHz; CDCl3): δ 8.62 (t, J=1.5 Hz, 1H), 8.25 (dt, J=7.7, 1.4 Hz, 1H), 8.18 (dt, J=7.8, 1.5 Hz, 1H), 7.57 (t, J=7.8 Hz, 1H), 3.96 (s, 3H), 3.36 (t, J=6.5 Hz, 2H), 2.84 (t, J=6.5 Hz, 2H). ¹³C-NMR (151 MHz; CDCl3): δ 197.0, 178.7, 166.3, 136.6, 134.2, 132.2, 130.7, 129.2, 129.0, 52.5, 33.3, 28.0. HRMS (ESI) m/z calcd for C₁₂H₁₁O₅ ([M−H]⁻) 235.0607; found 235.0608.

### Compound 143: 2′,3′-O-Isopropylidene-5′-O—(N-[4″-(3′″-[methoxycarbonyl]phenyl)-4″-oxobutanoyl]sulfamoyl)adenosine

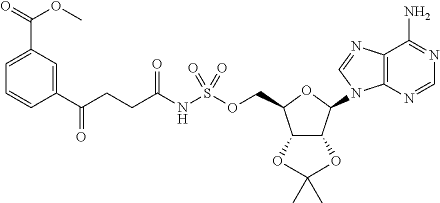 [SMILES: COC(=O)c1cccc(C(=O)CCC(=O)NS(=O)(=O)OC[C@H]2O[C@@H](n3cnc4c(N)ncnc43)[C@@H]3OC(C)(C)O[C@@H]32)c1]

Keto acid S5 (200 mg, 0.846 mmol, 1 equiv.), protected 5′-O-sulfamoyladenosine S6 (490 mg, 1.269 mmol, 1.5 equiv.), and DMAP (113.7 mg, 0.931 mmol, 1.1 equiv.) were dissolved in 5 mL CH₂Cl₂ and EDCI (645.6 mg, 3.386 mmol, 4.0 equiv.) was added. The reaction stirred for 12 hours, then diluted with 25 mL water, and extracted with CH₂Cl₂ (4×25 mL). The combined organic extracts were dried (Na₂SO₄), filtered through a pad of celite, and concentrated by rotary evaporation to afford the crude protected MSB-AMS S7 (995 mg, 158% crude yield), which was used without further purification.

### Compound 102: 5′-O—(N-[4″-(3′″-(Carboxyl)phenyl)-4″-oxobutanoyl]sulfamoyl)adenosine

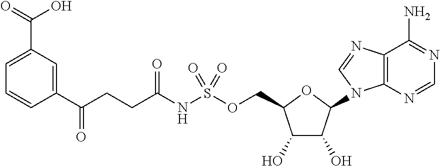 [SMILES: Nc1ncnc2c1ncn2[C@@H]1O[C@H](COS(=O)(=O)NC(=O)CCC(=O)c2cccc(C(=O)O)c2)[C@@H](O)[C@H]1O]

Crude protected MSB-AMS S7 (assumed quantitative yield: 512 mg, 0.846 mmol, 1 equiv.) and LiOH (81 mg, 3.386 mmol, 4 equiv.) were suspended in 5 mL MeOH/H₂O (9:1) and stirred for 4 hours at room temperature. The MeOH was removed by rotary evaporation and the crude residue was dissolved in 10 mL CH₂Cl₂ and cooled to 0° C. TFA (10 mL) was added and the reaction was stirred for 1 hours. Concentration by rotary evaporation, purification by preparative HPLC (5%→95% MeCN in H₂O with 0.01% TFA), and lyophilization yielded MSB-AMS (102) as white fluffy solid (65 mg, 14% over 3 steps). IR (ATR): 3134, 1698, 1614, 1508, 1468, 1421.64, 1375, 1288, 1187, 1133, 977, 940, 894, 799, 767, 722, 699, 639. ¹H-NMR (600 MHz; CDCl₃): δ 8.46 (d, J=1.2 Hz, 1H), 8.41 (s, 1H), 8.29 (s, 1H), 8.14-8.10 (m, 2H), 7.51 (t, J=7.8 Hz, 1H), 6.07 (d, J=5.4 Hz, 1H), 4.68-4.56 (m, 3H), 4.39 (t, J=4.1 Hz, 2H), 3.49-3.36 (m, 2H), 2.78-2.66 (m, 2H). ¹³C-NMR (151 MHz; CDCl₃): δ 201.7, 175.7, 171.2, 155.4, 152.7, 149.9, 145.5, 140.3, 137.4, 135.8, 135.0, 132.6, 122.6, 92.1, 86.3, 78.7, 74.8, 74.2, 52.1, 36.8, 33.2. HRMS (ESI) m/z calcd for C₂₁H₂₃N₆O₁₀S ([M+H]⁺) 551.1196; found 551.1204.

### Synthesis of a Nitro Analog (Compound 103)

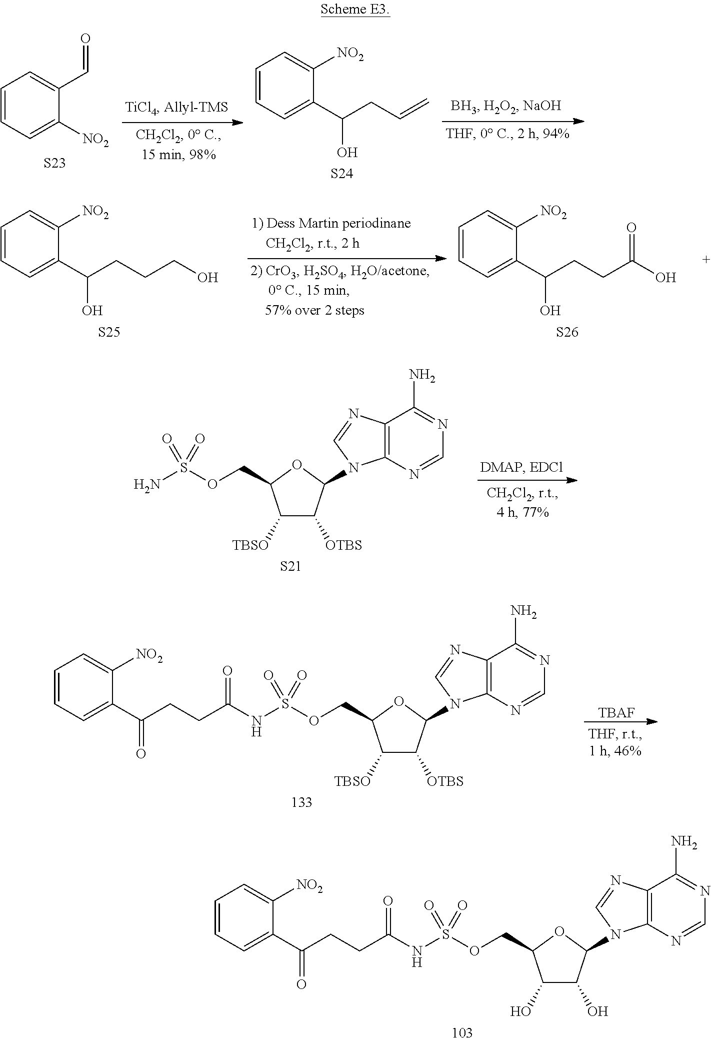

### 1-(2-Nitrophenyl)but-3-enol (S24)

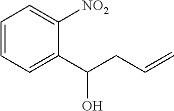

2-Nitrobenzaldehyde S23 (1 g, 6.617 mmol, 1 equiv.) was dissolved in CH₂Cl₂ (10 mL), cooled to 0° C., and TiCl₄ (3.3087 mL, 3.3087 mmol, 0.5 equiv., 1.0 M in THF) was added slowly over 10 minutes before being removed from the ice bath and stirred for 10 minutes. Allyl trimethylsilane (1.134 g, 1.578 mmol, 1.5 equiv.) was added quickly, then the reaction was stirred for 15 minutes, poured into Et₂O (100 mL) and the solution washed with saturated NaCl solution (100 mL). The organic layer was dried (Na₂SO₄), filtered, and concentrated by rotary evaporation. Purification by silica flash chromatography (10%→50% CH₂Cl₂ in hexanes) yielded the title product (S24) as a clear, red oil (1.252 g, 98%). IR (ATR): 3420.34, 3078.63, 2909.77, 1603.27, 1519.18, 1347.06, 1107.26, 1055.99, 992.67, 921.67, 854.77, 751.96, 699.88. ¹H-NMR (600 MHz): δ 7.93 (dd, J=8.2, 1.2, 1H), 7.83 (dd, J=7.9, 1.4, 1H), 7.65 (td, J=7.6, 1.1, 1H), 7.43 (ddd, J=8.3, 7.2, 1.2, 1H), 5.89 (dddd, J=16.9, 10.4, 7.9, 6.4, 1H), 5.31 (dd, J=8.3, 2.3, 1H), 5.22-5.20 (m, 1H), 5.19 (t, J=1.4, 1H), 2.71 (dddt, J=14.1, 6.3, 3.7, 1.4, 1H), 2.48 (s, 1H), 2.45-2.39 (m, 1H) ¹³C-NMR (150 MHz): δ 147.76, 139.27, 134.02, 133.52, 128.18, 128.13, 124.45, 119.17, 68.40, 42.92. HRMS (ESI) m/z calcd for C₁₀H₁₂NO₃ ([M+H]⁺) 194.0817; found 194.0830.

### 1-(2-Nitrophenyl)butane-1,4-diol (S25)

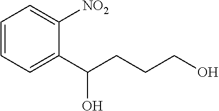 [SMILES: O=[N+]([O-])c1ccccc1C(O)CCCO]

Cyclohexene (475 mg, 5.78 mmol, 0.9 equiv.) was dissolved in THF (5 mL), cooled to 0° C., and BH₃ (7.7 mL, 7.70 mmol, 1.2 equiv., 1.0 M in THF) added before stirring for 10 minutes. Alkene S24 (1.24 g, 6.418 mmol, 1 equiv.) in THF (5 mL) was added drop wise before being returned to room temperature and stirred for 30 minutes. NaOH (2.58 mL, 9.63 mmol, 1.5 equiv., 3.75 M) was added drop wise followed by H₂O₂ (1.1219 mL, 11.23 mmol, 1.7 equiv., 30% solution) added slowly over 10 minutes. The reaction was stirred for 20 minutes, poured into Et₂O (200 mL), washed with saturated ammonium chloride (100 mL), and saturated NaCl (100 mL). The organic layer was dried (Na₂SO₄), filtered, and concentrated by rotary evaporation. Purification by silica flash chromatography (10%→30% EtOAc in hexanes) yielded the title product (S25) (765 mg, 56%) as a light red solid and starting material S24 (470 mg, 38%). IR (ATR): 3350.48, 2944.41, 2875.54, 1658.27, 1602.80, 1518.13, 1414.03, 1347.48, 1050.61, 1012.02, 961.06, 855.52, 749.12, 700.88. ¹H-NMR (600 MHz): δ 7.85 (dd, J=8.2, 1.3, 1H), 7.77 (dd, J=7.9, 1.3, 1H), 7.59 (td, J=7.6, 1.1, 1H), 7.36 (td, J=7.8, 1.2, 1H), 5.19 (d, J=8.4, 1H), 4.64 (s, 1H), 3.67 (d, J=7.1, 1H), 3.60-3.57 (m, 2H), 1.93-1.88 (m, 1H), 1.75-1.65 (m, 3H). ¹³C-NMR (150 MHz): δ 147.43, 140.58, 133.55, 128.00, 127.95, 124.30, 69.09, 62.49, 35.95, 29.35. HRMS (ESI) m/z calcd for C₁₀H₁₃NO₄ ([M+Na]⁺) 234.0742; found 234.0735.

### 4-(2-Nitrophenyl)-4-oxobutanoic acid (S26)

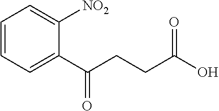 [SMILES: O=C(O)CCC(=O)c1ccccc1[N+](=O)[O-]]

1-(2-Nitrophenyl)butane-1,4-diol S25 (765 mg, 3.6219 mmol, 1 equiv.) in CH₂Cl₂ (10 mL) was added to a stirring solution of Dess-Martin periodinane (3.226 g, 7.606 mmol, 2 equiv.) in CH₂Cl₂ (15 mL) and stirred at room temperature for 2 hours. The reaction was diluted with Et₂O (100 mL), saturated sodium bicarbonate (75 mL), and sodium thiosulphate (5.727 g, 36.219 mmol, 7 equiv.) added. The reaction was then stirred vigorously until the solution became clear. The organic layer was then removed and washed with saturated sodium bicarbonate (50 mL) before concentrated by rotary evaporation and reconstituted in acetone (5 mL) and cooled to 0° C. Jones reagent [prepared as described with S15 using CrO₃ (1.81 g, 18.1 mmol, 5 equiv.) and conc. sulfuric acid (2.011 mL, 36.2 mmol, 10 equiv.) in water (4 mL)] was added drop wise to the crude aldehyde slowly over 30 minutes until the solution remained a persistent red color. The reaction was stirred for 15 minutes, quenched with isopropyl alcohol, diluted with water (50 mL), and extracted with Et₂O (4×50 mL). The combined organic extracts were dried (Na₂SO₄), filtered, and concentrated by rotary evaporation. Purification by silica flash chromatography (15%→30% EtOAc in hexanes with 1% AcOH) yielded the title product (S26) as a red tinged solid (460 mg, 57% over 2 steps). IR (ATR): 3112.58, 3081.72, 2946.01, 1782.82, 1695.14, 1604.92, 1522.37, 1458323, 1414.94, 1348.91, 1293.56, 1215.16, 1175.85, 1143.65, 1109.91, 1032.97, 992.20, 942.01, 856.38, 818.10, 748.18, 699.43. ¹H-NMR (600 MHz): δ 11.27 (s, 1H), 8.14 (dd, J=8.2, 0.7, 1H), 7.75 (td, J=7.5, 1.0, 1H), 7.64-7.61 (m, 1H), 7.49 (dd, J=7.6, 1.3, 1H), 3.15 (t, J=6.5, 2H), 2.90 (t, J=6.5, 2H). ¹³C-NMR (1506 MHz): δ 170.59, 170.30, 147.53, 146.88, 138.77, 138.43, 133.85, 133.80, 128.19, 128.124, 127.72, 124.29, 124.89, 124.52, 99.24, 98.90, 80.02, 33.30, 31.65, 31.53, 31.03, 21.46, 21.41. HRMS (ESI) m/z calcd for C₁₀H₉NO₅Na ([M+Na]⁺) 246.0378; found 246.0370.

### Compound 133: 2′,3′-O-TBS-5′-O—(N-[4-(2-nitrophenyl)-4-oxobutanoyl]sulfamoyl)adenosine

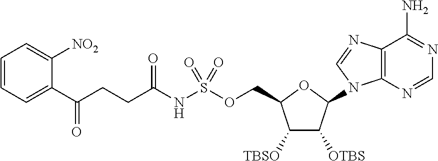 [SMILES: CC(C)(C)[Si](C)(C)O[C@@H]1[C@H](O[Si](C)(C)C(C)(C)C)[C@@H](COS(=O)(=O)NC(=O)CCC(=O)c2ccccc2[N+](=O)[O-])O[C@H]1n1cnc2c(N)ncnc21]

Keto acid S26 (100 mg, 0.4481 mmol, 1 equiv.), protected 5′-O-sulfamoyladenosine (386 mg, 0.6722 mmol, 1.5 equiv.), and DMAP (55 mg, 0.448 mmol, 1 equiv.) was dissolved in CH₂Cl₂ (25 mL) and EDCI (342 mg, 1.7924 mmol, 4 equiv.) added. The reaction was stirred for 4 hours, quenched with water (25 mL), and extracted with CH₂Cl₂ (5×25 mL). The combined organic extracts were dried (Na₂SO₄), filtered through a pad of celite, and concentrated by rotary evaporation to afford the crude protected nitro analogue 133 (443 mg, 127% crude yield), which was used without further purification.

### Compound 103: 5′-O—(N-[4-(2-Nitrophenyl)-4-oxobutanoyl]sulfamoyl)adenosine

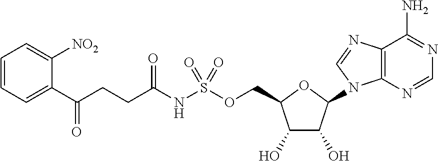 [SMILES: Nc1ncnc2c1ncn2[C@@H]1O[C@H](COS(=O)(=O)NC(=O)CCC(=O)c2ccccc2[N+](=O)[O-])[C@@H](O)[C@H]1O]

Crude protected nitro analogue 133 from previous step was dissolved in THF (10 mL) and cooled to 0° C. TBAF (1.34 mL, 1.34 mmol, 3 equiv., 1.0 M in THF) was added and allowed to stir for 1 hour. Concentration by rotary evaporation, purification by preparative HPLC (5%→95% MeCN in H₂O with 0.01% TFA), and lyophilization yielded the product (103) as a red fluffy solid (84 mg, 35% yield over 2 steps). IR (ATR): 3398, 2959, 2930, 2853, 1694, 1611, 1529, 1470, 1418, 1350, 1202, 1137, 1040, 836, 720. ¹H-NMR (600 MHz; MeOD): δ 8.48 (s, 1H), 8.35 (s, 1H), 8.08 (dd, J=8.2, 1.0 Hz, 1H), 7.78 (td, J=7.5, 1.1 Hz, 1H), 7.70-7.67 (m, 1H), 7.61 (dd, J=7.6, 1.3 Hz, 1H), 6.10 (d, J=4.9 Hz, 1H), 4.63 (t, J=5.0 Hz, 1H), 4.62-4.55 (m, 2H), 4.41 (t, J=4.9 Hz, 1H), 4.34 (q, J=3.8 Hz, 1H), 3.19-3.17 (m, 2H), 2.75 (t, J=6.2 Hz, 2H). ¹³C-NMR (150 MHz; MeOD/D₂O): δ 202.635, 172.516, 152.690, 150.516, 147.336, 146.732, 143.478, 137.917, 135.442, 132.367, 129.050, 125.491, 120.446, 90.364, 83.642, 75.914, 72.329, 71.700, 37.646, 30.835. HRMS (ESI) m/z calcd for C₂₀H₂₁N₇O₁₀SNa ([M+Na]⁺) 574.0968; found 574.0973.

### Synthesis of an Oxazole Analog (Compound 104)

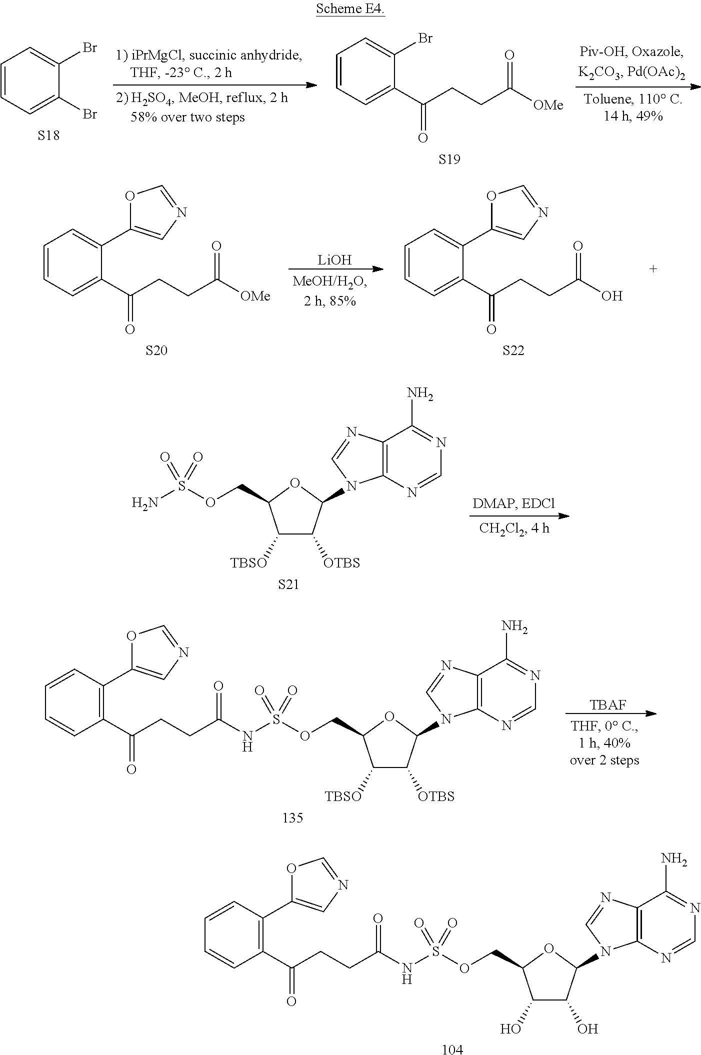

### Methyl 4-(2-bromophenyl)-4-oxobutanoate (S19)

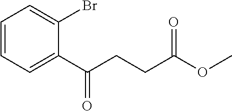

Isopropylmagnesium chloride (7.89 mL, 10.26 mmol, 1.1 equiv., 1.3 M in THF) was cooled to −23° C. and 1, 2 dibromobenzene S18 (2.2 g, 9.3 mmol, 1 equiv.) was added. The reaction was stirred for 45 minutes, then slowly transferred via cannula to a stirring solution of succinic anhydride (2.799 g, 27.977 mmol, 3.0 equiv.) in THF (20 mL) at −23° C. The reaction was stirred for 2 hours, then quenched with ammonium chloride, acidified with 50 mL 1 M KHSO₄, and extracted with dichloromethane (3×50 mL). The combined organic extracts were dried (Na₂SO₄), filtered, and concentrated by rotary evaporation. The crude material was dissolved in MeOH (50 mL) and cone. sulfuric acid (92 mg, 0.9326 mmol, 0.1 equiv.). The reaction was heated to reflux for 4 hours and cooled to room temperature. The reaction was reduced to approximately 10 mL by rotary evaporation, diluted with 50 mL saturated sodium bicarbonate and extracted with dichloromethane (5×50 mL). The combined organic extracts were dried (Na₂SO₄), filtered, and concentrated by rotary evaporation. Purification by silica flash chromatography (10% EtOAc in hexanes) yielded the product (S19) as a clear and colorless oil (1.45 g, 58% yield). IR (ATR): 2952, 1736, 1703, 1587, 1564, 1467, 1436, 1354, 1020, 1281, 1217, 1167, 1123, 1072, 1048, 1027, 993, 946, 906, 847, 753, 722, 684, 642. ¹H-NMR (600 MHz; CDCl₃): δ 7.61 (dd, J=8.0, 1.0 Hz, 1H), 7.49 (dd, J=7.6, 1.7 Hz, 1H), 7.38 (td, J=7.5, 1.1 Hz, 1H), 7.30 (td, J=7.7, 1.7 Hz, 1H), 3.71 (s, 3H), 3.24 (t, J=6.6 Hz, 2H), 2.78 (t, J=6.6 Hz, 2H). ¹³C-NMR (150 MHz; CDCl₃): δ 202.0, 173.0, 141.2, 133.7, 131.8, 128.8, 127.5, 118.7, 52.0, 37.4, 28.2. HRMS (ESI) m/z calcd for C₁₁H₁₂O₃Br ([M+H]⁺) 270.9970; found 270.9979.

### Methyl 4-(2-bromophenyl)-4-oxobutanoate (S20)

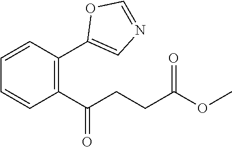 [SMILES: COC(=O)CCC(=O)c1ccccc1-c1cnco1]

Methyl 4-(2-bromophenyl)-4-oxobutanoate (130 mg, 0.4795 mmol, 1 equiv.), pivalic acid (20 mg, 0.1918 mmol, 0.4 equiv.), oxazole (66 mg, 0.959 mmol, 2.0 equiv.), Pd(OAc)₂ (11 mg, 0.048 mmol, 0.1 equiv.), RuPhos (45 mg, 0.0959 mmol, 0.2 equiv.), and K₂CO₃ (199 mg, 1.4385 mmol, 3.0 equiv.) were suspended in 2 mL toluene and stirred at 110° C. for 14 hours. The reaction was then poured into 5 mL H₂O and extracted with dichloromethane (4×5 mL), organics combined, dried over sodium sulfate and stripped of solvent under vacuum. The residue resolved by silica chromatography (15%->30% EtOAc/Hex) to yield the title product (60 mg, 49% yield) as a clear oil. IR (NaCl, Film): 1736.76, 1704.37, 1559.23, 1515.92, 1437.52, 1358.55, 1319.33, 1217.31, 1168.77, 1075.65, 1027.36, 987.43, 947.68, 919.55, 844.65, 779.24, 747.73, 716.72. ¹H-NMR (600 MHz; CDCl3): δ 7.99-7.97 (m, 1H), 7.71 (s, 1H), 7.55-7.50 (m, 2H), 7.44-7.43 (m, 1H), 7.22 (s, 1H), 3.11 (t, J=6.8 Hz, 2H), 2.82 (t, J=6.8 Hz, 2H). ¹³C-NMR (150 MHz): δ 204.638, 173.360, 160.180, 140.929, 139.064, 130.361, 130.002, 128.695, 128.136, 126.696, 123.891, 51.863, 38.086, 28.489. HRMS (ESI) m/z calcd for C₁₄H₁₃NO₄Na ([M+H]⁺) 282.0742; found 282.0736.

### 4-(2-(5-Oxazolyl)phenyl)-4-oxobutanoic acid (S22)

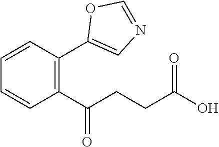 [SMILES: O=C(O)CCC(=O)c1ccccc1-c1cnco1]

Methyl ester S20 (50 mg, 0.1929 mmol, 1 equiv.) and LiOH (14 mg, 0.5787 mmol, 3.0 equiv.) was dissolved in MeOH/H₂O (2 mL, 10:1) and stirred at room temperature for 2 hours. The reaction was concentrated by rotary evaporation and purified by silica flash chromatography (25%→50% EtOAc in hexanes with 1% AcOH) to yield the product (S22) as an off white solid (40 mg, 85%). IR (ATR): 1703.45, 1584.21, 1559.62, 1398.48, 1359.78, 1220.19, 1165.26, 1106.34, 1075.18, 991.12, 916.02, 824.43, 777.84, 731.25. ¹H-NMR (600 MHz; CDCl₃): δ 7.99-7.97 (m, 1H), 7.71 (d, J=0.7 Hz, 1H), 7.53 (qdd, J=7.8, 7.4, 1.6 Hz, 2H), 7.43-7.41 (m, 1H), 7.23 (d, J=0.5 Hz, 1H), 3.11 (t, J=6.7 Hz, 2H), 2.86 (t, J=6.7 Hz, 2H). ¹³C-NMR (150 MHz): δ 204.359, 178.129, 160.181, 140.702, 139.153, 130.419, 130.131, 128.625, 128.248, 126.691, 123.891, 37.820, 28.507. HRMS (ESI) m/z calcd for C₁₃H₁₁NO₄Na ([M+Na]⁺) 268.0586; found 268.0578.

### Compound 135: 2′,3′-O-TBS-5′-O—(N-[4-(2-(5-oxazolyl)phenyl)-4-oxobutanoyl]sulfamoyl)adenosine

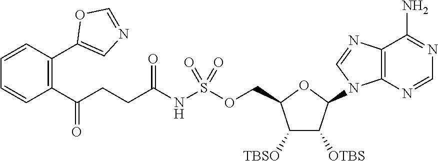 [SMILES: CC(C)(C)[Si](C)(C)O[C@@H]1[C@H](O[Si](C)(C)C(C)(C)C)[C@@H](COS(=O)(=O)NC(=O)CCC(=O)c2ccccc2-c2cnco2)O[C@H]1n1cnc2c(N)ncnc21]

Keto acid S22 (52 mg, 0.212 mmol, 1 equiv.), protected 5′-O-sulfamoyladenosine S21 (152 mg, 0.265 mmol, 1.25 equiv.) and DMAP (26 mg, 0.212 mmol, 1 equiv.) were dissolved in CH₂Cl₂ and EDCI (121 mg, 0.636 mmol, 3 equiv.) added. The reaction was stirred at room temperature for 4 hours, quenched with 20 mL water, extracted with dichloromethane (5×20 mL). The combined organic extracts were dried (Na₂SO₄), filtered through a pad of celite, and concentrated by rotary evaporation to afford the crude protected oxazole analogue 135 (240 mg, 141% crude yield), which was used without further purification.

### Compound 104: 5′-O—(N-[4-(2-(5-Oxazolyl)phenyl)-4-oxobutanoyl]sulfamoyl)adenosine

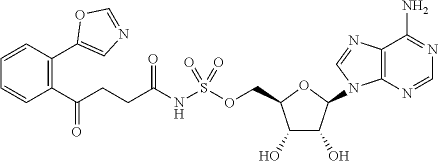 [SMILES: Nc1ncnc2c1ncn2[C@@H]1O[C@H](COS(=O)(=O)NC(=O)CCC(=O)c2ccccc2-c2cnco2)[C@@H](O)[C@H]1O]

Crude protected oxazole analogue 135 from previous step was dissolved in THF (2 mL), cooled to 0° C. and TBAF (0.3 mL, 0.2991 mmol, 3 equiv., 1.0 M in THF) was added before stirring for 1 hour. Concentration by rotary evaporation, purification by preparative HPLC (5%→95% MeCN in H₂O with 0.01% TFA), and lyophilization yielded the product (104) as a white fluffy solid (23 mg, 40% over 2 steps). IR (NaCl, Film): 3324.63, 3131.45, 2922.10, 2824.51, 1697.90, 1471.83, 1421.59, 1364.73, 1199.49, 1135.06, 978.54, 885.98, 830.56, 721.30. ¹H-NMR (600 MHz; MeOD): δ 8.48 (s, 1H), 8.34 (s, 1H), 7.95 (d, J=0.8 Hz, 1H), 7.91 (dd, J=7.6, 1.0 Hz, 1H), 7.61 (td, J=7.5, 1.5 Hz, 1H), 7.57 (td, J=7.5, 1.3 Hz, 1H), 7.53 (dd, J=7.6, 1.2 Hz, 1H), 7.26 (d, J=0.7 Hz, 1H), 6.10 (d, J=4.9 Hz, 1H), 4.62 (t, J=5.0 Hz, 1H), 4.58 (qd, J=11.0, 3.3 Hz, 2H), 4.40 (t, J=4.8 Hz, 1H), 4.33 (q, J=3.8 Hz, 1H), 3.13 (t, J=6.1 Hz, 2H), 2.71 (t, J=6.3 Hz, 2H). ¹³C-NMR (125 MHz): δ 205.844, 172.740, 161.911, 152.480, 150.178, 164.425, 143.537, 141.614, 141.433, 131.828, 131.740, 129.667, 129.259, 128.355, 125.382, 120.414, 90.289, 83.674, 75.885, 72.276, 71.692, 38.044, 31.082. HRMS (ESI) m/z calcd for C₂₃H₂₄N₇O₉S ([M+H]⁺) 574.1356; found 574.1367.

Synthesis of a tetrazole analog (Compound 105)

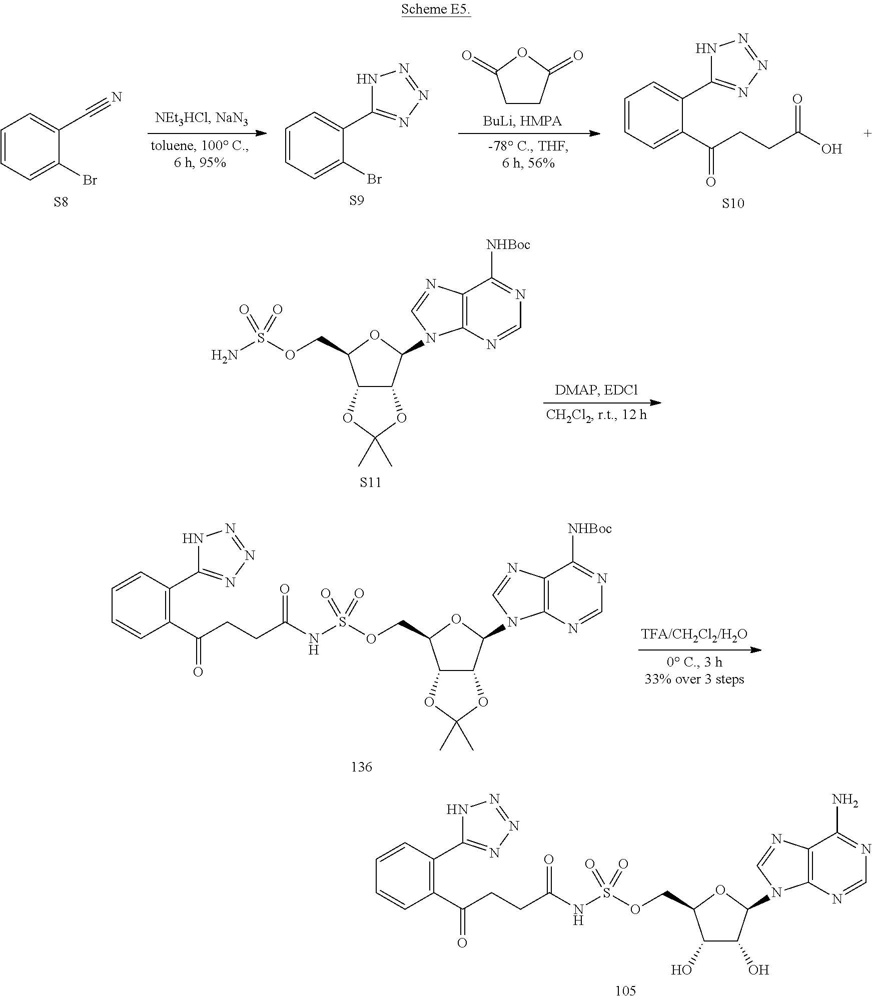

### 5-(2-Bromophenyl)-2H-tetrazole (S9)

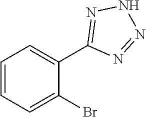 [SMILES: Brc1ccccc1-c1nn[nH]n1]

2-Bromobenzonitrile (S8) (1 g, 5.494 mmol, 1 equiv.), triethylamine hydrochloride (2.269 g, 16.482 mmol, 3.0 equiv.), and sodium azide (1.072 g, 16.482 mmol, 3.0 equiv.) was suspended in 20 mL toluene and stirred at 100° C. for 6 hours. The reaction was then cooled to room temperature, filtered through a celite pad, and concentrated under vacuum. The residue was reconstituted in 20 mL water, acidified with 1 M KHSO₄, and extracted with EtOAc (5×20 mL). The combined organic extracts were dried (Na₂SO₄), filtered, and concentrated by rotary evaporation. Purification by silica flash chromatography (0%→25% EtOAc in hexanes with 1% AcOH) yielded the title product S9 (1.175 g, 95% yield) as a white solid. IR (ATR): 2465, 1604, 1574, 1475, 1447, 1435, 1396, 1276, 1247, 1165, 1093, 1056, 1027, 1011, 995, 924, 879, 773, 7485, 712, 643. ¹H-NMR (600 MHz; MeOD): δ 7.83 (dd, J=8.0, 0.9 Hz, 1H), 7.69 (dd, J=7.6, 1.5 Hz, 1H), 7.56 (td, J=7.6, 1.2 Hz, 1H), 7.51 (td, J=7.8, 1.7 Hz, 1H). ¹³C-NMR (150 MHz; MeOD): δ 156.211, 134.966, 133.866, 133.025, 129.233, 127.560, 123.224. HRMS (ESI) m/z calcd for C₇H₆BrN₄ ([M+H]⁺) 224.9776; found 224.9781.

### 4-(2-(2H-Tetrazol-5-yl)phenyl)-4-oxobutanoic acid (S10)

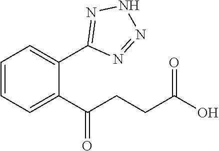 [SMILES: O=C(O)CCC(=O)c1ccccc1-c1nn[nH]n1]

Aryl bromide S9 (107 mg, 0.475 mmol, 1 equiv.) and HMPA (191.5 mg, 1.069 mmol, 2.25 equiv.) were dissolved in 0.5 mL THF before being cooled to −78° C. n-BuLi (0.668 mL, 1.069 mmol, 2.25 equiv., 1.6 M in THF) was added drop wise and the reaction stirred for 1 hour at −78° C. The reaction was added via cannula to a suspension of succinic anhydride (190 mg, 1.901 mmol, 4.0 equiv.) in 2 mL THF at −78° C., then stirred for 6 hours. The reaction was warmed to room temperature and quenched with 10 mL 1M HCl before being extracted with EtOAc (5×10 mL). The combined organic extracts were dried (Na₂SO₄), filtered, and concentrated by rotary evaporation. Purification by silica flash chromatography (25%→75% EtOAc in hexanes, 1% AcOH) yielded the product (S10) as a white crystalline solid (65 mg, 56%). IR (ATR): 2963.99, 2925.82, 1711.92, 1401.93, 1368.48, 1176.46, 1101.81, 990.78, 778.35, 755.75. ¹H-NMR (600 MHz; MeOD): δ 7.96-7.95 (m, 1H), 7.72 (dq, J=6.0, 3.0 Hz, 3H), 3.19 (t, J=6.4 Hz, 2H), 2.67 (t, J=6.4 Hz, 2H). ¹³C-NMR (150 MHz; MeOD): δ 203.563, 176.568, 140.785, 132.760, 132.282, 131.786, 129.928, 124.609, 37.299, 29.125. HRMS (ESI) m/z calcd for C₁₁H₉N₄O₃ ([M+H]⁺) 269.0651; found 269.0668.

### Compound 136: 6-N-t-Butoxycarbonyl-2′,3′-O-isopropylidene-5′-O—(N-[4-(2-(2H-tetrazol-5-yl) phenyl)-4-oxobutanoyl]sulfamoyl)adenosine

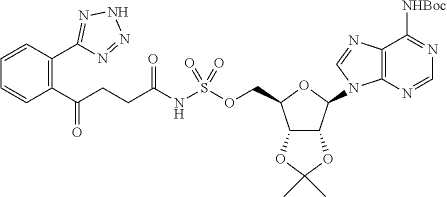 [SMILES: CC(C)(C)OC(=O)Nc1ncnc2c1ncn2[C@@H]1O[C@H](COS(=O)(=O)NC(=O)CCC(=O)c2ccccc2-c2nn[nH]n2)[C@H]2OC(C)(C)O[C@H]21]

Keto acid S10 (100 mg, 0.406 mmol, 1 equiv.), protected 5′-O-sulfamoyladenosine S11 (296 mg, 0.609 mmol, 1.5 equiv.) and DMAP (50 mg, 0.406 mmol, 1 equiv.) were suspended in 25 mL CH₂Cl₂ and EDCI (311 mg, 1.624 mmol, 4 equiv.) added. The reaction was stirred for 3 hours at room temperature before being quenched with 25 mL water and extracted with dichloromethane (5×25 mL). The combined organic extracts were dried (Na₂SO₄), filtered through a pad of celite, and concentrated by rotary evaporation to afford the crude protected tetrazole analogue 136 (473 mg, 163% crude yield), which was used without further purification.

### Compound 105: 5′-O—(N-[4-(2-(2H-Tetrazol-5-yl)phenyl)-4-oxobutanoyl]sulfamoyl)adenosine

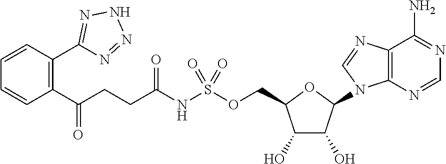 [SMILES: Nc1ncnc2c1ncn2[C@@H]1O[C@H](COS(=O)(=O)NC(=O)CCC(=O)c2ccccc2-c2nn[nH]n2)[C@@H](O)[C@H]1O]

Crude protected tetrazole AMS analogue 136 was dissolved in 15 mL DCM and 1 mL H₂O, cooled to 0° C. TFA (15 mL) was added and the reaction stirred for 3 hours while returning to room temperature. Concentration by rotary evaporation, purification by preparative HPLC (5%→95% MeCN in H₂O with 0.01% TFA), and lyophilization yielded tetrazole analogue 105 as a fluffy white solid (78 mg, 33% over two steps). IR (ATR): 3321.36, 3114.54, 2907.72, 2823.70, 1692.64, 1615.08, 1479.12, 1424.42, 1363.02, 1201.44, 1120.65, 975.22, 871.81, 729.62. ¹H-NMR (600 MHz; MeOD): δ 8.45 (s, 1H), 8.33 (s, 1H), 7.95-7.93 (m, 1H), 7.71-7.68 (m, 3H), 6.09 (d, J=5.1 Hz, 1H), 4.62 (t, J=5.1 Hz, 1H), 4.55-4.48 (m, 2H), 4.36 (t, J=4.7 Hz, 1H), 4.32 (q, J=3.8 Hz, 1H), 3.25 (td, J=6.1, 2.4 Hz, 2H), 2.67 (t, J=6.1 Hz, 2H). ¹³C-NMR (151 MHz; MeOD): δ 203.1, 172.8, 153.1, 150.3, 147.4, 143.2, 140.2, 133.0, 132.5, 131.8, 130.1, 124.2, 120.5, 90.2, 83.7, 75.8, 72.3, 71.7, 36.6, 30.9. HRMS (ESI) m/z calcd for C₂₁H₂₃N₁₀O₈S ([M+H]⁺) 575.1421; found 575.1436.

### Synthesis of a Squaric Acid Analog (Compound 106)

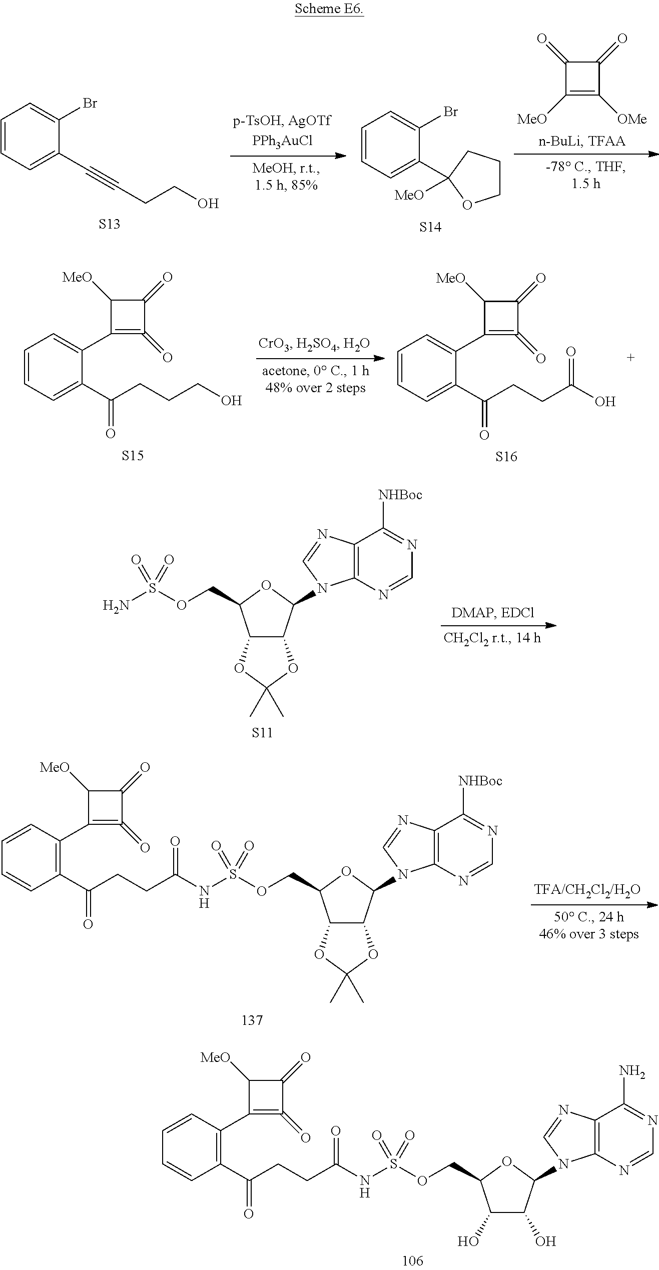

### 2-(2-Bromophenyl)-2-methoxytetrahydrofuran (S14)

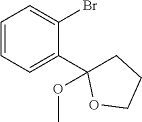

Alkyne S13 (5.699 g, 25.3197 mmol, 1 equiv.) and p-toluenesulfonic acid (482 mg, 2.532 mmol, 0.1 equiv.) was dissolved in 250 mL MeOH and cooled to 0° C. PPh₃AuCl (125 mg, 0.2532 mmol, 0.01 equiv.) and AgOTf (65 mg, 0.2532 mmol, 0.01 equiv.) was added and the reaction stirred for 2 hours at 0° C. The reaction was diluted with 500 mL saturated sodium bicarbonate and extracted with dichloromethane (3×500 mL). The combined organic extracts were dried (Na₂SO₄), filtered, and concentrated by rotary evaporation. Purification by silica flash chromatography (0%->10% EtOAc in hexanes) yielded the product (S14) as a clear and colorless oil (6.5 g, 99%). IR (ATR): 3063, 2976, 2946, 2885, 2820, 1589, 1567, 1470, 1418, 1266, 1237, 1182, 1134, 1098, 1048, 1020, 936, 852, 755. ¹H-NMR (600 MHz): δ 7.78 (dd, J=7.8, 1.8 Hz, 1H), 7.61 (dd, J=7.9, 1.2 Hz, 1H), 7.30-7.28 (m, 1H), 7.15 (td, J=7.6, 1.8 Hz, 1H), 4.07 (dtd, J=33.1, 8.0, 6.1 Hz, 2H), 3.00 (s, 3H), 2.76 (ddd, J=12.9, 8.5, 4.4 Hz, 1H), 2.21-2.14 (m, 1H), 2.02 (ddd, J=12.9, 9.7, 7.4 Hz, 1H), 1.97-1.91 (m, 1H). ¹³C-NMR (150 MHz): δ 139.550, 134.449, 129.415, 129.408, 126.856, 121.129, 108.608, 67.158, 49.615, 38.026, 24.726. HRMS (ESI) m/z calcd for C₁₁H₁₄BrO₂ ([M+H]⁺) 257.0177; found 257.0158.

### 3-(2-(4-Hydroxybutanoyl)phenyl)-4-methoxycyclobut-3-ene-1,2-dione (S15)

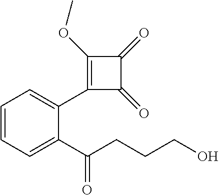 [SMILES: COc1c(-c2ccccc2C(=O)CCCO)c(=O)c1=O]

Aryl bromide S14 (145 mg, 0.5639 mmol, 1 equiv.) was dissolved in 0.5 mL THF and cooled to −78° C. n-BuLi (0.4053 mL, 0.6485 mmol, 1.15 equiv., 1.6 M in THF) was added drop wise and the reaction stirred for 1 hours. Dimethyl squarate (160 mg, 1.128 mmol, 2.0 equiv.) in 1 mL THF was added drop wise at −78° C., and the reaction stirred for 1.5 hours. Trifluoroacetic anhydride (0.120 mL, 0.8459 mmol, 1.5 equiv.) was added drop wise and the reaction stirred for 20 minutes. The reaction was quenched with 1 M HCl (5 mL) and warmed to 0° C. before extracting with CH₂Cl₂ (5×5 mL). The combined organic extracts were dried (Na₂SO₄), filtered, diluted with 25 mL acetone, and reduced in volume to approximately 10 mL by rotary evaporation at 0° C. The reaction was diluted with 25 mL acetone and reduced in volume to approximately 5 mL by rotary evaporation at 0° C. The crude product S15 in acetone was used immediately in the next step without further purification.

### 4-(2-(2-Methoxy-3,4-dioxocyclobut-1-enyl)phenyl)-4-oxobutanoic acid (S16)

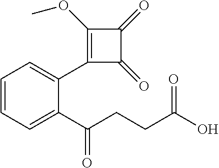 [SMILES: COc1c(-c2ccccc2C(=O)CCC(=O)O)c(=O)c1=O]

Jones reagent was prepared by dissolving CrO₃ (280 mg, 2.8075 mmol, 5.0 equiv.) in 1.5 mL H₂O and cooling to 0° C. Concentrated sulfuric acid (0.4679 mL, 8.4225 mmol, 15 equiv.) was added drop wise and the solution allowed to stir for 15 minutes. The Jones reagent was added drop wise slowly to the stirring solution of crude alcohol S15 in 5 mL at 0° C. until the reaction remained a persistent bright red (˜30 minutes). The reaction was stirred for 15 minutes and quenched with isopropyl alcohol before being diluted with 10 mL water, and extracted with EtOAc (3×10 mL). The combined organic extracts were dried (Na₂SO₄), filtered, and concentrated by rotary evaporation. Purification by silica flash chromatography (50% EtOAc in hexanes with 1% AcOH) yielded the product (S16) as a white solid (77 mg, 48% over 2 steps). IR (ATR): 3072.53, 2963.68, 1789.61, 1755.81, 1691.27, 1599.17, 1489.17, 1454.42, 1369.88, 1218.75, 1167.88, 11033.84, 927.99, 812.74, 763.30, 613.87. ¹H-NMR (600 MHz): δ 7.82 (dd, J=7.6, 0.8 Hz, 1H), 7.73 (dd, J=7.6, 1.0 Hz, 1H), 7.62 (td, J=7.6, 1.2 Hz, 1H), 7.58 (td, J=7.6, 1.2 Hz, 1H), 4.50 (s, 3H), 3.35 (t, J=6.4 Hz, 2H), 2.85 (t, J=6.4 Hz, 2H). ¹³C-NMR (150 MHz): δ 201.040, 194.598, 192.453, 191.455, 176.055, 138.487, 131.764, 131.298, 128.863, 128.137, 124.563, 61.708, 35.351, 28.091. HRMS (ESI) m/z calcd for C₁₅H₁₁O₆ ([M−H]⁻) 287.0556; found 287.0556.

### Compound 137: 6-N-t-Butoxycarbonyl-2′,3′-O-isopropylidene-5′-O—(N-[4-(2-(2-methoxy-3,4-dioxocyclobut-1-enyl)phenyl)-4-oxobutanoyl]sulfamoyl)adenosine

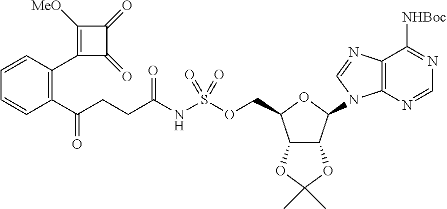 [SMILES: COc1c(-c2ccccc2C(=O)CCC(=O)NS(=O)(=O)OC[C@H]2O[C@@H](n3cnc4c(NC(=O)OC(C)(C)C)ncnc43)[C@@H]3OC(C)(C)O[C@@H]32)c(=O)c1=O]

Keto acid S16 (69 mg, 0.2394 mmol, 1 equiv.), protected 5′-O-sulfamoyladenosine S11 (146 mg, 0.2993 mmol, 1.25 equiv.) and DMAP (29 mg, 0.2394 mmol, 1 equiv.) was suspended in 1 mL dichloromethane and EDCI (184 mg, 0.9576 mmol, 4.0 equiv.) added. The reaction was stirred at room temperature for 4 hours, quenched with 1 mL water, diluted with 4 mL saturated sodium chloride, and extracted with dichloromethane (5×5 mL). The combined organic extracts were dried (Na₂SO₄), filtered through a pad of celite, and concentrated by rotary evaporation to afford the crude protected squarate analogue 137 (353 mg, 195% crude yield), which was used without further purification.

### Compound 106: 5′-O—(N-[4-(2-(2-Methoxy-3,4-dioxocyclobut-1-enyl)phenyl)-4-oxobutanoyl]sulfamoyl)adenosine

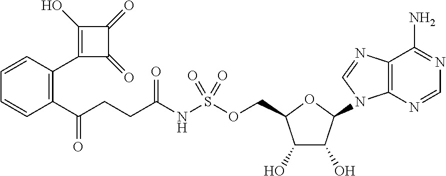 [SMILES: Nc1ncnc2c1ncn2[C@@H]1O[C@H](COS(=O)(=O)NC(=O)CCC(=O)c2ccccc2-c2c(O)c(=O)c2=O)[C@@H](O)[C@H]1O]

Crude protected squaric acid analogue 137 was dissolved in 3 mL DCM and 0.2 mL H₂O. TFA (2 mL) was added and the reaction heated to 50° C. for 24 hours before being returned to room temperature. Concentration by rotary evaporation, purification by preparative HPLC (5%→95% MeCN in H₂O with 0.01% TFA), and lyophilization yielded the product (106) as a white fluffy solid (55 mg, 46% over 2 steps). IR (ATR): 3321.36, 3124.24, 2972.35, 2930.34, 1695.87, 1453.50, 1359.78, 1205.24, 1123.98, 978.46, 881.51, 758.71, 916.70. ¹H-NMR (600 MHz; DMSO-d6/D₂O): δ 8.54 (s, 1H), 8.40 (s, 1H), 7.92-7.89 (m, 1H), 7.51 (td, J=7.6, 1.3 Hz, 1H), 7.44 (dd, J=7.7, 1.1 Hz, 1H), 7.33 (td, J=7.5, 1.2 Hz, 1H), 5.98 (d, J=5.1 Hz, 1H), 4.56 (t, J=4.9 Hz, 1H), 4.51 (dd, J=11.0, 3.2 Hz, 1H), 4.44 (dd, J=11.0, 5.4 Hz, 1H), 4.21 (td, J=7.0, 3.7 Hz, 2H), 3.09 (t, J=6.5 Hz, 2H), 2.70 (t, J=6.6 Hz, 2H). ¹³C-NMR (150 MHz): δ 215.520, 202.677, 195.756, 195.592, 175.892, 170.894, 148.415, 141.365, 137.499, 130.121, 127.738, 127.505, 127.429, 126.619, 125.220, 118.756, 87.782, 81.503, 73.371, 71.326, 69.900, 39.932, 35.952, 29.967. HRMS (ESI) m/z calcd for C₂₄H₂₃N₆O₁₁S ([M+H]⁺) 603.1146; found 603.1146.

### Synthesis of a Lactone Analog (Compound 107)

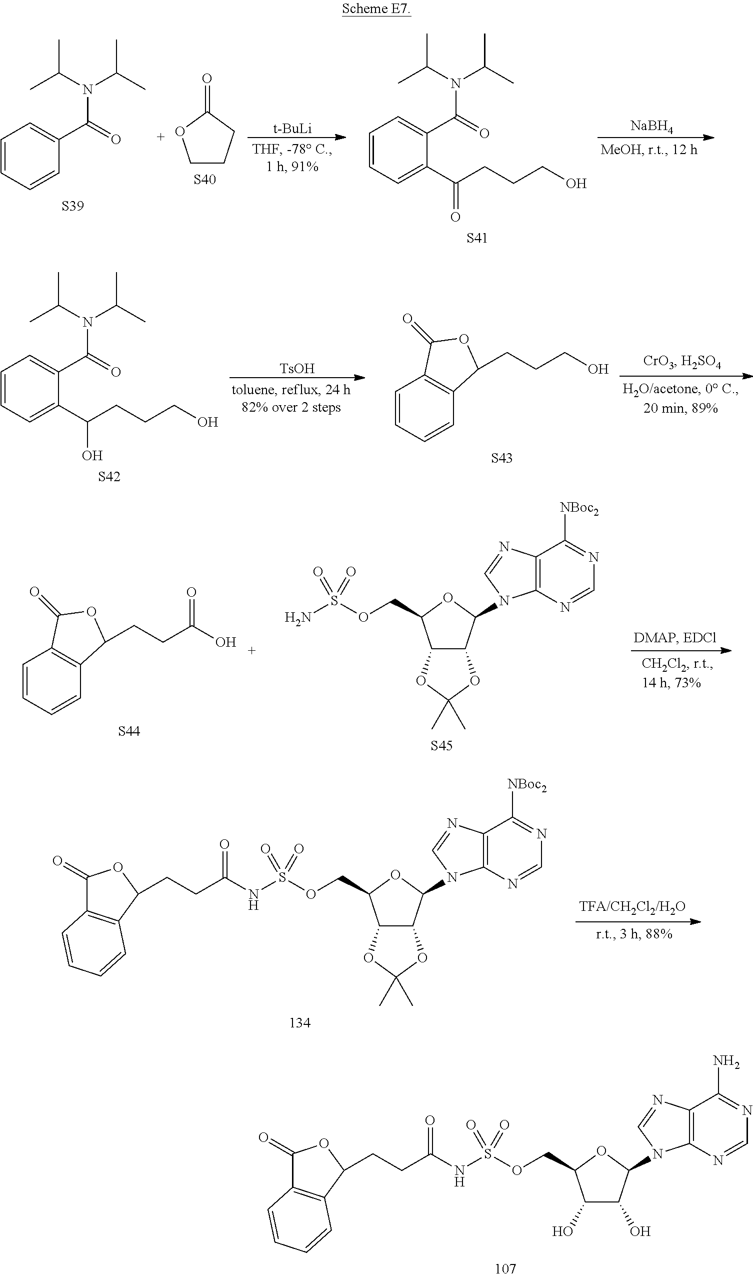

### 2-(4-Hydroxybutanoyl)-N,N-diisopropylbenzamide (S41)

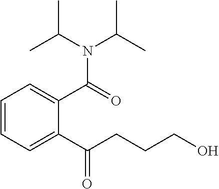 [SMILES: CC(C)N(C(=O)c1ccccc1C(=O)CCCO)C(C)C]

N,N-Diisopropylbenzamide (S39) (2 g, 9.742 mmol, 1 equiv.) was dissolved in dry THF (75 mL), cooled to −78° C., and t-BuLi (6.35 mL, 10.81 mmol, 1.11 equiv., 1.7 M in THF) was added. The reaction was stirred for 45 minutes, then γ-butyrolactone (S40) (1.023 g, 11.89 mmol, 1.22 equiv.) was added drop wise. The reaction was stirred for 1 hour while returning to room temperature, then quenched with saturated ammonium chloride (75 mL) and extracted with ethyl acetate (5×75 mL). The combined organic extracts were dried (Na₂SO₄), filtered, and concentrated by rotary evaporation. Purification by silica flash chromatography (100% EtOAc) yielded the product (S41) as a clear and colorless oil (2.570 g, 91%). IR (ATR): 3392.03, 3063.48, 2971.43, 2933.90, 2876.05, 2239.51, 1771.67, 1688.89, 1615.28, 1438.39, 1370.16, 1343.38, 1212.67, 1163.10, 1035.36, 919.87, 773.75, 749.77. ¹H-NMR (500 MHz): δ 7.7457 (d, J=7.68, 1H), 7.4867 (t, J=7.46, 1H), 7.4035 (t, J=7.57, 1H), 7.1973 (d, J=7.43, 1H), 3.6422 (m, 3H), 3.5089 (p, J=6.78, 1H), 3.0421 (t, J=6.87, 1H), 2.8090 (m, 1H), 1.9310 (p, J=6.39, 6.20, 2H), 1.5585 (d, J=6.78, 6H), 1.1351 (d, J=6.58, 6H). ¹³C-NMR (125 MHz): δ δ 202.2687, 170.5203, 138.8097, 136.1131, 131.6439, 128.4759, 128.1557, 126.1526, 61.3600, 51.2894, 45.7528, 36.7801, 26.9920, 20.2568. HRMS (ESI) m/z calcd for C₁₇H₂₆NO₃ ([M+H]⁺) 292.1913; found 292.1934.

### 2-(1,4-Dihydroxybutyl)-N,N-diisopropylbenzamide (S42)

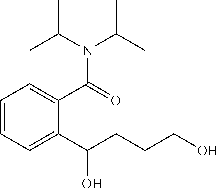 [SMILES: CC(C)N(C(=O)c1ccccc1C(O)CCCO)C(C)C]

Aryl ketone S41 (2 g, 7.035 mmol, 1 equiv.) was dissolved in MeOH (80 mL) and NaBH₄ (397 mg, 10.5 mmol, 1.5 equiv.) added. The reaction was stirred for 12 hours at room temperature, then quenched with 1 M HCl (20 mL), diluted with saturated sodium chloride (75 mL), and extracted with ethyl acetate (5×50 mL). The combined organic extracts were dried (Na₂SO₄), filtered, and concentrated by rotary evaporation afford the crude diol S42 (2.99 g, 145% crude yield), which was used without further purification.

### 3-(3-Hydroxypropyl)isobenzofuranone (S43)

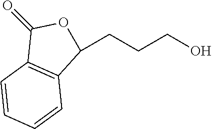 [SMILES: O=C1OC(CCCO)c2ccccc21]

Crude diol S42 was dissolved in toluene (230 mL) and p-toluenesulfonic acid (12 mg, 0.070 mmol, 0.01 equiv.) was added. The reaction was heated to reflux for 24 hours, then cooled to room temperature and concentrated by rotary evaporation. Purification by silica flash chromatography (100% EtOAc) yielded the product (S43) as a greasy white solid (1.53 g, 82% over two steps). IR (ATR): 3425.88, 3056.06, 2946.34, 2874.74, 2256.23, 1758.74, 1614.32, 1467.50, 1350.13, 1287.83, 1214.05, 1057.69, 953.94, 753.64, 740.83. ¹H-NMR (500 MHz): δ 7.8844 (d, J=7.69, 1H), 7.6821 (t, J=7.51, 1H), 7.5312 (t, J=7.50, 1H), 7.4643 (d, J=7.50, 1H), 5.5513 (q, J=3.55, 3.98, 3.95, 1H), 3.9096 (m, 2H), 2.2382 (m, 1H), 1.9565 (s, 1H), 1.7800 (m, 3H). ¹³C-NMR (125 MHz): δ 170.6723, 149.8942, 134.1130, 129.1655, 126.0218, 125.7034, 121.8157, 81.2551, 62.0275, 31.3511, 31.2139, 27.9039. HRMS (ESI) m/z calcd for C₁₁H₁₂O₃Na ([M+Na]⁺) 215.0684; found 215.0689.

### 3-(3-Oxo-1,3-dihydroisobenzofuran-1-yl)propanoic acid (S44)

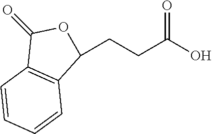 [SMILES: O=C(O)CCC1OC(=O)c2ccccc21]

Alcohol S43 (400 mg, 2.09 mmol, 1 equiv.) was dissolved in acetone (20 mL), cooled 0° C., then jones reagent (prepared as previously described using CrO₃ (1.044 g, 10.459 mmol, 5 equiv.), H₂O (13.3 mL), and conc. sulfuric acid (1.33 mL)) was added drop wise over 25 minutes until a deep red color persisted. The reaction was stirred for 20 minutes, then quenched with isopropyl alcohol, diluted with H₂O (80 mL), and extracted with ethyl acetate (5×80 mL). The combined organic extracts were dried (Na₂SO₄), filtered, and concentrated by rotary evaporation. Purification by silica flash chromatography (100% EtOAc with 1% AcOH) yielded the product (S44) as a greasy white solid (380 mg, 89%). IR (NaCl, Film): 3057.78, 2931.03, 2663.35, 2255.14, 1759.78, 1614.42, 1598.88, 1467.20, 1415.70, 1349.37, 1287.76, 1214.86, 1167.32, 1085.32, 1065.61, 1033.64, 937.72, 758.64, 741.61. ¹H-NMR (500 MHz): δ 10.589 (Br, 1H), 7.8353 (d, J=7.53, 1H), 7.6293 (t, J=7.53, 1H), 7.4838 (t, J=7.53, 1H), 7.4044 (d, J=7.59, 1H), 5.4924 (q, J=3.04, 5.75, 2.49, 1H), 2.5646 (m, 1H), 2.4345 (m, 2H), 1.9184 (m, 1H). ¹³C-NMR (125 MHz): δ 178.116, 170.2475, 149.0646, 134.2717, 129.4815, 126.0303, 125.9295, 121.8258, 29.7038, 29.6006, 29.1911. HRMS (ESI) m/z calcd for C₁₁H₁₀O₄Na ([M+Na]⁺) 229.0477; found 229.0470.

### Compound 134: 6-N-Bis-t-butoxycarbonyl-2′,3′-O-isopropylidene-5′-O—(N-[3-(3-oxo-1,3-dihydroisobenzofuran-1-yl)propanoyl]sulfamoyl)adenosine

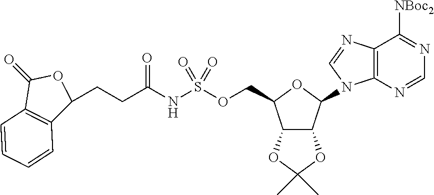

Propionic acid S44 (40 mg, 0.194 mmol, 1 equiv.), protected 5′-O-sulfamoyladenosine S45 (141 mg, 0.291 mmol, 1.5 equiv.) and DMAP (24 mg, 0.194 mmol, 1 equiv.) dissolved in CH₂Cl₂ (4 mL) and EDCI (511.8 mg, 2.67 mmol, 3 equiv.) added. The reaction was stirred 14 hours, then quenched with water (25 mL) and extracted with CH₂Cl₂ (5×25 mL). The combined organic extracts were dried (Na₂SO₄), filtered, and concentrated by rotary evaporation. Purification by silica flash chromatography (10% MeOH in CH₂Cl₂) yielded the product (134) as a white solid (96 mg, 73%). IR (NaCl, Film): 2981.78, 2932.38, 2853.86, 2254.21, 1763.16, 1600.73, 1578.55, 1495.72, 1454.04, 1371.02, 1339.07, 1286.45, 1257.42, 1212.39, 1141.90, 1112.33, 1082.21, 1033.77, 951.36, 914.35, 849.58, 794.86, 776.64, 734.16, 695.90, 646.50. ¹H-NMR (600 MHz; MeOD): δ 8.86 (d, J=1.3, 1H), 8.78 (s, 1H), 7.83 (d, J=7.7, 1H), 7.74 (td, J=7.5, 1.0, 1H), 7.62 (dd, J=7.7, 0.8, 1H), 7.56 (t, J=7.5, 1H), 6.37 (d, J=2.9, 1H), 5.63 (dd, J=8.2, 3.5, 1H), 5.43 (dd, J=6.1, 2.9, 1H), 5.17 (dd, J=6.1, 2.6, 1H), 4.57 (td, J=4.2, 2.7, 1H), 4.31 (qd, J=10.7, 4.3, 2H), 2.44-2.33 (m, 3H), 1.98-1.93 (m, 1H), 1.59 (s, 3H), 1.37 (s, 19H), 1.35 (s, 3H). ¹³C-NMR (150 MHz; MeOD): δ 172.52, 154.34, 153.29, 151.62, 151.53, 151.1, 146.92, 135.72, 130.48, 130.42, 127.02, 126.33, 123.65, 115.60, 92.21, 85.92, 85.68, 85.53, 83.04, 82.66, 70.10, 35.13, 31.83, 28.05, 27.55, 25.58. HRMS (ESI) m/z calcd for C₃₄H₄₃N₆O₁₃S ([M+H]⁺) 775.2609; found 775.2607.

### Compound 107: 5′-O—(N-[3-(3-Oxo-1,3-dihydroisobenzofuran-1-yl)propanoyl]sulfamoyl) adenosine

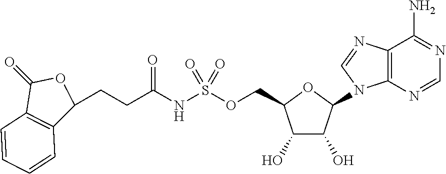 [SMILES: Nc1ncnc2c1ncn2[C@@H]1O[C@H](COS(=O)(=O)NC(=O)CCC2OC(=O)c3ccccc32)[C@@H](O)[C@H]1O]

TFA (1.5 mL) was added drop wise to a stirring solution of the protected adenosine (40 mg, 0.0593 mmol, 1 equiv.) in dichloromethane (1.5 mL) and water (0.25 mL) at 0° C. and allowed to stir for 1 hour. The reaction was then allowed to return to room temperature while stirring for 3 hours before being stripped of solvent under vacuum. The residue was resolved by silica chromatography (10%->20% MeOH/EtOAc) to give the product (28 mg, 88%) as a white solid. IR (NaCl, Film): 3343.90, 2921.08, 2852.40, 1751.95, 1684.92, 1603.68, 1469.95, 1420.01, 1363.97, 1292.14, 1208.21, 1139.71, 1049.90, 842.45, 802.15, 723.77. ¹H-NMR (600 MHz; MeOD): δ 1-H NMR (600 MHz; MeOD): δ 8.51 (s, 1H), 8.17 (s, 1H), 7.82 (d, J=7.7, 1H), 7.70 (td, J=7.5, 1.0, 1H), 7.58 (dd, J=7.7, 0.8, 1H), 7.55 (t, J=7.5, 1H), 6.07 (d, J=5.8, 1H), 5.61 (dd, J=8.3, 3.4, 1H), 4.64 (t, J=5.4, 1H), 4.38 (dd, J=5.0, 3.3, 1H), 4.34 (dd, J=11.7, 3.8, 1H), 4.29 (dt, J=7.9, 3.6, 2H), 3.34 (s, 1H), 2.46-2.33 (m, 3H), 1.96-1.91 (m, 1H). ¹³C-NMR (150 MHz; MeOD): δ 181.49, 172.68, 157.32, 153.70, 151.69, 150.89, 141.18, 135.58, 130.37, 126.98, 126.25, 123.60, 120.19, 89.17, 84.65, 82.85, 76.25, 72.34, 69.21, 35.45, 32.28. HRMS (ESI) m/z calcd for C₂₁H₂₃O₉N₆S ([M+H]⁺) 535.1247; found 535.1238.

### Synthesis of a Lactam Analog (Compound 108)

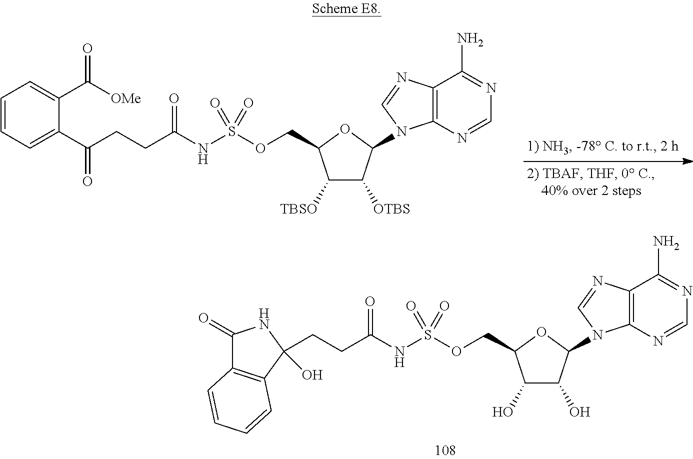

### Compound 108: 5′-O—(N-[3-(1-Hydroxy-3-oxoisoindolin-1-yl)propanoyl]sulfamoyl) adenosine

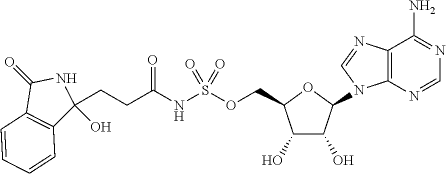 [SMILES: Nc1ncnc2c1ncn2[C@@H]1O[C@H](COS(=O)(=O)NC(=O)CCC2(O)NC(=O)c3ccccc32)[C@@H](O)[C@H]1O]

bis-TBS protected MeOSB-AMS (55 mg, 0.0694 mmol, 1 equiv.) prepared via previously described methods^{(1,2,3)} was placed in a 15 mL pressure vessel and cooled to −78° C. 5 mL anhydrous ammonia was then condensed into the pressure vessel and sealed before being allowed to return to room temperature to stir for 2 hours. The reaction was then cooled to −78° C., placed under cycling argon, and allowed to slowly return to room temperature to remove the ammonia. The reaction was then placed under high vacuum for 30 minutes before being re-suspended in 5 mL THF and cooled to 0° C. TBAF (0.208 mL, 0.208 mmol, 3.0 equiv., 1.0M in THF) was added drop wise and the solution allowed to stir for 1 hour before being stripped of solvent under vacuum. The product was isolated by silica chromatography (10%->20% MeOH/EtOAc) as the tetrabutylammonium salt (15 mg, 40% over two steps). IR (NaCl, Film): 3327.55, 3190.17, 2963.89, 2876.08, 1706.64, 1654.02, 1599.02, 1471.31, 1419.65, 1364.45, 1298.17, 1223.26, 1148.39, 1088.07, 943.83, 884.15, 834.63, 800.76, 747.36, 718.19, 641.96. ¹H-NMR (600 MHz): δ 8.52 (s, 1H), 8.19 (s, 1H), 7.69-7.67 (m, 1H), 7.62 (tdd, J=7.4, 2.2, 1.1, 1H), 7.59 (d, J=7.5, 1H), 7.49 (tt, J=7.4, 1.1, 1H), 6.09-6.08 (m, 1H), 4.64 (td, J=5.4, 3.3, 1H), 4.37 (dd, J=5.0, 3.2, 1H), 4.31-4.23 (m, 3H), 3.24-3.21 (m, 12H), 2.49-2.43 (m, 1H), 2.35-2.23 (m, 2H), 2.13-2.05 (m, 1H), 1.67-1.62 (m, 12H), 1.40 (sextet, J=7.4, 12H), 1.01 (t, J=7.4, 18H). ¹³C-NMR (150 MHz): δ 181.984, 171.550, 161.541, 157.354, 153.924, 150.961, 150.592, 141.151, 134.007, 132.450, 130.490, 124.059, 123.486, 120.170, 89.184, 84.716, 76.411, 72.393, 68.883, 59.534, 36.168, 35.235, 24.845, 20.788, 14.021. HRMS (ESI) m/z calcd for C₂₁H₂₄N₇O₉S ([M+H]⁺) 550.1356; found 550.1362.

### Synthesis of a Difluoroindanone-3-Ol Analog (Compound 109; Racemic Synthesis)

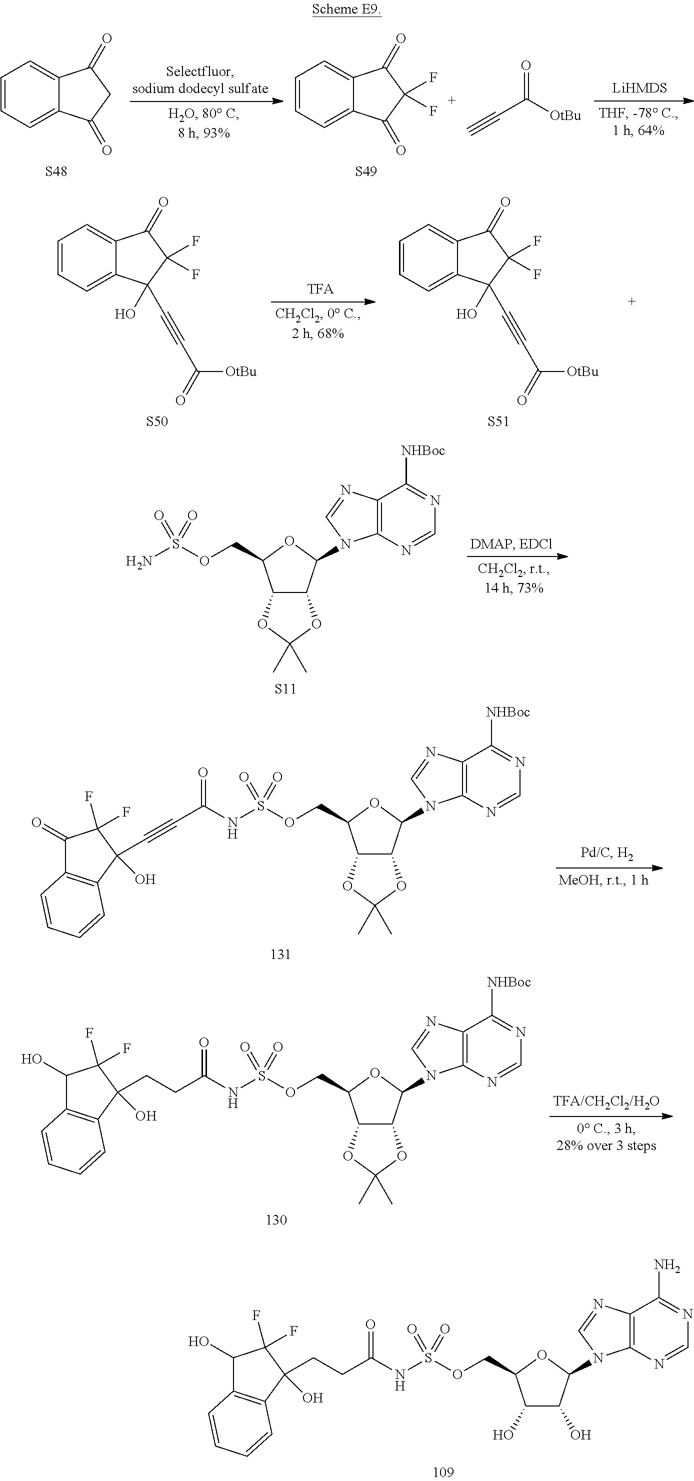

### 2,2-Difluoro-indene-1,3-dione (S49)

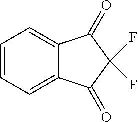 [SMILES: O=C1c2ccccc2C(=O)C1(F)F]

SelectFluor (24.24 g, 68.43 mmol, 2 equiv.), 1,3 indandione S48 (5.0 g, 34.2 mmol, 1.0 equiv.) and sodium dodecyl sulfate (99 mg, 0.342 mmol, 0.01 equiv.) were suspended in water (80 mL). The reaction was heated to 80° C. for 8 hours, then cooled to room temperature and extracted with Et₂O (5×80 mL). The combined organic extracts were dried (Na₂SO₄), filtered, and concentrated by rotary evaporation. Purification by sublimation (200 mTorr, 150° C.) yielded the product S49 as bright white crystals (5.82 g, 93%). IR (NaCl, Film): 3479, 3098, 1728, 1583, 1302, 1185, 1090, 1088, 733. ¹H-NMR (500 MHz; CDCl3): δ 8.16 (dtdd, J=5.1, 3.2, 2.3, 0.0 Hz, 2H), 8.11-8.07 (m, 2H). ¹³C-NMR (125 MHz): δ 185.923, 139.372, 138.276, 125.088, 102.538. ¹⁹F-NMR (471 MHz; CDCl₃): δ −124.843. HRMS (ESI) m/z calcd for C₉H₅F₂O₂ ([M+H]⁺) 183.0258; found 183.0232.

### tert-Butyl 3-(2,2-difluoro-1-hydroxy-3-oxo-2,3-dihydro-indenyl)propiolate (S50)

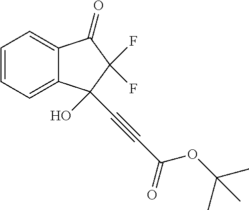 [SMILES: CC(C)(C)OC(=O)C#CC1(O)c2ccccc2C(=O)C1(F)F]

LiHMDS (13.72 mL, 13.72 mmol, 1.25 equiv., 1.0 M in THF) was cooled to −78° C. and t-butyl propiolate (1.522 g, 12.07 mmol, 1.1 equiv.) in THF (10 mL) was added drop wise over 10 minutes. The reaction was stirred for 1 hour, then added via cannula over 30 minutes to a stirring solution of di-ketone S49 (2 g, 10.98 mmol, 1 equiv.) in THF (10 mL) at −78° C. The reaction was stirred for 1 hour, then quenched with saturated ammonium chloride (50 mL) and extracted with CH₂Cl₂ (5×50 mL). The combined organic extracts were dried (Na₂SO₄), filtered, and concentrated by rotary evaporation. Purification by silica flash chromatography (20% EtOAc in hexanes) yielded the product (S50) as a clear and colorless oil (2.767 g, 82%). IR (NaCl, Film): 2988.34, 2211.10, 1757.05, 1712.43, 1606.01, 1474.78, 1400.54, 1375.08, 1262.00, 1221.18, 1154.93, 1020.93, 900.80, 843.49, 758.41, 717.83, 652.39. ¹H-NMR (500 MHz; CDCl3): δ 7.93 (dd, J=7.6, 0.9 Hz, 1H), 7.90-7.87 (m, 2H), 7.67 (td, J=7.5, 0.9 Hz, 1H), 4.17 (s, 1H), 1.50 (s, 9H). ¹³C-NMR (125 MHz): δ 187.830, 157.996, 151.862, 148.999, 138.156, 131.910, 131.165, 126.282, 125.159, 85.122, 82.060, 77.523, 71.065, 27.933. ¹⁹F-NMR (471 MHz; CDCl₃): δ 111.190, −111.762, −125.772, −126.348. HRMS (ESI) m/z calcd for C₁₆H₁₅F₂O₄Na ([M+Na]⁺) 331.0758; found 331.0764.

### 3-(2,2-Difluoro-1-hydroxy-3-oxo-2,3-dihydro-indenyl)propiolic acid (S51)

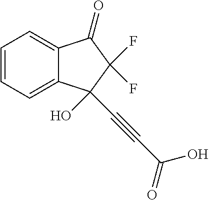 [SMILES: O=C(O)C#CC1(O)c2ccccc2C(=O)C1(F)F]

t-Butyl ester S50 (400 mg, 1.298 mmol, 1 equiv.) was dissolved in CH₂Cl₂/H₂O (5 mL, 10:1) and cooled to 0° C., then TFA (5 mL) was added. The reaction was stirred for 2 hours, then concentrated by rotary evaporation. Purification by silica flash chromatography (10% MeOH in CH₂Cl₂) yielded the product (S51) as a white semi-solid (225 mg, 69%). IR (NaCl, Film): 3410.08, 1752.32, 1689.66, 1605.21, 1370.48, 1276.66, 1201.28, 1141.1082, 1024.08, 937.93, 902.86, 851.60, 767.86, 716.11, 648.97. ¹H-NMR (500 MHz; DMSO-d6): δ 8.06-8.03 (m, 1H), 7.96-7.94 (m, 2H), 7.80-7.77 (m, 1H). ¹³C-NMR (125 MHz; DMSO-d6): δ 188.385, 153.757, 150.586, 138.920, 131.916, 129.758, 126.061, 124.830, 113.974, 83.152, 77.101, 70.006. ¹⁹F-NMR (471 MHz; CDCl₃): δ −113.646, −114.207, −128.356, −128.915. HRMS (ESI) m/z calcd for C₂₄H₁₁F₄O₈ ([2M−H]⁻) 503.0390; found 503.0394.

### Compound 131: 6-N-t-Butoxycarbonyl-2′,3′-O-isopropylidene-5′-O—(N-[3-(2,2-difluoro-1-hydroxy-3-oxo-2,3-dihydro-indenyl)propioloyl]sulfamoyl)adenosine

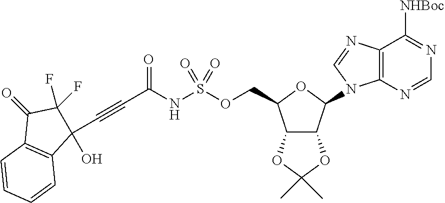 [SMILES: CC(C)(C)OC(=O)Nc1ncnc2c1ncn2[C@@H]1O[C@H](COS(=O)(=O)NC(=O)C#CC2(O)c3ccccc3C(=O)C2(F)F)[C@H]2OC(C)(C)O[C@H]21]

Propiolic acid S51 (110 mg, 0.4362 mmol, 1 equiv.), protected 5′-O-sulfamoyladenosine S11 (265 mg, 0.5452 mmol, 1.25 equiv.) and DMAP (53 mg, 0.4362 mmol, 1.0 equiv.) was dissolved in CH₂Cl₂ (5 mL) and EDCI (335 mg, 1.7448 mmol, 4.0 equiv.) was added. The reaction was stirred for 4 hours, then quenched with 30 mL water, and extracted with CH₂Cl₂ (5×25 mL). The combined organic extracts were dried (Na₂SO₄), filtered, and concentrated by rotary evaporation to afford the crude product 131 (427 mg, 136% crude yield), which was used without further purification.

### Compound 130: 6-N-t-Butoxycarbonyl-2′,3′-O-isopropylidene-5′-O—(N-[3-(2,2-difluoro-1,3-dihydroxy-2,3-dihydro-1H-inden-1-yl)propanoyl]sulfamoyl)adenosine

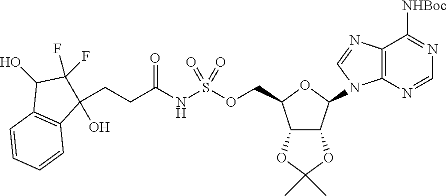 [SMILES: CC(C)(C)OC(=O)Nc1ncnc2c1ncn2[C@@H]1O[C@H](COS(=O)(=O)NC(=O)CCC2(O)c3ccccc3C(O)C2(F)F)[C@H]2OC(C)(C)O[C@H]21]

Crude product 131 from previous step and 10% Pd/C (463.5 mg, 0.435 mmol, 1 equiv.) were suspended in solution of MeOH/NEt₃ (40 mL, 9:1). The reaction was then stirred vigorously under H₂ balloon for 1 hour before being diluted with EtOAc (50 mL), filtered through a celite pad, and concentrated by rotary evaporation to afford the crude mixture of a single side-chain diastereomer products 130 (510 mg, 151% crude yield), which was used without further purification.

### Compound 109: 5′-O—(N-[3-(2,2-Difluoro-1-hydroxy-3-oxo-2,3-dihydro-indenyl)propioloyl]sulfamoyl)adenosine

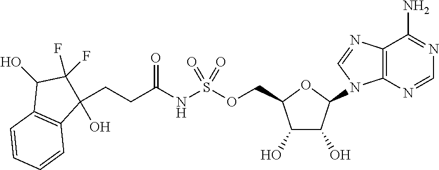 [SMILES: Nc1ncnc2c1ncn2[C@@H]1O[C@H](COS(=O)(=O)NC(=O)CCC2(O)c3ccccc3C(O)C2(F)F)[C@@H](O)[C@H]1O]

Crude product 130 was suspended in CH₂Cl₂ (5 mL) and water (0.25 mL), then cooled to 0° C. and TFA (5 mL) added. The reaction was stirred for 1 hour at 0° C., then allowed to stir for 3 hours while returning to room temperature. Concentration by rotary evaporation, purification by preparative HPLC (5%→95% MeCN in H₂O with 0.01% TFA), and lyophilization yielded a mixture of a single diastereomer side-chain products (109) as a fluffy white solid (71 mg, 28% over 3 steps). IR (NaCl, Film): 3173, 2927, 1693, 1664, 1466, 1415, 1357, 1189, 1134, 1072, 872, 791, 717. ¹H-NMR (500 MHz; MeOD): δ 8.47 (s, 1H), 8.35 (d, J=1.8 Hz, 1H), 7.45-7.37 (m, 4H), 6.11-6.09 (m, 1H), 5.13-5.10 (m, 1H), 4.65-4.62 (m, 1H), 4.58-4.50 (m, 2H), 4.42-4.39 (m, 1H), 4.32-4.30 (m, 1H), 2.63 (td, J=7.8, 2.8 Hz, 2H), 2.32-2.13 (m, 2H). ¹³C-NMR (150 MHz): δ 173.449, 150.229, 147.018, 143.991, 143.408, 140.036, 139.286, 130.700, 130.631, 127.093, 126.441, 124.932, 120.495, 90.396, 83.610, 79.622, 75.770, 74.931, 72.261, 71.609, 31.317, 31.146. ¹⁹F-NMR (471 MHz; CDCl₃): δ −120.084, −120.581, −130.679, −131.147. HRMS (ESI) m/z calcd for C22H₂₅F₂N₆O₉S ([M+H]⁺) 587.1372; found 587.1349.

### Stereoselective Synthesis of a Difluoroindanone-3-Ol Analogs (2)

To assess the activity of the individual stereoisomers of Compound 109, stereoselective synthesis was developed leveraging enzymatic kinetic resolution. The individual stereoisomers of Compound 109 (Also 2 herein) were then evaluated in biochemical, computational, and cell culture studies to assess selectivity and mechanisms of action (vide infra).

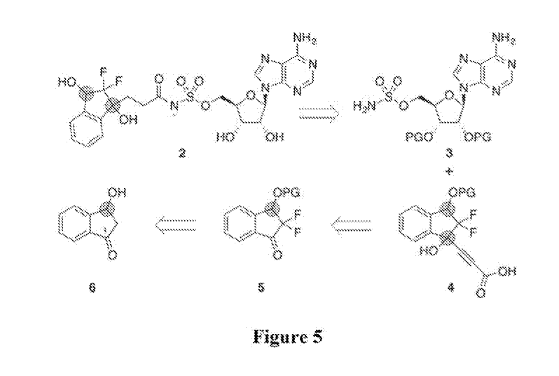

In the synthesis of 109 exemplifed above, a racemic difluoroindanol side chain bearing a ketone at the C3 position was coupled to the AMS scaffold, with the ketone undergoing non-stereoselective reduction during a subsequent hydrogenation step (see, e.g., Matarlo et al. Biochemistry 2015, 54, 6514-6524). To access the individual diastereomers of Compound 109 (2) in a stereoselective fashion, an alternative retrosynthetic approach can be used in which both the C1 and C3 stereocenters of the side chains 4 are be set prior to coupling to the AMS scaffold 3 (See, e.g., FIG. 5). C1 stereochemistry can be set via diastereoselective transformations of protected ketoalcohol 5, with absolute stereochemistry at C3 established in 3-hydroxy-1-indanone 6.

To access both enantiomers of 3-hydroxy-1-indanone (6), an enzymatic kinetic resolution with vinyl acetate and Amano Lipase PS (Burkholderia cepacia, formerly Pseudomonas cepacia) can be carried out. See, e.g., Joly, S.; Nair, M. S. Tetrahedron: Asymmetry 2001, 12, 2283-2287. At 50% conversion, the reaction provided the starting alcohol (3S)-6 in 46% yield and >98% ee (Chiracel OB-H) and the enantiomeric acetate (3R)-7 in 43% yield and >98% ee, corresponding to an E value of >200 (Scheme E12). Scheme E12 shows synthesis of syn-difluoroindanediol inhibitors (1R,3S)-2 and (1S,3R)-2. Yields in parentheses are for synthesis of (1S,3R)-2, prepared analogously from alcohol (3S)-6. Compound 12: 2′,3′-bis(t-butyldimethylsilyl)-5′-O-sulfamoyladenosine.

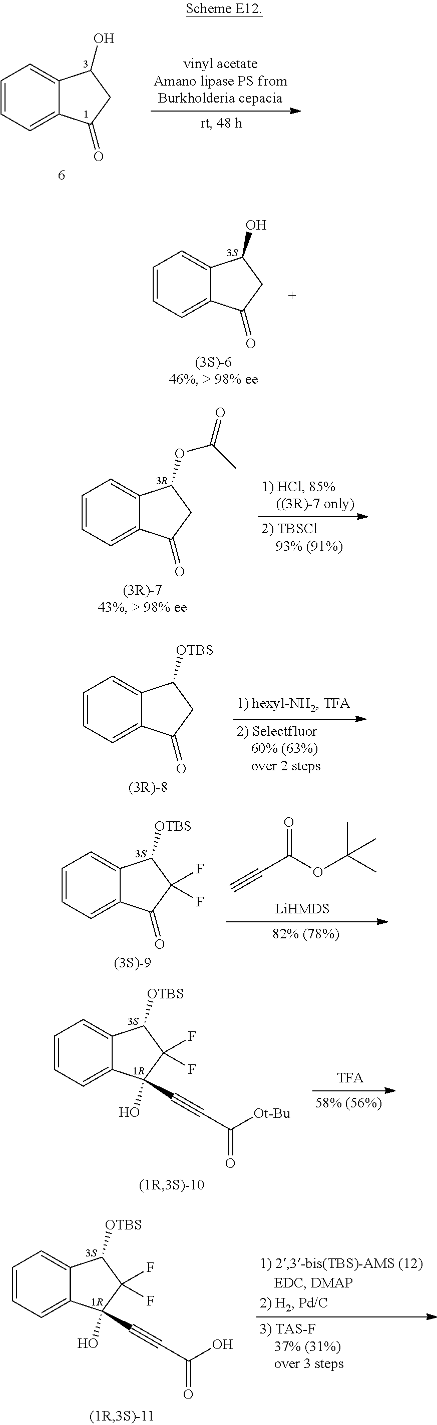

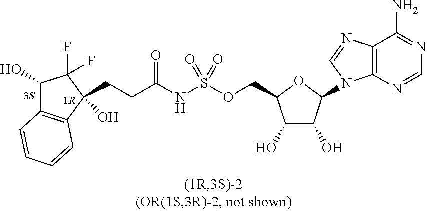 [SMILES: Nc1ncnc2c1ncn2[C@@H]1O[C@H](COS(=O)(=O)NC(=O)CC[C@@]2(O)c3ccccc3[C@H](O)C2(F)F)[C@@H](O)[C@H]1O]

With the C3 stereochemistry established, synthesis of the syn-difluoroindanediol inhibitors (1R,3S)-2 commenced with conversion of the acetate (3R)-7 to TBS ether (3R)-8. Conversion to a Schiff base then allowed mild fluorination with Selectfluor to provide α-difluoroketone (3S)-9 (see, e.g., Bertozzi et al. J. Am. Chem. Soc. 2010, 132, 11799-11805). Propiolate addition under optimized conditions provided syn-diol (1R,3S)-10 (>20:1 dr). The t-butyl ester was cleaved, and the resulting acid was coupled to protected AMS scaffold 12 (see, e.g., Lu et al. Bioorg. Med. Chem. Lett. 2008, 18, 5963-5966; Lu et al. ChemBioChem 2012, 13, 129-136; Matarlo et al. Biochemistry 2015, 54, 6514-6524). Hydrogenation of the alkyne and global deprotection provided syn-difluoroindanediol (1R,3S)-2. The other syn-diol diastereomer (1S,3R)-2 was synthesized analogously from the enantiomeric alcohol (3S)-6. Absolute and relative stereochemistry was confirmed by X-ray crystallographic analysis of the diol obtained via desilylation of TBS ether (1S,3R)-11.

To access the corresponding anti-difluorindanediol inhibitor (1R,3R)-2, an oxidation/re-reduction approach was used, starting from protected syn-diol intermediate (1R,3S)-10 to afford anti-diol intermediate (1R,3R)-15 (Scheme E13). This anti-diol exhibited a ¹H-NMR shift of 5.41 ppm for C3-H, compared to 5.11 ppm for the epimeric syn-diol obtained by desilylation of (1S,3R)-11 above. Coupling to protected AMS scaffold 12, alkyne hydrogenation, and global deprotection afford anti-difluoroindanediol (1R,3R)-2. The other anti-diol diastereomer (1S,3S)-2 was synthesized analogously from the enantiomeric protected syn-diol intermediate (1S,3R)-10. Scheme E13 shows synthesis of anti-difluoroindanediol inhibitors (1R,3R)-2 and (1S,3S)-2. Yields in parentheses are for synthesis of (1S,3S)-2.

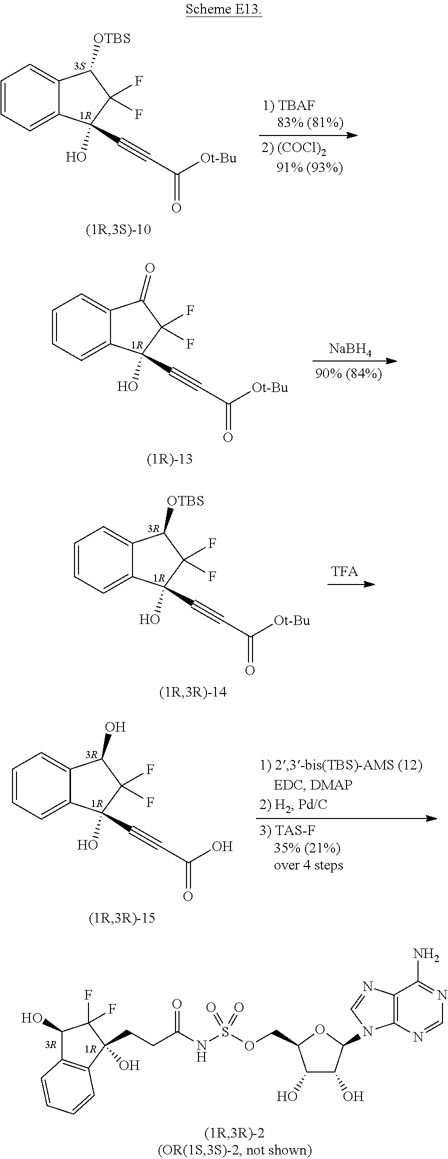 [SMILES: CC(C)(C)OC(=O)C#C[C@@]1(O)c2ccccc2[C@H](O[Si](C)(C)C(C)(C)C)C1(F)F ; CC(C)(C)OC(=O)C#C[C@@]1(O)c2ccccc2C(=O)C1(F)F ; "NaBH4" ; CC(C)(C)OC(=O)C#C[C@@]1(O)c2ccccc2[C@@H](O[Si](C)(C)C(C)(C)C)C1(F)F ; O=C(O)C#C[C@@]1(O)c2ccccc2[C@@H](O)C1(F)F ; Nc1ncnc2c1ncn2[C@@H]1O[C@H](COS(=O)(=O)NC(=O)CC[C@@]2(O)c3ccccc3[C@@H](O)C2(F)F)[C@@H](O)[C@H]1O]

### Experimental Procedures for Stereoselective Synthesis of a Difluoroindanone-3-Ols (2)

### General Methods

Reagents were obtained from Aldrich Chemical or Acros Organics and used without further purification. Optima or HPLC grade solvents were obtained from Fisher Scientific, degassed with Ar, and purified on a solvent drying system as described. See, e.g., Pangborn, A. B.; Giardello, M. A.; Grubbs, R. H.; Rosen, R. K.; Timmers, F. J. Organometallics 1996, 15, 1518-1520. Reactions were performed in flame-dried glassware under positive Ar pressure with magnetic stirring.

TLC was performed on 0.25 mm E. Merck silica gel 60 F254 plates and visualized under UV light (254 nm) or by staining with potassium permanganate (KMnO4), cerium ammonium molybdenate (CAM), or iodine (12). Silica flash chromatography was performed on E. Merck 230-400 mesh silica gel 60. Preparative scale HPLC purification was carried out on a Waters 2545 HPLC with 2996 diode array detector using an Atlantis Prep C18 reverse phase column (10 Å˜150 mm, 5 μm) with UV detection at 254 nm using a flow rate of 20 mL/min and a gradient of 5-30 MeCN in 0.1% aqueous TFA over 10 min. Samples were lyophilized using a Labconco Freezone 2.5 instrument.

IR spectra were recorded on a Bruker Optics Tensor 27 FTIR spectrometer with Pike technologies MIRacle ATR (attenuated total reflectance, ZnSe crystal) accessory and peaks reported in cm⁻¹. NMR spectra were recorded on a Bruker Avance III 500 instrument at 24° C. in CDCl₃ unless otherwise indicated. Spectra were processed using Bruker TopSpin or nucleomatica iNMR (www.inmr.net) software, and chemical shifts are expressed in ppm relative to TMS (1H, 0 ppm) or residual solvent signals: CDCl₃ (1H, 7.24 ppm; ¹³C, 77.23 ppm), CD₃OD (¹H, 3.31 ppm; ¹³C, 49.15 ppm), D₂O (1H, 4.80 ppm); coupling constants are expressed in Hz. Mass spectra were obtained on a Waters Acuity SQD LC-MS by electrospray (ESI) ionization or atmospheric pressure chemical ionization (AP-CI).

### Enzymatic kinetic resolution of 3-hydroxy-1-indanone

### (S)-3-Hydroxy-1-indanone (6) and (R)-3-oxo-1-indanyl acetate (7)

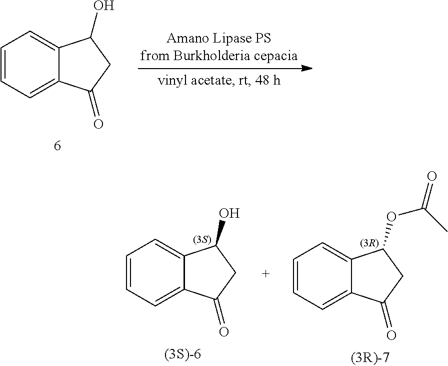

3-Hydroxy-1-indanone (1 g, 6.7 mmol, 1 equiv) prepared as previously described (see, e.g., Ruan, Jiwu; Iggo, Jonathan; Xiao, Jianliang. Org. Lett. 2011, 13, 268-271) and Amano Lipase PS from Burkholderia cepacia (1.5 g, Sigma Aldrich) were suspended in vinyl acetate (80 mL) and stirred at rt for 48 h. Filtration through a pad of celite, concentration by rotary evaporation, and purification by silica flash chromatography (20→60% EtOAc in hexanes) yielded (3S)-3-hydroxy-1-indanone 8 (455 mg, >98% ee, 46% yield) and (R)-3-oxo-1-indanyl acetate 9 (604 mg, >98% ee, 47% yield). Enantiomeric excess was determined by analytical HPLC (Chiralcel: OB-H, 4.6 mm×150 mm, 5 μm particle size, 5% isopropanol in hexanes, 1 mL/minute). See, e.g., Chen, C. S.; Fujimoto, Y.; Girdaukas, G.; Sih, C. J. J. Am. Chem. Soc. 1982, 104, 7294-7299. E=Ln[(1−c)(1−ee)]/Ln[(1−c)(1+ee)]. (3S)-3-hydroxy-1-indanone: tᵣₑₜ=25 min; (3R)-3-hydroxy-1-indanone: tᵣₑₜ=23 min; (3R)-3-oxo-1-indanyl acetate: tᵣₑₜ=20 min; (3S)-3-oxo-1-indanyl acetate: tᵣₑₜ=23 min.

(R)-3-oxo-1-indanyl acetate (7): IR (ATR): 3075, 2936, 1718, 1605, 1466, 1433, 1402, 1372, 1341, 1280, 1228, 1164, 1096, 1065, 989, 965, 945, 869, 763, 734, 681, 634, 607. ¹H-NMR (600 MHz; CDCl₃): δ 7.78 (d, J=7.7 Hz, 1H), 7.70-7.67 (m, 2H), 7.55-7.52 (m, 1H), 6.36 (dd, J=7.0, 2.6 Hz, 1H), 3.19 (dd, J=19.1, 7.0 Hz, 1H), 2.66 (dd, J=19.1, 2.7 Hz, 1H), 2.14 (s, 3H). ¹³C-NMR (126 MHz; CDCl₃): δ 202.1, 171.0, 151.5, 137.1, 135.3, 130.0, 126.9, 123.4, 69.9, 43.9, 21.1. HRMS (ESI) m/z calcd for C₁₁H₁₀O₃Na ([M+H]⁺) 213.0528; found 213.0522.

(S)-3-hydroxy-1-indanone (6): IR (ATR): 3393, 2917, 1698, 1605, 1465, 1396, 1332, 1279, 1242, 1211, 1176, 1153, 1099, 1044, 993, 960, 903, 811, 759, 728, 644. ¹H-NMR (600 MHz; CDCl₃): δ 7.71-7.70 (m, 2H), 7.68 (td, J=7.3, 1.2 Hz, 1H), 7.48-7.46 (m, 1H), 5.41 (td, J=6.7, 2.9 Hz, 1H), 3.21 (dd, J=6.7, 1.4 Hz, 1H), 3.08 (dd, J=18.8, 6.8 Hz, 1H), 2.59 (dd, J=18.8, 2.9 Hz, 1H). ¹³C-NMR (126 MHz; CDCl₃): δ 203.8, 155.3, 136.3, 135.4, 129.5, 126.0, 123.2, 68.4, 47.1. HRMS (ESI) m/z calcd for C₉H₈O₂Na ([M+Na]⁺) 171.0422; found 171.0419.

### Synthesis of 1R, 3S-syn-Difluoroindanediol (1R,3S)-2

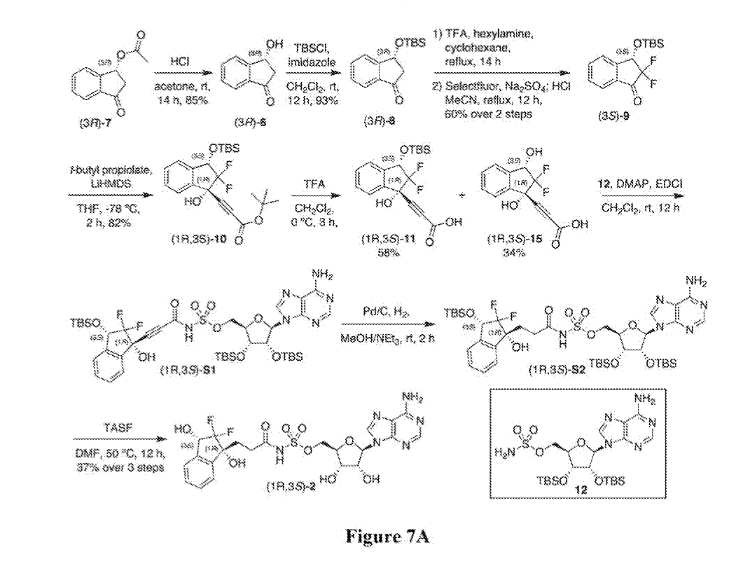

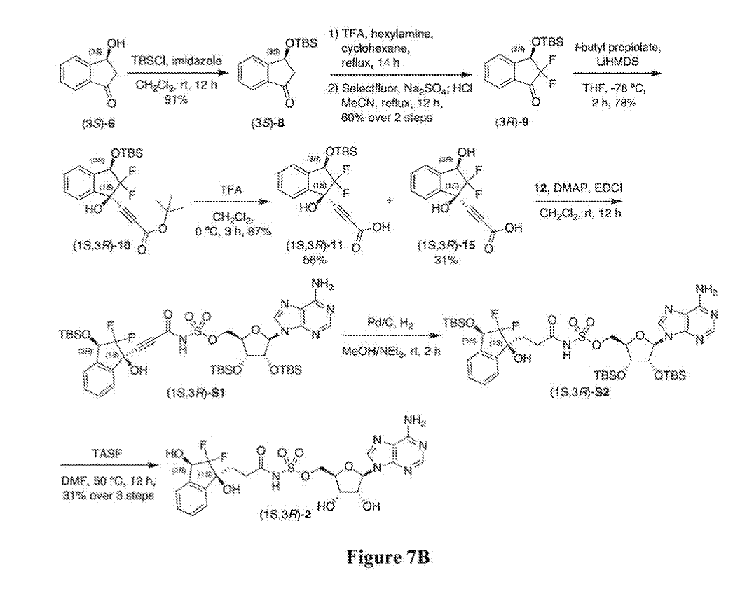

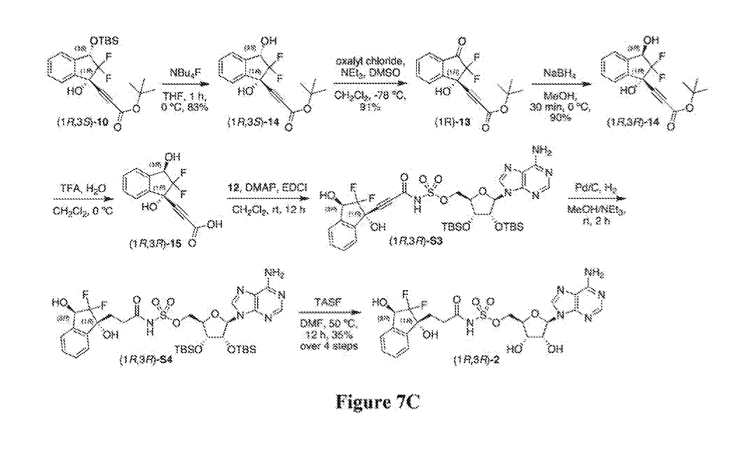

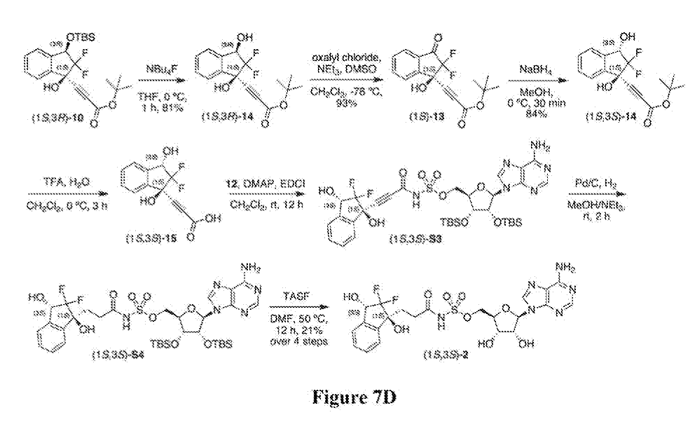

See FIG. 7A for a scheme of the synthesis exemplified below.

### (R)-3-Hydroxy-1-indanone (6)

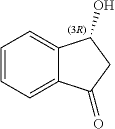 [SMILES: O=C1C[C@@H](O)c2ccccc21]

(R)-3-Oxo-1-indanyl acetate 7 (550 mg, 2.891 mmol, 1 equiv.) was dissolved in 20 mL acetone then 6 M HCl (20 mL) was added. The mixture was stirred at rt for 14 h, then poured into satd aq NaHCO₃ (150 mL) and extracted with CH₂Cl₂ (4×75 mL). The combined organic extracts were dried (Na₂SO₄), filtered, and concentrated by rotary evaporation. Purification by silica flash chromatography (30%→70% EtOAc in hexanes) yielded the alcohol (3R)-6 as a pale yellow solid (365 mg, 85%). IR (ATR): 3404, 2914, 1715, 1600, 1466, 1401, 1340, 1275, 1243, 1203, 1152, 1037, 896, 759, 730. ¹H-NMR (500 MHz; CDCl₃): δ 7.70-7.69 (m, 1H), 7.67-7.65 (m, 2H), 7.46-7.44 (m, 1H), 5.37 (dd, J=6.8, 2.9 Hz, 1H), 3.74 (s, 1H), 3.04 (dd, J=18.8, 6.8 Hz, 1H), 2.56 (dd, J=18.8, 3.0 Hz, 1H). ¹³C-NMR (126 MHz; CDCl₃): δ 204.0, 155.4, 136.2, 135.4, 129.4, 126.0, 123.2, 68.3, 47.1. HRMS (ESI) m/z calcd for C₉H₈O₂Na ([M+Na]⁺) 171.0422; found 171.0428.

### (R)-3-((t-Butyldimethylsilyl)oxy)-1-indanone (8)

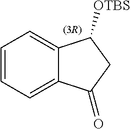 [SMILES: CC(C)(C)[Si](C)(C)O[C@@H]1CC(=O)c2ccccc21]

(R)-3-Hydroxy-1-indanone ((R)-6) (310 mg, 2.092 mmol, 1 equiv.) was dissolved in 5 mL CH₂Cl₂ and imidazole (370 mg, 5.439 mmol, 2.6 equiv.) was added. TBSCl (410 mg, 2.719 mmol, 1.3 equiv.) was added and the reaction mixture was stirred at rt for 12 h, then diluted with 50 mL water and extracted with CH₂Cl₂ (4×50 mL). The combined organic extracts were dried (Na₂SO₄), filtered, and concentrated by rotary evaporation. Purification by silica flash chromatography (0%→30% EtOAc in hexanes) yielded the silyl ether (3R)-8 as a yellow tinged oil (510 mg, 93%). IR (ATR): 2955, 2930, 2886, 1857, 1720, 1605, 1464, 1390, 1351, 1279, 1254, 1216, 1161, 1106, 1078, 1046, 1006, 961, 933, 856, 837, 809, 776, 759, 741, 720, 668. ¹H-NMR (500 MHz; CDCl₃): δ 7.74 (d, J=7.7 Hz, 1H), 7.68-7.66 (m, 1H), 7.61 (d, J=7.6 Hz, 1H), 7.46 (t, J=7.4 Hz, 1H), 5.39 (dd, J=6.6, 3.4 Hz, 1H), 3.07 (dd, J=18.3, 6.7 Hz, 1H), 2.60 (dd, J=18.3, 3.4 Hz, 1H), 0.96 (s, 9H), 0.23 (s, 3H), 0.19 (s, 3H). ¹³C-NMR (126 MHz; CDCl₃): δ 203.1, 156.0, 136.3, 135.1, 129.0, 125.8, 123.0, 68.9, 47.9, 25.8, 18.2, −4.4, −4.6. HRMS (ESI) m/z calcd for C₁₅H₂₂O₂NaSi ([M+Na]⁺) 285.1287; found 285.1280.

### (S)-3-((t-Butyldimethylsilyl)oxy)-2,2-difluoro-1-indanone (9)

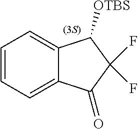 [SMILES: CC(C)(C)[Si](C)(C)O[C@H]1c2ccccc2C(=O)C1(F)F]

Ketone (3R)-8 (266 mg, 1.013 mmol, 1 equiv.) was dissolved in 25 mL toluene, then hexylamine (0.535 mL, 4.052 mmol, 4 equiv.) was added and the reaction mixture was heated to reflux for 14 h. The reaction was then cooled to rt, concentrated by rotary evaporation, and placed under high vacuum (˜60 mTorr) for 1 h. The crude imine was dissolved in acetonitrile (10 mL) and Selectfluor (753 mg, 2.125 mmol, 2.1 equiv.) and sodium sulfate (144 mg, 1.012 mmol, 1 equiv.) were added, then the reaction mixture was heated to reflux. The reaction was stirred for 12 h, then cooled to rt, diluted with 1 M HCl (50 mL) and extracted with CH₂Cl₂ (4×50 mL). The combined organic extracts were dried (Na₂SO₄), filtered, and concentrated by rotary evaporation. Purification by silica flash chromatography (0%→50% CH₂Cl₂ in hexanes) yielded the difluoroindanone (3S)-9 as a deep yellow tinged oil (180 mg, 60%). IR (ATR): 2956, 2932, 2888, 2860, 1745, 1608, 1472, 1362, 1299, 1256, 1230, 1184, 1143, 1101, 1075, 1007, 927, 895, 838, 780, 740, 698, 670, 648. ¹H-NMR (500 MHz; CDCl₃): δ 7.67 (d, J=7.7 Hz, 1H), 7.62 (td, J=7.6, 1.1 Hz, 1H), 7.45 (dt, J=7.8, 0.8 Hz, 1H), 7.40-7.37 (m, 1H), 5.05 (dd, J=12.8, 3.5 Hz, 1H), 0.80 (s, 9H), 0.10 (s, 3H), 0.06 (s, 3H). ¹³C-NMR (126 MHz; CDCl₃): δ 189.6, 150.4, 137.5, 132.3, 130.4, 126.2, 124.7, 114.9, 71.8, 25.7, 18.4, −4.6,

−5.1. ¹⁹F-NMR (471 MHz; CDCl₃): δ δ −116.48 (d, J=278.6 Hz, 1F), −123.42 (d, J=279.3 Hz, 1F). HRMS (ESI) m/z calcd for C₁₅H₂₀O₃F₂SiNa ([M+Na]+) 321.1098; found 321.1094.

### t-Butyl 3-((1R,3S)-3-(t-butyldimethylsilyloxy)-2,2-difluoro-1-hydroxy-1-indanyl)propio-late (10)

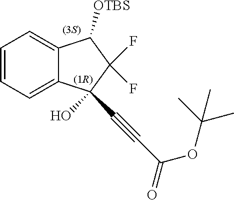 [SMILES: CC(C)(C)OC(=O)C#C[C@@]1(O)c2ccccc2[C@H](O[Si](C)(C)C(C)(C)C)C1(F)F]

Lithium bis(trimethylsilyl)amide (6.5 mL, 6.492 mmol, 1.0 M in THF, 1.55 equiv.) was cooled to −78° C., then t-butyl propiolate (793 mg, 6.283 mmol, 1.5 equiv.) in 3 mL THF was added and the mixture was stirred for 45 min. The solution was then added via cannula over 10 min to ketone (3R)-9 (1.25 g, 4.189 mmol, 1 equiv.) in 5 mL THF at −78° C. and stirred for 2 h. The reaction was quenched with satd aq NH₄Cl (50 mL), warmed to rt, and extracted with EtOAc (4×50 mL). The combined organic extracts were dried (Na₂SO₄), filtered, and concentrated by rotary evaporation. Purification by silica flash chromatography (50%→100% CH₂Cl₂ in hexanes) yielded the ester (1R,3S)-10 as a yellow tinged oil (1.778 g, 82%). IR (ATR): 3394, 2956, 2932, 2888, 2859, 2245, 1762, 1473, 1395, 1371, 1258, 1205, 1153, 1113, 1040, 1013, 909, 888, 839, 791, 751, 732, 695, 672, 657. ¹H-NMR (500 MHz; CDCl₃): δ 7.63-7.60 (m, 1H), 7.48-7.44 (m, 2H), 7.39-7.37 (m, 1H), 5.19 (dd, J=8.0, 6.4 Hz, 1H), 1.49 (s, 9H), 0.95 (s, 9H), 0.24 (s, 6H). ¹³C-NMR (126 MHz; CDCl₃): δ 151.8, 139.4, 139.1, 130.9, 130.3, 124.9, 124.4, 124.2, 84.2, 80.6, 78.2, 74.9, 74.4, 28.0, 25.7, 18.2, −4.6, −4.9. ¹⁹F-NMR (471 MHz; CDCl₃): δ −115.04 (d, J=222.3 Hz, 1F), −128.51 (d, J=223.3 Hz, 1F). HRMS (ESI) m/z calcd for C₂₂H₃₀O₄F₂SiNa ([M+Na]⁺) 441.1779; found 447.1774.

### 3-((1R,3S)-3-(t-Butyldimethylsilyloxy)-2,2-difluoro-1-hydroxy-1-indanyl)propiolic acid (11)

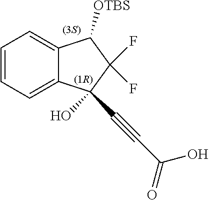 [SMILES: CC(C)(C)[Si](C)(C)O[C@H]1c2ccccc2[C@](O)(C#CC(=O)O)C1(F)F]

Ester (1R,3S)-10 (485 mg, 1.142 mmol, 1 equiv.) was dissolved in 5 mL CH₂Cl₂ and cooled to 0° C., then 5 mL TFA was added and the reaction mixture was stirred for 3 h. Concentration by rotary evaporation at 0° C. and purification by silica flash chromatography (50%→100% EtOAc in hexanes) yielded the acid (1R,3S)-11 as a white cotton type solid (245 mg, 58%) as well as the corresponding desilated diol (1R,3S)-15 as a white solid (100 mg, 34%).

(1R,3S)-11: IR (ATR): 2957, 2932, 2887, 2860, 2249, 1700, 1472, 1364, 1247, 1150, 1095, 1010, 910, 892, 839, 782, 760, 733, 687, 652, 625. ¹H-NMR (500 MHz; CDCl₃): δ 7.62-7.60 (m, 1H), 7.50-7.45 (m, 2H), 7.40-7.38 (m, 1H), 5.18 (dd, J=8.2, 5.8 Hz, 1H), 0.95 (s, 9H), 0.24 (s, 6H). ¹³C-NMR (126 MHz; CDCl₃): δ 156.1, 139.17, 138.99, 131.2, 130.4, 125.1, 124.5, 124.0, 83.3, 78.5, 75.1, 74.5, 25.7, 18.2, −4.66, −4.85. ¹⁹F-NMR (471 MHz; CDCl₃): δ −114.45 (d, J=227.1 Hz, 1F), −128.02 (d, J=224.9 Hz, 1F). HRMS (ESI) m/z calcd for C₁₈H₂₂F₂O₄SiNa ([M+H]⁺) 391.1153; found 391.1110.

(1R,3S)-15: IR (ATR): 3374, 2521, 2246, 1698, 1466, 1369, 1271, 1228, 1178, 1159, 1109, 1067, 1001, 910, 886, 582, 796, 758, 731, 682, 656, 632. ¹H-NMR (500 MHz; MeOD): δ 7.56-7.54 (m, 1H), 7.50-7.46 (m, 3H), 5.19 (t, J=8.6 Hz, 1H). ¹³C-NMR (126 MHz; CDCl₃): δ 155.6, 141.0, 140.0, 131.6, 131.1, 126.8, 126.0, 124.9, 83.3, 80.4, 75.1, 74.6. ¹⁹F-NMR (471 MHz; CDCl₃): δ −118.63 (d, J=224.7 Hz, 1F), −131.89 (d, J=221.8 Hz, 1F). HRMS (ESI) m/z calcd for Cl₂H₈F₂O₄Na ([M+H]⁺) 277.0288; found 277.0291.

### 2′,3′-O-(t-Butyldimethylsilyl)-5′-O—(N-[3″-((1R,3S)-3′″-(t-Butyldimethylsilyloxy)-2′″,2′″-difluoro-1′″-hydroxy-1′″-indanyl)propioloyl]sulfamoyl)adenosine (S1)

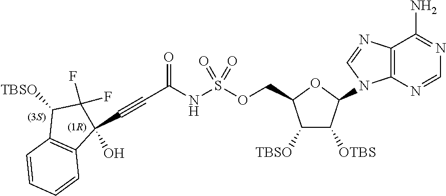

Propiolic acid (1R,3S)-11 (245 mg, 0.665 mmol, 1 equiv), protected 5′-O-sulfamoyladenosine 12 (573 mg, 0.997 mmol, 1.5 equiv) prepared as previously described (see, e.g., Ferreras, J. A.; Ryu, J. S.; Di Lello, F.; Tan, D. S.; Quadri, L. E. N. Nat. Chem. Biol. 2005, 1, 29-32) and DMAP (81 mg, 0.665 mmol, 1.0 equiv.) was dissolved in CH₂Cl₂ (5 mL) and EDCI (510 mg, 2.659 mmol, 4.0 equiv) was added. The reaction was stirred for 12 h, quenched with 25 mL 1 M KHSO₄, and extracted with CH₂Cl₂ (5×25 mL). The combined organic extracts were dried (Na₂SO₄), filtered, and concentrated by rotary evaporation. The reside was reconstituted in CH₂Cl₂, loaded into a pad of silica and washed with 100 mL CH₂Cl₂, then eluted with 15% MeOH/CH₂Cl₂ (200 mL) to afford the crude propiolyl-sulfamate (1R,3S)-S1 (499 mg), which was used without further purification.

### 2′,3′-O-(t-Butyldimethylsilyl)-5′-O—(N-[3″-((1R,3S)-3′″-(t-Butyldimethylsilyloxy)-2′″,2′″-difluoro-1′″-hydroxy-1′″-indanyl)propanoyl]sulfamoyl)adenosine (S2)

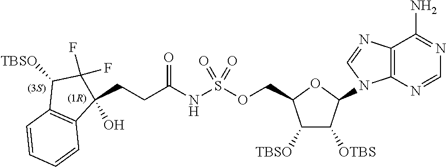

Crude propiolyl-sulfamate (1R,3S)-S1 (499 mg, 0.540 mmol, 1 equiv.) from previous step and 10% Pd/C (575 mg, 0.540 mmol, 1 equiv) were suspended in solution of MeOH/NEt₃ (50 mL, 9:1). The reaction was then stirred vigorously under H₂ balloon for 2 h, then diluted with EtOAc (50 mL), filtered through a celite pad, and concentrated by rotary evaporation to afford the crude propanoyl-sulfamate (1R,3S)-S2 (500 mg), which was used without further purification.

### 5′-O—(N-[3″-((1R,3S)-2′″,2′″-difluoro-1′″,3′″-dihydroxy-1′″-indanyl)propanoyl]sulfamoyl)adenosine (2)

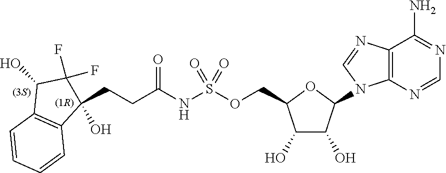 [SMILES: Nc1ncnc2c1ncn2[C@@H]1O[C@H](COS(=O)(=O)NC(=O)CC[C@@]2(O)c3ccccc3[C@H](O)C2(F)F)[C@@H](O)[C@H]1O]

Crude propanoyl-sulfamate (1R,3S)-S2 (500 mg, 0.538 mmol, 1 equiv.) was suspended in DMF (5 mL), then TASF (592 mg, 2.151 mmol, 4.0 equiv.) was added and the reaction mixture was stirred for 12 h at 50° C. Concentration by rotary evaporation, purification by preparative HPLC (5%→30% MeCN in H₂O with 0.1% TFA), and lyophilization yielded the syn-difluoroindanediol (1R,3S)-2 as a fluffy white solid (144 mg, 37% over 3 steps). N.B.: HPLC fractions were stored at 0° C. until just prior to pooling and freezing (dry-ice bath) for lyophilization. IR (ATR): 3340, 2504, 2245, 2074, 1684, 1558, 1474 1421, 1377, 1201, 1140, 1043, 979, 882, 842, 800, 724, 645. ¹H-NMR (500 MHz; CD₃OD): δ 8.46 (s, 1H), 8.35 (s, 1H), 7.44-7.37 (m, 4H), 6.09 (d, J=4.8 Hz, 1H), 5.12 (dd, J=11.6, 7.5 Hz, 1H), 4.63 (t, J=5.0 Hz, 1H), 4.54-4.48 (m, 2H), 4.39 (t, J=4.9 Hz, 1H), 4.30-4.28 (m, 1H), 2.61 (ddd, J=16.2, 10.0, 5.9 Hz, 1H), 2.47 (ddd, J=16.3, 9.9, 6.1 Hz, 1H), 2.16-2.09 (m, 1H), 1.83 (ddd, J=14.7, 9.4, 5.6 Hz, 1H). ¹³C-NMR (126 MHz; CD₃OD): δ 173.2, 150.2, 147.05, 147.03, 143.4, 142.9, 139.3, 130.4, 130.1, 125.2, 124.8, 120.5, 90.3, 83.6, 79.4, 75.8, 74.2, 72.3, 71.6, 49.5, 31.6, 30.9. ¹⁹F-NMR (471 MHz; CD₃OD): δ −128.07 (d, J=225.3 Hz, 1F), −130.99 (d, J=225.2 Hz, 1F). HRMS (ESI) m/z calcd for C₂₂H₂₅N₆O₉F2S ([M+H]⁺) 587.1372; found 587.1364.

### Synthesis of 1S,3R-syn-Difluoroindanediol (1S,3R)-2

See FIG. 7B for a scheme corresponding to the synthesis exemplified below.

### (S)-3-((t-Butyldimethylsilyl)oxy)-1-indanone (8)

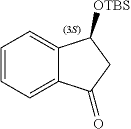 [SMILES: CC(C)(C)[Si](C)(C)O[C@H]1CC(=O)c2ccccc21]

(S)-3-Hydroxy-1-indanone 6 (720 mg, 4.859 mmol, 1 equiv.) was dissolved in 10 mL CH₂Cl₂ and imidazole (860 mg, 12.63 mmol, 2.6 equiv.) was added. TBSCl (952 mg, 6.316 mmol, 1.3 equiv.) was added and the reaction mixture was stirred at rt for 12 h, then diluted with 50 mL water and extracted with CH₂Cl₂ (4×50 mL). The combined organic extracts were dried (Na₂SO₄), filtered, and concentrated by rotary evaporation. Purification by silica flash chromatography (0%→30% EtOAc in hexanes) yielded the silyl ether (3S)-8 as a yellow tinged oil (1.13 g, 91%). IR (ATR): 2955, 2930, 2886, 2857, 1720, 1606, 1464, 1390, 1361, 1279, 1254, 1216, 1161, 1106, 1079, 1046, 1006, 961, 9334, 857, 837, 809, 776, 759, 719, 668. ¹H-NMR (500 MHz; CDCl₃): δ 7.74 (d, J=7.7 Hz, 1H), 7.68-7.65 (m, 1H), 7.61 (d, J=7.7 Hz, 1H), 7.46 (t, J=7.4 Hz, 1H), 5.39 (dd, J=6.6, 3.4 Hz, 1H), 3.06 (dd, J=18.3, 6.7 Hz, 1H), 2.60 (dd, J=18.3, 3.4 Hz, 1H), 0.96 (d, J=5.4 Hz, 9H), 0.23 (d, J=5.8 Hz, 3H), 0.19 (d, J=5.9 Hz, 3H). ¹³C-NMR (126 MHz; CDCl₃): δ 203.1, 156.0, 136.3, 135.1, 129.0, 125.8, 123.0, 68.9, 47.9, 25.8, 18.2, −4.4, −4.6. HRMS (ESI) m/z calcd for C₁₅H₂₃O₂Si ([M+H]⁺) 263.1467; found 263.1465.

### (R)-3-((t-Butyldimethylsilyl)oxy)-2,2-difluoro-1-indanone (9)

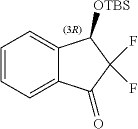 [SMILES: CC(C)(C)[Si](C)(C)O[C@@H]1c2ccccc2C(=O)C1(F)F]

Ketone (3S)-8 (1 g, 3.814 mmol, 1 equiv.) was dissolved in 80 mL cyclohexane, then hexylamine (2 mL, 15.25 mmol, 4 equiv.) and trifluoroacetic acid (0.015 mL, 0.19 mmol, 0.05 equiv.) were added and the reaction mixture was heated to reflux for 14 h. The reaction was then cooled to rt, diluted with 75 mL toluene, concentrated by rotary evaporation, and placed under high vacuum (˜60 mTorr) for 1 h. The crude imine was dissolved in acetonitrile (50 mL), then Selectfluor (2.83 g, 7.99 mmol, 2.1 equiv.) and sodium sulfate (378 mg, 2.663 mmol, 0.7 equiv.) were added and the reaction mixture was heated to reflux for 12 h. The reaction was cooled to rt, diluted with 1 M HCl (150 mL) and extracted with CH₂Cl₂ (4×100 mL). The combined organic extracts were dried (Na₂SO₄), filtered, and concentrated by rotary evaporation. Purification by silica flash chromatography (25%→75% CH₂Cl₂ in hexanes) yielded the difluoroindanone (3R)-9 as a yellow tinged oil (710 mg, 63%). IR (ATR): 2958, 2933, 2890, 2862, 1748, 1610, 1474, 1364, 1301, 1258, 1232, 1186, 1145, 1103, 1076, 1008, 929, 897, 840, 782, 741, 700, 672, 650. ¹H-NMR (500 MHz; CDCl₃): δ 7.85 (d, J=7.8 Hz, 1H), 7.82 (t, J=7.5 Hz, 1H), 7.64 (d, J=7.8 Hz, 1H), 7.57 (t, J=7.5 Hz, 1H), 5.24 (dd, J=12.8, 3.5 Hz, 1H), 0.98 (s, 9H), 0.29 (s, 3H), 0.25 (s, 3H). ¹³C-NMR (126 MHz; CDCl₃): δ 189.6, 150.4, 137.6, 132.3, 130.4, 126.2, 124.6, 114.93, 114.91, 71.8, 25.68, 18.3, −4.6, −5.1. ¹⁹F-NMR (471 MHz; CDCl₃): δ −116.52 (d, J=279.6 Hz, 1F), −123.46 (d, J=279.6 Hz, 1F). HRMS (ESI) m/z calcd for C₁₅H₂₀O₃F₂SiNa ([M+Na]+) 321.1098; found 321.1103.

### t-Butyl 3-((1S,3R)-3-(tert-butyldimethylsilyloxy)-2,2-difluoro-1-hydroxy-1-indanyl)propiolate (10)

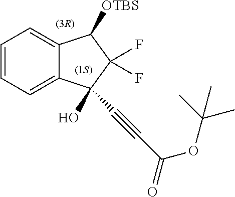 [SMILES: CC(C)(C)OC(=O)C#C[C@]1(O)c2ccccc2[C@@H](O[Si](C)(C)C(C)(C)C)C1(F)F]

Lithium bis(trimethylsilyl)amide (4.95 mL, 4.95 mmol, 1.0 M in THF, 1.55 equiv.) was cooled to −78° C., then t-butyl propiolate (604 mg, 4.789 mmol, 1.5 equiv.) in 3 mL THF was added and the reaction mixture was stirred for 45 min. The solution was then added via cannula over 10 min to ketone (3R)-9 (953 mg, 3.193 mmol, 1 equiv.) in 5 mL THF at −78° C. and stirred for 2 h. The reaction was quenched with satd aq NH₄Cl (50 mL), warmed to rt, and extracted with EtOAc (4×50 mL). The combined organic extracts were dried (Na₂SO₄), filtered, and concentrated by rotary evaporation. Purification by silica flash chromatography (50%→100% CH₂Cl₂ in hexanes) yielded the ester (1S,3R)-10 as a clear viscous oil (1.05 g, 78%). IR (ATR): 3400, 2956, 2932, 2888, 2860, 2248, 1710, 1473, 1395, 1371, 1258, 1204, 1153, 1114, 1039, 1013, 909, 888, 838, 781, 751, 732, 695, 672, 657. ¹H-NMR (500 MHz; CDCl₃): δ 7.63-7.60 (m, 1H), 7.47-7.45 (m, 2H), 7.38-7.37 (m, 1H), 5.19 (dd, J=8.0, 6.3 Hz, 1H), 2.96 (d, J=2.3 Hz, 1H), 1.49 (s, 9H), 0.95 (s, 9H), 0.24 (s, 6H). ¹³C-NMR (126 MHz; CDCl₃): δ 151.8, 139.5, 139.1, 130.9, 130.3, 124.9, 124.4, 124.2, 84.2, 80.6, 78.2, 74.9, 74.4, 28.0, 25.7, 18.2, −4.65, −4.84. ¹⁹F-NMR (471 MHz; CDCl₃): δ δ −115.05 (d, J=224.7 Hz, 1F), −128.46 (d, J=224.6 Hz, 1F). HRMS (ESI) m/z calcd for C₂₂H₃₀O₄F₂SiNa ([M+Na]⁺) 441.1779; found 441.1785.

### 3-((1S,3R)-3-(t-Butyldimethylsilyloxy)-2,2-difluoro-1-hydroxy-1-indanyl)propiolic acid (11)

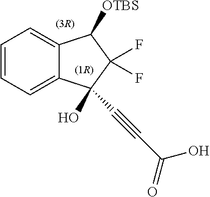 [SMILES: CC(C)(C)[Si](C)(C)O[C@@H]1c2ccccc2[C@@](O)(C#CC(=O)O)C1(F)F]

Ester (1S,3R)-10 (950 mg, 2.26 mmol, 1 equiv.) was dissolved in 10 mL CH₂Cl₂ and cooled to 0° C., then 10 mL TFA was added and the reaction mixture was stirred for 3 h. Concentration by rotary evaporation at 0° C. and purification by silica flash chromatography (50%→100% EtOAc in hexanes) yielded the acid (1S,3R)-11 as a white cotton type solid (465 mg, 56%), along with the corresponding desilated congener (1S,3R)-15 as a white solid (176 mg, 31%).

(1S,3R)-11: IR (ATR): 2958, 2934, 2893, 2862, 2253, 1701, 1474, 1365, 1249, 1152, 1095, 1010, 912, 893, 841, 783, 764, 733, 688, 653, 626. ¹H-NMR (500 MHz; CDCl₃): δ 7.61-7.60 (m, 1H), 7.50-7.45 (m, 2H), 7.40-7.38 (m, 1H), 5.18 (dd, J=8.2, 5.8 Hz, 1H), 0.94 (s, 9H), 0.24 (s, 6H). ¹³C-NMR (126 MHz; CDCl₃): δ 156.3, 139.2, 139.0, 131.2, 130.5, 125.1, 124.5, 124.0, 83.4, 78.5, 75.1, 74.5, 25.7, 18.2, −4.66, −4.85. ¹⁹F-NMR (471 MHz; CDCl₃): 114.45 (d, J=224.6 Hz, 1F), −127.98 (d, J=224.6 Hz, 1F). HRMS (ESI) m/z calcd for C₁₈H₂₂O₄F₂NaSi ([M+H]⁺) 391.1153; found 391.1154.

(1S,3R)-15: IR (ATR): 3354, 2502, 2246, 1697, 1466, 1271, 1228, 1178, 1159, 1109, 1066, 1000, 974, 909, 886, 851, 795, 759, 730, 683, 655, 631. ¹H-NMR (500 MHz; MeOD): δ 7.56-7.54 (m, 1H), 7.50-7.46 (m, 3H), 5.18 (t, J=8.6 Hz, 1H). ¹³C-NMR (126 MHz; MeOD): δ 155.6, 141.0, 140.0, 131.6, 131.1, 126.9, 126.0, 124.9, 83.3, 80.4, 75.1, 74.5. ¹⁹F-NMR (471 MHz; MeOD): δ −118.67 (d, J=221.5 Hz, 1F), −131.92 (d, J=224.7 Hz, 1F). HRMS (ESI) m/z calcd for C₂₄H₁₆O₈F₄ ([2M−H]⁻) 507.0703; found 507.0704.

### 2′,3′-O-(t-Butyldimethylsilyl)-5′-O—(N-[3″-((1S,3R)-3′″-(t-Butyldimethylsilyloxy)-2′″,2′″-difluoro-1′″-hydroxy-1′″-indanyl)propioloyl]sulfamoyl)adenosine (S1)

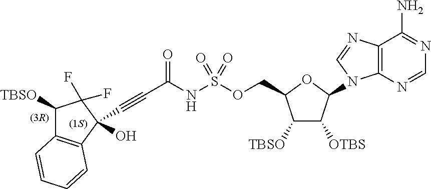 [SMILES: CC(C)(C)[Si](C)(C)O[C@@H]1[C@H](O[Si](C)(C)C(C)(C)C)[C@@H](COS(=O)(=O)NC(=O)C#C[C@]2(O)c3ccccc3[C@@H](O[Si](C)(C)C(C)(C)C)C2(F)F)O[C@H]1n1cnc2c(N)ncnc21]

Propiolic acid (1S,3R)-11 (250 mg, 0.678 mmol, 1 equiv), protected 5′-O-sulfamoyladenosine 12 (585 mg, 1.017 mmol, 1.5 equiv) prepared as previously described,³ and DMAP (83 mg, 0.678 mmol, 1.0 equiv.) was dissolved in CH₂Cl₂ (5 mL) and EDCI (520 mg, 2.714 mmol, 4.0 equiv) was added. The reaction was stirred for 12 h, then quenched with 25 mL 1 M KHSO₄, and extracted with CH₂Cl₂ (5×25 mL). The combined organic extracts were dried (Na₂SO₄), filtered, and concentrated by rotary evaporation. The reside was reconstituted in CH₂Cl₂, loaded into a pad of silica and washed with 100 mL CH₂Cl₂, then eluted with 15% MeOH/CH₂Cl₂ (200 mL) to afford the crude propiolyl-sulfamate (1S,3R)-S1 (480 mg), which was used without further purification.

### 2′,3′-O-(t-Butyldimethylsilyl)-5′-O—(N-[3″-((1S,3R)-3′″-(t-Butyldimethylsilyloxy)-2′″,2′″-difluoro-1′″-hydroxy-1′″-indanyl)propanoyl]sulfamoyl)adenosine (S2)

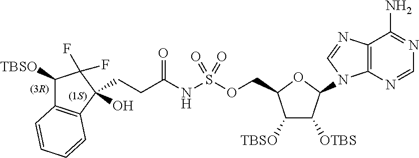 [SMILES: CC(C)(C)[Si](C)(C)O[C@@H]1[C@H](O[Si](C)(C)C(C)(C)C)[C@@H](COS(=O)(=O)NC(=O)CC[C@]2(O)c3ccccc3[C@@H](O[Si](C)(C)C(C)(C)C)C2(F)F)O[C@H]1n1cnc2c(N)ncnc21]

Crude propiolyl-sulfamate (1S,3R)-S1 (480 mg, 0.519 mmol, 1 equiv.) from previous step and 10% Pd/C (552 mg, 0.519 mmol, 1 equiv) were suspended in solution of MeOH/NEt₃ (50 mL, 9:1). The reaction was then stirred vigorously under H₂ balloon for 2 h, then diluted with EtOAc (50 mL), filtered through a celite pad, and concentrated by rotary evaporation to afford the crude propanoyl-sulfamate (1S,3R)-S2 (428 mg), which was used without further purification.

### 5′-O—(N-[3″-((1S,3R)-2′″,2′″-difluoro-1′″,3′″-dihydroxy-1′″-indanyl)propanoyl]-sulfamoyl)adenosine (2)

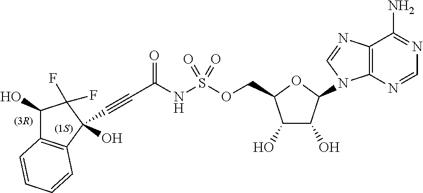 [SMILES: Nc1ncnc2c1ncn2[C@@H]1O[C@H](COS(=O)(=O)NC(=O)C#C[C@]2(O)c3ccccc3[C@@H](O)C2(F)F)[C@@H](O)[C@H]1O]

Crude propanoyl-sulfamate (1S,3R)-S2 (480 mg, 0.461 mmol, 1 equiv.) was suspended in DMF (5 mL), then TASF (507 mg, 1.841 mmol, 4.0 equiv.) was added and the reaction mixture was stirred for 12 h at 50° C. Concentration by rotary evaporation, purification by preparative HPLC (5%→30% MeCN in H₂O with 0.1% TFA), and lyophilization yielded the syn-difluoroindanediol (1S,3R)-2 as a fluffy white solid (123 mg, 31% over 3 steps). N.B.: HPLC fractions were stored at 0° C. until just prior to pooling and freezing (dry-ice bath) for lyophilization. IR (ATR): 3368, 2512, 2241, 2077, 1687, 1478, 1425, 1379, 1202, 1141, 1045, 980, 882, 803, 726, 645. ¹H-NMR (500 MHz; CD₃OD): δ 8.42 (s, 1H), 8.34 (s, 1H), 7.42-7.36 (m, 4H), 6.07-6.06 (m, 1H), 5.15-5.10 (m, 1H), 4.63-4.60 (m, 1H), 4.54-4.46 (m, 2H), 4.40-4.37 (m, 1H), 4.30-4.27 (m, 1H), 2.66-2.60 (m, 1H), 2.49-2.42 (m, 1H), 2.18-2.12 (m, 1H), 1.81-1.75 (m, 1H). ¹³C-NMR (126 MHz; CD₃OD): δ 173.2, 150.2, 147.01, 146.86, 143.4, 142.9, 139.3, 130.4, 130.1, 125.2, 124.9, 120.5, 90.3, 83.6, 79.4, 75.8, 74.2, 72.3, 71.6, 49.9, 31.6, 30.9. ¹⁹F-NMR (471 MHz; CD₃OD): δ −128.11 (d, J=225.3 Hz, 1F), −131.06 (d, J=224.7 Hz, 1F). HRMS (ESI) m/z calcd for C₂₂H₂₅N₆O₉F2S ([M+H]⁺) 587.1372; found 587.1353.

### Synthesis of 1R, 3R-anti-Difluoroindanediol (1R,3R)-2

See FIG. 7C for a scheme detailing the exemplary synthesis below.

### t-Butyl 3-((1R,3S)-2,2-difluoro-1,3-dihydroxy-1-indanyl)propiolate (14)

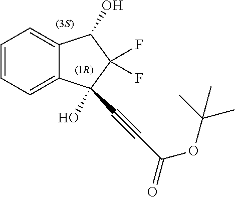 [SMILES: CC(C)(C)OC(=O)C#C[C@@]1(O)c2ccccc2[C@H](O)C1(F)F]

Silyl ether (1R,3S)-10 (470 mg, 1.107 mmol, 1.0 equiv.) was dissolved in 2 mL THF and cooled to 0° C., then tetrabutylammonium fluoride (1.217 mL, 1.217 mmol, 1.0 M in THF, 1.1 equiv.) was added, and the reaction mixture was stirred for 1 h. Concentration by rotary evaporation and purification by silica flash chromatography (30%→60% EtOAc in hexanes) yielded the diol (1R,3S)-14 as a white solid (285 mg, 83%). IR (ATR): 3377, 2984, 2936, 2249, 1707, 1459, 1396, 1372, 1281, 1232, 1152, 1110, 1067, 1003, 909, 838, 798, 754, 732, 682, 660, 649. ¹H-NMR (500 MHz; CDCl₃): δ 7.65-7.62 (m, 1H), 7.54-7.48 (m, 3H), 5.11 (dd, J=8.7, 4.0 Hz, 1H), 3.10 (s, 2H), 1.49 (s, 9H). ¹³C-NMR (126 MHz; CDCl₃): δ 152.0, 139.6, 138.6, 131.2, 130.7, 125.8, 124.7, 123.7, 84.6, 80.7, 78.3, 74.8, 74.2, 28.0. ¹⁹F-NMR (471 MHz; CDCl₃): δ δ −114.08 (d, J=232.9 Hz, 1F), −128.77 (d, J=232.6 Hz, 1F). HRMS (ESI) m/z calcd for C₁₆H₁₆O₄F₂Na ([M+H]⁺) 333.0914; found 333.0916.

### (R)-t-Butyl 3-(2,2-difluoro-1-hydroxy-3-oxo-1-indanyl)propiolate (13)

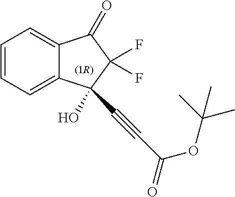 [SMILES: CC(C)(C)OC(=O)C#C[C@@]1(O)c2ccccc2C(=O)C1(F)F]

DMSO (227 mg, 2.9 mmol, 3.0 equiv.) was dissolved in 4 mL CH₂Cl₂, cooled to −78° C., and oxalyl chloride (184 mg, 1.450 mmol, 1.5 equiv.) was added and the reaction mixture was stirred for 10 min. Diol (1R,3S)-14 (300 mg, 0.967 mmol, 1.0 equiv.) in 1.5 mL CH₂Cl₂ was added dropwise, then the reaction mixture was stirred for 40 min. Triethylamine (0.675 mL, 4.834 mmol, 5.0 equiv.) was added and the reaction mixture was stirred for 40 min, then removed from the dry-ice bath and stirred for 10 min. The reaction was then quenched with satd aq NH₄Cl (30 mL), extracted with CH₂Cl₂ (4×20 mL), the combined organic extracts were dried (Na₂SO₄), filtered, and concentrated by rotary evaporation. Purification by silica flash chromatography (5%→25% EtOAc in hexanes) yielded the ketoalcohol (1R)-15 as a clear oil (272 mg, 91%). IR (ATR): 3410, 2985, 2938, 2244, 1752, 1712, 1604, 1471, 1397, 1372, 1286, 1222, 1193, 1152, 1101, 1041, 1017, 934, 910, 877, 837, 770, 755, 736, 712, 693, 649. ¹H-NMR (500 MHz; CDCl₃): δ 7.94-7.87 (m, 3H), 7.70-7.67 (m, 1H), 3.67 (s, 1H), 1.51 (s, 9H). ¹³C-NMR (126 MHz; CDCl₃): δ 187.6, 151.6, 148.8, 138.1, 132.0, 131.2, 126.3, 125.2, 113.5, 85.0, 82.2, 77.2, 71.1, 28.0. ¹⁹F-NMR (471 MHz; CDCl₃): δ −111.48 (d, J=271.1 Hz, 1F), −126.10 (d, J=271.2 Hz, 1F). HRMS (ESI) m/z calcd for C₁₆H₁₄O₄F₂Cl ([M+C1]⁻) 343.0549; found 343.0565.

### t-Butyl 3-((1R,3R)-2,2-difluoro-1,3-dihydroxy-1-indanyl)propiolate (14)

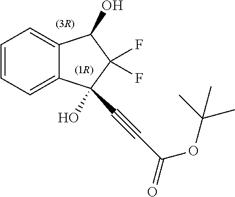 [SMILES: CC(C)(C)OC(=O)C#C[C@@]1(O)c2ccccc2[C@@H](O)C1(F)F]

Ketone (1R)-13 (300 mg, 0.941 mmol, 1 equiv.) was dissolved in 5 mL MeOH and cooled to 0° C., then NaBH₄ (11 mg, 0.282 mmol, 0.3 equiv.) was added in 4 portions over 5 min and the reaction mixture was stirred for 30 min. Acetone (0.1 mL) was added and the reaction mixture was stirred for 10 min, then 1 M phosphate buffer (pH 7.0, 20 mL) was added and the reaction mixture was stirred for an additional 10 min. The reaction was then extracted with EtOAc (4×15 mL), the combined organic extracts were dried (Na₂SO₄), filtered, and concentrated by rotary evaporation. Purification by silica flash chromatography (0%→100% EtOAc in CH₂Cl₂) yielded the anti-diol (1R,3R)-14 as a white solid (263 mg, 90%). IR (ATR): 3371, 2983, 2930, 2241, 1684, 1395, 1371, 1230, 1300, 1152, 1111, 1078, 1032, 1003, 913, 834, 752, 731, 649, 574. ¹H-NMR (500 MHz; CDCl₃): δ 7.63 (d, J=7.4 Hz, 1H), 7.52-7.49 (m, 2H), 7.48-7.45 (m, 1H), 5.41 (td, J=10.3, 6.4 Hz, 1H), 3.11 (d, J=1.5 Hz, 1H), 2.38 (dd, J=10.7, 2.1 Hz, 1H), 1.51 (s, 9H). ¹³C-NMR (126 MHz; CDCl₃): δ 151.8, 139.0, 137.6, 131.5, 130.2, 124.94, 124.74, 123.7, 84.6, 80.8, 77.8, 74.17, 74.06, 28.0. ¹⁹F-NMR (471 MHz; CDCl₃): δ −123.33 (d, J=225.3 Hz, 1F), −125.61 (d, J=226.3 Hz, 1F). HRMS (ESI) m/z calcd for C₁₆H₁₆O₄F₂Na ([M+H]⁺) 333.0914; found 333.0920.

### 3-((1R,3R)-2,2-difluoro-1,3-dihydroxy-1-indanyl)propiolic acid (15)

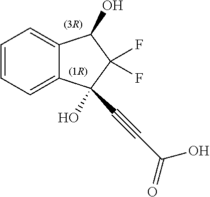 [SMILES: O=C(O)C#C[C@@]1(O)c2ccccc2[C@@H](O)C1(F)F]

Ester (1R,3R)-14 (185 mg, 0.593 mmol, 1 equiv.) was dissolved in 5 mL CH₂Cl₂ and cooled to 0° C., then 5 mL TFA was added and the reaction mixture was stirred for 3 h. Concentration by rotary evaporation at 0° C. gave crude acid (1R,3R)-15 (170 mg) used directly on the next step without further purification.

### 2′,3′-O-(t-Butyldimethylsilyl)-5′-O—(N-[3″-((1R,3R)-2′″,2′″-difluoro-1′″, 3-dihydroxy-1′″-indanyl)propioloyl]sulfamoyl)adenosine (S3)

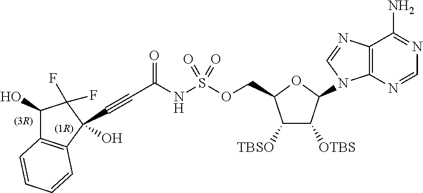 [SMILES: CC(C)(C)[Si](C)(C)O[C@@H]1[C@H](O[Si](C)(C)C(C)(C)C)[C@@H](COS(=O)(=O)NC(=O)C#C[C@@]2(O)c3ccccc3[C@@H](O)C2(F)F)O[C@H]1n1cnc2c(N)ncnc21]

Propiolic acid (1R,3R)-15 (assumed quantitative yield from previous step: 151 mg, 0.594 mmol, 1 equiv), protected 5′-O-sulfamoyladenosine 12 (427 mg, 0.723 mmol, 1.25 equiv) prepared as previously described,³ and DMAP (73 mg, 0.594 mmol, 1.0 equiv.) was dissolved in CH₂C12:MeCN (5 mL, 2:1) and EDCI (456 mg, 2.376 mmol, 4.0 equiv) was added. The reaction was stirred for 12 h, quenched with 15 mL 1 M KHSO₄, and extracted with EtOAc (5×15 mL). The combined organic extracts were dried (Na₂SO₄), filtered, and concentrated by rotary evaporation. The reside was reconstituted in CH₂Cl₂, loaded into a pad of silica and washed with 100 mL CH₂Cl₂, then eluted with 15% MeOH/CH₂Cl₂ (150 mL) to afford the crude propiolyl-sulfamate (1R,3R)-S3 (294 mg), which was used without further purification.

### 2′,3′-O-(t-Butyldimethylsilyl)-5′-O—(N-[3″-((1R,3R)-2′″,2′″-difluoro-1′″,3′″-dihydroxy-1′″-indanyl)propanoyl]sulfamoyl)adenosine (S4)

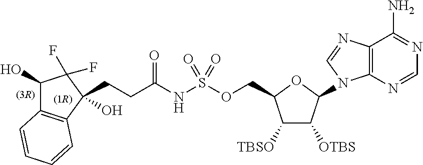 [SMILES: CC(C)(C)[Si](C)(C)O[C@@H]1[C@H](O[Si](C)(C)C(C)(C)C)[C@@H](COS(=O)(=O)NC(=O)CC[C@@]2(O)c3ccccc3[C@@H](O)C2(F)F)O[C@H]1n1cnc2c(N)ncnc21]

Crude propiolyl-sulfamate (1R,3R)-S3 (294 mg, 0.363 mmol, 1 equiv.) from previous step and 10% Pd/C (386 mg, 0.363 mmol, 1 equiv) were suspended in solution of MeOH/NEt₃ (40 mL, 9:1). The reaction was then stirred vigorously under H₂ balloon for 2 h, then diluted with EtOAc (15 mL), filtered through a celite pad, and concentrated by rotary evaporation to afford the crude propanoyl-sulfamate (1R,3R)-S4 (300 mg), which was used without further purification.

### 5′-O—(N-[3″-((1R,3R)-2′″,2′″-difluoro-1′″,3′″-dihydroxy-1′″-indanyl)propanoyl]-sulfamoyl)adenosine (2)

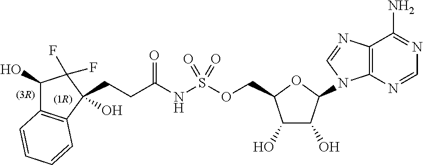 [SMILES: Nc1ncnc2c1ncn2[C@@H]1O[C@H](COS(=O)(=O)NC(=O)CC[C@@]2(O)c3ccccc3[C@@H](O)C2(F)F)[C@@H](O)[C@H]1O]

Crude propanoyl-sulfamate (1R,3R)-S4 (300 mg, 0.370 mmol, 1 equiv.) was suspended in DMF (1.5 mL), then TASF (306 mg, 1.109 mmol, 3.0 equiv.) was added and the reaction mixture was stirred for 12 h at 50° C. Concentration by rotary evaporation, purification by preparative HPLC (5%→30% MeCN in H₂O with 0.1% TFA), and lyophilization yielded the anti-difluoroindanediol (1R,3R)-2 as a fluffy white solid (75 mg, 35% over 4 steps). N.B.: HPLC fractions were stored at 0° C. until just prior to pooling and freezing (dry-ice bath) for lyophilization. IR (ATR): 3343, 2942, 2865, 2509, 2076, 1692, 1473, 1420, 1378, 1198, 1134, 976, 885, 835, 800, 765, 723, 680, 638. ¹H-NMR (500 MHz; CD₃OD): δ 8.47 (s, 1H), 8.34 (s, 1H), 7.45-7.40 (m, 4H), 6.10 (d, J=4.8 Hz, 1H), 5.12 (dd, J=9.7, 5.9 Hz, 1H), 4.64 (t, J=5.0 Hz, 1H), 4.57-4.51 (m, 2H), 4.41 (t, J=4.9 Hz, 1H), 4.31 (q, J=3.9 Hz, 1H), 2.63 (t, J=7.9 Hz, 2H), 2.32-2.13 (m, 2H). ¹³C-NMR (126 MHz; CD₃OD): δ 173.4, 150.2, 147.5, 147.3, 143.8, 143.3, 140.0, 130.70, 130.63, 126.4, 124.9, 120.5, 90.3, 83.6, 79.6, 75.8, 74.9, 72.3, 71.6, 49.3, 31.31, 31.13. ¹⁹F-NMR (471 MHz; CD₃OD): δ −120.31 (d, J=230.1 Hz, 1F), −130.90 (d, J=233.2 Hz, 1F). HRMS (ESI) m/z calcd for C₂₂H₂₅N₆O₉F2S ([M+H]⁺) 587.1372; found 587.1370.

### Synthesis of 1S,3S-anti-Difluoroindanediol (1S,3S)-2

See FIG. 7D for a scheme corresponding to the following synthesis.

### t-Butyl 3-((1S,3R)-2,2-difluoro-1,3-dihydroxy-1-indanyl)propiolate (14)

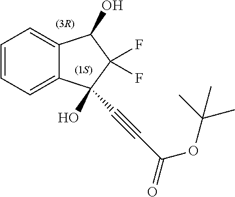 [SMILES: CC(C)(C)OC(=O)C#C[C@]1(O)c2ccccc2[C@@H](O)C1(F)F]

Silyl ether (1S,3R)-10 (681 mg, 1.604 mmol, 1.0 equiv.) was dissolved in 4 mL THF and cooled to 0° C., then tetrabutylammonium fluoride (1.764 mL, 1.764 mmol, 1.0 M in THF, 1.1 equiv.) was added and the reaction mixture was stirred for 1 h. Concentration by rotary evaporation and purification by silica flash chromatography (30%→60% EtOAc in hexanes) yielded the diol (1S,3R)-14 as a white solid (405 mg, 81%). IR (ATR): 3395, 2984, 2936, 2249, 1708, 1459, 1397, 1372, 1281, 1232, 1152, 1110, 1068, 1003, 909, 882, 839, 798, 756, 732, 696, 682, 659, 649. ¹H-NMR (500 MHz; CDCl₃): δ 7.64-7.61 (m, 1H), 7.53-7.47 (m, 3H), 5.11 (dd, J=8.7, 4.0 Hz, 1H), 3.05 (s, 2H), 1.48 (s, 9H). ¹³C-NMR (126 MHz; CDCl₃): δ 152.1, 139.6, 138.6, 131.2, 130.7, 125.8, 124.7, 123.7, 84.7, 80.6, 78.4, 74.8, 74.2, 28.0. ¹⁹F-NMR (471 MHz; CDCl₃): δ −114.00 (d, J=228.8 Hz, 1F), −128.71 (d, J=228.8 Hz, 1F). HRMS (ESI) m/z calcd for C₁₆H₁₆O₄F₂Na ([M+H]⁺) 374.0914; found 374.1198.

### X-Ray Crystallographic Analysis of Syn-Diol (1S,3R)-14

syn-Diol acid (1S,3R)-14 (10 mg, 0.0393 mmol, 1 equiv.) and (R)-α-methyl-4-nitrobenzylamine (6.9 mg, 0.0413 mmol, 1.05 equiv., Sigma Aldrich) were placed in a 4 mL glass sample vial and dissolved in 400 μL MeOH. The vial was placed in a 20 mL glass sample vial containing diethyl ether and the 20 mL vial sealed tightly. After 3 days at rt, clear needle shaped crystals were obtained.

A specimen of [C₈H₁₁N₂O₂][C₁₂H₇F₂O₄]*CH₃OH was used for X-ray crystallographic analysis at the University of Toledo Instrumentation Center at 120 K on a Bruker APEX Duo diffractometer using CuKα radiation (1.54178 Å) for absolute stereochemistry determination. The X-ray intensity data were measured. The integration of the data using a monoclinic unit cell yielded a total of 14285 reflections to a maximum 0 angle of 70.88° (0.82 Å resolution), of which 3562 were independent (average redundancy 4.010, completeness=95.5%, Rᵢₙₜ=2.21%, R_{sig}=2.00%) and 3536 (99.27%) were greater than 2σ(F²). The final cell constants of a=13.014(4) Å, b=9.450(3) Å, c=18.211(5) Å, β=98.828(8)°, volume=2213.1(11) Å³, are based upon the refinement of the XYZ-centroids of reflections above 20 σ(I).

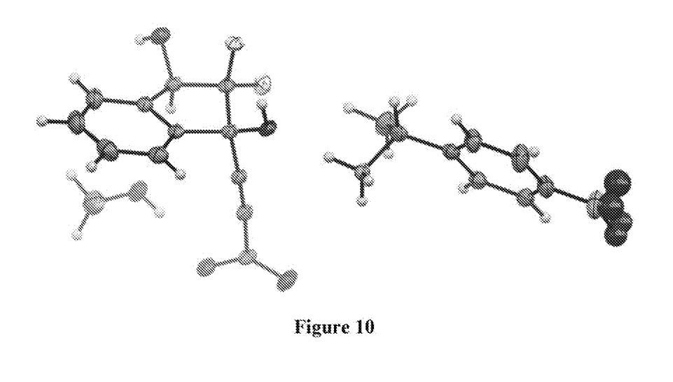

The structure was solved and refined using the Bruker SHELXTL Software Package, using the space group C 1 2 1, with Z=4 for the formula unit, C₂₁H₂₂F₂N₂O₇. The final anisotropic full-matrix least-squares refinement on F² with 377 variables converged at R1=3.05%, for the observed data and wR2=8.16% for all data. The goodness-of-fit was 1.338. The NO₂ group is disordered over two equally occupied positions (both shown in FIG. 10). The largest peak in the final difference electron density synthesis was 0.309 e⁻/Å³ and the largest hole was −0.335 e⁻/Å³ with an RMS deviation of 0.040 e⁻/Å³. On the basis of the final model, the calculated density was 1.358 g/cm³ and F(000), 944 e⁻.

### (S)-t-Butyl 3-(2,2-difluoro-1-hydroxy-3-oxo-1-indanyl)propiolate (13)

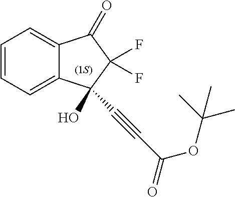 [SMILES: CC(C)(C)OC(=O)C#C[C@]1(O)c2ccccc2C(=O)C1(F)F]

DMSO (147 mg, 1.885 mmol, 3.0 equiv.) was dissolved in 2.5 mL CH₂Cl₂, cooled to −78° C., and oxalyl chloride (120 mg, 0.943 mmol, 1.5 equiv.) was added and the reaction mixture was stirred for 10 min. Diol (1S,3R)-14 (195 mg, 0.628 mmol, 1.0 equiv.) in 1 mL CH₂Cl₂ was added and the reaction mixture was stirred for 40 min. Triethylamine (0.438 mL, 3.142 mmol, 5.0 equiv.) was added and the reaction mixture was stirred for 40 min, then removed from the dry-ice bath and stirred for 10 min. The reaction was then quenched with satd aq NH₄Cl (20 mL), extracted with CH₂Cl₂ (4×15 mL), the combined organic extracts were dried (Na₂SO₄), filtered, and concentrated by rotary evaporation. Purification by silica flash chromatography (5%→25% EtOAc in hexanes) yielded the ketoalcohol (1S)-13 as a clear oil (180 mg, 93%). IR (ATR): 3411, 2986, 2939, 2246, 1753, 1713, 1606, 1473, 1398, 1374, 1287, 1223, 1194, 1153, 1103, 1043, 1019, 936, 911, 879, 839, 772, 756, 737, 713, 651. ¹H-NMR (500 MHz; CDCl₃): δ 7.94-7.87 (m, 3H), 7.68 (td, J=7.5, 1.0 Hz, 1H), 3.78 (s, 1H), 1.51 (s, 9H). ¹³C-NMR (126 MHz; CDCl₃): δ 187.7, 151.7, 148.9, 138.1, 132.0, 131.2, 126.3, 125.2, 113.5, 85.0, 82.2, 77.3, 71.1, 28.0. ¹⁹F-NMR (471 MHz; CDCl₃): δ −111.41 (d, J=268.0 Hz, 1F), −126.12 (d, J=270.6 Hz, 1F). HRMS (ESI) m/z calcd for C₁₆H₁₄O₄F₂Na ([M+H]⁺) 331.0758; found 331.0750.

### t-Butyl 3-((1S,3S)-2,2-difluoro-1,3-dihydroxy-1-indanyl)propiolate (16)

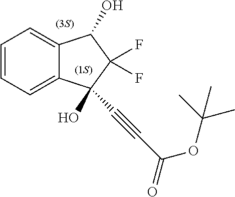 [SMILES: CC(C)(C)OC(=O)C#C[C@]1(O)c2ccccc2[C@H](O)C1(F)F]

Ketone (1S)-13 (200 mg, 0.649 mmol, 1 equiv.) was dissolved in 3 mL MeOH and cooled to 0° C., then NaBH₄ (7.4 mg, 0.195 mmol, 0.3 equiv.) was added in 4 portions over 5 min and the reaction mixture was stirred for 30 min. Acetone (0.1 mL) was added and the reaction mixture was stirred for 10 min, then 1 M phosphate buffer (pH 7.0, 15 mL) was added and the reaction mixture was stirred for an additional 10 min. The reaction was then extracted with EtOAc (4×10 mL), the combined organic extracts were dried (Na₂SO₄), filtered, and concentrated by rotary evaporation. Purification by silica flash chromatography (0%→10% EtOAc in CH₂Cl₂) yielded the anti-diol (1S,3S)-14 as a white solid (170 mg, 84%). IR (ATR): 3374, 2984, 2938, 2245, 1689, 1466, 1397, 1372, 1305, 1229, 1153, 1110, 1078, 1041, 1008, 911, 893, 837, 795, 756, 732, 696, 648. ¹H-NMR (500 MHz; CDCl₃): δ 7.63 (d, J=7.4 Hz, 1H), 7.51-7.45 (m, 3H), 5.40 (td, J=10.4, 6.4 Hz, 1H), 3.18 (s, 1H), 2.42 (dd, J=10.7, 2.5 Hz, 1H), 1.51 (s, 9H). ¹³C-NMR (126 MHz; CDCl₃): δ 151.8, 139.0, 137.6, 131.4, 130.2, 124.93, 124.73, 123.7, 84.6, 80.8, 77.9, 74.17, 74.05, 28.0. ¹⁹F-NMR (471 MHz; CDCl₃): δ −123.25 (d, J=228.4 Hz, 1F), −125.63 (d, J=229.1 Hz, 1F). HRMS (ESI) m/z calcd for C₁₆H₁₆O₄F₂Na ([M+H]⁺) 333.0914; found 333.0905.

### 3-((1S,3S)-2,2-difluoro-1,3-dihydroxy-1-indanyl)propiolic acid (15)

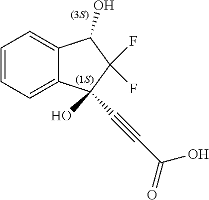 [SMILES: O=C(O)C#C[C@]1(O)c2ccccc2[C@H](O)C1(F)F]

Ester (1S,3S)-16 (135 mg, 0.435 mmol, 1 equiv.) was dissolved in 5 mL CH₂Cl₂ and cooled to 0° C., then 5 mL TFA was added and the reaction mixture was stirred for 3 h. Concentration by rotary evaporation at 0° C. gave crude acid (1S,3S)-17 (110 mg) used directly on the next step without further purification.

### 2′,3′-O-(t-Butyldimethylsilyl)-5′-O—(N-[3″-((1S,3S)-2′″,2′″-difluoro-1′″,3′″-dihydroxy-1′″-indanyl)propioloyl]sulfamoyl)adenosine (S3)

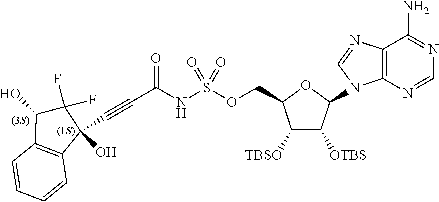 [SMILES: CC(C)(C)[Si](C)(C)O[C@@H]1[C@H](O[Si](C)(C)C(C)(C)C)[C@@H](COS(=O)(=O)NC(=O)C#C[C@]2(O)c3ccccc3[C@H](O)C2(F)F)O[C@H]1n1cnc2c(N)ncnc21]

Propiolic acid (1S,3S)-15 (assumed quantitative yield from previous step: 110 mg, 0.433 mmol, 1 equiv), protected 5′-O-sulfamoyladenosine 12 (373 mg, 0.541 mmol, 1.25 equiv) prepared as previously described,³ and DMAP (53 mg, 0.433 mmol, 1.0 equiv.) was dissolved in CH₂Cl₂:MeCN (5 mL, 2:1) and EDCI (332 mg, 1.730 mmol, 4.0 equiv) was added. The reaction was stirred for 12 h, then quenched with 15 mL 1 M KHSO₄, and extracted with EtOAc (5×15 mL). The combined organic extracts were dried (Na₂SO₄), filtered, and concentrated by rotary evaporation. The reside was reconstituted in CH₂Cl₂, loaded into a pad of silica and washed with 100 mL CH₂Cl₂, then eluted with 15% MeOH/CH₂Cl₂ (150 mL) to afford the crude propiolyl-sulfamate (1S,3S)-S3 (128 mg), which was used without further purification.

### 2′,3′-O-(t-Butyldimethylsilyl)-5′-O—(N-[3″-((1S,3S)-2′″,2′″-difluoro-1′″,3-dihydroxy-1′″-indanyl)propanoyl]sulfamoyl)adenosine (S4)

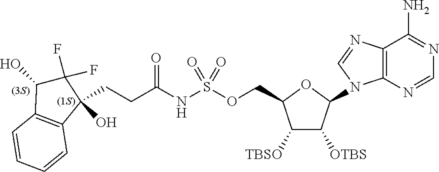 [SMILES: CC(C)(C)[Si](C)(C)O[C@@H]1[C@H](O[Si](C)(C)C(C)(C)C)[C@@H](COS(=O)(=O)NC(=O)CC[C@]2(O)c3ccccc3[C@H](O)C2(F)F)O[C@H]1n1cnc2c(N)ncnc21]

Crude propiolyl-sulfamate (1S,3S)-S3 (128 mg, 0.158 mmol, 1 equiv.) from previous step and 10% Pd/C (168 mg, 0.158 mmol, 1 equiv) were suspended in solution of MeOH/NEt₃ (15 mL, 9:1). The reaction was then stirred vigorously under H₂ balloon for 2 h, then diluted with EtOAc (15 mL), filtered through a celite pad, and concentrated by rotary evaporation to afford the crude propanoyl-sulfamate (1S,3S)-S4 (118 mg), which was used without further purification.

### 5′-O—(N-[3″-((1S,3S)-2′″,2′″-difluoro-1′″,3′″-dihydroxy-1′″-indanyl)propanoyl]sulfamoyl)adenosine (2)

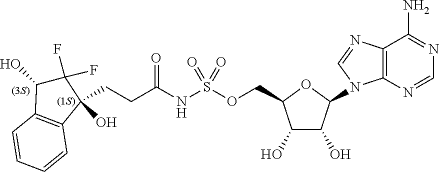 [SMILES: Nc1ncnc2c1ncn2[C@@H]1O[C@H](COS(=O)(=O)NC(=O)CC[C@]2(O)c3ccccc3[C@H](O)C2(F)F)[C@@H](O)[C@H]1O]

Crude propanoyl-sulfamate (1S,3S)-S4 (118 mg, 0.145 mmol, 1 equiv.) was suspended in DMF (1.5 mL), then TASF (120 mg, 0.434 mmol, 3.0 equiv.) was added and the reaction mixture was stirred for 12 h at 50° C. Concentration by rotary evaporation, purification by preparative HPLC (5%→30% MeCN in H₂O with 0.1% TFA), and lyophilization yielded the anti-difluoroindanediol (1S,3S)-2 as a fluffy white solid (53 mg, 21% over 4 steps). N.B.: HPLC fractions were stored at 0° C. until just prior to pooling and freezing (dry-ice bath) for lyophilization. IR (ATR): 3367, 2502, 2239, 2072, 1693, 1471, 1429, 1380, 1202, 1139, 980, 787, 801, 769, 724, 642. ¹H-NMR (500 MHz; CD₃OD): δ 8.46 (s, 1H), 8.33 (s, 1H), 7.45-7.39 (m, 4H), 6.09 (d, J=4.7 Hz, 1H), 5.11 (dd, J=9.7, 5.7 Hz, 1H), 4.63 (t, J=4.9 Hz, 1H), 4.58-4.50 (m, 2H), 4.40 (t, J=4.9 Hz, 1H), 4.32-4.30 (m, 1H), 3.34 (s, 1H), 2.86-2.83 (m, 1H), 2.63 (td, J=7.8, 2.3 Hz, 2H), 2.29-2.15 (m, 2H). ¹³C-NMR (126 MHz; CD₃OD): δ 173.5, 150.2, 147.43, 147.40, 143.8, 143.3, 140.0, 130.69, 130.63, 126.5, 125.0, 120.5, 90.3, 83.6, 79.6, 75.8, 74.9, 72.3, 71.6, 49.3, 31.3, 31.1. ¹⁹F-NMR (471 MHz; CD₃OD): δ −120.33 (d, J=233.1 Hz, 1F), −130.94 (d, J=232.3 Hz, 1F). HRMS (ESI) m/z calcd for C₂₂H₂₅N₆O₉F2S ([M+H]⁺) 587.1372; found 587.1366.

### Synthesis of a Boronic Acid Analog (Compound 139)

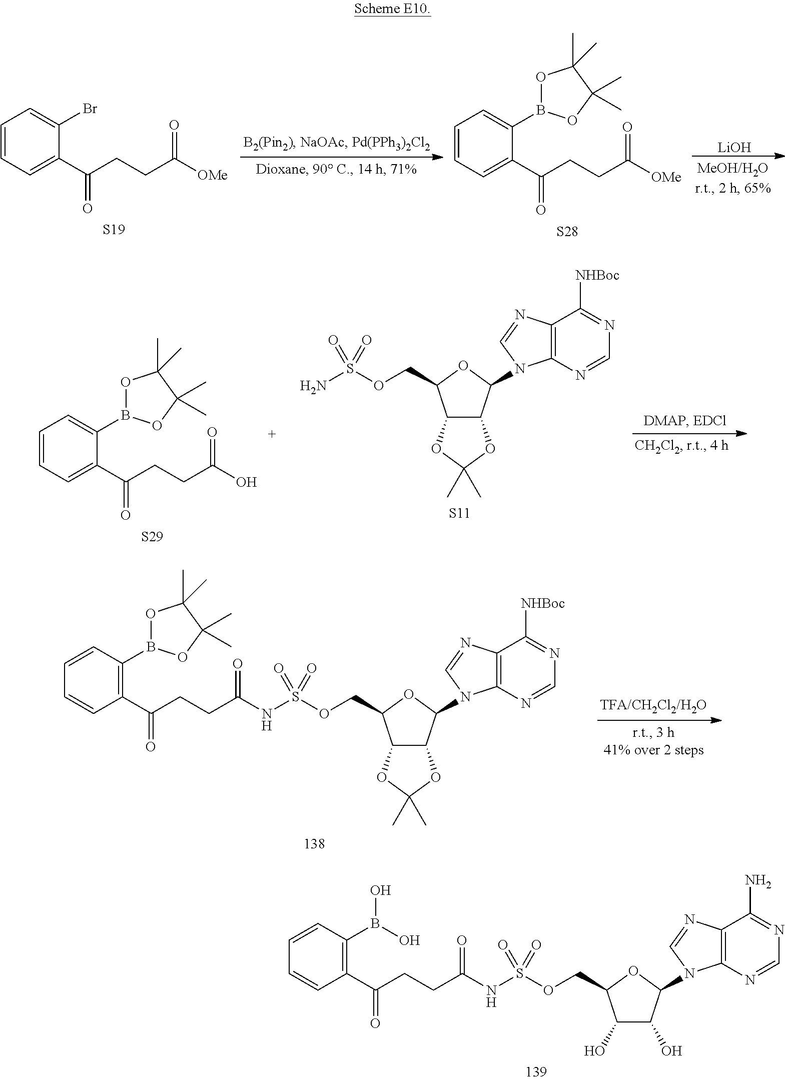

### Methyl 4-oxo-4-(2-(4,4,5,5-tetramethyl-1,3,2-dioxaborolan-2-yl)phenyl)butanoate (S28)

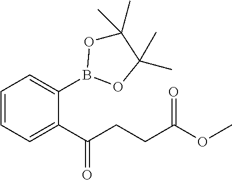 [SMILES: COC(=O)CCC(=O)c1ccccc1B1OC(C)(C)C(C)(C)O1]

Aryl bromide S19 (290 mg, 1.0697 mmol, 1 equiv.), B₂(Pin)₂ (340 mg, 1.3371 mmol, 1.25 equiv.), sodium acetate (395 mg, 4.8137 mmol, 4.5 equiv.), and Pd(PPh₃)₂Cl₂ (75 mg, 0.107 mmol, 0.1 equiv.) was suspended in degassed dioxane (10 mL) and stirred at 90° C. for 14 hours. Concentration by rotary evaporation and purification by silica flash chromatography (10%→20% EtOAc in hexanes) yielded the product (S28) as a clear semisolid (240 mg, 71%). IR (NaCl, Film): 2977.38, 1738.60, 1678.02, 1598.40, 1565.15, 1487.96, 1437.74, 1373.03, 1341.46, 1300.03, 1265.94, 1217.34, 1146.87, 1125.53, 1082.60, 1034.91, 961.65, 857.95, 754.73, 653.00. ¹H-NMR (600 MHz): δ 7.85 (d, J=7.8, 1H), 7.54-7.53 (m, 2H), 7.44 (ddd, J=7.8, 5.3, 3.4, 1H), 3.70 (s, 3H), 3.33 (t, J=7.0, 2H), 2.78 (t, J=7.0, 2H), 1.42 (s, 12H). ¹³C-NMR (150 MHz): δ 199.80, 173.23, 140.43, 132.46, 132.34, 129.01, 127.56, 83.81, 51.84, 33.024, 28.14, 24.88. HRMS (ESI) m/z calcd for C₁₇H₂₄BO₅ ([M+H]⁺) 319.1717; found 319.1729.

### 4-Oxo-4-(2-(4,4,5,5-tetramethyl-1,3,2-dioxaborolan-2-yl)phenyl)butanoic acid (S29)

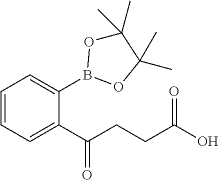 [SMILES: CC1(C)OB(c2ccccc2C(=O)CCC(=O)O)OC1(C)C]

Methyl ester S28 (80 mg, 0.2514 mmol, 1 equiv.) and LiOH (12 mg, 0.5028 mmol, 2.0 equiv.) were suspended in MeOH/H₂O (2 mL, 10:1) and stirred for 2 hours at room temperature. Concentration by rotary evaporation and purification by silica flash chromatography (10%→20% EtOAc in hexanes with 1% AcOH) yielded the product (S29) as a white oily solid (50 mg, 65%). IR (NaCl, Film): 2982.30, 1713.75, 1679.37, 1603.62, 1569.57, 1490.42, 1377.71, 1345.31, 1300.00, 1199.90, 1150.94, 1090.14, 1040.23, 964.71, 683.19, 757.54, 674.29, 654.16. ¹H-NMR (600 MHz): δ 7.83 (d, J=7.8 Hz, 1H), 7.53 (d, J=4.1 Hz, 2H), 7.44 (dt, J=8.3, 4.1 Hz, 1H), 3.32 (t, J=6.9 Hz, 2H), 2.82 (t, J=6.9 Hz, 2H), 1.42 (s, 11H). ¹³C-NMR (150 MHz): δ 199.70, 177.95, 140.37, 132.51, 132.396, 129.05, 128.25, 127.55, 83.88, 32.80, 24.86. HRMS (ESI) m/z calcd for C₁₆H₂₁BO₅Na ([M+Na]⁺) 327.1380; found 327.1359.

### Compound 138: 6-N-t-Butoxycarbonyl-2′,3′-O-isopropylidene-5′-O—(N-[4-oxo-4-(2-(4,4,5,5-tetramethyl-1,3,2-dioxaborolan-2-yl)phenyl)butanoyl]sulfamoyl)adenosine

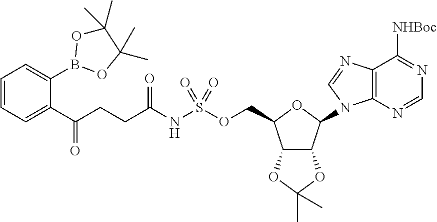 [SMILES: CC(C)(C)OC(=O)Nc1ncnc2c1ncn2[C@@H]1O[C@H](COS(=O)(=O)NC(=O)CCC(=O)c2ccccc2B2OC(C)(C)C(C)(C)O2)[C@H]2OC(C)(C)O[C@H]21]

Keto acid S29 (100 mg, 0.3288 mmol, 1 equiv.), protected 5′-O-sulfamoyladenosine S11 (240 mg, 0.4932 mmol, 1.5 equiv.) and DMAP (40 mg, 0.3288 mmol, 1 equiv.) were dissolved in CH₂Cl₂ (25 mL) and EDCI (251 mg, 1.315 mmol, 4 equiv.) added. The reaction was stirred at room temperature for 4 hours then quenched with water (30 mL) and extracted with dichloromethane (5×25 mL). The combined organic extracts were dried (Na₂SO₄), filtered through a pad of celite, and concentrated by rotary evaporation to afford the crude protected analogue 138 (322 mg, 127% crude yield), which was used without further purification.

### Compound 139: 5′-O—(N-[4-(2-Boronophenyl)-4-oxobutanoyl]sulfamoyl)adenosine

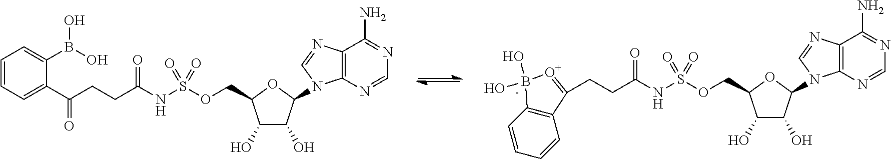

Crude protected boronic acid analogue 138 was dissolved in CH₂Cl₂ (2 mL) and water (0.2 mL) at 0° C. and TFA (2 mL) added. The reaction was stirred for 1 hours at 0° C., then warmed to room temperature and stirred for 3 hours. Concentration by rotary evaporation, purification by preparative HPLC (5%→95% MeCN in H₂O with 0.01% TFA), and lyophilization yielded the product (139) as a fluffy white solid (74 mg, 41%). IR (NaCl, Film): 3375.65, 2509.60, 1678.22, 1376.79, 1202.88, 1140.13, 978.57, 636.62. ¹H-NMR (600 MHz, MeOD/d-TFA): δ 8.49 (s, 1H), 8.35 (s, 1H), 8.06 (d, J=7.6 Hz, 1H), 7.62 (t, J=7.3 Hz, 1H), 7.51 (td, J=7.7, 1.1 Hz, 1H), 7.39 (d, J=7.1 Hz, 1H), 6.10 (d, J=4.9 Hz, 1H), 4.65 (q, J=5.4 Hz, 1H), 4.63-4.57 (m, 2H), 4.41 (t, J=4.8 Hz, 1H), 4.35 (dt, J=7.3, 3.6 Hz, 1H), 3.42-3.36 (m, 2H), 2.75 (t, J=6.2 Hz, 2H), 1.38 (s, 1H), 1.20 (s, 1H). ¹³C-NMR (150 MHz, MeOD/d-TFA): δ 203.749, 181.273, 155.558, 153.028, 149.146, 139.987, 138.573, 133.461, 131.048, 128.881, 128.838, 118.600, 87.245, 82.715, 75.504, 74.155, 70.484, 68.321, 32.796, 24.322. HRMS (ESI) m/z calcd for C₂₀H₂₄BN₆O₁₀S ([M+H]⁺) 551.1368; found 551.1387.

### Synthesis of an α-Benzyl Trifluoroethanol Analog (Compound 142)

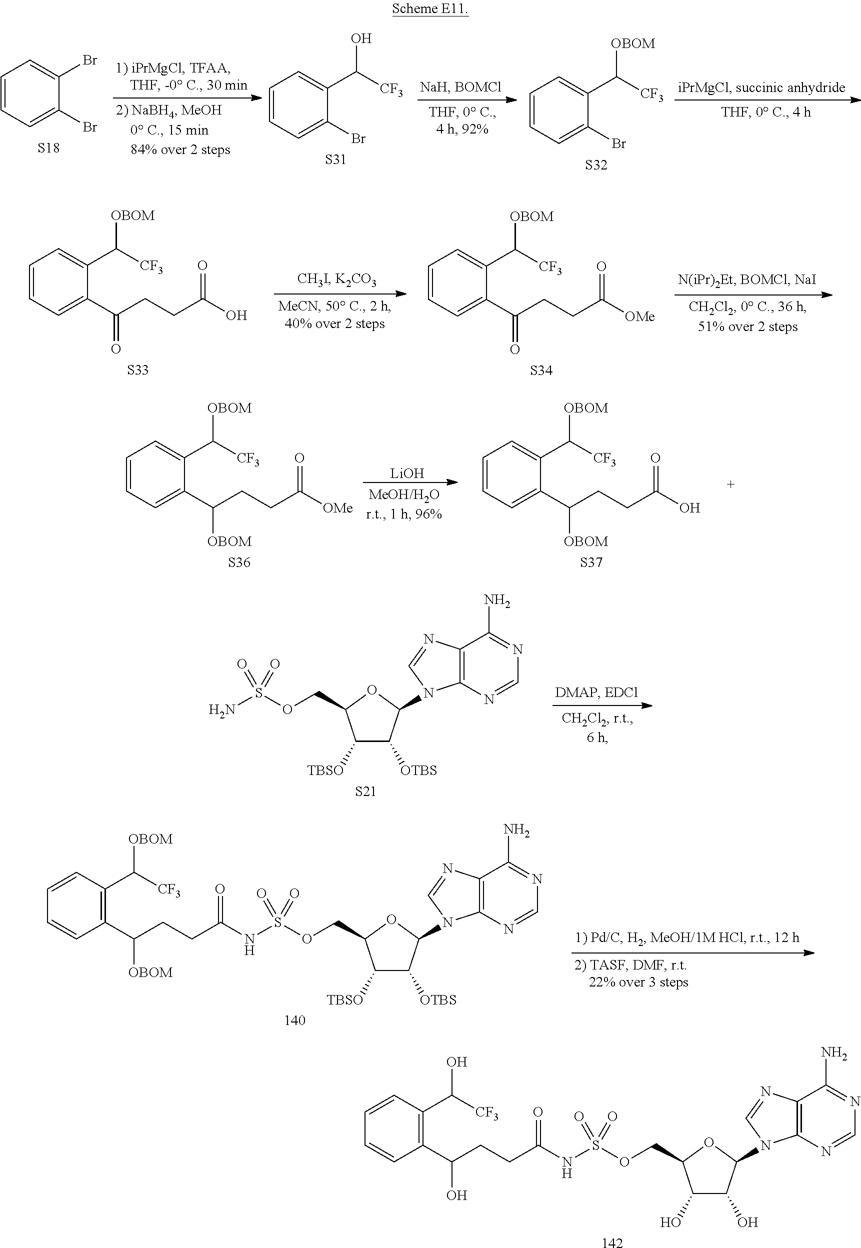

### 1-(2-Bromophenyl)-2,2,2-trifluoroethanol (S31)

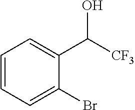 [SMILES: OC(c1ccccc1Br)C(F)(F)F]

Isopropylmagnesium bromide (40.75 mL, 52.98 mmol, 1.25 equiv., 1.3 M in THF) was cooled to 0° C., 1,2-dibromobenzene (10 g, 42.39 mmol, 1 equiv.) was added drop wise and allowed to stir for 1.5 hours. The solution was added drop wise via cannula over 30 minutes to a stirring solution of trifluoroacetic anhydride (32.63 g, 211.9 mmol, 5.0 equiv.) in THF (100 mL) at 0° C. The reaction was stirred for 30 minutes, quenched with saturated ammonium chloride (100 mL), diluted with water (200 mL) and extracted with Et₂O (3×200 mL). The combined organic extracts were dried (Na₂SO₄), filtered, and concentrated by rotary evaporation. The crude product was dissolved in MeOH (75 mL) and cooled to 0° C. NaBH₄ (1.9 g, 50.38 mmol, 1.25 equiv.) was added in three portions over 15 minutes. The reaction was stirred for 15 minutes before being quenched with 1 M HCl (250 mL) and extracted with CH₂Cl₂ (4×200 mL). The combined organic extracts were dried (Na₂SO₄), filtered, and concentrated by rotary evaporation. Purification by silica flash chromatography (0%→15% EtOAc in hexanes) yielded the product (S31) as a clear, colorless oil (8.6 g, 84% over two steps). IR (NaCl, Film): 3376.55, 1441.21, 1265.21, 1172.52, 1124.98, 1077.31, 1026.33, 874.25, 830.40, 757.09, 730.19, 701.26, 673.55, 623.71. ¹H-NMR (500 MHz; CDCl3): δ 7.68 (d, J=7.8 Hz, 1H), 7.60 (dd, J=8.0, 0.9 Hz, 1H), 7.40 (td, J=7.6, 0.6 Hz, 1H), 7.28-7.25 (m, 1H), 5.65-5.61 (m, 1H), 2.66 (d, J=4.1 Hz, 1H). ¹³C-NMR (126 MHz; CDCl3): δ 133.7, 133.0, 131.0, 129.3, 127.9, 124.3, 123.9, 77.3, 77.0, 76.8, 71.3. ¹⁹F-NMR (471 MHz; CDCl3): δ −77.6. HRMS (ESI) m/z calcd for C₂₁H₂₃N₆O₁₀S ([M−H]⁻) 551.1196; found 551.1204.

### 1-(1-((Benzyloxy)methoxy)-2,2,2-trifluoroethyl)-2-bromobenzene (S32)

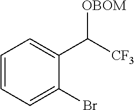

NaH (70 mg, 2.940 mmol, 1.5 equiv.) was suspended in THF (3 mL), cooled to 0° C., and trifluoroethanol analogue S31 (500 mg, 1.960 mmol, 1 equiv.) in THF (2 mL) was added drop wise. The reaction was stirred for 15 minutes, then BOMCl (613 mg, 3.920 mmol, 2.0 equiv.) in THF (2 mL) was added drop wise. The reaction was stirred for 4 hours, then quenched with saturated ammonium chloride (50 mL), and extracted with CH₂Cl₂ (4×50 mL). The combined organic extracts were dried (Na₂SO₄), filtered, and concentrated by rotary evaporation. Purification by silica flash chromatography (0%→15% EtOAc in hexanes) yielded the product (S32) as a clear, colorless oil (680 mg, 92%). IR (NaCl, Film): 2956.24, 2897.54, 1497.11, 1472.16, 1441.47, 1371.78, 1271.54, 1167.31, 1133.20, 1041.40, 979.59, 906.46, 845.74, 733.91, 698.58, 676.96, 625.81. ¹H-NMR (500 MHz; CDCl3): δ 7.65 (d, J=7.8 Hz, 1H), 7.59 (d, J=8.1 Hz, 1H), 7.38 (t, J=7.6 Hz, 1H), 7.30 (td, J=11.7, 5.8 Hz, 3H), 7.26-7.23 (m, 3H), 5.69 (q, J=6.5 Hz, 1H), 4.87 (d, J=6.9 Hz, 1H), 4.67 (dd, J=16.2, 9.3 Hz, 2H), 4.48 (d, J=11.6 Hz, 1H). ¹³C-NMR (126 MHz; CDCl₃): δ 136.9, 133.0, 132.7, 131.0, 130.0, 128.5, 128.08, 127.97, 127.82, 124.8, 124.1, 93.1, 73.9, 70.1. ¹⁹F-NMR (471 MHz; CDCl₃): δ −75.909. HRMS (ESI) m/z calcd for C₁₆H₁₄BrF₃O₂Na ([M+Na]⁺) 397.0027; found 397.0020.

### 4-Oxo-4-(2-(1-((benzyloxy)methoxy)-2,2,2-trifluoroethyl)phenyl)butanoic acid (S33)

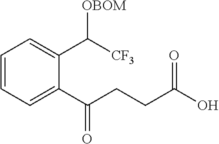

Isopropylmagnesium chloride (4.1 mL, 5.33 mmol, 2.0 equiv., 1.3 M in THF) was cooled to 0° C. and arylbromide S32 (1 g, 2.665 mmol, 1 equiv.) in THF (2.5 mL) was added drop wise. The reaction was stirred at 0° C. for 1 hour, then added drop wise via cannula to a stirring suspension of succinic anhydride (800 mg, 7.995 mmol, 3.0 equiv.) in THF (10 mL) at 0° C. The reaction was stirred for 6 hours while returning to room temperature, then quenched with 1 M HCl and extracted with EtOAc (4×100 mL). The combined organic extracts were dried (Na₂SO₄), filtered, and concentrated by rotary evaporation afford the crude acid S33 (1.4 g, 141% crude yield), which was used without further purification.

### Methyl 4-oxo-4-(2-(1-((benzyloxy)methoxy)-2,2,2-trifluoroethyl)phenyl)butanoate (S34)

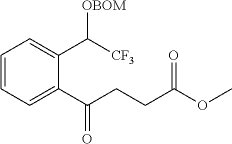

Keto acid S33 from previous step and K₂CO₃ (1.471 g, 10.65 mmol, 4 equiv.) were suspended in 25 mL MeCN before CH₃I (1.511 g, 10.65 mmol, 4 equiv.) added. The reaction was heated to 50° C. for 2 hours, then cooled to room temperature before being diluted with water (100 mL) and extracted with CH₂Cl₂ (4×100 mL). The combined organic extracts were dried (Na₂SO₄), filtered, and concentrated by rotary evaporation. Purification by silica flash chromatography (10%→30% EtOAc in hexanes) yielded the product (S34) as a clear, colorless oil (440 mg, 40%). IR (NaCl, Film): 2955.59, 2899.17, 1734.12, 1688.88, 1578.84, 1437.79, 1358.80, 1268.82, 1217.74, 1164.13, 1124.64, 1040.00, 977.93, 845.34, 735.31, 986.81, 628.11. ¹H-NMR (500 MHz; CDCl₃): δ 7.85 (d, J=7.8 Hz, 1H), 7.79 (d, J=7.8 Hz, 1H), 7.60-7.56 (m, 1H), 7.49 (td, J=7.6, 1.2 Hz, 1H), 7.31-7.24 (m, 3H), 7.20 (d, J=6.8 Hz, 2H), 6.16 (q, J=6.7 Hz, 1H), 4.80 (dd, J=62.2, 6.8 Hz, 2H), 4.54 (dd, J=86.9, 11.6 Hz, 2H), 3.68 (s, 3H), 3.25 (ddd, J=18.4, 7.3, 6.1 Hz, 1H), 3.09 (dt, J=18.4, 6.3 Hz, 1H), 2.77-2.65 (m, 2H). ¹³C-NMR (126 MHz; CDCl₃): δ 201.9, 173.1, 138.3, 137.1, 132.6, 131.9, 129.4, 129.1, 128.51, 128.38, 128.02, 127.83, 124.2, 93.6, 70.6, 69.9, 51.9, 36.3, 28.1. ¹⁹F-NMR (471 MHz; CDCl₃): δ −75.685. HRMS (ESI) m/z calcd for C₂₁H₂₁F₃O₅Na ([M+Na]⁺) 433.1239; found 433.1238.

### Methyl 4-(2-(1-((benzyloxy)methoxy)-2,2,2-trifluoroethyl)phenyl)-4-hydroxybutanoate (S35)

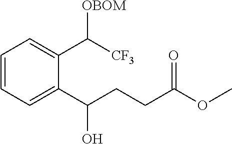

Aryl ketone S34 (158 mg, 0.385 mmol, 1 equiv.) was dissolved in MeOH (1 mL) and cooled to 0° C. and NaBH₄ (18 mg, 0.481 mmol, 1.25 equiv.) was added. The reaction was stirred for 1 hour, then acetone (0.5 mL) was added. The reaction was stirred for 10 minutes at 0° C., then phosphate buffer (10 mL, 0.5 M, pH 7.0) was added. The reaction was stirred for 10 minutes at 0° C., then extracted with CH₂Cl₂ (4×10 mL). The combined organic extracts were dried (Na₂SO₄), filtered, and reduced to 5 mL in volume by rotary evaporation at 0° C. The crude benzyl alcohol S35 solution was used immediately in the next step.

### Methyl 4-((benzyloxy)methoxy)-4-(2-(1-((benzyloxy)methoxy)-2,2,2-trifluoroethyl)phenyl)butanoate (S36)

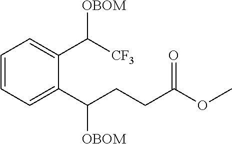

Crude benzyl alcohol S35 from previous step in 5 mL CH₂Cl₂ was cooled to 0° C., then NaI (23 mg, 0.1542 mmol, 0.1 equiv.) and BOMCl (241 mg, 1.542 mmol, 4.0 equiv.) were added quickly, followed by diisopropylethylamine (199 mg, 1.542 mmol, 4.0 equiv.) added drop wise. The reaction stirred for 36 hours at 0° C., then diluted with saturated sodium bicarbonate (10 mL) and extracted with CH₂Cl₂ (4×10 mL). The combined organic extracts were dried (Na₂SO₄), filtered, and concentrated by rotary evaporation. Purification by silica flash chromatography (0%→10% EtOAc in CH₂Cl₂) yielded the product (S36) as a clear, colorless oil (125 mg, 62% over 2 steps). IR (NaCl, Film): 3032.77, 2952.16, 1735.12, 1497.45, 1454.43, 1380.74, 1267.94, 1236.86, 1161.73, 1131.64, 1109.24, 1026.59, 979.43, 907.35, 844.31, 765.24, 736.34, 697.93, 624.95. ¹H-NMR (600 MHz; CDCl₃): δ 7.66 (t, J=8.7 Hz, 1H), 7.50 (ddd, J=16.6, 7.7, 1.3 Hz, 1H), 7.43-7.38 (m, 1H), 7.37-7.29 (m, 3H), 7.26 (td, J=4.9, 2.8 Hz, 5H), 7.25-7.20 (m, 2H), 7.13 (dd, J=7.3, 1.9 Hz, 1H), 5.62 (dq, J=28.0, 6.7 Hz, 1H), 5.03 (ddd, J=67.3, 9.9, 3.4 Hz, 1H), 4.83 (dd, J=64.6, 7.0 Hz, 1H), 4.74-4.59 (m, 4H), 4.57-4.50 (m, 1H), 4.48-4.42 (m, 2H), 2.56-2.37 (m, 2H), 2.16-2.06 (m, 1H), 2.01-1.80 (m, 1H). ¹³C-NMR (151 MHz; CDCl₃): δ 173.6, 141.9, 141.5, 137.9, 137.27, 137.10, 131.2, 130.5, 129.70, 129.66, 128.7, 128.38, 128.36, 128.09, 128.03, 127.85, 127.82, 127.80, 127.66, 127.63, 127.52, 126.5, 124.44, 124.37, 93.17, 93.02, 92.81, 92.69, 73.7, 73.3, 71.3, 71.0, 69.87, 69.83, 51.58, 51.53, 32.8, 32.5, 30.8, 30.1 ¹⁹F-NMR (471 MHz; CDCl₃): δ −74.964, −75.855. HRMS (ESI) m/z calcd for C₂₉H₃₁F₃O₆Na ([M+Na]⁺) 555.1970; found 555.1984.

### 4-((Benzyloxy)methoxy)-4-(2-(1-((benzyloxy)methoxy)-2,2,2-trifluoroethyl)phenyl)butanoic acid (S37)

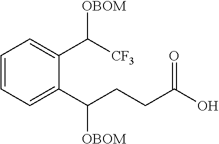

Methyl ester S36 (175 mg, 0.329 mmol, 1 equiv.) and LiOH (31 mg, 1.314 mmol, 4.0 equiv.) were suspended in MeOH:H₂O (4 mL, 9:1) and stirred at 50° C. for 1 hour. The reaction was returned to room temperature, diluted with 1 M KHSO₄ (15 mL) and extracted with EtOAc (4×15 mL). The combined organic extracts were dried (Na₂SO₄), filtered, and concentrated by rotary evaporation. Purification by silica flash chromatography (25%→50% EtOAc in hexanes) yielded the product (S37) as a white solid (164 mg, 96%). IR (NaCl, Film): 3035.21, 2953.60, 2892.31, 1709.31, 1498.78, 1455.93, 1383.82, 1270.22, 1165.18, 1133.87, 1039.66, 981.56, 910.35, 846.24, 767.15, 737.80, 699.92, 650.66. ¹H-NMR (600 MHz; CDCl₃): δ 7.65 (dd, J=14.5, 7.6 Hz, 1H), 7.52-7.47 (m, 1H), 7.43-7.38 (m, 1H), 7.37-7.33 (m, 1H), 7.31-7.21 (m, 8H), 7.19 (dd, J=8.5, 6.7 Hz, 1H), 7.11 (dd, J=7.3, 1.8 Hz, 1H), 5.60 (dquintet, J=15.5, 5.4 Hz, 1H), 5.03 (ddd, J=67.1, 9.8, 3.3 Hz, 1H), 4.80 (dd, J=41.6, 7.0 Hz, 1H), 4.68-4.66 (m, 2H), 4.64-4.60 (m, 1H), 4.59-4.57 (m, 1H), 4.54-4.52 (m, 1H), 4.50-4.42 (m, 2H), 2.56-2.40 (m, 2H), 2.17-2.03 (m, 1H), 1.99-1.80 (m, 1H). ¹³C-NMR (151 MHz; CDCl₃): δ 179.5, 141.7, 141.3, 137.8, 137.2, 137.0, 131.1, 130.4, 129.7, 128.8, 128.4, 128.17, 128.09, 128.03, 127.90, 127.85, 127.81, 127.69, 127.66, 127.52, 126.6, 124.40, 124.32, 92.99, 92.91, 92.83, 92.70, 73.7, 73.4, 71.12, 71.02, 69.94, 69.79, 32.3, 32.1, 30.8, 30.0. ¹⁹F-NMR (471 MHz; CDCl₃): δ −75.044, −75.900. HRMS (ESI) m/z calcd for C₂₈H₂₉F₃O₆Na ([M+Na]⁺) 541.1814; found 541.1837.

### Compound 140: 2′,3′-O-TBS-5′-O—(N-[4-((benzyloxy)methoxy)-4-(2-(1-(benzyloxy)methoxy)-2,2,2-trifluoroethyl)phenyl)butanoyl]sulfamoyl)adenosine

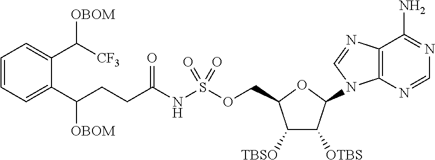

Keto acid S37 (164 mg, 0.316 mmol, 1 equiv.), protected sulfamoyladenosine S21 (227 mg, 0.395 mmol, 1.25 equiv.) and DMAP (38 mg, 0.316 mmol, 1 equiv.) were suspended in CH₂Cl₂ (5 mL) and EDCI (241 mg, 1.264 mmol, 4 equiv.) added. The reaction was stirred for 6 hours, then water (20 mL) added, and extracted with ethyl acetate (5×20 mL). The combined organic extracts were dried (Na₂SO₄), filtered, and concentrated by rotary evaporation afford the crude acid 140 (428 mg, 126% crude yield), which was used without further purification.

### Compound 142: 5′-O—(N-[4-Hydroxy-4-(2-(2,2,2-trifluoro-1-hydroxyethyl)phenyl) butanoyl]sulfamoyl)adenosine

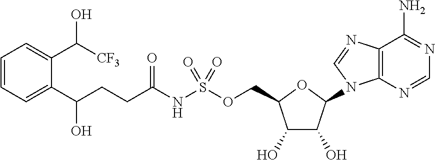 [SMILES: Nc1ncnc2c1ncn2[C@@H]1O[C@H](COS(=O)(=O)NC(=O)CCC(O)c2ccccc2C(O)C(F)(F)F)[C@@H](O)[C@H]1O]

Crude product 140 from the previous step and 10% Pd/C (33 mg, 0.032 mmol, 0.1 equiv.) was dissolved in MeOH (30 mL) and 1 M HCl (0.3 mL) added. The reaction was stirred under H₂ (balloon) for 12 hours at room temperature, then diluted with CH₂Cl₂ (70 mL), filtered through a pad of celite, and concentrated by rotary evaporation. The residue was suspended in DMF (5 mL) and TASF (260 mg, 0.944 mmol, 3.0 equiv.) in DMF (1.5 mL) added. The reaction was stirred for 12 hours, then concentration by rotary evaporation, purification by preparative HPLC (5%→95% MeCN in H₂O with 0.01% TFA), and lyophilization yielded the product (142) as a fluffy white solid (42 mg, 22% over three steps). IR (NaCl, Film): 3339, 2504, 1677, 1474, 1429, 1381, 1263, 1199, 1131, 1051, 978, 889, 834, 801, 768, 724, 706, 641, 613. ¹H-NMR (500 MHz; MeOD): δ 8.50 (d, J=4.1 Hz, 1H), 8.36 (s, 1H), 7.62-7.60 (m, 1H), 7.56-7.52 (m, 1H), 7.39-7.35 (m, 1H), 7.32-7.29 (m, 1H), 6.11 (dd, J=4.6, 2.1 Hz, 1H), 5.60-5.52 (m, 1H), 5.01 (dd, J=9.2, 3.4 Hz, 1H), 4.64 (dt, J=10.2, 5.0 Hz, 1H), 4.60-4.51 (m, 2H), 4.42 (td, J=5.0, 1.0 Hz, 1H), 4.32 (q, J=4.0 Hz, 1H), 2.62-2.54 (m, 1H), 2.50-2.44 (m, 1H), 2.01-1.96 (m, 1H), 1.83-1.74 (m, 1H). ¹³C-NMR (151 MHz; CDCl₃): δ 173.57, 162.11, 147.10, 144.96, 143.42, 133.44, 130.19, 128.97, 128.96, 128.33, 126.71, 126.66, 126.54, 90.46, 83.58, 75.84, 72.32, 71.60, 69.80, 68.24, 34.57, 33.02. HRMS (ESI) m/z calcd for C₂₂H₂₆F₃N₆O₉S ([M+H]⁺) 607.1434; found 607.1423.

### Enzyme Inhibition

The IC₅₀ values for the inhibition of E. coli MenE (ecMenE) by compounds 102-109 are reported in Table E1. Enzyme inhibition studies were performed using the MenE-MenB coupled reaction in which the MenE reaction is rate limiting (also described in Reference 1 and 2 which are incorporated herein by reference). Reaction mixtures contained OSB (60 μM), ATP (240 μM), CoA (240 μM), MtMenB (2.5 μM) and varying inhibitor concentrations (5-250 μM). Reactions were initiated by the addition of MenE (25 nM) and the production of DHNA-CoA was monitored at 392 nm (ε₃₉₂ 4000 M⁻¹ cm). Measurements were performed in triplicate for each compound. The m-succinylbenzoate analog (102), as well as the nitro (103) and oxazole (104) keto acid analogues, and the lactone (107) and lactam (108) lactol analogues, showed no inhibition of ecMenE up to a concentration of 100 μM. In contrast, tetrazole analogue 105 inhibited ecMenE with an IC₅₀ value of 2.2±0.4 μM while the squaric acid analogue 106 showed more potent inhibition with an IC₅₀ value of 0.17±0.05 μM. Interestingly, the difluoroindandiol analogue 109 also showed inhibition with an IC₅₀ value of 1.5±0.1 μM, indicating that both open and closed analogues are able to inhibit MenE. These data indicate a preference for a negative charge in the inhibitor close to the position in the enzyme likely occupied by the OSB-CoA carboxylate group.

TABLE E1
IC₅₀ of exemplary compound for inhibition of E. coli MenE.

| Compound | Average IC₅₀ for ecMenE (μM) | pKₐ |
|---|---|---|
| OSB-AMS | 0.025 ± 0.005 | 4 |
| 102 | >100 | 4 |
| 103 | >100 | — |
| 104 | >100 | — |
| 105 | 2.2 ± 0.4 | 3.4 |
| 106 | 0.17 ± 0.05 | 1.3 |
| 107 | >100 | — |
| 108 | >100 | — |
| 109 | 1.5 ± 1 | 11.5 |
| 139 | >100 | >14 |
| 144 | >100 | 11.5 |

### Antibacterial Activity

To assess the antibacterial potency of the OSB-AMP analogues, we determined their ability to inhibit growth of B. subtilis, MRSA, M. tuberculosis and E. coli. Minimum inhibitory concentrations (MICs) were determined using the Alamar blue assay (ATCC 6051). E. coli, B. subtilis (ATCC 6051), S. aureus (ATCC BAA-1762), and M. tuberculosis (H37Rv) were grown in LB, Miller Hinton, synthetic broth, or 7H10 media overnight at 37° C. in an orbital shaker. A calculated final inoculum of 1-2×10⁶ cells per well was transferred to fresh media and cultured to mid-log phase (OD₆₀₀˜0.5). 200 μL of cell solution is transferred per well and treated with 1 μL inhibitor at final concentrations ranging from 500-0.5 μg/mL. Minimum inhibitory concentration is the well with ˜90% cell death, as determined by the Alamar blue assay. Averages of triplicate MIC measurements are listed in Table E2.

E. coli was included as a control since this Gram-negative organism does not produce menaquinone under aerobic conditions, and as expected, no compounds inhibited the growth of E. coli up to a concentration of 500 μM. OSB-AMS had MIC values of 62.5, 31.25, and 125 μg/mL against B. subtilis, MRSA, and M. tuberculosis, respectively. Compound 106 did not show cellular activity against any bacteria tested. In contrast however, 109 had MIC values of 15.6 and 31.25 μg/mL against MRSA and B. subtilis, respectively, which may indicate increased rates of passive diffusion due to loss of one negative charge relative to OSB-AMS. Compound 109 also showed anti-tubercular activity at 15.6 μg/mL. The antibacterial activity of the compounds were assessed in the presence of menquinone-4 (MK4) (10 μM). All bacteria that were sensitive to the MenE inhibitors were rescued by supplementation with MK4, supporting the target specificity of the inhibitors.

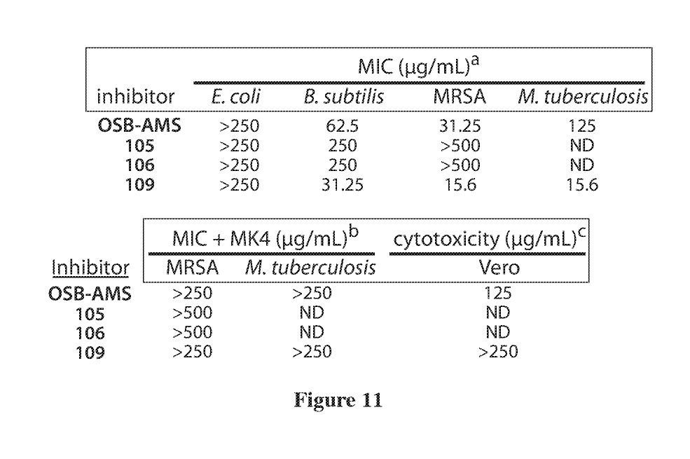

Growth rescue studies were performed by supplementing minimal medium (synthetic broth) with 10 μM menaquinone-4 (MK4) and following the same procedure (See FIG. 11)

TABLE E2
Antibacterial activity (MIC) of exemplary compounds.

| | E. coli | B. subtilis | MRSA | M. tuberculosis |
|---|---|---|---|---|
| | MIC | MIC | MIC | MIC |
| Compound | (μg/mL) | (μg/mL) | (μg/mL) | (μg/mL) |
| OSB-AMS | >500 | 62.5 | 31.25 | 125 |
| 102 | — | — | — | — |
| 103 | — | — | — | — |
| 104 | — | — | — | — |
| 105 | — | — | — | — |
| 106 | >500 | 250 | >500 | >500 |
| 107 | — | — | — | — |
| 108 | — | — | — | — |
| 109 | >500 | 31.25 | 15.6 | 15.6 |

### Cytotoxicity

To obtain insight into the potential cytotoxicity of our MenE inhibitors, the in vitro cytotoxicity of the compounds was evaluated using Vero monkey kidney cells. Briefly, 105 cells/well were aliquoted into 96-well culture plates in serum rich medium. The cells were incubated for 24-36 hours at 37° C. in 5% CO₂. The medium was then aspirated and replaced with 200 μL of serum-free fresh medium. Cells were incubated for 5 h at 37° C. in 5% C02, after which compounds dissolved in serum-free cell medium were added, giving a concentration range of 0.97-250 μg/mL. The cells were incubated for 24-36 hours at 37° C. in 5% C02. Cell death was assessed by incubating 20 μL of a cell suspension from each well with 20 μL of Trypan Blue for 5 min. The ratio of viable/dead cells was determined using a hemocytometer in which stained cells were scored as dead and nonstained cells were scored as viable. The cytotoxic concentration was defined as the minimum inhibitor concentration that gave ˜90% cell death. See FIG. 11 for cytotoxicity data.

Effect of OSB-AMS on Menaquinone Levels in S. aureus.

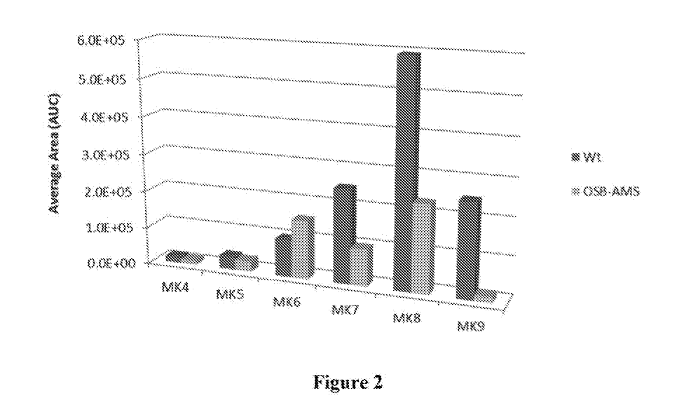

To provide direct insight into the mode of action of the MenE inhibitors, we analyzed the effect of OSB-AMS on menaquinone-levels in S. aureus by tandem MS (FIG. 2), as follows. Cultures of S. aureus ATCC BAA-1762 (5 mL in Synthetic Broth medium with 10% glucose) were incubated overnight in a 37° C. shaker in the presence or absence of OSB-AMS (15.6 μg/mL final concentration). The Blight and Dyer (1959) lipid extraction protocol was used to isolate the menaquinone-containing fraction from the cells.⁽⁵⁾ Briefly, 0.75 mL of 1:2 (v/v) CHCl₃:MeOH was added to 0.2 mL of culture. The mixture was vortexed thoroughly, and 0.25 mL of CHCl₃ was added followed by further vortexing after which 0.25 mL of H₂O was added. The mixture was then vortexed and centrifuged at 1000 rpm for 5 minutes at room temperature. The bottom phase was recovered, transferred to a glass vial and 200 μL was analyzed by APCI LC-MS/MS in positive ion mode using a Thermo TSQ Quantum Access (Thermo-Fisher) Triple Quadrupole Mass Spectrometer. Menaquinone levels were quantified using standard established for MK4 and MK9. Samples were introduced into the mass spectrometer by flow injection at 100 μL/min with 2:1 MeOH/CHCL₃ as the solvent. Multiple Reaction Monitoring (MRM) was performed at 30 eV. MK4, MK5 and MK6 were quantified using the standard curve for MK4 whereas MK7, MK8, and MK9 were quantified using MK9.

S. aureus contains a series of menaquinones that differ in the number of isoprene units that compose the side chain. Our data demonstrated that menaquinone-8 (MK8) was the major species with significant quantities of MK7 and MK9. Treatment of S. aureus with OSB-AMS resulted in a ˜3-5 fold decrease in the levels of the menaquinones, confirming that the antibacterial activity of this compound resulted from a direct effect on menaquinone biosynthesis.

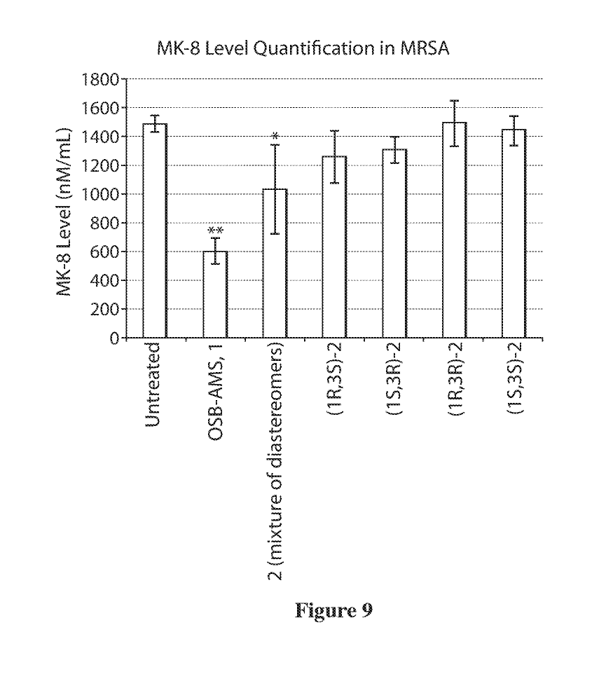

MRSA treated with OSB-AMS (1) showed a statistically significant 2.5-fold decrease in menaquinone-8, consistent with previous findings (FIG. 9). See, e.g., Matarlo et al. Biochemistry 2015, 54, 6514-6524. The mixture of four diastereomers 2 also elicited a smaller, but statistically significant, 31% decrease in menaquinone-8. However, none of the individual difluoroindanediol diastereomers caused a significant decrease in menaquinone-8. Taken together, these results suggest that even the MenE inhibitor (1R,3S)-2 may act via mechanisms other than inhibition of menaquinone biosynthesis.

### Role of a Conserved Arginine in Substrate Recognition and Enzyme Inhibition

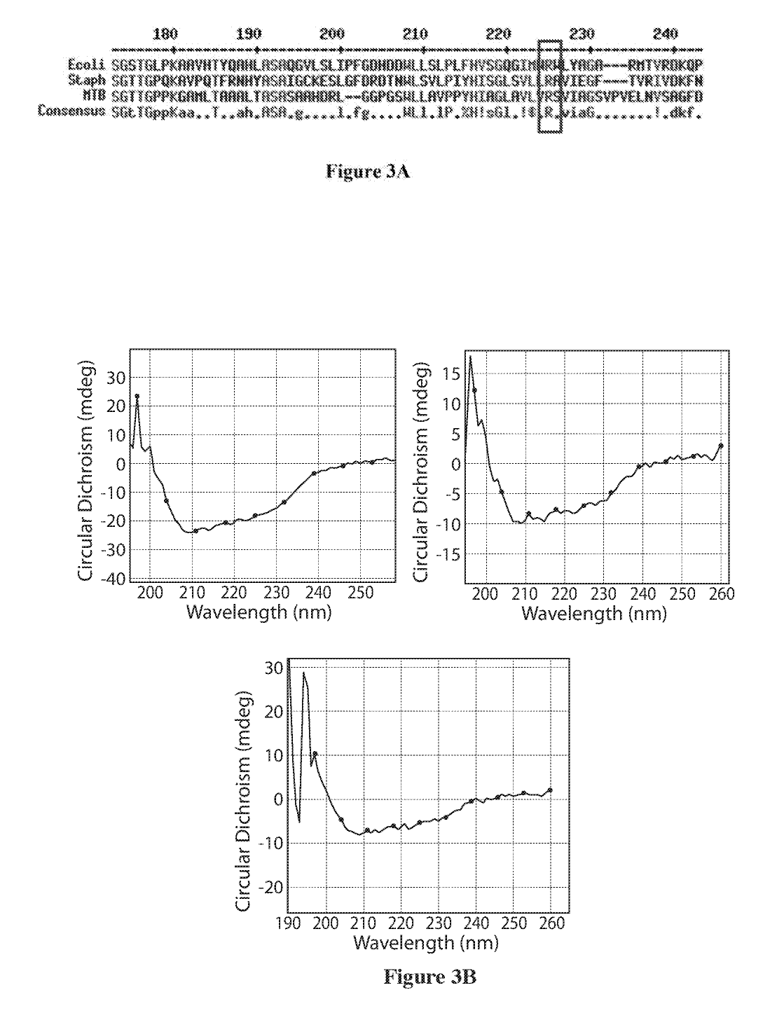

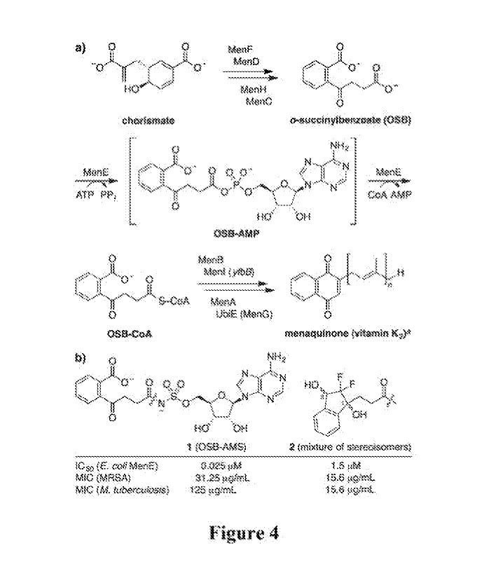

A docking model approach was used to identify a basic residue, Arg222, in the active site of saMenE within 3 Å of the OSB carboxylate⁽¹⁾. The details of the docking model for probing the interactions of ligands with S. aureus MenE are described in Reference 1 and incorporated herein by reference. Sequence alignment studies revealed that Arg222 is conserved in other MenE homologs and corresponds to Arg90 in M. tuberculosis (mtMenE) and Arg195 in E. coli (ecMenE) (FIG. 3A). The sequences of the proteins MenE from E. coli (K-12), S. aureus (RN4220), and M. tuberculosis (Erdman) were aligned using INRA Multalin⁽⁴⁾.

To explore the role of the conserved Arg and provide validation for the computational studies, we replaced Arg195 in ecMenE with Lys or Gln residues. The primers for cite directed R195K and R195Q mutagenesis of ecMenE are listed in Table E3.

TABLE E3
Primers for S. aureus MenE mutations.

| Mutation | Primers (forward, reverse) |
|---|---|
| R195K | GGAATTATGTGGAAGTGGTTATACGC (SEQ ID NO: 5) |
| | GCGTTAAACCACTTCCACATAATTCC (SEQ ID NO: 6) |
| R195Q | GGAATTATGTGGCAGTGGTTATACGC (SEQ ID NO: 7) |
| | GCGTATAACCACTGCCACATAATTC (SEQ ID NO: 8) |

Circular dichroism spectra of these mutants showed no significant alteration in the secondary structure (FIG. 3B). CD experiments were performed using a Chirascan CD spectrometer. MenE was diluted to 20 μM in pH 7.4 20 mM sodium phosphate buffer containing 150 mM sodium chloride and 1 mM magnesium chloride. Far-UV wavelength (196 nm to 260 nm) spectra were collected in a 1 mm cuvette with a 1 nm increment and averaged with 3 repetitions.

Analysis of the catalytic efficiency (k_{cat}/K_{M}) of the mutant enzymes compared to wild type MenE was performed using the MenE-MenB coupled assay described above. These studies revealed (see Table E4) that the k_{cat}/K_{M} value decreased by ˜93% for R193K MenE, while the R195Q mutant had no detectable activity. Further analysis demonstrated that the effect of the R193K mutation on activity was primarily a result of a 16-fold increase in the K_{M} value while the k_{cat} for product formation was unchanged.

TABLE E4
Catalytic Parameters and ITC data for the interaction of OSB-AMS with wild-type and Mutant ecMenE.

| | K_{M}^{OSB} | k_{cat} | k_{cat}/K_{M} | K_{d}^{OSB-}^{AMS} | ΔH | ΔG | ΔΔG |
|---|---|---|---|---|---|---|---|
| ecMenE | (μM)¹ | (min⁻¹)¹ | (μM⁻¹min⁻¹)¹ | (nM)² | (kcal/mol)² | (kcal/mol)² | (kcal/mol)² |
| wild-type | 1 ± 0.02 | 46 ± 0.1 | 46 ± 0.02 | 44 ± 11 | −2.0 ± 0.1 | −10.0 | |
| R195K | 16 ± 1.4 | 47 ± 0.3 | 3 ± 0.2 | 394 ± 36 | −2.5 ± 0.2 | −8.8 | 1.2 |
| R195Q | | Not Active | | 4500 ± 112 | −3.1 ± 0.1 | −7.3 | 2.7 |

To investigate the role of the conserved Arg in enzyme inhibition, the binding of OSB-AMS to ecMenE mutants by isothermal titration calorimetry (ITC). The direct binding of inhibitors to MenE was quantified using isothermal titration calorimetry (ITC). Measurements were made with a MicroCal VP-ITC instrument at 25° C. Inhibitor stock solutions (1 mM in NaHPO₄ buffer pH 7.4 containing 150 mM NaCl and 1 mM MgCl₂) were titrated in 4 μL increments into a 50 μM solution of MenE in pH 7.4 20 mM sodium phosphate buffer containing 150 mM sodium chloride and 1 mM magnesium chloride. The data were fit to a single binding site model with the Origin software package. Using this approach, R195K and R195Q mutations were shown to decrease the binding affinity of the inhibitor to ecMenE by ˜10 and ˜100 fold respectively (Table E4). The change in binding free energy (ΔΔG) is consistent with the removal of one (R195K) or two (R195Q) hydrogen bonds to the ligand consistent with the modeled structure in which the R195 guanadinium group makes two interactions with the OSB carboxylate, and thus also presumably with the carboxylate of OSB-AMS.

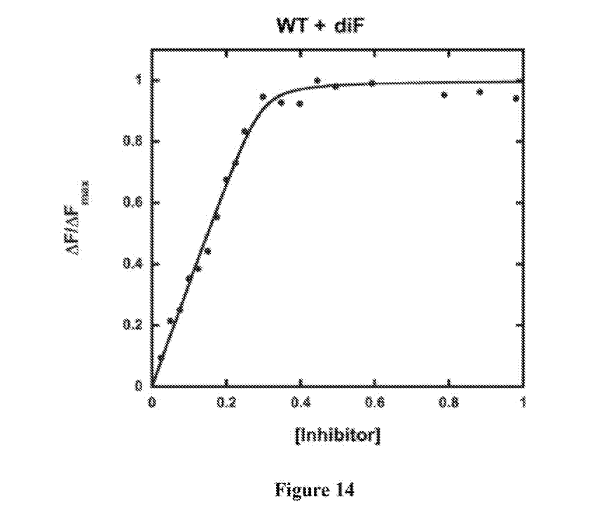

ITC experiments with difluoroindanediol 109 did not show a measurable change in enthalpy, and thus, ITC was unable to quantify the binding of this compound to the enzyme. Instead, to determine the K_{d} for 109, we used a direct binding assay in which the change in the intrinsic tryptophan fluorescence of ecMenE was monitored (see FIG. 14 for binding isotherm and data). A solution of 50 μM 11 was titrated into 300 nM ecMenE in 20 mM NaHPO₄ buffer (pH 7.4) containing 150 mM NaCl and 1 mM MgCl₂ at 25° C. The solution was stirred continuously, and fluorescence measurements were taken with a Quanta Master fluorimeter using excitation and emission wavelengths of 280 and 332 nm, respectively. Slit widths were optimized to 4 and 2 nm for excitation and emission, respectively. Data were corrected for the inner filter effect and then fit to the following equation using MATLAB:

$$\frac{Δ {F}_{i}}{Δ {F}_{max}} = \frac{[ E ] + [ I ] + {K}_{d} - \sqrt{{( [ E ] + [ I ] + {K}_{d} )}^{2} - r [ E ] [ I ]}}{2 [ E ]}$$

### Crystal Structures

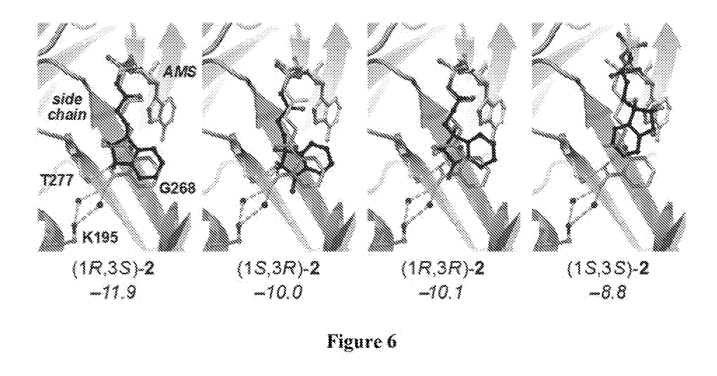

Crystal structures of Bacillus subtilis MenE (bsMenE), unliganded, and with ATP or AMP, have recently been reported (Chen et al., J. Biol. Chem. 2015, 290, 23971-23983). However, the reported crystal structures are not crystal structures of B. subtilis MenE with OSB or OSB-AMP. FIG. 6 of Chen et al. does not show the salt bridge to Arg195 that was observe in the E. coli structure with OSB-AMS. The model described in Chen et al. does not relate to what is shown structurally and biochemically with E. coli MenE at least because of Arg195. bsMenE does not include Arg195 but includes K205. Moreover, the residues L-L-G263 H-I-S-G199 described in Chen et al. are around the vicinity of the OSB moiety but do not actually interact with any OSB atoms. For example, S198, the closest residue, is at least 3.7 Å away from any OSB atoms, which is too far to form any interactions). Therefore, the findings in the present disclosure refute a key aspect of the model of OSB binding described in Chen et al. and are unexpected.

To underpin efforts to develop potent MenE inhibitors and extend the modeling studies with saMenE, the X-ray structure of MenE in complex with OSB-AMS (1) was obtained. The efforts were successful with the R195K mutant of ecMenE, resulting in a 2.4 Å resolution structure of R195K ecMenE cocrystallized with OSB-AMS (PDB entry 5C5H). The structure was determined by molecular replacement using the structures of saMenE and 4-chloroben-zoate:CoA ligase (CBAL) from Alcaligenes sp. AL3007 (PDB entries 3IPL and 1T5D, ˜29% sequence identity) as search models.

MenE is a member of the adenylate-forming enzyme superfamily in which ATP is used to activate a carboxylate for subsequent attack of a nucleophile. One of the best characterized members of this family is CBAL, which has been extensively studied by Gulick, Dunaway-Mariano, and colleagues. See, e.g., Wu, R., et al. Biochemistry (2008) 47, 8026-8039; Reger, A. S., et al. Biochemistry (2008) 47 (31), 8016-8025; Wu, R., et al. (2009) Biochemistry 48, 4115-4125. Both MenE and CBAL are comprised of a larger N-terminal domain and a smaller C-terminal domain, and structures of CBAL in complex with an adenylate inter-mediate as well as CoA thioester product analogue reveal that ligand binding causes the two domains to move relative to each other as the reaction proceeds. Domain alternation reconfigures the active site from a conformation that catalyzes acyl-adenylate formation to one that facilitates CoA binding and thioester formation. See, e.g., Branchini, B. R., et al. J. Am. Chem. Soc. (2011) 133, 11088-11091. Sundlov, J. A. et al. Biochemistry (2012) 51, 6493-6495; Bandarian, V. et al. Nat. Struct. Biol. (2002) 9, 53.

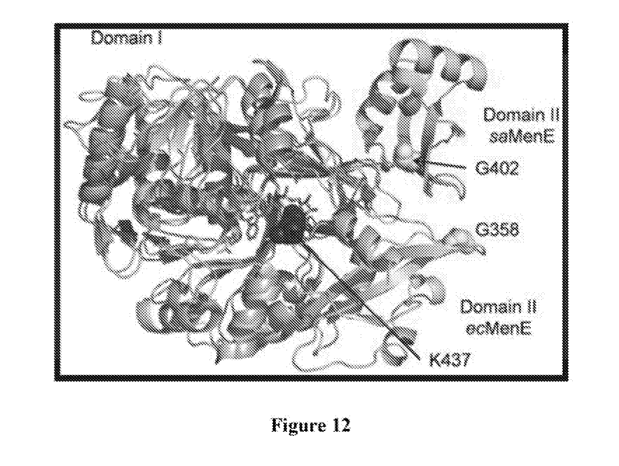

In FIG. 12, the structure of the OSB-AMS:ecMenE complex overlaid with that of apo saMenE (PDB entry 3IPL) is shown. These structures differ in the relative orientations of domains 1 and 2. However, both structures are representative of the adenylate-bound conformation observed for CBAL (PDB entry 3CW8), in which G408 in region A8 (399-GRVDDMIISG-408) is removed from the active site whereas K492 in region A10 (486-PKNALNK-492) is located in the active site. The corresponding residues in ecMenE (saMenE) are G358 (G402) and K437 (K483), and in FIG. 12, it can be seen that G358 and G402 are located away from the MenE binding site whereas K437 is close to the bound OSB-AMS in ecMenE. Note that K483 is disordered in the structure of apo saMenE.

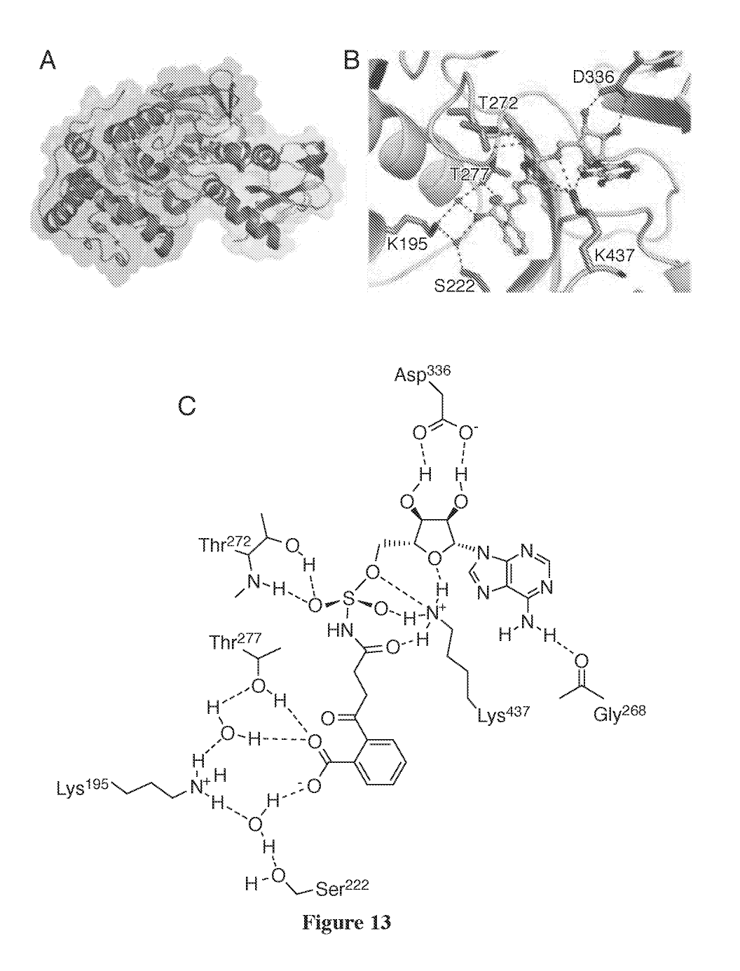

Residues that interact with OSB-AMS (1) are highlighted in FIG. 13 and include T142, H186, S188, K195 (R195), S222, T272, D336, R350, and K437, which are all conserved in E. coli, S. aureus, and M. tuberculosis MenE. Residues T142 (motif 1, A3, P-loop), T272 (motif II, A5), D336 (motif III, A7), R350 (A8, hinge), and K437 (A10) are components of the conserved sequence motifs in the adenylate-forming enzyme superfamily and are, thus, involved in the general chemical reaction that leads to acyl-adenylate formation. Residues S188, K195 (R195), S222, and T277 are clustered around the OSB portion of OSB-AMS and likely confer substrate specificity upon MenE. The electron density of the OSB-AMS ligand is well-defined and consistent with the keto acid isomer rather than the lactol isomer. In addition, the OSB carboxylate interacts with K195 via a water-mediated ionic bridge comprised of two conserved water molecules (FIG. 13). It is possible that R195 in wild-type ecMenE also participates in this water-mediated interaction, although a direct interaction with the OSB carbo-xylate cannot be ruled out. In either case, the X-ray structure is consistent with the previously reported model of OSB-AMS bound to saMenE as well as the site-directed mutagenesis studies. See, e.g., Lu, X., et al. Chem. Bio. Chem. (2012) 13, 129. In particular, the experimentally observed change in binding free energy (ΔΔG) for binding of OSB-AMS to ecMenE is consistent with the removal of one (R195K) or two (R195Q) water-mediated hydrogen bond interactions with the ligand, suggesting that the R195 guanidinium group in wild-type ecMenE makes two interactions with the OSB carboxylate moiety. These studies further support the notion that the OSB substrate binds to MenE as its open-chain keto acid isomer.

### Docking of Difluoroindanediols 2 (Compound 109)

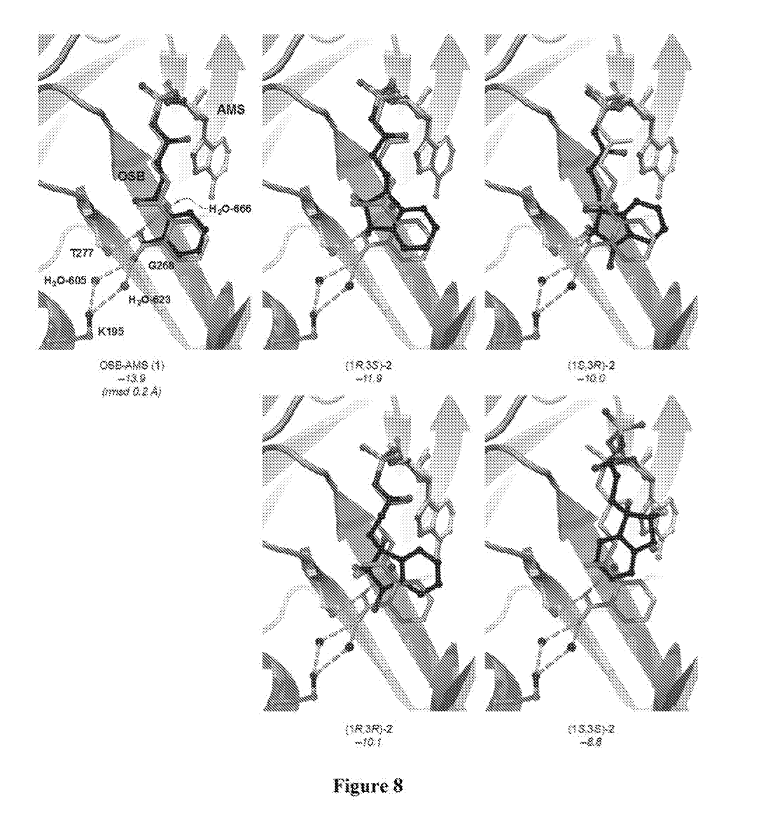

Computational docking (Glide, Schrödinger) using a recently reported cocrystal structure of E. coli MenE (R195K mutant) in complex with OSB-AMS (1) was carried ouy (See FIG. 6 and FIG. 8). See, e.g., Matarlo et al. Biochemistry 2015, 54, 6514-6524. Docking of OSB-AMS into the protein provided a ligand pose well-aligned with that observed in the cocrystal structure (rmsd 0.2 Å). In docking of the four diastereomeric difluoroindanediols 2, the adenosine region of each diasteromer bound in an orientation consistent with that of OSB-AMS, retaining key interactions and filling the adenosine binding pocket. However, in the side chain region, only the syn-difluoroindanediol (1R,3S)-2 filled the binding OSB pocket fully, overlapping well with the OSB aromatic ring of cocrystallized OSB-AMS. The secondary alcohol of the difluoroindanediol appeared poised to engage in hydrogen bonding with a conserved water H₂O-666 and the alcohol side chain of Thr-277, which both interact with the OSB carboxylate in cocrystallized OSB-AMS.

Notably, in earlier docking studies with unliganded S. aureus MenE, a Ser-302 side chain (Thr-178 in M. tuberculosis) that could interact with the OSB ketone of OSB-AMS was identified. See, e.g., Lu et al. ChemBioChem 2012, 13, 129-136. Although this alcohol side chain is absent in E. coli MenE (Gly-268), the docking studies herein suggest that the tertiary alcohol of the difluoroindanediol in (1R,3S)-2 may be positioned to interact with this side chain in S. aureus and M. tuberculosis MenE.

### Protein Preperation

The OSB-AMS*MenE co-crystal structure (PDB:5C5H) was processed using the Protein Preparation Wizard in the Schrödinger suit (v2015.3). Bond orders were assigned, hydrogen's added, and waters beyond 5 Å were deleted. The protonation and tautomeric states of the protein-ligand complex were generated using EPIK at pH 7.4. Hydrogen bond assignment and optimization was performed with PROPKA to sample hydrogen bonding and orientation of water molecules. Non-bridging waters (<2 hydrogen bonds) were removed. Geometric refinement was performed using OPLS_2005 force field restrained minimization to a heavy atom convergence of 0.3 Å.

### Ligand Preperation

Ligand preparation was performed using Ligprep in the Schrödinger suit (v2015.3). Lowest energy conformers were obtained using OPLS_2005 force field optimization. Ionization and tautomeric states were generated using EPIK at pH 7.4.

### Grid Generation

Using the Schrödinger suit (v2015.3) receptor grid generator, the receptor-binding site was defined as the area around the co-crystalized ligand with a cube grid of 10 Å side length. Nonpolar parts of the receptor were softened using Van der Waals radius scaling (factor 1.0 with partial cutoff of 0.25). No constraints were defined and rotations allowed for all hydroxyl groups in the defined binding pocket.

### Docking Using Soft Receptor

Using Glide (v5.3), ligands were docked to MenE using Glide XP docking precision. Flexible ligand sampling was used and EPIK state penalties applied to docking scores. Post-docking minimization was performed for all poses. See also FIG. 8.

TABLE E6
Docking scores and biochemical inhibition of E. coli MenE

| Inhibitor | Docking Score^{a} | E. coli MenE IC₅₀ |
|---|---|---|
| OSB-AMS (1) | −13.9 kcal/mol | 0.025 μM |
| (1R,3S)-2 | −11.9 kcal/mol | 5 μM |
| (1S,3R)-2 | −10.1 kcal/mol | >200 μM |
| (1R,3R)-2 | −10.0 kcal/mol | >200 μM |
| (1S,3S)-2 | −8.8 kcal/mol | >200 μM |

^{a}Docking scores expressed in kcal/mol but units are arbitrary.

### Biochemical Activity of Difluoroindanediols 2 (Compound 109)

The biochemical inhibitory activity of the four diastereomeric difluoroindanediols 2 against E. coli MenE were tested (Table E5). Consistent with the results of the docking studies above, the syn-difluoroindanediol (1R,3S)-2 was the most potent inhibitor (entry 2).

The (1R,3S)-2 diastereomer was also approximately 4-fold more potent than the mixture of all four diastereomers 2 (entry 1), suggesting that this single diastereomer is responsible for the observed inhibitory activity of the mixture.

The antimicrobial activity of the difluoroindanediols 2 against Bacillus subtilis, methicillin-resistant S. aureus (MRSA), and M. tuberculosis (Table 1) was also evaluated. Surprisingly, all four individual diastereomers exhibited MIC values similar to that of the mixture of diasteromers. When the cultures were complemented with exogenous menaquinone-4, a four-fold increase in MIC values was observed for the mixture of diastereomers (entry 1), while 2- to 4-fold increases were also observed for the MenE inhibitor (1R,3S)-2 (entry 2), consistent with a mechanism of action involving inhibition of menaquinone biosynthesis. Some rescue was also observed for the other syn-diastereomer (1S,3R)-2 in B. subtilis and M. tuberculosis (entry 3), while no rescue was observed for the anti diastereomers (entries 4,5). This suggests that the antimicrobial activity of the last three diastereomers results from other mechanisms of action.

TABLE E5
Biochemical, antimicrobial activity of
diastereomeric difluoroindanediols 2.

| | | | | | M. tuber- |
|---|---|---|---|---|---|
| | | MenE | B. subtilis | MRSA | culosis |
| En- | Inhib- | IC₅₀ | MIC | MIC | MIC |
| try | itor | (μM)^{a} | (μg/mL)^{b} | (μg/mL)^{b,c} | (μg/mL)^{b} |
| 1 | 2^{d} | 18.3 ± 3.7 | 15.6 (62.5) | 15.6 (62.5) | 15.6 (62.5) |
| 2 | (1R,3S)-2 | 5.0 ± 1.0 | 15.6 (31.2) | 15.6 (31.2) | 15.6 (62.5) |
| 3 | (1S,3R)-2 | >200 | 15.6 (31.2) | 31.2 (31.2) | 31.2 (62.5) |
| 4 | (1R,3R)-2 | >200 | 15.6 (15.6) | 15.6 (15.6) | 15.6 (31.2) |
| 5 | (1S,3S)-2 | >200 | 15.6 (15.6) | 15.6 (15.6) | 31.2 (31.2) |
| 6 | AMS^{e} | n.d.^{f} | 3.9 (3.9) | 1.9 (1.9) | 0.16 (0.32) |

^{a}E. coli MenE.
^{b}MIC values in parentheses determined with addition of exogenous menaquinone-4 (10 μg/mL).
^{c}MRSA = methicillin-resistant S. aureus.
^{d}Equimolar mixture of four diastereomers, prepared by the original synthetic route.
^{e}5′-O-sulfamoyladenosine.
^{f}n.d. = not determined.

### REFERENCES

1. Lu, X., Zhou, R., Sharma, I., Li, X., Kumar, G., Swaminathan, S., Tonge, P. J., and Tan, D. S. (2012) ChemBioChem 13, 129-136. Stable analogues of OSB-AMP: potent inhibitors of MenE, the o-succinylbenzoate-CoA synthetase from bacterial menaquinone biosynthesis.
2. Lu, X., Zhang, H., Tonge, P. J., and Tan, D. S. (2008) Bioorg. Med. Chem. Lett. 18, 5963-5966. Mechanism-based inhibitors of MenE, an acyl-CoA synthetase involved in bacterial menaquinone biosynthesis.
3. Cisar, J. S., Ferreras, J. A., Soni, R. K., Quadri, L. E., and Tan, D. S. (2007) J. Am. Chem. Soc. 129, 7752-7753. Exploiting ligand conformation in selective inhibition of non-ribosomal peptide synthetase amino acid adenylation with designed macrocyclic small molecules.
4. Corpet, F. (1988) Nucleic Acids Res. 16, 10881-10890. Multiple sequence alignment with hierarchical clustering.
5. Bligh, E. G., and Dyer, W. J. (1959) Can. J. Biochem. Physiol. 37, 911-917. A rapid method of total lipid extraction and purification.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any one of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A compound of Formula (I): 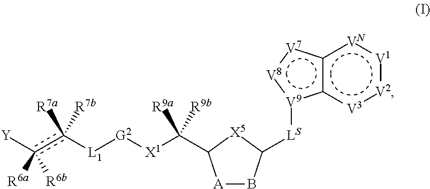
or a pharmaceutically acceptable salt, tautomer solvate, hydrate, polymorph, co-crystal, tautomer, stereoisomer, prodrug, or isotopically labeled derivative thereof, wherein:
G² is —S(═O)₂—, —P(═O)(R^{e})—, —P(═O)(OR^{e})—, —P(═O)(N(R^{e})₂)—, —P(═S)(R^{e})—, —P(═S)(OR^{e})—, —P(═S)(N(R^{e})₂)—, —Si(OR^{e})₂—, —C(═O)—, —C(═S)—, —C(═NR^{f})—, —(CH₂)ₕ—, 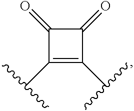 [SMILES: *c1c(*)c(=O)c1=O]
or optionally substituted monocyclic 5- or 6-membered heteroarylene, wherein 1, 2, 3, or 4 atoms in the heteroarylene ring system are independently oxygen, nitrogen, or sulfur;
A-B is —(R^{A})₂C—C(R^{B})₂— or —R^{A}C═CR^{B}—, wherein each occurrence of R^{A} is independently hydrogen, halogen, optionally substituted alkyl, optionally substituted acyl, —OR^{S1}, or —N(R^{e})₂, and each occurrence of R^{B} is independently hydrogen, halogen, optionally substituted alkyl, optionally substituted acyl, —OR^{S2}, or —N(R^{e})₂;
X⁵ is —O—, —S—, —C(R^{d})₂—, or —NR^{f}—;
Y is of formula: 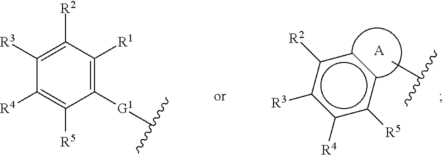
G¹ is —C(R^{G1})(R^{G2})—, —C(═O)—, —C(═S)—, —C(═NR^{f})—, —C(═C(R^{G1})(R^{G2}))—, or —C(OR^{G1})(OR^{G2})—;
each of R^{G1} and R^{G2} is independently hydrogen, halogen, optionally substituted alkyl, optionally substituted alkenyl, optionally substituted alkynyl, —OR^{e}, or —N(R^{e})₂, or R^{G1} and R^{G2} are joined to form an optionally substituted carbocyclic ring or optionally substituted heterocyclic ring;
Ring A is an optionally substituted carbocyclic, optionally substituted heterocyclic, optionally substituted aryl, or optionally substituted heteroaryl ring;
L¹ is a bond or of formula: 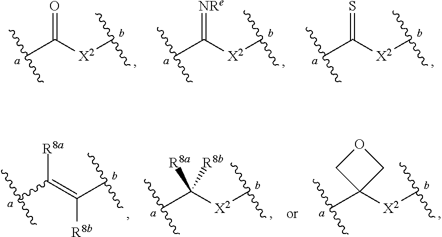
wherein L is oriented such that the position labeled a is attached a carbon atom and the position labeled b is attached to G²;
X¹ is a bond, —O—, —C(R^{d})₂—, —(CH₂)_{q}—, or —NR^{f}—;
X² is a bond, —O—, —C(R^{d})₂—, —(CH₂)ₜ—, or —NR^{f}—;
R¹ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted boronyl, —NO₂, —CN, —OR^{e}, —N(R^{e})₂, —C(═NR^{e})R^{e}, —C(═NR^{e})OR^{e}, —C(═NR^{e})N(R^{e})₂, —C(═O)R^{e}, —C(═O)OR^{e}, —C(═O)N(R^{e})₂, —NR^{e}C(═O)R^{e}, —NR^{e}C(═O)OR^{e}, —NR^{e}C(═O)N(R^{e})₂, —OC(═O)R^{e}, —OC(═O)OR^{e}, or —OC(═O)N(R^{e})₂;
each of R², R³, and R⁴ are independently hydrogen, halogen, optionally substituted C₁₋₆ alkyl, optionally substituted acyl, —NO₂, —CN, —OR^{e}, or —N(R^{e})₂;
R⁵ is hydrogen, halogen, optionally substituted C₁₋₆ alkyl, —NO₂, —CN, —OR^{e}, or —N(R^{e})₂;
each of R^{6a} and R^{6b} is independently hydrogen, halogen, or optionally substituted C₁₋₆ alkyl;
each of R^{7a} and R^{7b} is independently hydrogen, halogen, or optionally substituted C₁₋₆ alkyl;
each of R^{8a} and R^{8b} is independently hydrogen, halogen, or optionally substituted C₁₋₆ alkyl;
each of R^{9a} and R^{9b} is independently hydrogen, halogen, optionally substituted C₁₋₆ alkyl, —OR^{e}, or —N(R^{e})₂;
each of R^{S1} and R^{S2} is independently hydrogen, optionally substituted C₁₋₆ alkyl, optionally substituted acyl, or an oxygen protecting group, or R^{S1} and R^{S2} are joined to form an optionally substituted heterocyclic ring;
L^{S} is a bond, —O—, —NR^{f}—, optionally substituted alkylene, optionally substituted alkenylene, optionally substituted alkynylene, optionally substituted acylene, or optionally substituted arylene;
each of V¹, V², V³, V⁷, V⁸, and V⁹ is independently N, NR^{V}, or CR^{V};
each occurrence of R^{V} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —NO₂, —CN, —OR^{e}, or —N(R^{e})₂;
V^{N} is N, NR^{N}, or CR^{N};
R^{N} is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —NO₂, —CN, —OR^{e}, or —N(R^{Na})₂;
each occurrence of R^{Na} independently hydrogen, optionally substituted C₁₋₆ alkyl, optionally substituted acyl, or a nitrogen protecting group, or both R^{Na} are joined to form and optionally substituted heterocyclic or optionally substituted heteroaryl ring;
each occurrence of R^{d} is independently hydrogen, halogen, optionally substituted C₁₋₆ alkyl, —OR^{e}, or —N(R^{e})₂;
each occurrence of R^{e} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, an oxygen protecting group when attached to an oxygen atom, a nitrogen protecting group when attached to a nitrogen atom, or two R^{e} are joined to form and optionally substituted heterocyclic or optionally substituted heteroaryl ring;
each R^{f} is independently hydrogen, optionally substituted C₁₋₆ alkyl, optionally substituted acyl, or a nitrogen protecting group;
each of h, q, and t is independently 1, 2, or 3; [symbol]
is a single, double, or triple bond, wherein R^{6b} and R^{7b} are absent when [symbol] is a double bond, and R^{6a}, R^{6b}, R^{7a}, and R^{7b} are absent when [symbol] is a triple bond; and [symbol]
indicates that each bond of the ring is a single or double bond.
provided the compound is not of formula:

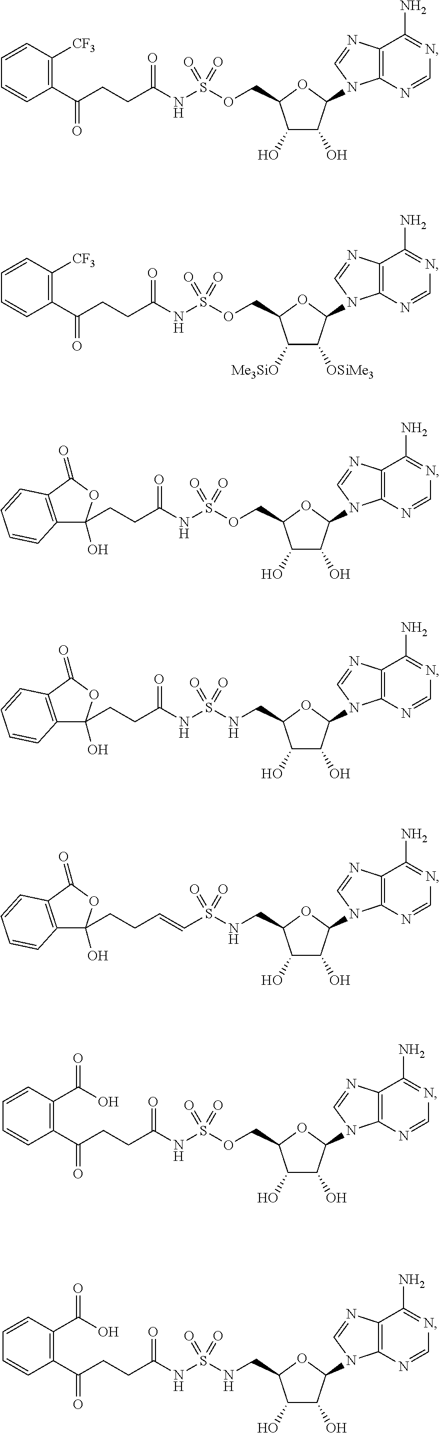

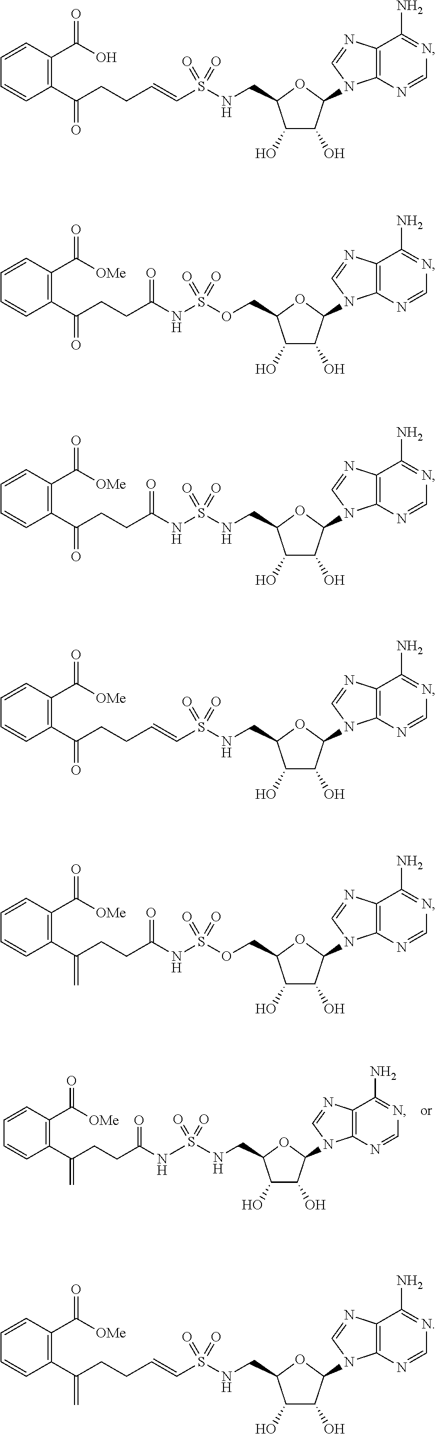

2. The compound of claim 1, wherein the compound is of Formula (II): 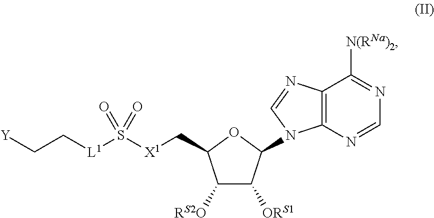
or a pharmaceutically acceptable salt, stereoisomer, or tautomer thereof.

3. The compound of claim 1, wherein the compound is of the formula: 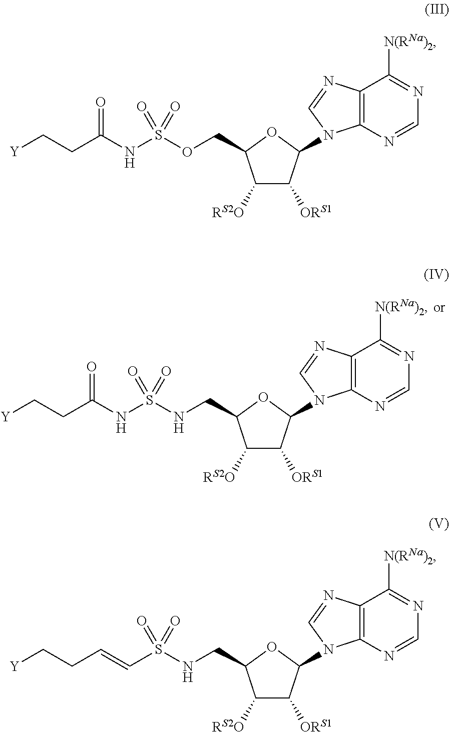
or a pharmaceutically acceptable salt, stereoisomer, or tautomer thereof.

4-5. (canceled)

6. The compound of claim 1, wherein the compound is of Formula (VI): 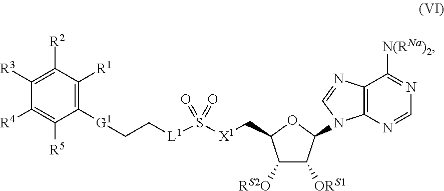
or a pharmaceutically acceptable salt, stereoisomer, or tautomer thereof.

7. (canceled)

8. The compound of claim 1, wherein the compound is of Formula (VII): 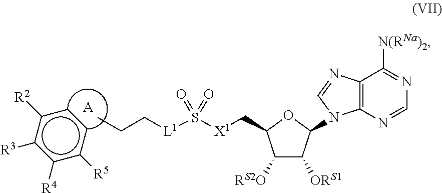
or a pharmaceutically acceptable salt, stereoisomer, or tautomer thereof.

9-10. (canceled)

11. The compound of claim 1, wherein Y is:

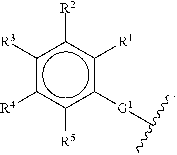

12-27. (canceled)

28. The compound of claim 1, wherein Y is:

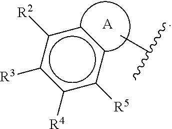

29-43. (canceled)

44. The compound of claim 1, wherein L¹ is:

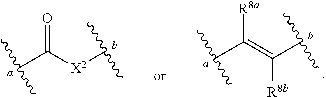

45-48. (canceled)

49. The compound of claim 1, wherein X¹ is —O— or —NH—.

50-51. (canceled)

52. The compound of claim 1, wherein the compound is of formula: 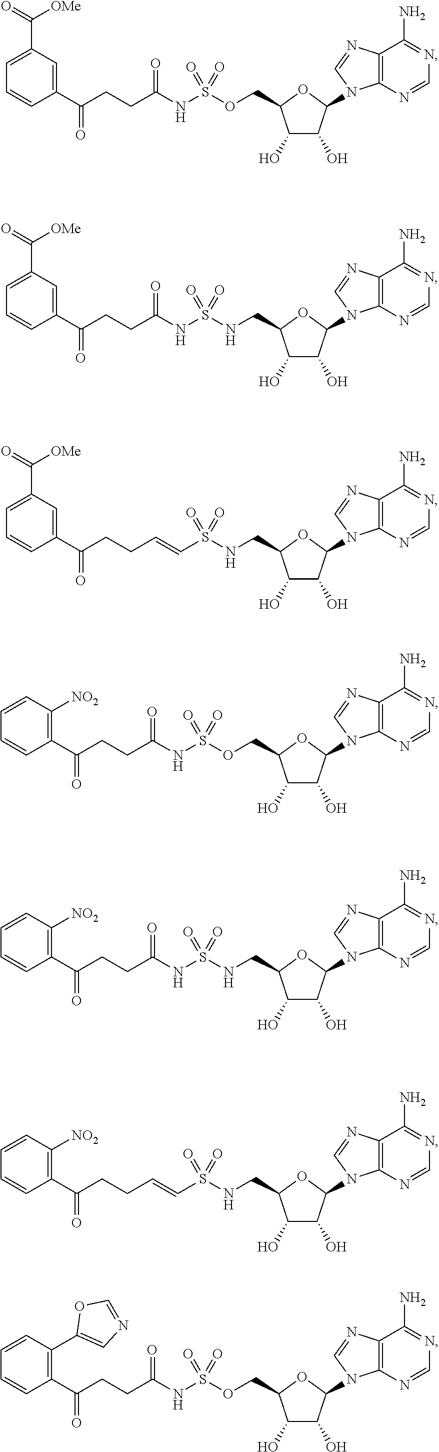 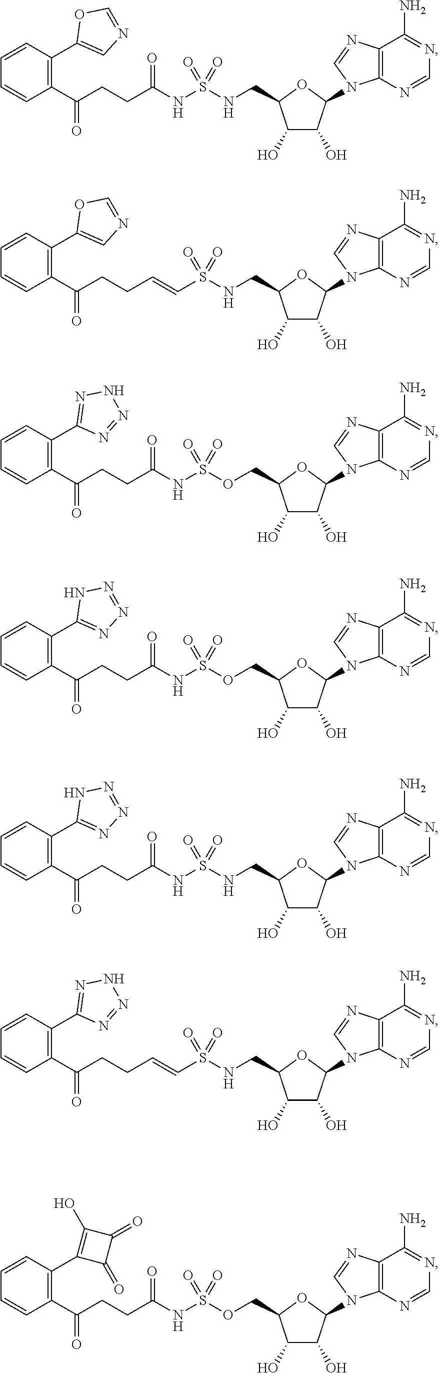 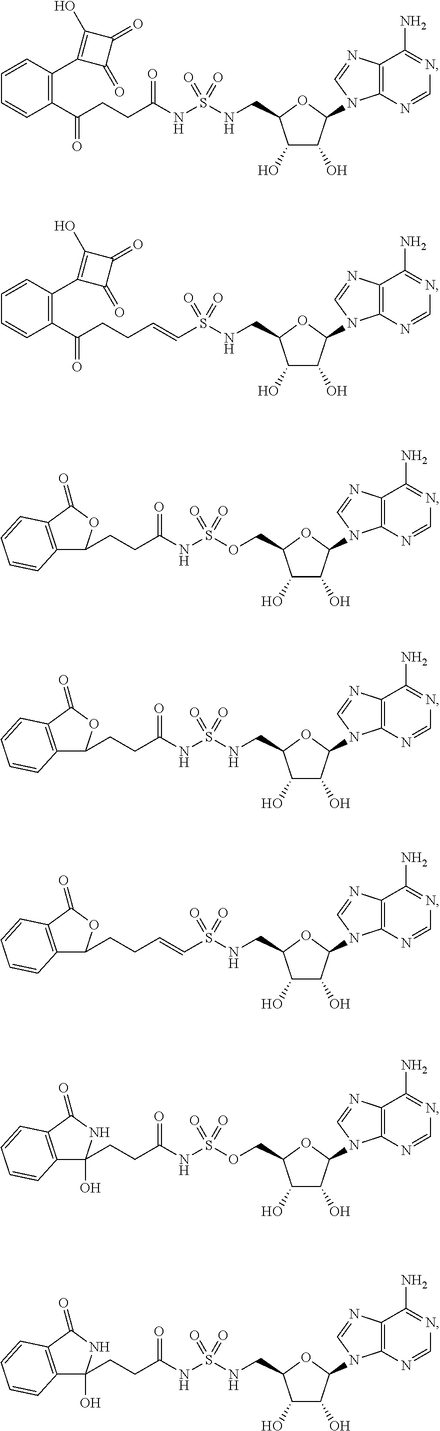 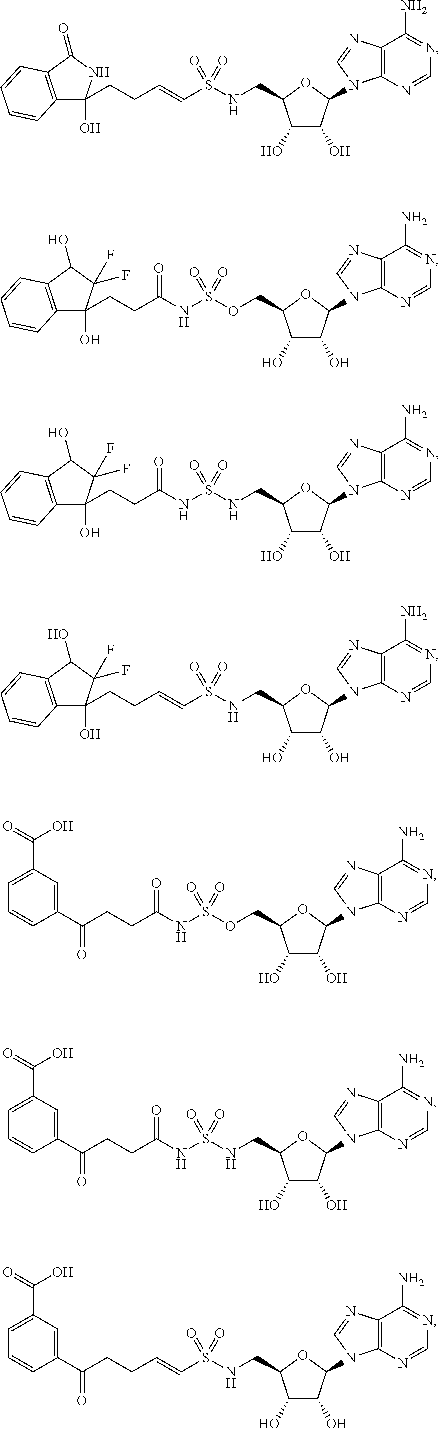 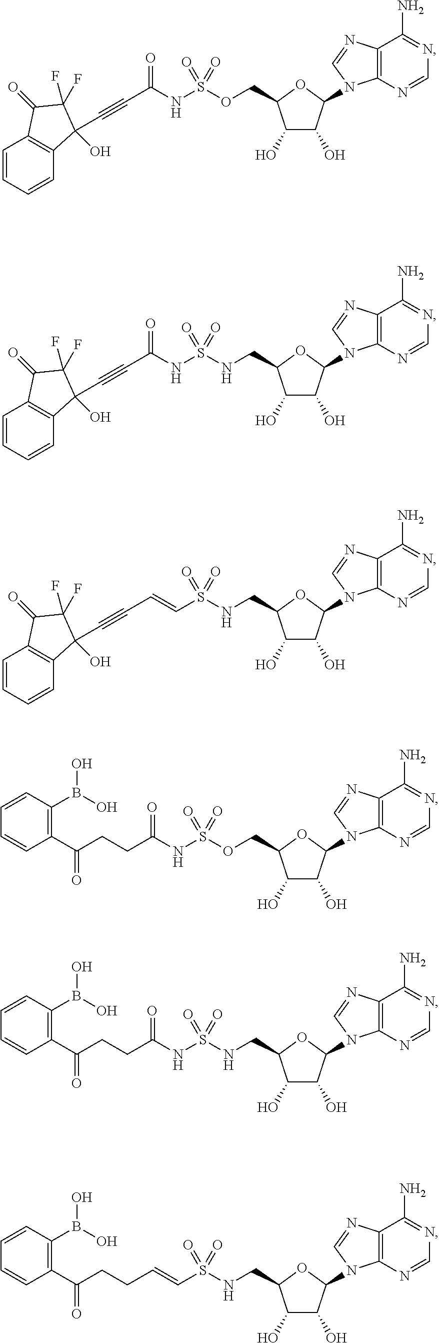 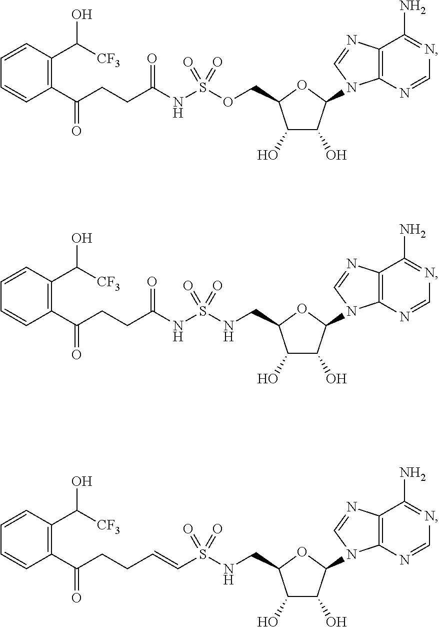 [SMILES: Nc1ncnc2c1ncn2[C@@H]1O[C@H](COS(=O)(=O)NC(=O)CCC(=O)c2ccccc2C(O)C(F)(F)F)[C@@H](O)[C@H]1O ; Nc1ncnc2c1ncn2[C@@H]1O[C@H](CNS(=O)(=O)NC(=O)CCC(=O)c2ccccc2C(O)C(F)(F)F)[C@@H](O)[C@H]1O ; Nc1ncnc2c1ncn2[C@@H]1O[C@H](CNS(=O)(=O)/C=C/CCC(=O)c2ccccc2C(O)C(F)(F)F)[C@@H](O)[C@H]1O]
or a pharmaceutically acceptable salt, stereoisomer, or tautomer thereof.

53. The compound of claim 1, wherein the compound is of formula: 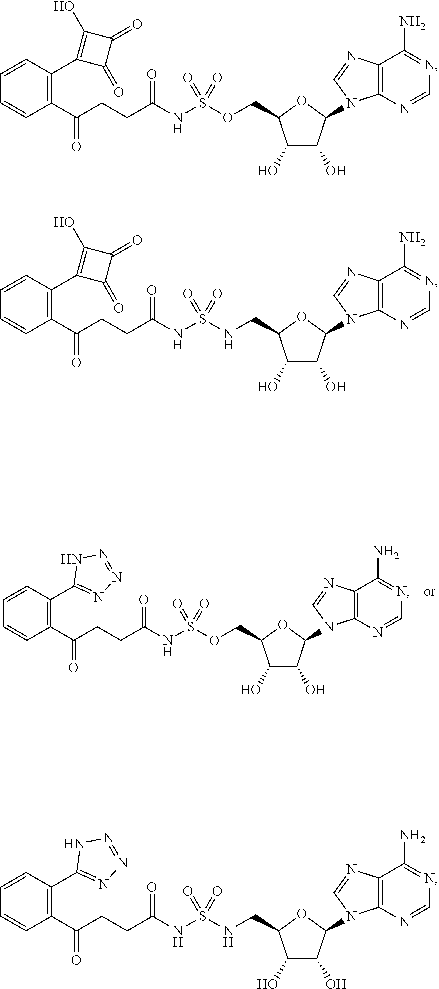 [SMILES: Nc1ncnc2c1ncn2[C@@H]1O[C@H](COS(=O)(=O)NC(=O)CCC(=O)c2ccccc2-c2c(O)c(=O)c2=O)[C@@H](O)[C@H]1O ; Nc1ncnc2c1ncn2[C@@H]1O[C@H](CNS(=O)(=O)NC(=O)CCC(=O)c2ccccc2-c2c(O)c(=O)c2=O)[C@@H](O)[C@H]1O ; Nc1ncnc2c1ncn2[C@@H]1O[C@H](COS(=O)(=O)NC(=O)CCC(=O)c2ccccc2-c2nnn[nH]2)[C@@H](O)[C@H]1O ; Nc1ncnc2c1ncn2[C@@H]1O[C@H](CNS(=O)(=O)NC(=O)CCC(=O)c2ccccc2-c2nnn[nH]2)[C@@H](O)[C@H]1O]
or a pharmaceutically acceptable salt, stereoisomer, or tautomer thereof.

54. (canceled)

55. The compound of claim 1, wherein the compound is of formula: 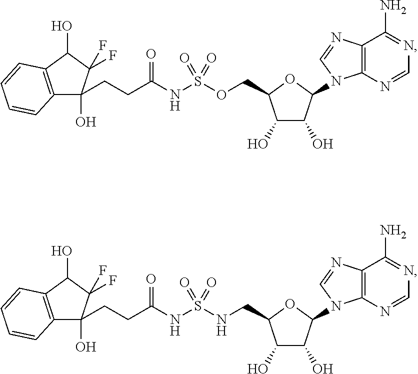 [SMILES: Nc1ncnc2c1ncn2[C@@H]1O[C@H](COS(=O)(=O)NC(=O)CCC2(O)c3ccccc3C(O)C2(F)F)[C@@H](O)[C@H]1O ; Nc1ncnc2c1ncn2[C@@H]1O[C@H](CNS(=O)(=O)NC(=O)CCC2(O)c3ccccc3C(O)C2(F)F)[C@@H](O)[C@H]1O]
or a pharmaceutically acceptable salt, stereoisomer, or tautomer thereof.

56. (canceled)

57. The compound of claim 1, wherein the compound is selected from the group consisting of: 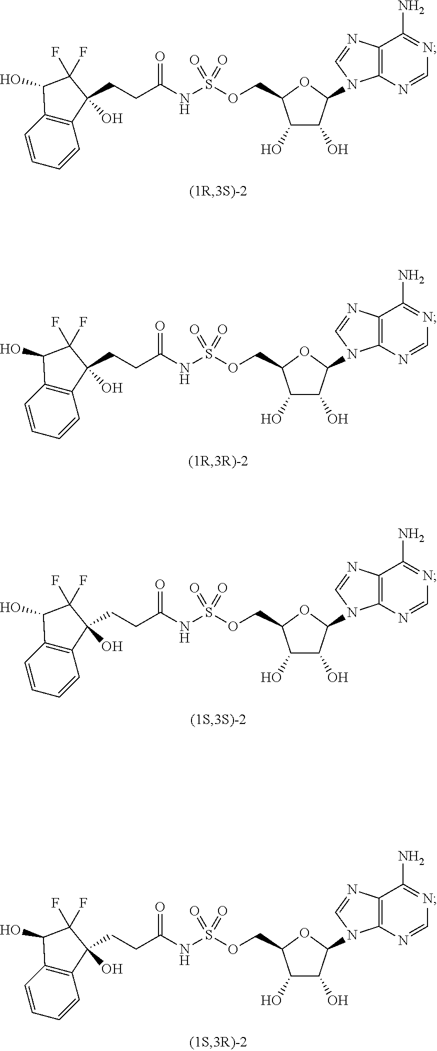 [SMILES: Nc1ncnc2c1ncn2[C@@H]1O[C@H](COS(=O)(=O)NC(=O)CC[C@@]2(O)c3ccccc3[C@H](O)C2(F)F)[C@@H](O)[C@H]1O ; Nc1ncnc2c1ncn2[C@@H]1O[C@H](COS(=O)(=O)NC(=O)CC[C@@]2(O)c3ccccc3[C@@H](O)C2(F)F)[C@@H](O)[C@H]1O ; Nc1ncnc2c1ncn2[C@@H]1O[C@H](COS(=O)(=O)NC(=O)CC[C@]2(O)c3ccccc3[C@H](O)C2(F)F)[C@@H](O)[C@H]1O ; Nc1ncnc2c1ncn2[C@@H]1O[C@H](COS(=O)(=O)NC(=O)CC[C@]2(O)c3ccccc3[C@@H](O)C2(F)F)[C@@H](O)[C@H]1O]
and tautomers thereof; and pharmaceutically acceptable salts thereof.

58. (canceled)

59. A pharmaceutical composition comprising a compound of claim 1, or a pharmaceutically acceptable salt, solvate, hydrate, polymorph, co-crystal, tautomer, stereoisomer, prodrug, or isotopically labeled derivative thereof, and a pharmaceutically acceptable excipient.

60. A method of treating an infectious disease comprising administering an effective amount of a compound of claim 1, or a pharmaceutically acceptable salt, solvate, hydrate, polymorph, co-crystal, tautomer, stereoisomer, prodrug, or isotopically labeled derivative thereof, to a subject in need thereof.

61-76. (canceled)

77. A method of inhibiting menaquinone biosynthesis in an infectious microorganism, the method comprising contacting the infectious microorganism with a compound of claim 1, or a pharmaceutically acceptable salt, solvate, hydrate, polymorph, co-crystal, tautomer, stereoisomer, prodrug, or isotopically labeled derivative thereof.

78. A method of inhibiting menaquinone biosynthesis in an infection in a subject, the method comprising administering to the subject a compound of claim 1, or a pharmaceutically acceptable salt, solvate, hydrate, polymorph, co-crystal, tautomer, stereoisomer, prodrug, or isotopically labeled derivative thereof.

79. A method of inhibiting o-succinylbenzoate-CoA synthetase (MenE) in an infectious microorganism, the method comprising contacting the infectious microorganism with a compound of claim 1, or a pharmaceutically acceptable salt, solvate, hydrate, polymorph, co-crystal, tautomer, stereoisomer, prodrug, or isotopically labeled derivative thereof.

80. A method of inhibiting o-succinylbenzoate-CoA synthetase (MenE) in an infection in a subject, the method comprising administering to the subject a compound of claim 1, or a pharmaceutically acceptable salt, solvate, hydrate, polymorph, co-crystal, tautomer, stereoisomer, prodrug, or isotopically labeled derivative thereof.

81. A kit for treating an infectious disease comprising a container, a compound of claim 1, or a pharmaceutically acceptable salt, solvate, hydrate, polymorph, co-crystal, tautomer, stereoisomer, prodrug, or isotopically labeled derivative thereof, and instructions for administering to a subject in need thereof.
